SEQUENCE LISTING (559 sequences)

SEQ ID NO: 1 - protein, 546 aa, synthetic construct
  REGION 1..546: note = Uniprot Q9IH63 with signal sequence
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 2 - protein, 492 aa, synthetic construct
  REGION 1..492: note = NiV-F backbone deltaCT
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIV

SEQ ID NO: 3 - protein, 26 aa, synthetic construct
  REGION 1..26: note = Signal Peptide
MVVILDKRCYCNLLILILMISECSVG

SEQ ID NO: 4 - protein, 28 aa, synthetic construct
  REGION 1..28: note = NivF_CT-28 (full-length)
EKKRNTYSRLEDRRVRPTSSGDLYYIGT

SEQ ID NO: 5 - protein, 27 aa, synthetic construct
  REGION 1..27: note = NivF_CT-27
EKKRNTYSRLEDRRVRPTSSGDLYYIG

SEQ ID NO: 6 - protein, 26 aa, synthetic construct
  REGION 1..26: note = NivF_CT-26
EKKRNTYSRLEDRRVRPTSSGDLYYI

SEQ ID NO: 7 - protein, 25 aa, synthetic construct
  REGION 1..25: note = NivF_CT-25
EKKRNTYSRLEDRRVRPTSSGDLYY

SEQ ID NO: 8 - protein, 24 aa, synthetic construct
  REGION 1..24: note = NivF_CT-24
EKKRNTYSRLEDRRVRPTSSGDLY

SEQ ID NO: 9 - protein, 23 aa, synthetic construct
  REGION 1..23: note = NivF_CT-23
EKKRNTYSRLEDRRVRPTSSGDL

SEQ ID NO: 10 - protein, 22 aa, synthetic construct
  REGION 1..22: note = NivF_CT-22
EKKRNTYSRLEDRRVRPTSSGD

SEQ ID NO: 11 - protein, 21 aa, synthetic construct
  REGION 1..21: note = NivF_CT-21
EKKRNTYSRLEDRRVRPTSSG

SEQ ID NO: 12 - protein, 20 aa, synthetic construct
  REGION 1..20: note = NivF_CT-20
EKKRNTYSRLEDRRVRPTSS

SEQ ID NO: 13 - protein, 19 aa, synthetic construct
  REGION 1..19: note = NivF_CT-19
EKKRNTYSRLEDRRVRPTS

SEQ ID NO: 14 - protein, 18 aa, synthetic construct
  REGION 1..18: note = NivF_CT-18
EKKRNTYSRLEDRRVRPT

SEQ ID NO: 15 - protein, 17 aa, synthetic construct
  REGION 1..17: note = NivF_CT-17
EKKRNTYSRLEDRRVRP

SEQ ID NO: 16 - protein, 16 aa, synthetic construct
  REGION 1..16: note = NivF_CT-16
EKKRNTYSRLEDRRVR

SEQ ID NO: 17 - protein, 15 aa, synthetic construct
  REGION 1..15: note = NivF_CT-15
EKKRNTYSRLEDRRV

SEQ ID NO: 18 - protein, 14 aa, synthetic construct
  REGION 1..14: note = NivF_CT-14
EKKRNTYSRLEDRR

SEQ ID NO: 19 - protein, 13 aa, synthetic construct
  REGION 1..13: note = NivF_CT-13
EKKRNTYSRLEDR

SEQ ID NO: 20 - protein, 12 aa, synthetic construct
  REGION 1..12: note = NivF_CT-12
EKKRNTYSRLED

SEQ ID NO: 21 - protein, 11 aa, synthetic construct
  REGION 1..11: note = NivF_CT-11
EKKRNTYSRLE

SEQ ID NO: 22 - protein, 10 aa, synthetic construct
  REGION 1..10: note = NivF_CT-10
EKKRNTYSRL

SEQ ID NO: 23 - protein, 9 aa, synthetic construct
  REGION 1..9: note = NivF_CT-9
EKKRNTYSR

SEQ ID NO: 24 - protein, 8 aa, synthetic construct
  REGION 1..8: note = NivF_CT-8
EKKRNTYS

SEQ ID NO: 25 - protein, 7 aa, synthetic construct
  REGION 1..7: note = NivF_CT-7
EKKRNTY

SEQ ID NO: 26 - protein, 6 aa, synthetic construct
  REGION 1..6: note = NivF_CT-06
EKKRNT

SEQ ID NO: 27 - protein, 5 aa, synthetic construct
  REGION 1..5: note = NivF_CT-5
EKKRN

SEQ ID NO: 28 - protein, 4 aa, synthetic construct
  REGION 1..4: note = NivF_CT-4
EKKR

SEQ ID NO: 29-31 - skipped (3 entries)

SEQ ID NO: 32 - protein, 23 aa, synthetic construct
  REGION 1..23: note = YSRL
EYSRLEDRRVRPTSSGDLYYIGT

SEQ ID NO: 33 - protein, 24 aa, synthetic construct
  REGION 1..24: note = YSRL
EKKRNTEDRRVRPTSSGDLYYIGT

SEQ ID NO: 34 - protein, 25 aa, synthetic construct
  REGION 1..25: note = YSRL
EKKRNTYSRLRVRPTSSGDLYYIGT

SEQ ID NO: 35 - protein, 6 aa, synthetic construct
  REGION 1..6: note = YSRL
EKYSRL

SEQ ID NO: 36 - protein, 7 aa, synthetic construct
  REGION 1..7: note = YSRL
EKKYSRL

SEQ ID NO: 37 - protein, 12 aa, synthetic construct
  REGION 1..12: note = NIvF_CT-6_AU1
EKKRNTDTYRYI

SEQ ID NO: 38 - protein, 15 aa, synthetic construct
  REGION 1..15: note = NIvF_CT-6_HA
EKKRNTYPYDVPDYA

SEQ ID NO: 39 - protein, 8 aa, synthetic construct
  REGION 1..8: note = DKQTLL
EKDKQTLL

SEQ ID NO: 40 - protein, 7 aa, synthetic construct
  REGION 1..7: note = DKQTLL
EDKQTLL

SEQ ID NO: 41 - protein, 12 aa, synthetic construct
  REGION 1..12: note = DKQTLL
EKKRNTDKQTLL

SEQ ID NO: 42 - protein, 30 aa, synthetic construct
  REGION 1..30: note = DKQTLL
EKKRNTDKQTLLEDRRVRPTSSGDLYYIGT

SEQ ID NO: 43 - protein, 6 aa, synthetic construct
  REGION 1..6: note = NPVY
EKNPVY

SEQ ID NO: 44 - protein, 8 aa, synthetic construct
  REGION 1..8: note = FDNPVY
EKFDNPVY

SEQ ID NO: 45 - protein, 10 aa, synthetic construct
  REGION 1..10: note = NPVY
EKKRNTNPVY

SEQ ID NO: 46 - protein, 12 aa, synthetic construct
  REGION 1..12: note = FDNPVY
EKKRNTFDNPVY

SEQ ID NO: 47 - protein, 28 aa, synthetic construct
  REGION 1..28: note = NPVY
EKKRNTNPVYEDRRVRPTSSGDLYYIGT

SEQ ID NO: 48 - protein, 30 aa, synthetic construct
  REGION 1..30: note = FDNPVY
EKKRNTFDNPVYEDRRVRPTSSGDLYYIGT

SEQ ID NO: 49 - protein, 8 aa, synthetic construct
  REGION 1..8: note = YFIPIN
EKYFIPIN

SEQ ID NO: 50 - protein, 12 aa, synthetic construct
  REGION 1..12: note = YFIPIN
EKKRNTYFIPIN

SEQ ID NO: 51 - protein, 30 aa, synthetic construct
  REGION 1..30: note = YFIPIN
EKKRNTYFIPINEDRRVRPTSSGDLYYIGT

SEQ ID NO: 52 - protein, 28 aa, synthetic construct
  REGION 1..28: note = HeVF_CT_28_FL
EKKRGNYSRLDDRQVRPVSNGDLYYIGT

SEQ ID NO: 53 - protein, 6 aa, synthetic construct
  REGION 1..6: note = CT-06_K1A
EAKRNT

SEQ ID NO: 54 - protein, 6 aa, synthetic construct
  REGION 1..6: note = CT-02_YSRL_K1A
EAYSRL

SEQ ID NO: 55 - protein, 28 aa, synthetic construct
  REGION 1..28: note = CT-28_K1A
EAKRNTYSRLEDRRVRPTSSGDLYYIGT

SEQ ID NO: 56 - protein, 10 aa, synthetic construct
  REGION 1..10: note = CT-10_K1A
EAKRNTYSRL

SEQ ID NO: 57 - protein, 528 aa, synthetic construct
  REGION 1..528: note = F3F5_Q393L_CT-10_K1A
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEAKRNTYSRL

SEQ ID NO: 58 - protein, 524 aa, synthetic construct
  REGION 1..524: note = F3F5_Q393L_CT-06_K1A
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEAKRNT

SEQ ID NO: 59 - protein, 27 aa, synthetic construct
  REGION 1..27: note = HeVF_CT_27
EKKRGNYSRLDDRQVRPVSNGDLYYIG

SEQ ID NO: 60 - protein, 26 aa, synthetic construct
  REGION 1..26: note = HeVF_CT_26
EKKRGNYSRLDDRQVRPVSNGDLYYI

SEQ ID NO: 61 - protein, 25 aa, synthetic construct
  REGION 1..25: note = HeVF_CT_25
EKKRGNYSRLDDRQVRPVSNGDLYY

SEQ ID NO: 62 - protein, 24 aa, synthetic construct
  REGION 1..24: note = HeVF_CT_24
EKKRGNYSRLDDRQVRPVSNGDLY

SEQ ID NO: 63 - protein, 23 aa, synthetic construct
  REGION 1..23: note = HeVF_CT_23
EKKRGNYSRLDDRQVRPVSNGDL

SEQ ID NO: 64 - protein, 22 aa, synthetic construct
  REGION 1..22: note = HeVF_CT_22
EKKRGNYSRLDDRQVRPVSNGD

SEQ ID NO: 65 - protein, 21 aa, synthetic construct
  REGION 1..21: note = HeVF_CT_21
EKKRGNYSRLDDRQVRPVSNG

SEQ ID NO: 66 - protein, 20 aa, synthetic construct
  REGION 1..20: note = HeVF_CT_20
EKKRGNYSRLDDRQVRPVSN

SEQ ID NO: 67 - protein, 19 aa, synthetic construct
  REGION 1..19: note = HeVF_CT_19
EKKRGNYSRLDDRQVRPVS

SEQ ID NO: 68 - protein, 18 aa, synthetic construct
  REGION 1..18: note = HeVF_CT_18
EKKRGNYSRLDDRQVRPV

SEQ ID NO: 69 - protein, 17 aa, synthetic construct
  REGION 1..17: note = HeVF_CT_17
EKKRGNYSRLDDRQVRP

SEQ ID NO: 70 - protein, 16 aa, synthetic construct
  REGION 1..16: note = HeVF_CT_16
EKKRGNYSRLDDRQVR

SEQ ID NO: 71 - protein, 15 aa, synthetic construct
  REGION 1..15: note = HeVF_CT_15
EKKRGNYSRLDDRQV

SEQ ID NO: 72 - protein, 14 aa, synthetic construct
  REGION 1..14: note = HeVF_CT_14
EKKRGNYSRLDDRQ

SEQ ID NO: 73 - protein, 13 aa, synthetic construct
  REGION 1..13: note = HeVF_CT_13
EKKRGNYSRLDDR

SEQ ID NO: 74 - protein, 12 aa, synthetic construct
  REGION 1..12: note = HeVF_CT_12
EKKRGNYSRLDD

SEQ ID NO: 75 - protein, 11 aa, synthetic construct
  REGION 1..11: note = HeVF_CT_11
EKKRGNYSRLD

SEQ ID NO: 76 - protein, 10 aa, synthetic construct
  REGION 1..10: note = HeVF_CT_10
EKKRGNYSRL

SEQ ID NO: 77 - protein, 9 aa, synthetic construct
  REGION 1..9: note = HeVF_CT_9
EKKRGNYSR

SEQ ID NO: 78 - protein, 8 aa, synthetic construct
  REGION 1..8: note = HeVF_CT_8
EKKRGNYS

SEQ ID NO: 79 - protein, 7 aa, synthetic construct
  REGION 1..7: note = HeVF_CT_7
EKKRGNY

SEQ ID NO: 80 - protein, 6 aa, synthetic construct
  REGION 1..6: note = HeVF_CT_6
EKKRGN

SEQ ID NO: 81 - protein, 5 aa, synthetic construct
  REGION 1..5: note = HeVF_CT_5
EKKRG

SEQ ID NO: 82 - protein, 47 aa, synthetic construct
  REGION 1..47: note = BatPVF_CT_47_FL
RRYNKYTPLINSDPSSRRSTIQDVYIIPNPGEHSIRSAARSIDRDRD

SEQ ID NO: 83 - protein, 14 aa, synthetic construct
  REGION 1..14: note = BatPVF_CT_14
RRYNKYTPLINSDP

SEQ ID NO: 84 - protein, 13 aa, synthetic construct
  REGION 1..13: note = BatPVF_CT_13
RRYNKYTPLINSD

SEQ ID NO: 85 - protein, 12 aa, synthetic construct
  REGION 1..12: note = BatPVF_CT_12
RRYNKYTPLINS

SEQ ID NO: 86 - protein, 11 aa, synthetic construct
  REGION 1..11: note = BatPVF_CT_11
RRYNKYTPLIN

SEQ ID NO: 87 - protein, 10 aa, synthetic construct
  REGION 1..10: note = BatPVF_CT_10
RRYNKYTPLI

SEQ ID NO: 88 - protein, 9 aa, synthetic construct
  REGION 1..9: note = BatPVF_CT_9
RRYNKYTPL

SEQ ID NO: 89 - protein, 8 aa, synthetic construct
  REGION 1..8: note = BatPVF_CT_8
RRYNKYTP

SEQ ID NO: 90 - protein, 7 aa, synthetic construct
  REGION 1..7: note = BatPVF_CT_7
RRYNKYT

SEQ ID NO: 91 - protein, 6 aa, synthetic construct
  REGION 1..6: note = BatPVF_CT_6
RRYNKY

SEQ ID NO: 92 - protein, 5 aa, synthetic construct
  REGION 1..5: note = BatPVF_CT_5
RRYNK

SEQ ID NO: 93 - protein, 4 aa, synthetic construct
  REGION 1..4: note = BatPVF_CT_4
RRYN

SEQ ID NO: 94-95 - skipped (2 entries)

SEQ ID NO: 96 - protein, 29 aa, synthetic construct
  REGION 1..29: note = CeVF_CT_29_FL
DDPDYYNDYKRERINGKASKSNNIYYVGD

SEQ ID NO: 97 - protein, 14 aa, synthetic construct
  REGION 1..14: note = CeVF_CT_14
DDPDYYNDYKRERI

SEQ ID NO: 98 - protein, 13 aa, synthetic construct
  REGION 1..13: note = CeVF_CT_13
DDPDYYNDYKRER

SEQ ID NO: 99 - protein, 12 aa, synthetic construct
  REGION 1..12: note = CeVF_CT_12
DDPDYYNDYKRE

SEQ ID NO: 100 - protein, 11 aa, synthetic construct
  REGION 1..11: note = CeVF_CT_11
DDPDYYNDYKR

SEQ ID NO: 101 - protein, 10 aa, synthetic construct
  REGION 1..10: note = CeVF_CT_10
DDPDYYNDYK

SEQ ID NO: 102 - protein, 9 aa, synthetic construct
  REGION 1..9: note = CeVF_CT_9
DDPDYYNDY

SEQ ID NO: 103 - protein, 8 aa, synthetic construct
  REGION 1..8: note = CeVF_CT_8
DDPDYYND

SEQ ID NO: 104 - protein, 7 aa, synthetic construct
  REGION 1..7: note = CeVF_CT_7
DDPDYYN

SEQ ID NO: 105 - protein, 6 aa, synthetic construct
  REGION 1..6: note = CeVF_CT_6
DDPDYY

SEQ ID NO: 106 - protein, 5 aa, synthetic construct
  REGION 1..5: note = CeVF_CT_5
DDPDY

SEQ ID NO: 107 - protein, 4 aa, synthetic construct
  REGION 1..4: note = CeVF_CT_4
DDPD

SEQ ID NO: 108-110 - skipped (3 entries)

SEQ ID NO: 111 - protein, 38 aa, synthetic construct
  REGION 1..38: note = HPIV2F_CT_38_FL
KLVSQIHQFRSLAATTMFHRENPAFFSKNNHGNIYGIS

SEQ ID NO: 112 - protein, 14 aa, synthetic construct
  REGION 1..14: note = HPIV2F_CT_14
KLVSQIHQFRSLAA

SEQ ID NO: 113 - protein, 13 aa, synthetic construct
  REGION 1..13: note = HPIV2F_CT_13
KLVSQIHQFRSLA

SEQ ID NO: 114 - protein, 12 aa, synthetic construct
  REGION 1..12: note = HPIV2F_CT_12
KLVSQIHQFRSL

SEQ ID NO: 115 - protein, 11 aa, synthetic construct
  REGION 1..11: note = HPIV2F_CT_11
KLVSQIHQFRS

SEQ ID NO: 116 - protein, 10 aa, synthetic construct
  REGION 1..10: note = HPIV2F_CT_10
KLVSQIHQFR

SEQ ID NO: 117 - protein, 9 aa, synthetic construct
  REGION 1..9: note = HPIV2F_CT_9
KLVSQIHQF

SEQ ID NO: 118 - protein, 8 aa, synthetic construct
  REGION 1..8: note = HPIV2F_CT_8
KLVSQIHQ

SEQ ID NO: 119 - protein, 7 aa, synthetic construct
  REGION 1..7: note = HPIV2F_CT_7
KLVSQIH

SEQ ID NO: 120 - protein, 6 aa, synthetic construct
  REGION 1..6: note = HPIV2F_CT_6
KLVSQI

SEQ ID NO: 121 - protein, 5 aa, synthetic construct
  REGION 1..5: note = HPIV2F_CT_5
KLVSQ

SEQ ID NO: 122 - protein, 4 aa, synthetic construct
  REGION 1..4: note = HPIV2F_CT_4
KLVS

SEQ ID NO: 123-124 - skipped (2 entries)

SEQ ID NO: 125 - protein, 33 aa, synthetic construct
  REGION 1..33: note = MeaslesF_CT_33_FL
RGRCNKKGEQVGMSRPGLKPDLTGTSKSYVRSL

SEQ ID NO: 126 - protein, 14 aa, synthetic construct
  REGION 1..14: note = MeaslesF_CT_14
RGRCNKKGEQVGMS

SEQ ID NO: 127 - protein, 13 aa, synthetic construct
  REGION 1..13: note = MeaslesF_CT_13
RGRCNKKGEQVGM

SEQ ID NO: 128 - protein, 12 aa, synthetic construct
  REGION 1..12: note = MeaslesF_CT_12
RGRCNKKGEQVG

SEQ ID NO: 129 - protein, 11 aa, synthetic construct
  REGION 1..11: note = MeaslesF_CT_11
RGRCNKKGEQV

SEQ ID NO: 130 - protein, 10 aa, synthetic construct
  REGION 1..10: note = MeaslesF_CT_10
RGRCNKKGEQ

SEQ ID NO: 131 - protein, 9 aa, synthetic construct
  REGION 1..9: note = MeaslesF_CT_9
RGRCNKKGE

SEQ ID NO: 132 - protein, 8 aa, synthetic construct
  REGION 1..8: note = MeaslesF_CT_8
RGRCNKKG

SEQ ID NO: 133 - protein, 7 aa, synthetic construct
  REGION 1..7: note = MeaslesF_CT_7
RGRCNKK

SEQ ID NO: 134 - protein, 6 aa, synthetic construct
  REGION 1..6: note = MeaslesF_CT_6
RGRCNK

SEQ ID NO: 135 - protein, 5 aa, synthetic construct
  REGION 1..5: note = MeaslesF_CT_5
RGRCN

SEQ ID NO: 136 - protein, 4 aa, synthetic construct
  REGION 1..4: note = MeaslesF_CT_4
RGRC

SEQ ID NO: 137 - protein, 550 aa, synthetic construct
  REGION 1..550: note = Full MV-F
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 138-140 - skipped (3 entries)

SEQ ID NO: 141 - protein, 26 aa, synthetic construct
  REGION 1..26: note = NDVF_CT_26_FL
KQKAQQKTLLWLGNNTLDQMRATTKM

SEQ ID NO: 142 - protein, 14 aa, synthetic construct
  REGION 1..14: note = NDVF_CT_14
KQKAQQKTLLWLGN

SEQ ID NO: 143 - protein, 13 aa, synthetic construct
  REGION 1..13: note = NDVF_CT_13
KQKAQQKTLLWLG

SEQ ID NO: 144 - protein, 12 aa, synthetic construct
  REGION 1..12: note = NDVF_CT_12
KQKAQQKTLLWL

SEQ ID NO: 145 - protein, 11 aa, synthetic construct
  REGION 1..11: note = NDVF_CT_11
KQKAQQKTLLW

SEQ ID NO: 146 - protein, 10 aa, synthetic construct
  REGION 1..10: note = NDVF_CT_10
KQKAQQKTLL

SEQ ID NO: 147 - protein, 9 aa, synthetic construct
  REGION 1..9: note = NDVF_CT_9
KQKAQQKTL

SEQ ID NO: 148 - protein, 8 aa, synthetic construct
  REGION 1..8: note = NDVF_CT_8
KQKAQQKT

SEQ ID NO: 149 - protein, 7 aa, synthetic construct
  REGION 1..7: note = NDVF_CT_7
KQKAQQK

SEQ ID NO: 150 - protein, 6 aa, synthetic construct
  REGION 1..6: note = NDVF_CT_6
KQKAQQ

SEQ ID NO: 151 - protein, 5 aa, synthetic construct
  REGION 1..5: note = NDVF_CT_5
KQKAQ

SEQ ID NO: 152 - protein, 4 aa, synthetic construct
  REGION 1..4: note = NDVF_CT_4
KQKA

SEQ ID NO: 153-155 - skipped (3 entries)

SEQ ID NO: 156 - protein, 42 aa, synthetic construct
  REGION 1..42: note = SeVF_CT_42_FL
RLRRSMLMGNPDDRIPRDTYTLEPKIRHMYTNGGFDAMAEKR

SEQ ID NO: 157 - protein, 14 aa, synthetic construct
  REGION 1..14: note = SeVF_CT_14
RLRRSMLMGNPDDR

SEQ ID NO: 158 - protein, 13 aa, synthetic construct
  REGION 1..13: note = SeVF_CT_13
RLRRSMLMGNPDD

SEQ ID NO: 159 - protein, 12 aa, synthetic construct
  REGION 1..12: note = SeVF_CT_12
RLRRSMLMGNPD

SEQ ID NO: 160 - protein, 11 aa, synthetic construct
  REGION 1..11: note = SeVF_CT_11
RLRRSMLMGNP

SEQ ID NO: 161 - protein, 10 aa, synthetic construct
  REGION 1..10: note = SeVF_CT_10
RLRRSMLMGN

SEQ ID NO: 162 - protein, 9 aa, synthetic construct
  REGION 1..9: note = SeVF_CT_9
RLRRSMLMG

SEQ ID NO: 163 - protein, 8 aa, synthetic construct
  REGION 1..8: note = SeVF_CT_8
RLRRSMLM

SEQ ID NO: 164 - protein, 7 aa, synthetic construct
  REGION 1..7: note = SeVF_CT_7
RLRRSML

SEQ ID NO: 165 - protein, 6 aa, synthetic construct
  REGION 1..6: note = SeVF_CT_6
RLRRSM

SEQ ID NO: 166 - protein, 5 aa, synthetic construct
  REGION 1..5: note = SeVF_CT_5
RLRRS

SEQ ID NO: 167 - protein, 4 aa, synthetic construct
  REGION 1..4: note = SeVF_CT_4
RLRR

SEQ ID NO: 168-169 - skipped (2 entries)

SEQ ID NO: 170 - protein, 34 aa, synthetic construct
  REGION 1..34: note = BaEVwt_CT
NRLTAFINDKLNIIHAMVLTQQYQVLRTDEEAQD

SEQ ID NO: 171 - protein, 23 aa, synthetic construct
  REGION 1..23: note = Cocal_CT
RYRYQGSNNKRIYNDIEMSRFRK

SEQ ID NO: 172 - protein, 5 aa, synthetic construct
  REGION 1..5: note = EboV-GP_CT
CKFVF

SEQ ID NO: 173 - protein, 31 aa, synthetic construct
  REGION 1..31: note = GaLV_CT
KLVQFINDRISAVKILVLRQKYQALENEGNL

SEQ ID NO: 174 - protein, 16 aa, synthetic construct
  REGION 1..16: note = GaLV-RLess_CT
KLVQFINDRISAVKIL

SEQ ID NO: 175 - protein, 39 aa, synthetic construct
  REGION 1..39: note = LCMV_GP_CT
THRHIKGGSCPKPHRLTNKGICSCGAFKVPGVKTVWKRR

SEQ ID NO: 176 - protein, 16 aa, synthetic construct
  REGION 1..16: note = MLV-A-RLess_CT
RLVQFVKDRISVVQAL

SEQ ID NO: 177 - protein, 16 aa, synthetic construct
  REGION 1..16: note = BaEVRLess_CT
NRLTAFINDKLNIIHA

SEQ ID NO: 178 - protein, 32 aa, synthetic construct
  REGION 1..32: note = MLV-A_CT
RLVQFVKDRISVVQALVLTQQYHQLKPIEYEP

SEQ ID NO: 179 - protein, 29 aa, synthetic construct
  REGION 1..29: note = VSV-G_CT
RVGIHLCIKLKHTKKRQIYTDIEMNRLGK

SEQ ID NO: 180 - protein, 151 aa, synthetic construct
  REGION 1..151: note = HIV-1_gp41_LLIR-LLP1-LLP3-LLP2-gp41-CT-N_FL
NRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 181 - protein, 147 aa, synthetic construct
  REGION 1..147: note = HIV-1_gp41_LLP1-LLP3-LLP2-gp41-CT-N
NRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLE

SEQ ID NO: 182 - protein, 118 aa, synthetic construct
  REGION 1..118: note = HIV-1_gp41_LLP3-LLP2-gp41-CT-N
NRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVA

SEQ ID NO: 183 - protein, 80 aa, synthetic construct
  REGION 1..80: note = HIV-1_gp41_LLP2-gp41-CT-N
NRVRQGYSPLSFQTHLPIPRGPDRPEGIEEEGGERDRDRSIRLVNGSLALIWDDLRSLCLFSYHRLRDLLLIVTRIVELL

SEQ ID NO: 184 - protein, 47 aa, synthetic construct
  REGION 1..47: note = HIV-1_gp41_gp41-CT-N
NRVRQGYSPLSFQTHLPIPRGPDRPEGIEEEGGERDRDRSIRLVNGS

SEQ ID NO: 185 - protein, 104 aa, synthetic construct
  REGION 1..104: note = HIV-1_gp41_LLIR-LLP1-LLP3-LLP2
LALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 186 - protein, 100 aa, synthetic construct
  REGION 1..100: note = HIV-1_gp41_LLP1-LLP3-LLP2
LALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLE

SEQ ID NO: 187 - protein, 71 aa, synthetic construct
  REGION 1..71: note = HIV-1_gp41_LLP3-LLP2
LALIWDDLRSLCLFSYHRLRDLLLIVTRIVELLGRRGWEALKYWWNLLQYWSQELKNSAVNLLNATAIAVA

SEQ ID NO: 188 - protein, 33 aa, synthetic construct
  REGION 1..33: note = HIV-1_gp41_LLP2
LALIWDDLRSLCLFSYHRLRDLLLIVTRIVELL

SEQ ID NO: 189 - protein, 67 aa, synthetic construct
  REGION 1..67: note = HIV-1_gp41_LLIR-LLP1-LLP3
WEALKYWWNLLQYWSQELKNSAVNLLNATAIAVAEGTDRVIEVLQAAYRAIRHIPRRIRQGLERILL

SEQ ID NO: 190 - protein, 63 aa, synthetic construct
  REGION 1..63: note = HIV-1_gp41_LLP1-LLP3
WEALKYWWNLLQYWSQELKNSAVNLLNATAIAVAEGTDRVIEVLQAAYRAIRHIPRRIRQGLE

SEQ ID NO: 191 - protein, 34 aa, synthetic construct
  REGION 1..34: note = HIV-1_gp41_LLP3
WEALKYWWNLLQYWSQELKNSAVNLLNATAIAVA

SEQ ID NO: 192 - protein, 29 aa, synthetic construct
  REGION 1..29: note = HIV-1_gp41_LLIR-LLP1
RVIEVLQAAYRAIRHIPRRIRQGLERILL

SEQ ID NO: 193 - protein, 25 aa, synthetic construct
  REGION 1..25: note = HIV-1_gp41_LLP1
RVIEVLQAAYRAIRHIPRRIRQGLE

SEQ ID NO: 194 - protein, 47 aa, synthetic construct
  REGION 1..47: note = CD29_CT
KLLMIIHDRREFAKFEKEKMNAKWDTGENPIYKSAVTTVVNPKYEGK

SEQ ID NO: 195 - protein, 53 aa, synthetic construct
  REGION 1..53: note = CD29_CT
EKKRNTKLLMIIHDRREFAKFEKEKMNAKWDTGENPIYKSAVTTVVNPKYEGK

SEQ ID NO: 196 - protein, 10 aa, synthetic construct
  REGION 1..10: note = CD63_CT_1to11
AVEGGMKCVK

SEQ ID NO: 197 - protein, 9 aa, synthetic construct
  REGION 1..9: note = CD63_CT_73to81
CGACKENYC

SEQ ID NO: 198 - protein, 13 aa, synthetic construct
  REGION 1..13: note = CD63_CT_225to238
VEYGSRISKVLCC

SEQ ID NO: 199 - protein, 16 aa, synthetic construct
  REGION 1..16: note = CD63_CT_1to11
EKKRNTAVEGGMKCVK

SEQ ID NO: 200 - protein, 15 aa, synthetic construct
  REGION 1..15: note = CD63_CT_73to81
EKKRNTCGACKENYC

SEQ ID NO: 201 - protein, 19 aa, synthetic construct
  REGION 1..19: note = CD63_CT_225to238
EKKRNTVEYGSRISKVLCC

SEQ ID NO: 202 - protein, 35 aa, synthetic construct
  REGION 1..35: note = VSV-G_CT
EKKRNTRVGIHLCIKLKHTKKRQIYTDIEMNRLGK

SEQ ID NO: 203 - protein, 22 aa, synthetic construct
  REGION 1..22: note = BaEVRLess_CT
EKKRNTNRLTAFINDKLNIIHA

SEQ ID NO: 204 - protein, 29 aa, synthetic construct
  REGION 1..29: note = Cocal_CT
EKKRNTRYRYQGSNNKRIYNDIEMSRFRK

SEQ ID NO: 205 - protein, 11 aa, synthetic construct
  REGION 1..11: note = EboV-GP_CT
EKKRNTCKFVF

SEQ ID NO: 206 - protein, 37 aa, synthetic construct
  REGION 1..37: note = GaLV_CT
EKKRNTKLVQFINDRISAVKILVLRQKYQALENEGNL

SEQ ID NO: 207 - protein, 22 aa, synthetic construct
  REGION 1..22: note = GaLV-RLess_CT
EKKRNTKLVQFINDRISAVKIL

SEQ ID NO: 208 - protein, 38 aa, synthetic construct
  REGION 1..38: note = MLV-A_CT
EKKRNTRLVQFVKDRISVVQALVLTQQYHQLKPIEYEP

SEQ ID NO: 209 - protein, 22 aa, synthetic construct
  REGION 1..22: note = MLV-A-RLess_CT
EKKRNTRLVQFVKDRISVVQAL

SEQ ID NO: 210 - protein, 12 aa, synthetic construct
  REGION 1..12: note = Paramyxo_attachment_CTs
KSDLLGENIKKM

SEQ ID NO: 211 - protein, 12 aa, synthetic construct
  REGION 1..12: note = Paramyxo_attachment_CTs
KSDLLGDNIKKM

SEQ ID NO: 212 - protein, 16 aa, synthetic construct
  REGION 1..16: note = Paramyxo_attachment_CTs
RDIMLHERNIVIRSGM

SEQ ID NO: 213-214 - skipped (2 entries)

SEQ ID NO: 215 - protein, 6 aa, synthetic construct
  REGION 1..6: note = Paramyxo_attachment_CTs
KSDLLG

SEQ ID NO: 216 - protein, 6 aa, synthetic construct
  REGION 1..6: note = Paramyxo_attachment_CTs
RDIMLH

SEQ ID NO: 217 - protein, 16 aa, synthetic construct
  REGION 1..16: note = Paramyxo_attachment_CTs
EKKRKSDLLGENIKKM

SEQ ID NO: 218 - protein, 20 aa, synthetic construct
  REGION 1..20: note = Paramyxo_attachment_CTs
EKKRRDIMLHERNIVIRSGM

SEQ ID NO: 219 - protein, 18 aa, synthetic construct
  REGION 1..18: note = Paramyxo_attachment_CTs
EKKRNTKSDLLGDNIKKM

SEQ ID NO: 220 - protein, 22 aa, synthetic construct
  REGION 1..22: note = Paramyxo_attachment_CTs
EKKRNTRDIMLHERNIVIRSGM

SEQ ID NO: 221 - protein, 22 aa, synthetic construct
  REGION 1..22: note = Paramyxo_attachment_CTs
EKKRNTYSRLKSDLLGDNIKKM

SEQ ID NO: 222 - protein, 26 aa, synthetic construct
  REGION 1..26: note = Paramyxo_attachment_CTs
EKKRNTYSRLRDIMLHERNIVIRSGM

SEQ ID NO: 223 - protein, 59 aa, synthetic construct
  REGION 1..59: note = NIV CT and TM
LISMLSMIILYVLSIASLCIGLITFISFIIVEKKRNTYSRLEDRRVRPTSSGDLYYIGT

SEQ ID NO: 224 - protein, 524 aa, synthetic construct
  REGION 1..524: note = NNTHDSRRHKR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDSRRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 225 - protein, 524 aa, synthetic construct
  REGION 1..524: note = NNTHDLVRHKR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 226 - protein, 524 aa, synthetic construct
  REGION 1..524: note = NNTHDLVRHKR/LAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 227 - protein, 524 aa, synthetic construct
  REGION 1..524: note = NNTHDLVRHRR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVRHRRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 228 - protein, 524 aa, synthetic construct
  REGION 1..524: note = NNTHDLVRHRR/LAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVRHRRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 229 - protein, 524 aa, synthetic construct
  REGION 1..524: note = NNGHDSRRHKR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNGHDSRRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 230 - protein, 524 aa, synthetic construct
  REGION 1..524: note = NNGHDLVRHKR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNGHDLVRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 231 - protein, 524 aa, synthetic construct
  REGION 1..524: note = NNGHDLVRHKR/LAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNGHDLVRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 232 - protein, 524 aa, synthetic construct
  REGION 1..524: note = NNGHDLVRHRR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNGHDLVRHRRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 233 - protein, 524 aa, synthetic construct
  REGION 1..524: note = NNGHDLVRHRR/LAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNGHDLVRHRRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 234 - protein, 524 aa, synthetic construct
  REGION 1..524: note = QNTHDSRRHKR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDSRRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 235 - protein, 524 aa, synthetic construct
  REGION 1..524: note = QNTHDLVRHKR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 236 - protein, 524 aa, synthetic construct
  REGION 1..524: note = QNTHDLVRHKR/LAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 237 - protein, 524 aa, synthetic construct
  REGION 1..524: note = QNTHDLVRHRR/FAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVRHRRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 238 - protein, 524 aa, synthetic construct
  REGION 1..524: note = QNTHDLVRHRR/LAGV
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVRHRRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 239 - protein, 524 aa, synthetic construct
  REGION 1..524: note = VGDVK
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 240 - protein, 524 aa, synthetic construct
  REGION 1..524: note = RNHNR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLRNHNRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 241 - protein, 524 aa, synthetic construct
  REGION 1..524: note = RRHKR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLRRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 242 - protein, 524 aa, synthetic construct
  REGION 1..524: note = RRQKR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLRRQKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 243 - protein, 524 aa, synthetic construct
  REGION 1..524: note = GRQGR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLGRQGRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 244 - protein, 524 aa, synthetic construct
  REGION 1..524: note = TRQKR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLTRQKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 245 - protein, 524 aa, synthetic construct
  REGION 1..524: note = EIQSR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLEIQSRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 246 - protein, 524 aa, synthetic construct
  REGION 1..524: note = VPQSR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVPQSRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 247 - protein, 524 aa, synthetic construct
  REGION 1..524: note = NPQSR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLNPQSRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 248 - protein, 524 aa, synthetic construct
  REGION 1..524: note = PRTKR
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLPRTKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 249 - protein, 524 aa, synthetic construct
  REGION 1..524: note = HevF_signal_sequence
MATQEVRLKCLLCGIIVLVLSLEGLGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 250 - protein, 520 aa, synthetic construct
  REGION 1..520: note = CevF_signal_sequence
MSNKRTTVLIIISYTLFYLNNAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 251 - protein, 521 aa, synthetic construct
  REGION 1..521: note = HPIV2_signal_sequence
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 252 - protein, 522 aa, synthetic construct
  REGION 1..522: note = MevF_signal_sequence
MGLKVNVSAIFMAVLLTLQTPTGQILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 253 - protein, 529 aa, synthetic construct
  REGION 1..529: note = NDV-F_signal_sequence
MRSRSSTRIPVPLMLIIRIALTLSCIRLTSSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 254 - protein, 525 aa, synthetic construct
  REGION 1..525: note = SevF_signal_sequence
MTAYIQRSQCISTSLLVVLTTLVSCQIILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 255 - protein, 529 aa, synthetic construct
  REGION 1..529: note = HIV1-Env_signal_sequence
MRVKGIRKNYQHLWRWGIMLLGMLMICSAADILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 256 - protein, 516 aa, synthetic construct
  REGION 1..516: note = BaEV_signal_sequence
MGFTTKIIFLYNLVLVYAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 257 - protein, 515 aa, synthetic construct
  REGION 1..515: note = Cocal_signal_sequence
MNFLLLTFIVLPLCSHAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 258 - protein, 531 aa, synthetic construct
  REGION 1..531: note = EboV_signal_sequence
MGVTGILQLPRDRFKRTSFFLWVIILFQRTFSIILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 259 - protein, 541 aa, synthetic construct
  REGION 1..541: note = GaLV_signal_sequence
MVLLPGSMLLTSNLHHLRHQMSPGSWKRLIILLSCVFGGGGTSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 260 - protein, 534 aa, synthetic construct
  REGION 1..534: note = MLV-A_signal_sequence
MARSTLSKPLKNKVNPRGPLIPLILLMLRGVSTASPILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 261 - protein, 514 aa, synthetic construct
  REGION 1..514: note = VSVG_signal_sequence
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 262 - protein, 526 aa, synthetic construct
  REGION 1..526: note = Prolactin_signal_sequence
MNIKGSPWKGSLLLLLVSNLLLCQSVAPILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 263 - protein, 518 aa, synthetic construct
  REGION 1..518: note = IgGk-L_signal_sequence
METPAQLLFLLLLWLPDTTGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 264 - protein, 516 aa, synthetic construct
  REGION 1..516: note = Albumin_signal_sequence
MKWVTFISLLFLFSSAYSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 265 - protein, 524 aa, synthetic construct
  REGION 1..524: note = CD5_signal_sequence
MPMGSLQPLATLYLLGMLVASCLGRLILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 266 - protein, 513 aa, synthetic construct
  REGION 1..513: note = Trypsinogen_signal_sequence
MNPLLILTFVAAALAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 267 - protein, 518 aa, synthetic construct
  REGION 1..518: note = IL2_signal_sequence
MYRMQLLSCIALSLALVTNSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 268 - protein, 524 aa, synthetic construct
  REGION 1..524: note = NivF_TM_S490A_dec_fusogenicity
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLIAMLSMIILYVLSIASLCIGLITFISFIIVEKKRNT

SEQ ID NO: 269 - protein, 524 aa, synthetic construct
  REGION 1..524: note = NivF_TM_Y498A_dec_fusogenicity
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILAVLSIASLCIGLITFISFIIVEKKRNT

SEQ ID NO: 270 - protein, 524 aa, synthetic construct
  REGION 1..524: note = HevF_TM_FL
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIAALCIGLITFISFVIVEKKRNT

SEQ ID NO: 271 - protein, 524 aa, synthetic construct
  REGION 1..524: note = NivF_TM_HevF_S504A
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIAALCIGLITFISFIIVEKKRNT

SEQ ID NO: 272 - protein, 524 aa, synthetic construct
  REGION 1..524: note = NivF_TM_HevF_I516V
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFVIVEKKRNT

SEQ ID NO: 273 - protein, 524 aa, synthetic construct
  REGION 1..524: note = F3
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 274 - protein, 524 aa, synthetic construct
  REGION 1..524: note = F5
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 275 - protein, 524 aa, synthetic construct
  REGION 1..524: note = F4F5
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDQTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 276 - protein, 524 aa, synthetic construct
  REGION 1..524: note = F2F4F5
MVVILDKRCYCNLLILILMISECSVGILHYEKLSKIGLVKGVTRKYKIKSNPLTKDIVIKMIPNVSQMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDQTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 277 - protein, 498 aa, synthetic construct
  REGION 1..498: note = NivF_TM_S490A_dec_fusogenicity
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLIAMLSMIILYVLSIASLCIGLITFISFIIVEKKRNT

SEQ ID NO: 278 - protein, 524 aa, synthetic construct
  REGION 1..524: note = F2F3
MVVILDKRCYCNLLILILMISECSVGILHYEKLSKIGLVKGVTRKYKIKSNPLTKDIVIKMIPNVSQMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 279 - protein, 524 aa, synthetic construct
  REGION 1..524: note = F2F5
MVVILDKRCYCNLLILILMISECSVGILHYEKLSKIGLVKGVTRKYKIKSNPLTKDIVIKMIPNVSQMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 280 - protein, 524 aa, synthetic construct
  REGION 1..524: note = F3F5
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 281 - protein, 546 aa, synthetic construct
  REGION 1..546: note = FL017HevF488NivF_full-length
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIASLCIGLITFISFIIVEKKRNTYSRLEDRRVRPTSSGDLYYIGT

SEQ ID NO: 282 - protein, 524 aa, synthetic construct
  REGION 1..524: note = FL018HevF488NivF_CT-06
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIASLCIGLITFISFIIVEKKRNT

SEQ ID NO: 283 - protein, 528 aa, synthetic construct
  REGION 1..528: note = FL019HevF488NivF_CT-10
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIASLCIGLITFISFIIVEKKRNTYSRL

SEQ ID NO: 284 - protein, 498 aa, synthetic construct
  REGION 1..498: note = NivF_TM_Y498A_dec_fusogenicity
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILAVLSIASLCIGLITFISFIIVEKKRNT

SEQ ID NO: 285 - protein, 498 aa, synthetic construct
  REGION 1..498: note = HevF_TM_FL
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIAALCIGLITFISFVIVEKKRNT

SEQ ID NO: 286 - protein, 5 aa, synthetic construct
  REGION 1..5: note = Linker
GGGGS

SEQ ID NO: 287 - protein, 6 aa, synthetic construct
  REGION 1..6: note = Linker
GGGGGS

SEQ ID NO: 288 - protein, 5 aa, synthetic construct
  REGION 1..5: note = Linker, n = 1-6
  REPEAT 1..5: note = repeat 1-6 times
GGGGS

SEQ ID NO: 289 - protein, 6 aa, synthetic construct
  REGION 1..6: note = Linker, n = 1-6
  REPEAT 1..6: note = repeat 1-6
GGGGGS

SEQ ID NO: 290 - protein, 524 aa, synthetic construct
  REGION 1..524: note = F2F3F5
MVVILDKRCYCNLLILILMISECSVGILHYEKLSKIGLVKGVTRKYKIKSNPLTKDIVIKMIPNVSQMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 291 - protein, 524 aa, synthetic construct
  REGION 1..524: note = R109L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVLLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 292 - protein, 524 aa, synthetic construct
  REGION 1..524: note = Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 293 - protein, 524 aa, synthetic construct
  REGION 1..524: note = R109L_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVLLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 294 - protein, 546 aa, synthetic construct
  REGION 1..546: note = FL012 HevF_full-length
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 295 - protein, 524 aa, synthetic construct
  REGION 1..524: note = FL_013HevF_CT-06
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGN

SEQ ID NO: 296 - protein, 528 aa, synthetic construct
  REGION 1..528: note = FL014 HevF_CT-10
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRL

SEQ ID NO: 297 - protein, 511 aa, synthetic construct
  REGION 1..511: note = FL057_CT060
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVRGR

SEQ ID NO: 298 - protein, 515 aa, synthetic construct
  REGION 1..515: note = FL063_CT060
MYRMQLLSCIALSLALVTNSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVRGR

SEQ ID NO: 299 - protein, 514 aa, synthetic construct
  REGION 1..514: note = FL007_FL057_CT060
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 300 - protein, 514 aa, synthetic construct
  REGION 1..514: note = FL007_FL018
MKCLLYLAFLFIGVNCILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIASLCIGLITFISFIIVEKKRNT

SEQ ID NO: 301 - protein, 568 aa, synthetic construct
  REGION 1..568: note = (E501 A, W504A, Q530A, E533A) NiV G protein (Gc34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQC

SEQ ID NO: 302 - protein, 524 aa, synthetic construct
  REGION 1..524: note = (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 303 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 304 - protein, 602 aa, synthetic construct
  REGION 1..602: note = Uniprot Q9IH62
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 305 - protein, 602 aa, synthetic construct
  REGION 1..602: note = Full-length NiVG with (E501 A, W504A, Q530A, E533A)
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 306 - protein, 497 aa, synthetic construct
  REGION 1..497: note = mature NiV-F CT_5
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRN

SEQ ID NO: 307 - protein, 492 aa, synthetic construct; identical to SEQ ID NO: 2
  REGION 1..492: note = mature MeaslesF_CT_7

SEQ ID NO: 308 - protein, 501 aa, synthetic construct
  REGION 1..501: note = mature NDVF_CT_9
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQKTL

SEQ ID NO: 309 - protein, 498 aa, synthetic construct
  REGION 1..492: note = mature NDVF_CT_6
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 310 - protein, 524 aa, synthetic construct
  REGION 1..524: note = mature HeVF_CT_6
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 311 - protein, 15 aa, synthetic construct
  REGION 1..15: note = NiV-F cleavage site
NNTHDLVGDVRLAGV

SEQ ID NO: 312 - protein, 5 aa, synthetic construct
  REGION 1..5: note = Furin cleavage seq
  VARIANT 2: note = X is any amino acid
RXRKR

SEQ ID NO: 313 - protein, 15 aa, synthetic construct
  REGION 1..15: note = Modified protease site
NNTHDSRRHKRFAGV

SEQ ID NO: 314 - protein, 15 aa, synthetic construct
  REGION 1..15: note = Modified protease site
NNTHDLVRHKRFAGV

SEQ ID NO: 315 - protein, 15 aa, synthetic construct
  REGION 1..15: note = Modified protease site
NNTHDLVRHKRFAGV

SEQ ID NO: 316 - protein, 15 aa, synthetic construct
  REGION 1..15: note = Modified protease site
NNTHDLVRHRRFAGV

SEQ ID NO: 317 - protein, 15 aa, synthetic construct
  REGION 1..15: note = Modified protease site
NNTHDLVRHRRLAGV

SEQ ID NO: 318 - protein, 15 aa, synthetic construct
  REGION 1..15: note = Modified protease site
NNGHDSRRHKRFAGV

SEQ ID NO: 319 - protein, 15 aa, synthetic construct
  REGION 1..15: note = Modified protease site
NNGHDLVRHKRFAGV

SEQ ID NO: 320 - protein, 15 aa, synthetic construct
  REGION 1..15: note = Modified protease site
NNGHDLVRHKRLAGV

SEQ ID NO: 321 - protein, 15 aa, synthetic construct
  REGION 1..15: note = Modified protease site
NNGHDLVRHRRFAGV

SEQ ID NO: 322 - protein, 15 aa, synthetic construct
  REGION 1..15: note = Modified protease site
NNGHDLVRHRRLAGV

SEQ ID NO: 323 - protein, 15 aa, synthetic construct
  REGION 1..15: note = Modified protease site
QNTHDSRRHKRFAGV

SEQ ID NO: 324 - protein, 15 aa, synthetic construct
  REGION 1..15: note = Modified protease site
QNTHDLVRHKRFAGV

SEQ ID NO: 325 - protein, 15 aa, synthetic construct
  REGION 1..15: note = Modified protease site
QNTHDLVRHKRLAGV

SEQ ID NO: 326 - protein, 15 aa, synthetic construct
  REGION 1..15: note = Modified protease site
QNTHDLVRHRRFAGV

SEQ ID NO: 327 - protein, 15 aa, synthetic construct
  REGION 1..15: note = Modified protease site
QNTHDLVRHRRLAGV

SEQ ID NO: 328 - protein, 37 aa, synthetic construct
  REGION 1..37: note = Modified protease site
LISMLSMIILYVLSIASLCIGLITFISFIIVEKKRNT

SEQ ID NO: 329 - protein, 5 aa, synthetic construct
  REGION 1..5: note = Modified protease site
VGDVK

SEQ ID NO: 330 - protein, 5 aa, synthetic construct
  REGION 1..5: note = Modified protease site
RNHNR

SEQ ID NO: 331 - protein, 5 aa, synthetic construct
  REGION 1..5: note = Modified protease site
RRHKR

SEQ ID NO: 332 - protein, 5 aa, synthetic construct
  REGION 1..5: note = Modified protease site
RRQKR

SEQ ID NO: 333 - protein, 5 aa, synthetic construct
  REGION 1..5: note = Modified protease site
GRQGR

SEQ ID NO: 334 - protein, 5 aa, synthetic construct
  REGION 1..5: note = Modified protease site
TRQKR

SEQ ID NO: 335 - protein, 5 aa, synthetic construct
  REGION 1..5: note = Modified protease site
EIQSR

SEQ ID NO: 336 - protein, 5 aa, synthetic construct
  REGION 1..5: note = Modified protease site
VPQSR

SEQ ID NO: 337 - protein, 5 aa, synthetic construct
  REGION 1..5: note = Modified protease site
NPQSR

SEQ ID NO: 338 - protein, 5 aa, synthetic construct
  REGION 1..5: note = Modified protease site
PRTKR

SEQ ID NO: 339 - protein, 5 aa, synthetic construct
  REGION 1..5: note = Modified protease site
VGDVR

SEQ ID NO: 340 - protein, 26 aa, synthetic construct
  REGION 1..26: note = HevF_signal_sequence
MATQEVRLKCLLCGIIVLVLSLEGLG

SEQ ID NO: 341 - protein, 22 aa, synthetic construct
  REGION 1..22: note = CevF_signal_sequence
MSNKRTTVLIIISYTLFYLNNA

SEQ ID NO: 342 - protein, 23 aa, synthetic construct
  REGION 1..23: note = HPIV2_signal_sequence
MHHLHPMIVCIFVMYTGIVGSDA

SEQ ID NO: 343 - protein, 24 aa, synthetic construct
  REGION 1..24: note = MevF_signal_sequence
MGLKVNVSAIFMAVLLTLQTPTGQ

SEQ ID NO: 344 - protein, 31 aa, synthetic construct
  REGION 1..31: note = NDV-F_signal_sequence
MRSRSSTRIPVPLMLIIRIALTLSCIRLTSS

SEQ ID NO: 345 - protein, 27 aa, synthetic construct
  REGION 1..27: note = SevF_signal_sequence
MTAYIQRSQCISTSLLVVLTTLVSCQI

SEQ ID NO: 346 - protein, 31 aa, synthetic construct
  REGION 1..31: note = HIV1-Env_signal_sequence
MRVKGIRKNYQHLWRWGIMLLGMLMICSAAD

SEQ ID NO: 347 - protein, 18 aa, synthetic construct
  REGION 1..18: note = BaEV_signal_sequence
MGFTTKIIFLYNLVLVYA

SEQ ID NO: 348 - protein, 17 aa, synthetic construct
  REGION 1..17: note = Cocal_signal_sequence
MNFLLLTFIVLPLCSHA

SEQ ID NO: 349 - protein, 33 aa, synthetic construct
  REGION 1..33: note = EboV_signal_sequence
MGVTGILQLPRDRFKRTSFFLWVIILFQRTFSI

SEQ ID NO: 350 - protein, 43 aa, synthetic construct
  REGION 1..43: note = GaLV_signal_sequence
MVLLPGSMLLTSNLHHLRHQMSPGSWKRLIILLSCVFGGGGTS

SEQ ID NO: 351 - protein, 36 aa, synthetic construct
  REGION 1..36: note = MLV-A_signal_sequence
MARSTLSKPLKNKVNPRGPLIPLILLMLRGVSTASP

SEQ ID NO: 352 - protein, 17 aa, synthetic construct
  REGION 1..17: note = VSVG_signal_sequence
MKCLLYLAFLFIGVNCI

SEQ ID NO: 353 - protein, 28 aa, synthetic construct
  REGION 1..28: note = Prolactin_signal_sequence
MNIKGSPWKGSLLLLLVSNLLLCQSVAP

SEQ ID NO: 354 - protein, 20 aa, synthetic construct
  REGION 1..20: note = IgGk-L_signal_sequence
METPAQLLFLLLLWLPDTTG

SEQ ID NO: 355 - protein, 18 aa, synthetic construct
  REGION 1..18: note = Albumin_signal_sequence
MKWVTFISLLFLFSSAYS

SEQ ID NO: 356 - protein, 26 aa, synthetic construct
  REGION 1..26: note = CD5_signal_sequence
MPMGSLQPLATLYLLGMLVASCLGRL

SEQ ID NO: 357 - protein, 15 aa, synthetic construct
  REGION 1..15: note = Trypsinogen_signal_sequence
MNPLLILTFVAAALA

SEQ ID NO: 358 - protein, 20 aa, synthetic construct
  REGION 1..20: note = IL2_signal_sequence
MYRMQLLSCIALSLALVTNS

SEQ ID NO: 359 - protein, 31 aa, synthetic construct
  REGION 1..31: note = NivF_TM_S490A
LIAMLSMIILYVLSIASLCIGLITFISFIIV

SEQ ID NO: 360 - protein, 31 aa, synthetic construct
  REGION 1..31: note = NivF_TM_Y498A
LISMLSMIILAVLSIASLCIGLITFISFIIV

SEQ ID NO: 361 - protein, 31 aa, synthetic construct
  REGION 1..31: note = HevF_TM_FL
LISMLSMIILYVLSIAALCIGLITFISFVIV

SEQ ID NO: 362 - protein, 31 aa, synthetic construct
  REGION 1..31: note = NivF_TM_HevF_S504A
LISMLSMIILYVLSIAALCIGLITFISFIIV

SEQ ID NO: 363 - protein, 31 aa, synthetic construct
  REGION 1..31: note = NivF_TM_HevF_I516V
LISMLSMIILYVLSIASLCIGLITFISFVIV

SEQ ID NO: 364 - protein, 520 aa, synthetic construct
  REGION 1..520: note = Nipah virus NiV-F F0 (aa 27-546)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 365 - protein, 83 aa, synthetic construct
  REGION 1..83: note = Nipah virus NiV-F F2 (aa 27-109 of SEQ ID NO:1)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 366 - protein, 437 aa, synthetic construct
  REGION 1..437: note = Nipah virus NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 367 - protein, 524 aa, synthetic construct
  REGION 1..524: note = F3F5_Q393L_CT-06_K1A
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 368 - protein, 524 aa, synthetic construct
  REGION 1..524: note = F3F5_Q393L_CT-10_K1A
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 369 - protein, 524 aa, synthetic construct
  REGION 1..524: note = VSVG_signal_sequence_ MeaslesF_CT_3
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 370 - protein, 524 aa, synthetic construct
  REGION 1..524: note = IL2_signal_sequence_MeaslesF_CT_3
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 371 - protein, 521 aa, synthetic construct
  REGION 1..521: note = F3F5_ VSVG_signal_sequence_ MeaslesF_CT_3
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 372 - protein, 514 aa, synthetic construct
  REGION 1..514: note = F3F5_ HevF488NivF_CT-06
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 373 - protein, 524 aa, synthetic construct
  REGION 1..524: note = F5_ Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVKQKAQQ

SEQ ID NO: 374 - protein, 524 aa, synthetic construct
  REGION 1..524: note = F5_ Q393L_ VGDVK
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRGN

SEQ ID NO: 375 - protein, 524 aa, synthetic construct
  REGION 1..524: note = F5__ VGDVK
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVKQKAQQ

SEQ ID NO: 376 - protein, 524 aa, synthetic construct
  REGION 1..524: note = Q393L_ VGDVK
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRGN

SEQ ID NO: 377 - protein, 524 aa, synthetic construct
  REGION 1..524: note = F5_ Q393L_ HPIV2_signal_sequence
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVKQKAQQ

SEQ ID NO: 378 - protein, 524 aa, synthetic construct
  REGION 1..524: note = F5_ Q393L_ VSVG_signal_sequence
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 379 - protein, 604 aa, synthetic construct
  REGION 1..604: note = Hendra virus G protein Uniprot O89343
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 380 - protein, 622 aa, synthetic construct
  REGION 1..622: note = gb:JQ001776:8170
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 381 - protein, 632 aa, synthetic construct
  REGION 1..632: note = gb:NC_025256:9117
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 382 - protein, 625 aa, synthetic construct
  REGION 1..625: note = gb:NC_025352:8716-11257
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 383 - protein, 409 aa, synthetic construct
  REGION 1..409: note = Nipah virus NiV F F1 deltaCT
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIV

SEQ ID NO: 384 - protein, 498 aa, synthetic construct
  REGION 1..498: note = mature HeVF_CT_6
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 385 - protein, 498 aa, synthetic construct
  REGION 1..498: note = NNTHDSRRHKR/FAGV
ILHYEKLSKIGLVKGVTRKYKIKSNPLTKDIVIKMIPNVSNMSQCTGSVMENYKTRLNGILTPIKGALEIYKNNTHDSRRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 386 - protein, 498 aa, synthetic construct
  REGION 1..498: note = NNTHDLVRHKR/FAGV
ILHYEKLSKIGLVKGVTRKYKIKSNPLTKDIVIKMIPNVSNMSQCTGSVMENYKTRLNGILTPIKGALEIYKNNTHDLVRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 387 - protein, 498 aa, synthetic construct
  REGION 1..498: note = NNTHDLVRHKR/LAGV
ILHYEKLSKIGLVKGVTRKYKIKSNPLTKDIVIKMIPNVSNMSQCTGSVMENYKTRLNGILTPIKGALEIYKNNTHDLVRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 388 - protein, 498 aa, synthetic construct
  REGION 1..498: note = NNTHDLVRHRR/FAGV
ILHYEKLSKIGLVKGVTRKYKIKSNPLTKDIVIKMIPNVSNMSQCTGSVMENYKTRLNGILTPIKGALEIYKNNTHDLVRHRRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 389 - protein, 498 aa, synthetic construct
  REGION 1..498: note = NNTHDLVRHRR/LAGV
ILHYEKLSKIGLVKGVTRKYKIKSNPLTKDIVIKMIPNVSNMSQCTGSVMENYKTRLNGILTPIKGALEIYKNNTHDLVRHRRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 390 - protein, 498 aa, synthetic construct
  REGION 1..498: note = NNGHDSRRHKR/FAGV
ILHYEKLSKIGLVKGVTRKYKIKSNPLTKDIVIKMIPNVSNMSQCTGSVMENYKTRLNGILTPIKGALEIYKNNGHDSRRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 391 - protein, 498 aa, synthetic construct
  REGION 1..498: note = NNGHDLVRHKR/FAGV
ILHYEKLSKIGLVKGVTRKYKIKSNPLTKDIVIKMIPNVSNMSQCTGSVMENYKTRLNGILTPIKGALEIYKNNGHDLVRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 392 - protein, 498 aa, synthetic construct
  REGION 1..498: note = NNGHDLVRHKR/LAGV
ILHYEKLSKIGLVKGVTRKYKIKSNPLTKDIVIKMIPNVSNMSQCTGSVMENYKTRLNGILTPIKGALEIYKNNGHDLVRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 393 - protein, 498 aa, synthetic construct
  REGION 1..498: note = NNGHDLVRHRR/FAGV
ILHYEKLSKIGLVKGVTRKYKIKSNPLTKDIVIKMIPNVSNMSQCTGSVMENYKTRLNGILTPIKGALEIYKNNGHDLVRHRRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 394 - protein, 498 aa, synthetic construct
  REGION 1..498: note = NNGHDLVRHRR/LAGV
ILHYEKLSKIGLVKGVTRKYKIKSNPLTKDIVIKMIPNVSNMSQCTGSVMENYKTRLNGILTPIKGALEIYKNNGHDLVRHRRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 395 - protein, 498 aa, synthetic construct
  REGION 1..498: note = QNTHDSRRHKR/FAGV
ILHYEKLSKIGLVKGVTRKYKIKSNPLTKDIVIKMIPNVSNMSQCTGSVMENYKTRLNGILTPIKGALEIYKQNTHDSRRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 396 - protein, 498 aa, synthetic construct
  REGION 1..498: note = QNTHDLVRHKR/FAGV
ILHYEKLSKIGLVKGVTRKYKIKSNPLTKDIVIKMIPNVSNMSQCTGSVMENYKTRLNGILTPIKGALEIYKQNTHDLVRHKRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 397 - protein, 498 aa, synthetic construct
  REGION 1..498: note = QNTHDLVRHKR/LAGV
ILHYEKLSKIGLVKGVTRKYKIKSNPLTKDIVIKMIPNVSNMSQCTGSVMENYKTRLNGILTPIKGALEIYKQNTHDLVRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 398 - protein, 498 aa, synthetic construct
  REGION 1..498: note = QNTHDLVRHRR/FAGV
ILHYEKLSKIGLVKGVTRKYKIKSNPLTKDIVIKMIPNVSNMSQCTGSVMENYKTRLNGILTPIKGALEIYKQNTHDLVRHRRFAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 399 - protein, 498 aa, synthetic construct
  REGION 1..498: note = QNTHDLVRHRR/LAGV
ILHYEKLSKIGLVKGVTRKYKIKSNPLTKDIVIKMIPNVSNMSQCTGSVMENYKTRLNGILTPIKGALEIYKQNTHDLVRHRRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 400 - protein, 498 aa, synthetic construct
  REGION 1..498: note = VGDVK
ILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 401 - protein, 498 aa, synthetic construct
  REGION 1..498: note = RNHNR
ILHYEKLSKIGLVKGVTRKYKIKSNPLTKDIVIKMIPNVSNMSQCTGSVMENYKTRLNGILTPIKGALEIYKNNTHDLRNHNRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 402 - protein, 498 aa, synthetic construct
  REGION 1..498: note = RRHKR
ILHYEKLSKIGLVKGVTRKYKIKSNPLTKDIVIKMIPNVSNMSQCTGSVMENYKTRLNGILTPIKGALEIYKNNTHDLRRHKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 403 - protein, 498 aa, synthetic construct
  REGION 1..498: note = RRQKR
ILHYEKLSKIGLVKGVTRKYKIKSNPLTKDIVIKMIPNVSNMSQCTGSVMENYKTRLNGILTPIKGALEIYKNNTHDLRRQKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 404 - protein, 498 aa, synthetic construct
  REGION 1..498: note = GRQGR
ILHYEKLSKIGLVKGVTRKYKIKSNPLTKDIVIKMIPNVSNMSQCTGSVMENYKTRLNGILTPIKGALEIYKNNTHDLGRQGRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 405 - protein, 498 aa, synthetic construct
  REGION 1..498: note = TRQKR
ILHYEKLSKIGLVKGVTRKYKIKSNPLTKDIVIKMIPNVSNMSQCTGSVMENYKTRLNGILTPIKGALEIYKNNTHDLTRQKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 406 - protein, 498 aa, synthetic construct
  REGION 1..498: note = EIQSR
ILHYEKLSKIGLVKGVTRKYKIKSNPLTKDIVIKMIPNVSNMSQCTGSVMENYKTRLNGILTPIKGALEIYKNNTHDLEIQSRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 407 - protein, 498 aa, synthetic construct
  REGION 1..498: note = VPQSR
ILHYEKLSKIGLVKGVTRKYKIKSNPLTKDIVIKMIPNVSNMSQCTGSVMENYKTRLNGILTPIKGALEIYKNNTHDLVPQSRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 408 - protein, 498 aa, synthetic construct
  REGION 1..498: note = NPQSR
ILHYEKLSKIGLVKGVTRKYKIKSNPLTKDIVIKMIPNVSNMSQCTGSVMENYKTRLNGILTPIKGALEIYKNNTHDLNPQSRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 409 - protein, 498 aa, synthetic construct
  REGION 1..498: note = PRTKR
ILHYEKLSKIGLVKGVTRKYKIKSNPLTKDIVIKMIPNVSNMSQCTGSVMENYKTRLNGILTPIKGALEIYKNNTHDLPRTKRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 410 - protein, 498 aa, synthetic construct
  REGION 1..498: note = F3
ILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 411 - protein, 498 aa, synthetic construct
  REGION 1..498: note = F5
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 412 - protein, 498 aa, synthetic construct
  REGION 1..498: note = F4F5
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDQTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 413 - protein, 498 aa, synthetic construct
  REGION 1..498: note = F2F4F5
ILHYEKLSKIGLVKGVTRKYKIKSNPLTKDIVIKMIPNVSQMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDQTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 414 - protein, 498 aa, synthetic construct
  REGION 1..498: note = F2F5
ILHYEKLSKIGLVKGVTRKYKIKSNPLTKDIVIKMIPNVSQMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 415 - protein, 498 aa, synthetic construct
  REGION 1..498: note = F3F5
ILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 416 - protein, 498 aa, synthetic construct; identical to SEQ ID NO: 410
  REGION 1..498: note = F3

SEQ ID NO: 417 - protein, 498 aa, synthetic construct
  REGION 1..498: note = R109L
ILHYEKLSKIGLVKGVTRKYKIKSNPLTKDIVIKMIPNVSNMSQCTGSVMENYKTRLNGILTPIKGALEIYKNNTHDLVGDVLLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 418 - protein, 498 aa, synthetic construct
  REGION 1..498: note = Q393L
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 419 - protein, 498 aa, synthetic construct
  REGION 1..498: note = R109L_Q393L
ILHYEKLSKIGLVKGVTRKYKIKSNPLTKDIVIKMIPNVSNMSQCTGSVMENYKTRLNGILTPIKGALEIYKNNTHDLVGDVLLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 420 - protein, 498 aa, synthetic construct
  REGION 1..498: note = NivF_TM_HevF_S504A
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIAALCIGLITFISFIIVEKKRNT

SEQ ID NO: 421 - protein, 498 aa, synthetic construct
  REGION 1..498: note = NivF_TM_HevF_I516V
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFVIVEKKRNT

SEQ ID NO: 422 - protein, 498 aa, synthetic construct
  REGION 1..498: note = HPIV2_signal_sequence_CT06_Q393L
ILHYEKLSKIGLVKGVTRKYKIKSNPLTKDIVIKMIPNVSQMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 423 - protein, 521 aa, synthetic construct
  REGION 1..521: note = VSVG_signal_sequence_CT06_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 424 - protein, 514 aa, synthetic construct
  REGION 1..514: note = VGDVK (HeV)_HPIV2_signal_sequence_CT06_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 425 - protein, 521 aa, synthetic construct
  REGION 1..521: note = VGDVK (HeV)_VSVG_signal_sequence_CT06_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 426 - protein, 514 aa, synthetic construct
  REGION 1..514: note = NDVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 427 - protein, 524 aa, synthetic construct; identical to SEQ ID NO: 378
  REGION 1..524: note = VGDVK (HeV)_NDVF_CT_6_Q393L

SEQ ID NO: 428 - protein, 524 aa, synthetic construct
  REGION 1..524: note = HPIV2_signal_sequence-NDVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 429 - protein, 521 aa, synthetic construct
  REGION 1..521: note = VSVG_signal_sequence_NDVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 430 - protein, 514 aa, synthetic construct
  REGION 1..514: note = VGDVK (HeV)_HPIV2_signal_sequence_NDVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 431 - protein, 521 aa, synthetic construct
  REGION 1..521: note = VGDVK (HeV)_VSVG_signal_sequence_NDVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 432 - protein, 514 aa, synthetic construct
  REGION 1..514: note = HeVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 433 - protein, 524 aa, synthetic construct
  REGION 1..524: note = VGDVK (HeV)_ HeVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 434 - protein, 524 aa, synthetic construct
  REGION 1..524: note = HPIV2_signal_sequence_HeVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 435 - protein, 521 aa, synthetic construct
  REGION 1..521: note = VSVG_signal_sequence_HeVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 436 - protein, 514 aa, synthetic construct
  REGION 1..514: note = VGDVK (HeV)_HPIV2_signal_sequence_HeVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 437 - protein, 521 aa, synthetic construct
  REGION 1..521: note = VGDVK (HeV)_VSVG_signal_sequence_HeVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 438 - protein, 514 aa, synthetic construct
  REGION 1..514: note = F3_CT06_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKNNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 439 - protein, 524 aa, synthetic construct
  REGION 1..524: note = F3_VGDVK (HeV)_CT06_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 440 - protein, 524 aa, synthetic construct
  REGION 1..524: note = F3-_HPIV2_signal_sequence_CT06_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 441 - protein, 521 aa, synthetic construct
  REGION 1..521: note = F3-_VSVG_signal_sequence_CT06_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 442 - protein, 514 aa, synthetic construct
  REGION 1..514: note = F3_VGDVK (HeV)_HPIV2_signal_sequence_CT06_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 443 - protein, 521 aa, synthetic construct
  REGION 1..521: note = F3_VGDVK (HeV)_VSVG_signal_sequence_CT06_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 444 - protein, 514 aa, synthetic construct
  REGION 1..514: note = F3-_NDVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 445 - protein, 524 aa, synthetic construct
  REGION 1..524: note = F3_VGDVK (HeV)_NDVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 446 - protein, 524 aa, synthetic construct
  REGION 1..524: note = F3_HPIV2_signal_sequence_NDVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 447 - protein, 521 aa, synthetic construct
  REGION 1..521: note = F3_VSVG_signal_sequence_NDVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 448 - protein, 514 aa, synthetic construct
  REGION 1..514: note = F3_VGDVK (HeV)_HPIV2_signal_sequence_NDVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 449 - protein, 521 aa, synthetic construct
  REGION 1..521: note = F3_VGDVK (HeV)_VSVG_signal_sequence_NDVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 450 - protein, 514 aa, synthetic construct
  REGION 1..514: note = F3_HeVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVKQKAQQ

SEQ ID NO: 451 - protein, 524 aa, synthetic construct
  REGION 1..524: note = F3_VGDVK (HeV)_HeVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 452 - protein, 524 aa, synthetic construct
  REGION 1..524: note = F3_HPIV2_signal_sequence_HeVF_CT_6_Q393L
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 453 - protein, 521 aa, synthetic construct
  REGION 1..521: note = F3_VSVG_signal_sequence_HeVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 454 - protein, 514 aa, synthetic construct
  REGION 1..514: note = F3_VGDVK (HeV)_HPIV2_signal_sequence_HeVF_CT_6_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 455 - protein, 521 aa, synthetic construct
  REGION 1..521: note = F3_VGDVK (HeV)_VSVG_signal_sequence_HeVF_CT_6_Q393L
MHHLHPMIVCIFVMYTGIVGSDAILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 456 - protein, 514 aa, synthetic construct
  REGION 1..514: note = HPIV2_signal_sequence_CT06_Q393L
MKCLLYLAFLFIGVNCILHYEKLSKIGLVKGVTRKYKIKSNPLTKDIVIKMIPNVSNMSQCTGSVMENYKTRLNGILTPIKGALEIYKQNTHDLVGDVKLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCLCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRGN

SEQ ID NO: 457 - protein, 524 aa, synthetic construct
  REGION 1..524: note = N1N2_CT06
MVVILDKRCYCNLLILILMISECSVGILHYEKLSKIGLVKGVTRKYKIKSNPLTKDIVIKMIPQVSQMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 458 - protein, 524 aa, synthetic construct
  REGION 1..524: note = N1N2N3_CT06
MVVILDKRCYCNLLILILMISECSVGILHYEKLSKIGLVKGVTRKYKIKSNPLTKDIVIKMIPQVSQMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 459 - protein, 524 aa, synthetic construct
  REGION 1..524: note = N1N2N3N5_CT06
MVVILDKRCYCNLLILILMISECSVGILHYEKLSKIGLVKGVTRKYKIKSNPLTKDIVIKMIPQVSQMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 460 - protein, 524 aa, synthetic construct
  REGION 1..524: note = N1N3_CT06
MVVILDKRCYCNLLILILMISECSVGILHYEKLSKIGLVKGVTRKYKIKSNPLTKDIVIKMIPQVSNMSQCTGSVMENYKTRLNGILTPIKGALEIYKQNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 461 - protein, 524 aa, synthetic construct
  REGION 1..524: note = N1N2N5_CT06
MVVILDKRCYCNLLILILMISECSVGILHYEKLSKIGLVKGVTRKYKIKSNPLTKDIVIKMIPQVSQMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 462 - protein, 524 aa, synthetic construct
  REGION 1..524: note = N1N3N5_CT06
MVVILDKRCYCNLLILILMISECSVGILHYEKLSKIGLVKGVTRKYKIKSNPLTKDIVIKMIPQVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMQQSLQQSKDYIKEAQRLLDTVNPSLISMLSMIILYVLSIASLCIGLITFISFIIVEKKRNT

SEQ ID NO: 463 - protein, 524 aa, synthetic construct; identical to SEQ ID NO: 462
  REGION 1..524: note = N1N5_CT06

SEQ ID NO: 464 - protein, 524 aa, synthetic construct; identical to SEQ ID NO: 290
  REGION 1..524: note = N2N3N5_CT06

SEQ ID NO: 465 - protein, 21 aa, synthetic construct
  REGION 1..21: note = CD8alpha signal peptide
MALPVTALLLPLALLLHAARP

SEQ ID NO: 466 - protein, 20 aa, synthetic construct
  REGION 1..20: note = IgK signal peptide
METDTLLLWVLLLWVPGSTG

SEQ ID NO: 467 - protein, 22 aa, synthetic construct
  REGION 1..22: note = GMCSFR-alpha (CSF2RA) signal peptide
MLLLVTSLLLCELPHPAFLLIP

SEQ ID NO: 468 - protein, 45 aa, synthetic construct
  REGION 1..45: note = CD8alpha hinge domain
TTTPAPRPPTPAPTIASQPLSLRPEACRPAAGGAVHTRGLDFACD

SEQ ID NO: 469 - protein, 39 aa, synthetic construct
  REGION 1..39: note = CD28 hinge domain
IEVMYPPPYLDNEKSNGTIIHVKGKHLCPSPLFPGPSKP

SEQ ID NO: 470 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 471 - protein, 12 aa, synthetic construct
  REGION 1..12: note = IgG4 hinge domain
ESKYGPPCPPCP

SEQ ID NO: 472 - protein, 12 aa, synthetic construct
  REGION 1..12: note = IgG4 hinge domain
ESKYGPPCPSCP

SEQ ID NO: 473 - protein, 24 aa, synthetic construct
  REGION 1..24: note = CD8alpha transmembrane domain
IYIWAPLAGTCGVLLLSLVITLYC

SEQ ID NO: 474 - protein, 27 aa, synthetic construct
  REGION 1..27: note = CD28 transmembrane domain
FWVLVVVGGVLACYSLLVTVAFIIFWV

SEQ ID NO: 475 - protein, 28 aa, synthetic construct
  REGION 1..28: note = CD28 transmembrane domain
MFWVLVVVGGVLACYSLLVTVAFIIFWV

SEQ ID NO: 476 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 477 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 478 - protein, 112 aa, synthetic construct
  REGION 1..112: note = CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 479 - protein, 112 aa, synthetic construct
  REGION 1..112: note = CD3zeta signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 480 - protein, 245 aa, synthetic construct
  REGION 1..245: note = Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 481 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 482 - protein, 6 aa, synthetic construct
  REGION 1..6: note = Anti-CD19 FMC63 scFv light chain CDR1
QDISKY

SEQ ID NO: 483 - skipped

SEQ ID NO: 484 - protein, 9 aa, synthetic construct
  REGION 1..9: note = Anti-CD19 FMC63 scFv light chain CDR3
QQGNTLPYT

SEQ ID NO: 485 - protein, 18 aa, synthetic construct
  REGION 1..18: note = Whitlow linker
GSTSGSGKPGSGEGSTKG

SEQ ID NO: 486 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 487 - protein, 8 aa, synthetic construct
  REGION 1..8: note = Anti-CD19 FMC63 scFv heavy chain CDR1
GVSLPDYG

SEQ ID NO: 488 - protein, 7 aa, synthetic construct
  REGION 1..7: note = Anti-CD19 FMC63 scFv heavy chain CDR2
IWGSETT

SEQ ID NO: 489 - protein, 14 aa, synthetic construct
  REGION 1..14: note = Anti-CD19 FMC63 scFv heavy chain CDR3
AKHYYYGGSYAMDY

SEQ ID NO: 490 - protein, 242 aa, synthetic construct
  REGION 1..242: note = Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 491 - protein, 15 aa, synthetic construct
  REGION 1..15: note = 3xG4S linker
GGGGSGGGGSGGGGS

SEQ ID NO: 492 - DNA, 1467 bp, synthetic construct
  source 1..1467: mol_type = other DNA
  misc_feature 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 493 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 494 - DNA, 1458 bp, synthetic construct
  source 1..1458: mol_type = other DNA
  misc_feature 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 495 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 496 - DNA, 1383 bp, synthetic construct
  source 1..1383: mol_type = other DNA
  misc_feature 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 497 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 498 - DNA, 1467 bp, synthetic construct
  source 1..1467: mol_type = other DNA
  misc_feature 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 499 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 500 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 501 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 502 - protein, 10 aa, synthetic construct
  REGION 1..10: note = Anti-CD20 Leu16 scFv light chain CDR1
RASSSVNYMD

SEQ ID NO: 503 - protein, 7 aa, synthetic construct
  REGION 1..7: note = Anti-CD20 Leu16 scFv light chain CDR2
ATSNLAS

SEQ ID NO: 504 - protein, 9 aa, synthetic construct
  REGION 1..9: note = Anti-CD20 Leu16 scFv light chain CDR3
QQWSFNPPT

SEQ ID NO: 505 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 506 - protein, 5 aa, synthetic construct
  REGION 1..5: note = Anti-CD20 Leu16 scFv heavy chain CDR1
SYNMH

SEQ ID NO: 507 - protein, 17 aa, synthetic construct
  REGION 1..17: note = Anti-CD20 Leu16 scFv heavy chain CDR2
AIYPGNGDTSYNQKFKG

SEQ ID NO: 508 - protein, 13 aa, synthetic construct
  REGION 1..13: note = Anti-CD20 Leu16 scFv heavy chain CDR3
SNYYGSSYWFFDV

SEQ ID NO: 509 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 510 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 511 - protein, 10 aa, synthetic construct
  REGION 1..10: note = Anti-CD22 m971 scFv heavy chain CDR1
GDSVSSNSAA

SEQ ID NO: 512 - protein, 9 aa, synthetic construct
  REGION 1..9: note = Anti-CD22 m971 scFv heavy chain CDR2
TYYRSKWYN

SEQ ID NO: 513 - protein, 14 aa, synthetic construct
  REGION 1..14: note = Anti-CD22 m971 scFv heavy chain CDR3
AREVTGDLEDAFDI

SEQ ID NO: 514 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 515 - protein, 6 aa, synthetic construct
  REGION 1..6: note = Anti-CD22 m971 scFv light chain CDR1
QTIWSY

SEQ ID NO: 516 - skipped

SEQ ID NO: 517 - protein, 9 aa, synthetic construct
  REGION 1..9: note = Anti-CD22 m971 scFv light chain CDR3
QQSYSIPQT

SEQ ID NO: 518 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 519 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 520 - protein, 10 aa, synthetic construct
  REGION 1..10: note = Anti-CD22 m971-L7 scFv heavy chain CDR1
GDSVSSNSVA

SEQ ID NO: 521 - protein, 9 aa, synthetic construct
  REGION 1..9: note = Anti-CD22 m971-L7 scFv heavy chain CDR2
TYYRSTWYN

SEQ ID NO: 522 - protein, 14 aa, synthetic construct
  REGION 1..14: note = Anti-CD22 m971-L7 scFv heavy chain CDR3
AREVTGDLEDAFDI

SEQ ID NO: 523 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 524 - protein, 6 aa, synthetic construct
  REGION 1..6: note = Anti-CD22 m971-L7 scFv light chain CDR1
QTIWSY

SEQ ID NO: 525 - skipped

SEQ ID NO: 526 - protein, 9 aa, synthetic construct
  REGION 1..9: note = Anti-CD22 m971-L7 scFv light chain CDR3
QQSYSIPQT

SEQ ID NO: 527 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 528 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 529 - protein, 15 aa, synthetic construct
  REGION 1..15: note = Anti-BCMA C11D5.3 scFv light chain CDR1
RASESVSVIGAHLIH

SEQ ID NO: 530 - protein, 7 aa, synthetic construct
  REGION 1..7: note = Anti-BCMA C11D5.3 scFv light chain CDR2
LASNLET

SEQ ID NO: 531 - protein, 9 aa, synthetic construct
  REGION 1..9: note = Anti-BCMA C11D5.3 scFv light chain CDR3
LQSRIFPRT

SEQ ID NO: 532 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 533 - protein, 5 aa, synthetic construct
  REGION 1..5: note = Anti-BCMA C11D5.3 scFv heavy chain CDR1
DYSIN

SEQ ID NO: 534 - protein, 17 aa, synthetic construct
  REGION 1..17: note = Anti-BCMA C11D5.3 scFv heavy chain CDR2
WINTETREPAYAYDFRG

SEQ ID NO: 535 - protein, 8 aa, synthetic construct
  REGION 1..8: note = Anti-BCMA C11D5.3 scFv heavy chain CDR3
DYSYAMDY

SEQ ID NO: 536 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 537 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 538 - protein, 15 aa, synthetic construct
  REGION 1..15: note = Anti-BCMA C12A3.2 scFv light chain CDR1
RASESVTILGSHLIY

SEQ ID NO: 539 - protein, 7 aa, synthetic construct
  REGION 1..7: note = Anti-BCMA C12A3.2 scFv light chain CDR2
LASNVQT

SEQ ID NO: 540 - protein, 9 aa, synthetic construct
  REGION 1..9: note = Anti-BCMA C12A3.2 scFv light chain CDR3
LQSRTIPRT

SEQ ID NO: 541 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 542 - protein, 5 aa, synthetic construct
  REGION 1..5: note = Anti-BCMA C12A3.2 scFv heavy chain CDR1
HYSMN

SEQ ID NO: 543 - protein, 17 aa, synthetic construct
  REGION 1..17: note = Anti-BCMA C12A3.2 scFv heavy chain CDR2
RINTESGVPIYADDFKG

SEQ ID NO: 544 - protein, 8 aa, synthetic construct
  REGION 1..8: note = Anti-BCMA C12A3.2 scFv heavy chain CDR3
DYLYSLDF

SEQ ID NO: 545 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 546 - protein, 8 aa, synthetic construct
  REGION 1..8: note = Anti-BCMA FHVH33 CDR1
GFTFSSYA

SEQ ID NO: 547 - protein, 8 aa, synthetic construct
  REGION 1..8: note = Anti-BCMA FHVH33 CDR2
ISGSGDYI

SEQ ID NO: 548 - protein, 14 aa, synthetic construct
  REGION 1..14: note = Anti-BCMA FHVH33 CDR3
AKEGTGANSSLADY

SEQ ID NO: 549 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 550 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 551 - protein, 6 aa, synthetic construct
  REGION 1..6: note = Anti-BCMA CT103A scFv light chain CDR1
QSISSY

SEQ ID NO: 552 - skipped

SEQ ID NO: 553 - protein, 8 aa, synthetic construct
  REGION 1..8: note = Anti-BCMA CT103A scFv light chain CDR3
QQKYDLLT

SEQ ID NO: 554 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 555 - protein, 10 aa, synthetic construct
  REGION 1..10: note = Anti-BCMA CT103A scFv heavy chain CDR1
GGSISSSSYY

SEQ ID NO: 556 - protein, 7 aa, synthetic construct
  REGION 1..7: note = Anti-BCMA CT103A scFv heavy chain CDR2
ISYSGST

SEQ ID NO: 557 - protein, 11 aa, synthetic construct
  REGION 1..11: note = Anti-BCMA CT103A scFv heavy chain CDR3
ARDRGDTILDV

SEQ ID NO: 558 - DNA, 1503 bp, synthetic construct
  source 1..1503: mol_type = other DNA
  misc_feature 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 559 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0059239 A1
(43) Pub. Date: Feb. 20, 2025

(54) MODIFIED PARAMYXOVIRIDAE FUSION GLYCOPROTEINS

(71) Applicant: Sana Biotechnology, Inc., Seattle, WA (US)

(72) Inventors: Christopher BANDORO, Brisbane, CA (US); Lauren Pepper MACKENZIE, Belmont, MA (US); Jagesh Vijaykumar SHAH, Lexington, MA (US); Kyle Marvin TRUDEAU, Boston, MA (US)

(73) Assignee: Sana Biotechnology, Inc., Seattle, WA (US)

(21) Appl. No.: 18/720,534

(22) PCT Filed: Dec. 16, 2022
(86) PCT No.: PCT/US2022/081870

§ 371 Date: Jun. 14, 2024

Related U.S. Application Data
(60) Provisional application No. 63/408,821, filed on Sep. 21, 2022.
(60) Provisional application No. 63/291,323, filed on Dec. 17, 2021.

(51) Int. Cl. C07K 14/005; C12N 15/86

(52) U.S. Cl. CPC C07K 14/005; C12N 15/86; C12N 2740/15023; C12N 2740/15043; C12N 2760/18222

(57) ABSTRACT

Provided herein are lipid particles, such as lentiviral particles, that incorporate or are pseudotyped with a variant Nipah Virus F (NiV-F) envelope glycoprotein, and in some aspects also an attachment glycoprotein (G) protein such as a NiV-G protein or a biologically active portion or variant thereof. Also provided are polynucleotides encoding the variant NiV-F and producer cells for preparation of the lipid particles, such as lentiviral particles, containing the variant NiV-F proteins, as well as methods for preparing and using the lipid particles, such as lentiviral particles.

### CROSS-REFERENCE TO RELATED APPLICATIONS

This application claims priority to U.S. Provisional Patent Application No. 63/291,323, filed Dec. 17, 2021, entitled “MODIFIED PARAMYXOVIRIDAE FUSION GLYCOPROTEINS,” and U.S. Provisional Patent Application No. 63/408,821, filed Sep. 21, 2022, entitled “MODIFIED PARAMYXOVIRIDAE FUSION GLYCOPROTEINS,” each of which is herein incorporated by reference in its entirety for all purposes.

### REFERENCE TO AN ELECTRONIC SEQUENCE LISTING

The present application is being filed along with a Sequence Listing in electronic format. The Sequence Listing is provided as a file entitled 18615_2004540.XML created Dec. 14, 2022 which is 786,990 bytes bytes in size. The information in the electronic format of the Sequence Listing is incorporated by reference in its entirety.

### FIELD

The present disclosure relates to variant Nipah Virus F (NiV-F) envelope glycoproteins and lipid particles, such as lentiviral particles, that incorporate or are pseudotyped with a such variant glycoproteins, and in some aspects also a glycoprotein (G) protein such as a NiV-G protein or a biologically active portion or variant thereof. The present disclosure also provides polynucleotides encoding the variant NiV-F and producer cells for preparation of the lipid particles, such as lentiviral particles, containing the variant NiV-F proteins, as well as methods for preparing and using the lipid particles, such as lentiviral particles.

### BACKGROUND

Lipid particles, including viral-based particles like virus-like particles and viral vectors such as lentiviral particles, are commonly used for delivery of exogenous agents to cells. For various particles, such as lentiviral vector particles, the host range can be altered by pseudotyping with a heterologous envelope protein or modified envelope protein. The efficient preparation and production of particles with certain heterologous or modified pseudotyped envelope proteins may not always be efficient, such as due to effects of the envelope protein on low titer of the produced lentiviral vector particles. Improved lipid particles, including virus-like particles and viral vectors, that are able to be produced with a higher titer and with efficient transduction efficiency of desirable cells are needed. The provided disclosure addresses this need.

### SUMMARY

Provided herein is a lipid particle, comprising: (a) a lipid bilayer; (b) a paramyxovirus glycoprotein (G protein) or a biologically active portion thereof; and (c) a variant Nipah virus F glycoprotein (NiV-F) comprising a modified cytoplasmic tail, wherein the modified cytoplasmic tail comprises: (i) a heterologous cytoplasmic tail or a truncated portion thereof from a glycoprotein from another virus or a virus-associated protein, wherein the variant NiV-F is a chimeric protein; (ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or (iii) a modified NiV-F cytoplasmic that comprises a modified endocytosis motif, wherein the G protein or the biologically active portion thereof and the variant NiV-F protein are exposed on the outside of the lipid bilayer.

In some of any of the provided embodiments, the lipid bilayer is derived from a membrane of a host cell used for producing a retroviral vector or retrovirus-like particle, optionally a lentiviral vector or lentiviral-like particle. In some of any of the provided embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

Provided herein is a pseudotyped lentiviral particle, comprising: (a) a paramyxovirus glycoprotein (G) or a biologically active portion thereof; and (b) a variant Nipah virus F glycoprotein (NiV-F) comprising a modified cytoplasmic tail, wherein the modified cytoplasmic tail comprises: (i) a heterologous cytoplasmic tail or a truncated portion thereof from a glycoprotein from another virus or a virus-associated protein, wherein the variant NiV-F is a chimeric protein; (ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or (iii) a modified NiV-F cytoplasmic that comprises a modified endocytosis motif, wherein the G protein or the biologically active portion thereof and the variant NiV-F protein are exposed on the outside of the lipid bilayer.

In some of any of the provided embodiments, the variant NiV-F protein exhibits fusogenic activity with a target cell upon binding of the G protein to a target molecule on the target cell. In some of any of the provided embodiments, the variant NiV-F protein comprises an F0 precursor or is a proteolytically cleaved form thereof comprising F1 and F2 subunits. In some of any of the provided embodiments, the proteolytically cleaved form is a cathepsin L cleavage product. In some of any of the provided embodiments, the variant Niv-F protein comprises a modified F1 subunit containing the modified cytoplasmic tail and an F2 subunit in which (1) the F1 subunit is a modified F1 composed of the sequence of the modified cytoplasmic tail directly linked to the C-terminus of the sequence set forth in SEQ ID NO:383, and (2) the F2 subunit is set forth as SEQ ID NO:365.

In some of any of the provided embodiments, the variant NiV-F comprises in order from N-terminus to C-terminus an extracellular domain, a transmembrane domain and the modified cytoplasmic tail. In some of any of the provided embodiments, the extracellular domain and transmembrane domain of the variant NiV-F are from a wild-type NiV-F or a biologically active variant thereof.

In some of any of the provided embodiments, the extracellular domain and transmembrane domain of the variant NiV-F comprise the sequence set forth in SEQ ID NO:2, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:2. In some of any of the provided embodiments, the extracellular domain and transmembrane domain of the variant NiV-F comprise the sequence set forth in SEQ ID NO:2. In some of any of the provided embodiments, the modified cytoplasmic tail is a truncated NiV-F cytoplasmic tail that has a deletion of from 23 to 27 contiguous amino acid residues at or near the C-terminus of the wild-type Nipah virus F protein cytoplasmic tail set forth in SEQ ID NO:4. In some of any of the provided embodiments, the modified cytoplasmic tail is a truncated NiV-G cytoplasmic tail that has a deletion of at or about 23 amino acid residues at or near the C-terminus of the wild-type Nipah virus cytoplasmic tail set forth in SEQ ID NO: 4.

In some of any of the provided embodiments, the modified cytoplasmic tail is a truncated NiV-F cytoplasmic tail set forth in SEQ ID NO:27. In some of any of the provided embodiments, the variant NiV-F comprises the sequence set forth in SEQ ID NO:306, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:306. In some of any of the provided embodiments, the variant NiV-F comprises the sequence set forth in SEQ ID NO:306.

In some of any of the provided embodiments, the variant NiV-F is a chimeric protein and the modified cytoplasmic tail comprises a heterologous cytoplasmic tail or a truncated portion thereof from a glycoprotein from another virus. In some of any of the provided embodiments, the other virus is a member of the Kingdom Orthornavirae. In some of any of the provided embodiments, the other virus is a member of the family Paramyxoviridae, Rhabdoviridae, Arenaviridae, or Retroviridae. In some of any of the provided embodiments, the other virus is a member of the family Paramyxoviridae. In some of any of the provided embodiments, the other virus is a Hendra virus, Cedar virus, Canine distemper virus, Parainfluenza virus, Measles virus, Newcastle disease virus, or Sendai virus.

In some of any of the provided embodiments, the other virus is Measles virus and the glycoprotein is a Measles virus fusion (F) protein (MvF). In some of any of the provided embodiments, the heterologous cytoplasmic tail is a truncated MvF cytoplasmic tail that has a deletion of up to 32 contiguous amino acid residues at or near the C-terminus of the wild-type MvF cytoplasmic tail set forth in SEQ ID NO: 125. In some of any of the provided embodiments, the heterologous cytoplasmic tail is a truncated MvF cytoplasmic tail that has a deletion of at or about or up to 26 contiguous amino acid residues at or near the C-terminus of the wild-type MvF cytoplasmic tail set forth in SEQ ID NO: 125. In some of any of the provided embodiments, the modified cytoplasmic tail comprises the heterologous cytoplasmic tail set forth in SEQ ID NO:133. In some of any of the provided embodiments, the variant NiV-F comprises the sequence of amino acids set forth in SEQ ID NO:307, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:307. In some of any of the provided embodiments, the variant NiV-F comprises the sequence of amino acids set forth in SEQ ID NO: 307.

In some of any of the provided embodiments, the other virus is Newcastle Disease Virus (NDV) and the glycoprotein is a NDV F protein. In some of any of the provided embodiments, the heterologous cytoplasmic tail is a truncated NDV F protein cytoplasmic tail that has a deletion of up to 25 contiguous amino acid residues at or near the C-terminus of the wild-type NDV F protein cytoplasmic tail set forth in SEQ ID NO: 141. In some of any of the provided embodiments, the heterologous cytoplasmic tail is a truncated NDV F protein cytoplasmic tail that has a deletion of at or about or up to 17 contiguous amino acid residues at or near the C-terminus of the wild-type NDV F protein cytoplasmic tail set forth in SEQ ID NO: 141. In some of any of the provided embodiments, the modified cytoplasmic tail comprises the heterologous cytoplasmic tail set forth in SEQ ID NO:147.

In some of any of the provided embodiments, the variant NiV-F comprises the sequence of amino acids set forth in SEQ ID NO:308, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:308. In some of any of the provided embodiments, the variant NiV-F comprises the sequence of amino acids set forth in SEQ ID NO: 308.

In some of any of the provided embodiments, the heterologous cytoplasmic tail is a truncated NDV F protein cytoplasmic tail that has a deletion of at or about or up to 20 contiguous amino acid residues at or near the C-terminus of the wild-type NDV F protein cytoplasmic tail set forth in SEQ ID NO: 141. In some of any of the provided embodiments, the modified cytoplasmic tail comprises the heterologous cytoplasmic tail set forth in SEQ ID NO:150.

In some of any of the provided embodiments, the variant NiV-F comprises the sequence of amino acids set forth in SEQ ID NO:309, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:309. In some of any of the provided embodiments, the variant NiV-G comprises the sequence of amino acids set forth in SEQ ID NO:309.

In some of any of the provided embodiments, the other virus is Hendra virus (HeV) and the glycoprotein is a HeV F protein. In some of any of the provided embodiments, the heterologous cytoplasmic tail is a truncated HeV F cytoplasmic tail that has a deletion of up to 26 contiguous amino acid residues at or near the C-terminus of the wild-type HeV F cytoplasmic tail set forth in SEQ ID NO: 58. In some of any of the provided embodiments, the heterologous cytoplasmic tail is a truncated HeV F cytoplasmic tail that has a deletion of at or about or up to 22 contiguous amino acid residues at or near the C-terminus of the wild-type HeV F cytoplasmic tail set forth in SEQ ID NO: 58. In some of any of the provided embodiments, the modified cytoplasmic tail comprises the heterologous cytoplasmic tail set forth in SEQ ID NO:80. In some of any of the provided embodiments, the variant NiV-F comprises the sequence of amino acids set forth in SEQ ID NO:310, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:310. In some of any of the provided embodiments, the variant NiV-F comprises the sequence of amino acids set forth in SEQ ID NO: 310.

In some of any of the provided embodiments, the modified cytoplasmic tail comprises replacement of the attachment motif of the NiV-F cytoplasmic tail with a cytoplasmic tail or a truncated portion thereof form the attachment protein of another paramyxovirus. In some of any of the provided embodiments, the paramyxovirus is a Nipah virus, Hendra virus, or Measles virus. In some of any of the provided embodiments, the attachment protein is a G protein, H protein or HN protein. In some of any of the provided embodiments, the modified cytoplasmic tail comprises the heterologous cytoplasmic tail set forth in any one of SEQ ID NOS: 210-222.

In some of any of the provided embodiments, the lipid particle further comprises a modified ectodomain comprising a modified protease cleavage site. In some of any of the provided embodiments, the modified cleavage site comprises replacement of the cleavage sequence NNTHDLVGDVRLAGV (SEQ ID NO:311) with a modified furin cleavage sequence set forth in any one of SEQ ID NOS: 313-327. In some of any of the provided embodiments, the modified cleavage site comprises replacement of the cleavage sequence VGDVR (SEQ ID NO:339) with a modified cleavage sequence from another paramyxovirus, optionally wherein the modified cleavage sequence from another paramyxovirus comprises a cathepsin L cleavage site.

Provided herein is a lipid particle, comprising: (a) a lipid bilayer; (b) a paramyxovirus glycoprotein (G) or a biologically active portion thereof; and (c) a variant Nipah virus F glycoprotein (NiV-F) comprising a modified ectodomain, wherein the modified ectodomain comprises a modified cleavage site selected from: (i) replacement of the cleavage sequence NNTHDLVGDVRLAGV (SEQ ID NO:311) with a modified furin cleavage sequence; or (ii) replacement of the cleavage sequence VGDVR (SEQ ID NO:339) with a modified cleavage sequence from another paramyxovirus, optionally wherein the modified cleavage sequence from another paramyxovirus comprises a cathepsin L cleavage site.

In some of any of the provided embodiments, the lipid bilayer is derived from a membrane of a host cell used for producing a retroviral vector or retrovirus-like particle, optionally a lentiviral vector or lentiviral-like particle. In some of any of the provided embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

Provided herein is a pseudotyped lentiviral particle, comprising: (a) a paramyxovirus glycoprotein (G) or a biologically active portion thereof; and (b) a variant Nipah virus F glycoprotein (NiV-F) comprising a modified ectodomain, wherein the modified ectodomain comprises a modified cleavage site selected from: (i) replacement of the cleavage sequence NNTHDLVGDVRLAGV (SEQ ID NO:311) with a modified furin cleavage sequence; or; or (ii) replacement of the cleavage sequence VGDVR (SEQ ID NO:329) VGDVR (SEQ ID NO:339) with a modified cleavage sequence from another paramyxovirus, optionally wherein the cleavage sequence is a cathepsin L cleavage site.

In some of any of the provided embodiments, the variant NiV-F protein comprises an F0 precursor or is a proteolytically cleaved form thereof comprising F1 and F2 subunits. In some of any of the provided embodiments, the replacement cleavage sequence comprises a cathepsin L cleavage site and the proteolytically cleaved form is a cathepsin L cleavage product. In some of any of the provided embodiments, the replacement cleavage sequence comprises a modified furin cleavage site and the proteolytically cleaved from is a furin cleavage product. In some of any of the provided embodiments, the replacement is in the sequence of amino acids set forth in SEQ ID NO: 1 or SEQ ID NO:320, or a mature form thereof lacking the signal peptide (amino acids 1-26). In some of any of the provided embodiments, the furin cleavage sequence is set forth in any one of SEQ ID NOS: 313-327.

In some of any of the provided embodiments, the variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26). In some of any of the provided embodiments, the modified cleavage site from another paramyxovirus is set forth in any one of SEQ ID NOS: 329-338. In some of any of the provided embodiments, the variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26). In some of any of the provided embodiments, the variant NiV-F comprises the sequence of amino acids set forth in SEQ ID NO:239, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:239, or is a mature form thereof lacking the signal peptide (e.g. amino acids 1-26). In some of any of the provided embodiments, the variant NiV-F comprises the sequence of amino acids set forth in SEQ ID NO: 239.

In some of any of the provided embodiments, the variant NiV-F comprises a heterologous signal sequence compared to the signal sequence of wild-type NiV-F. In some of any of the provided embodiments, the variant NiV-F is encoded by a polynucleotide sequence comprising a sequence that encodes a heterologous signal sequence compared to the encoded signal sequence of wild-type NiV-F.

Provided herein is a lipid particle, comprising: (a) a lipid bilayer; (b) a paramyxovirus glycoprotein (G) or a biologically active portion thereof; and (c) a variant Nipah virus F glycoprotein (NiV-F) comprising a heterologous signal sequence, or encoded by a polynucleotide sequence comprising a sequence that encodes a heterologous signal sequence, compared to the wild-type signal sequence of NiV-F.

In some of any of the provided embodiments, the lipid bilayer is derived from a membrane of a host cell used for producing a retroviral vector or retrovirus-like particle, optionally a lentiviral vector or lentiviral-like particle. In some of any of the provided embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

Provided herein is a pseudotyped lentiviral particle, comprising: (a) a paramyxovirus glycoprotein (G) or a biologically active portion thereof; and (b) a variant Nipah virus F glycoprotein (NiV-F) comprising a heterologous signal sequence, or encoded by a polynucleotide sequence comprising a sequence that encodes a heterologous signal sequence, compared to the wild-type signal sequence of NiV-F.

In some of any of the provided embodiments, the variant NiV-F protein comprises an F0 precursor or is a proteolytically cleaved form thereof comprising F1 and F2 subunits. In some of any of the provided embodiments, the proteolytically cleaved form is a cathepsin L cleavage product. In some of any of the provided embodiments, the heterologous signal sequence is from another virus or is a mammalian signal sequence. In some of any of the provided embodiments, the other virus is a paramyxovirus, optionally a henipavirus.

In some of any of the provided embodiments, the heterologous signal sequence is a HevF signal sequence, a CevF signal sequence, a HPIV2 signal sequence, a MevF signal sequence, a NDV-F signal sequence, or a SevF signal sequence. In some of any of the provided embodiments, the heterologous signal sequence is set forth in any one of SEQ ID NOS: 340-345. In some of any of the provided embodiments, the other virus is Parainfluenza virus and the heterologous signal sequence is the Human Parainfluenza Virus 2 (HPIV2) signal sequence, optionally set forth in SEQ ID NO: 342. In some of any of the provided embodiments, the variant NiV-F comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251. In some of any of the provided embodiments, the variant NiV-F is encoded by a nucleotide sequence that encodes a variant NiV-F that comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.

In some of any of the provided embodiments, the variant NiV-F comprises the sequence of amino acids set forth in SEQ ID NO: 251. In some of any of the provided embodiments, the other virus is HIV-1, Baboon endogenous retrovirus, Cocal virus, Ebola virus, Gibon ape leukemia virus, Murine leukemia virus, or Vesicular stomatitis virus. In some of any of the provided embodiments, the heterologous signal sequence is a HIV1-Env signal sequence, BaEV signal sequence, Cocal signal sequence, EboV signal sequence, GaLV signal sequence, MLV-A signal sequence, or VSVG signal sequence. In some of any of the provided embodiments, the heterologous signal sequence is set forth in any one of SEQ ID NOS: 346-352. In some of any of the provided embodiments, the other virus is Vesicular stomatitis virus (VSV) and the heterologous signal sequence is the VSV G glycoprotein (VSV-G) signal sequence, optionally set forth in SEQ ID NO: 352. In some of any of the provided embodiments, the variant NiV-F comprises the sequence of amino acids set forth in SEQ ID NO:261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261. In some of any of the provided embodiments, the variant NiV-F is encoded by a nucleotide sequence that encodes a variant NiV-F that comprises the sequence of amino acids set forth in SEQ ID NO:261 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261. In some of any of the provided embodiments, the variant NiV-F comprises the sequence of amino acids set forth in SEQ ID NO: 261.

In some of any of the provided embodiments, the mammalian signal sequence is a signal sequence from a human protein. In some of any of the provided embodiments, the heterologous signal sequence is a Prolactin signal sequence, IgGk-L signal sequence, Albumin signal sequence, CD5 signal sequence, Trypsinogen signal sequence orIL2 signal sequence. In some of any of the provided embodiments, the heterologous signal sequence is set forth in any one of SEQ ID NOS: 353-358. In some of any of the provided embodiments, the variant NiV-F comprises a heterologous or modified transmembrane domain compared to the transmembrane domain of wild-type NiV-F.

Provided herein is a lipid particle, comprising: (a) a lipid bilayer; (b) a paramyxovirus glycoprotein (G) or a biologically active portion thereof; and (c) a variant Nipah virus F glycoprotein (NiV-F) comprising a heterologous or modified transmembrane domain compared to wild-type NiV-F.

In some of any of the provided embodiments, the lipid bilayer is derived from a membrane of a host cell used for producing a retroviral vector or retrovirus-like particle, optionally a lentiviral vector or lentiviral-like particle. In some of any of the provided embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

Provided herein is a pseudotyped lentiviral particle, comprising: (a) a paramyxovirus glycoprotein (G) or a biologically active portion thereof; and (b) a variant Nipah virus F glycoprotein (NiV-F) comprising a heterologous or modified transmembrane domain compared to wild-type NiV-F.

In some of any of the provided embodiments, the variant NiV-F protein comprises an F0 precursor or is a proteolytically cleaved form thereof comprising F1 and F2 subunits. In some of any of the provided embodiments, the proteolytically cleaved form is a cathepsin L cleavage product. In some of any of the provided embodiments, the transmembrane domain is a heterologous transmembrane domain from Hendra virus, optionally wherein the transmembrane domain is set forth in SEQ ID NO: 361. In some of any of the provided embodiments, the transmembrane domain is a modified transmembrane domain containing the amino acid replacement S490A, Y498A, S504A or I516V, corresponding to numbering of positions set forth in SEQ ID NO:1, optionally wherein the transmembrane domain is set forth in any one of SEQ ID NOS: 359, 360, 362 or 363. In some of any of the provided embodiments, the variant NiV-F comprises the sequence of amino acids set forth in any one of SEQ ID NO: 268-272 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 268-272.

In some of any of the provided embodiments, the particle further comprises a hyperfusogenic mutation. In some of any of the provided embodiments, the hyperfusogenic mutation is introduced at a N-glycosylation site with respect to amino acid positions 64, 67, 99, 414 and/or 464 of SEQ ID NO. 1. In some of any of the provided embodiments, the hyperfusogenic mutation is one or more of N64Q, N67Q, N99Q, N414Q and/or N464Q, with reference to numbering set forth in SEQ ID NO:1 In some of any of the provided embodiments, the one or more mutations are selected from the group consisting of N64Q, N67Q, N99Q, N414Q, N464Q, N67Q/N99Q, N67Q/N414Q, N67Q/N464Q, N99Q/N414Q, N99Q/N464Q, N414Q/N464Q, N67Q/N99Q/N414Q, N67Q/N414Q/N464Q, N99Q/N414Q/N464Q, or N67Q/N99Q/N414Q/N464Q, N64Q/N99Q, N64Q/N99Q/N464Q, N64Q/N67Q/N99Q, and N64Q/N67Q/N99Q/N464Q.

In some of any of the provided embodiments, the hyperfusogenic mutation is introduced at a hexamer stability site with respect to amino acid positions 393 and/or 109 of SEQ ID NO. 1. In some of any of the provided embodiments, the hyperfusogenic mutation is one or more of R109L, Q393L or R109L and Q393L, with reference to numbering set forth in SEQ ID NO:1.

Provided herein is a lipid particle, comprising: (a) a lipid bilayer; (b) a paramyxovirus glycoprotein (G) or a biologically active portion thereof; and (c) a variant Nipah virus F glycoprotein (NiV-F) comprising a hyperfusogenic mutation selected from: a mutation that is introduced at a N-glycosylation site with respect to amino acid positions 64, 67, 99, 414 and/or 464 of SEQ ID NO. 1; and/or a mutation that is introduced at a hexamer stability site with respect to amino acid positions 393 and/or 109 of SEQ ID NO. 1.

In some of any of the provided embodiments, the lipid bilayer is derived from a membrane of a host cell used for producing a retroviral vector or retrovirus-like particle, optionally a lentiviral vector or lentiviral-like particle. In some of any of the provided embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

Provided herein is a pseudotyped lentiviral particle, comprising: (a) a paramyxovirus glycoprotein (G) or a biologically active portion thereof; and (b) a variant Nipah virus F glycoprotein (NiV-F) comprising a a hyperfusogenic mutation selected from: a mutation that is introduced at a N-glycosylation site with respect to amino acid positions 64, 67, 99, 414, and/or 464 of SEQ ID NO. 1; and/or a mutation that is introduced at a hexamer stability site with respect to amino acid positions 393 and/or 109 of SEQ ID NO. 1.

In some of any of the provided embodiments, the variant NiV-F protein comprises an F0 precursor or is a proteolytically cleaved form thereof comprising F1 and F2 subunits. In some of any of the provided embodiments, the proteolytically cleaved form is a cathepsin L cleavage product. In some of any of the provided embodiments, the particle comprises a mutation N67Q, N99Q, N414Q or N464Q or any combination thereof; and/or a mutation R109L or Q393L or a combination thereof.

In some of any of the provided embodiments, the hyperfusogenic mutation is in the sequence of amino acids set forth in SEQ ID NO:1, SEQ ID NO:2, SEQ ID NO:302 or SEQ ID NO:303. In some of any of the provided embodiments, the variant NiV-F comprises the sequence of amino acids set forth in SEQ ID NO:247, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:247. In some of any of the provided embodiments, the variant NiV-F comprises the sequence of amino acids set forth in SEQ ID NO: 247.

In some of any of the provided embodiments, the variant NiV-F comprises the sequence of amino acids set forth in SEQ ID NO:292, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:292.

In some of any of the provided embodiments, the variant NiV-F comprises the sequence of amino acids set forth in SEQ ID NO: 292. In some of any of the provided embodiments, the G protein or the biologically active portion thereof is a wild-type Nipah virus G (NiV-G) protein or a Hendra virus G protein or is a functionally active variant or biologically active portion thereof. In some of any of the provided embodiments, the G protein or the biologically active portion thereof is a wild-type NiV-G protein or a functionally active variant or a biologically active portion thereof. In some of any of the provided embodiments, the G protein is a biologically active portion of wild-type NiV-G that is truncated in which the G protein lacks up to 34 contiguous amino acids at or near the N-terminus of the wild-type NiV-G set forth in SEQ ID NO:305.

In some of any of the provided embodiments, the G-protein is a NiV-G functionally active variant or a biologically active portion thereof that exhibits reduced binding to Ephrin B2 or Ephrin B3. In some of any of the provided embodiments, the NiV-G functionally active variant or a biologically active portion thereof protein comprises: one or more amino acid substitutions corresponding to amino acid substitutions selected from the group consisting of E501A, W504A, Q530A and E533A with reference to numbering set forth in SEQ ID NO:305. In some of any of the provided embodiments, the NiV-G functionally active variant or a biologically active portion thereof comprises amino acid substitutions E501A, W504A, Q530A and E533A with reference to numbering set forth in SEQ ID NO:305.

In some of any of the provided embodiments, the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301. In some of any of the provided embodiments, the NiV-G functionally active variant or a biologically active portion thereof comprises the amino acid sequence set forth in SEQ ID NO: 301.

In some of any of the provided embodiments, the G protein is linked to a binding domain that binds to a target cell surface molecule on a target cell. In some of any of the provided embodiments, the binding domain is attached to the C-terminus of the G protein. In some of any of the provided embodiments, the cell surface molecule is a protein, glycan, lipid or low molecular weight molecule. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells. In some of any of the provided embodiments, the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell.

In some of any of the provided embodiments, the target cell is a hepatocyte. In some of any of the provided embodiments, the binding domain binds to a cell surface molecule selected from the group consisting of ASGR1, ASGR2 and TM4SF. In some of any of the provided embodiments, the target cell is a T cell. In some of any of the provided embodiments, the binding domain binds to a cell surface molecule selected from the group consisting of CD3, CD4 or CD8. In some of any of the provided embodiments, the binding domain is an antibody or antigen-binding fragment, a single domain antibody or a DARPin. In some of any of the provided embodiments, the binding domain is a single domain antibody that is a VHH or is a single chain variable fragment (scFv). In some of any of the provided embodiments, the binding domain is attached to the G protein via a linker. In some of any of the provided embodiments, the linker is a peptide linker. In some of any of the provided embodiments, the peptide linker is 2 to 65 amino acids in length. In some of any of the provided embodiments, the peptide linker is a flexible linker that comprises GS, GGS, GGGGS (SEQ ID NO:287), GGGGGS (SEQ ID NO:288) or combinations thereof. In some of any of the provided embodiments, the peptide linker is selected from: (GGS)n, wherein n is 1 to 10; (GGGGS)n (SEQ ID NO:289), wherein n is 1 to 10; or (GGGGGS)n (SEQ ID NO:290), wherein n is 1 to 6.

In some of any of the provided embodiments, the particle is replication defective. In some of any of the provided embodiments, the particle is prepared by a method comprising transducing a producer cell with packaging plasmids that encode a Gag-pol, Rev, Tat and the variant NiVG and the F protein.

In some of any of the provided embodiments, the particle further comprises a viral nucleic acid. In some of any of the provided embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3). In some of any of the provided embodiments, the particle is devoid of viral genomic DNA. In some of any of the provided embodiments, the particle is produced as a preparation with increased titer compared to a reference particle preparation that is similarly produced but with incorporation of the full-length NiV-F protein set forth in SEQ ID NO: 1, optionally wherein the lipid particle or pseudotyped lentiviral particle is a lentivirus vector. In some of any of the provided embodiments, the particle is produced as a preparation with increased titer compared to a reference particle preparation that is similarly produced but with incorporation of the truncated NiV-F protein comprising the sequence set forth in SEQ ID NO:303 or encoded by a polynucleotide that encodes the sequence set forth in SEQ ID NO:302, optionally wherein the lipid particle or psuedotyped lentiviral particle is a lentivirus vector.

In some of any of the provided embodiments, the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more. In some of any of the provided embodiments, the titer is increased by about or greater than about 2.0-fold. In some of any of the provided embodiments, the titer is increased by about or greater than about 3.0-fold. In some of any of the provided embodiments, the titer is increased by about or greater than about 4.0-fold. In some of any of the provided embodiments, the titer is increased by about or greater than about 5.0-fold.

In some of any of the provided embodiments, the particle further comprises an exogenous agent for delivery to a target cell. In some of any of the provided embodiments, the exogenous agent is present in the lumen. In some of any of the provided embodiments, the exogenous agent is a protein or a nucleic acid, optionally wherein the nucleic acid is a DNA or RNA. In some of any of the provided embodiments, the exogenous agent is a nucleic acid encoding a cargo for delivery to the target cell. In some of any of the provided embodiments, the exogenous agent is or encodes a therapeutic agent or a diagnostic agent. In some of any of the provided embodiments, the exogenous agent encodes a membrane protein, optionally wherein the membrane protein is an antigen receptor for targeting cells expressed by or associated with a disease or condition. In some of any of the provided embodiments, the membrane protein is a chimeric antigen receptor (CAR). In some of any of the provided embodiments, the target cell is a T cell. In some of any of the provided embodiments, the exogenous agent is a nucleic acid comprising a payload gene for correcting a genetic deficiency, optionally a genetic deficiency in the target cell, optionally wherein the genetic deficiency is associated with a liver cell or a hepatocyte. In some of any of the provided embodiments, binding of the G protein to a cell surface molecule on the target cell mediates fusion of the particle with the target cell and delivery of the exogenous agent to the target cell. In some of any of the provided embodiments, at or greater than 10%, 20%, 30%, 40%, 50%, 60% of the target cells are delivered the exogenous agent.

In some of any of the provided embodiments, delivery of the exogenous cell to the target cell is increased compared to a reference particle preparation that is similarly produced but in which the NiV-F protein is the full-length NiV-F protein set forth in SEQ ID NO: 1, optionally wherein the lipid particle or pseudotyped lentiviral particle is a lentivirus vector. In some of any of the provided embodiments, delivery of the exogenous cell to the target cell is increased compared to a reference particle preparation that is similarly produced but in which the F protein is the truncated NiV-F comprising the sequence set forth in SEQ ID NO:303 or encoded by a polynucleotide that encodes the sequence set forth in SEQ ID NO:302, optionally wherein the lipid particle or pseudotyped lentiviral particle is a lentivirus vector. In some of any of the provided embodiments, the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

Provided herein is a polynucleotide comprising a nucleic acid molecule encoding a variant Nipah virus F glycoprotein (NiV-F) comprising a modified cytoplasmic tail, wherein the modified cytoplasmic tail comprises: (i) a heterologous cytoplasmic tail or a truncated portion thereof from a glycoprotein from another virus or a virus-associated protein, wherein the variant NiV-F is a chimeric protein; (ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or (iii) a modified NiV-F cytoplasmic that comprises a modified endocytosis motif.

In some of any of the provided embodiments, the variant NiV-F protein comprises an F0 precursor or is a proteolytically cleaved form thereof comprising F1 and F2 subunits. In some of any of the provided embodiments, the proteolytically cleaved form is a cathepsin L cleavage product. In some of any of the provided embodiments, the variant Niv-F protein protein comprises a modified F1 subunit containing the modified cytoplasmic tail and an F2 subunit in which (1) the F1 subunit is a modified F1 composed of the sequence of the modified cytoplasmic tail directly linked to the C-terminus of the sequence set forth in SEQ ID NO:383, and (2) the F2 subunit is set forth as SEQ ID NO:365.

In some of any of the provided embodiments, the variant NiV-F comprises in order from N-terminus to C-terminus the an extracellular domain, a transmembrane domain and the modified cytoplasmic tail. In some of any of the provided embodiments, the extracellular domain and transmembrane domain of the variant NiV-F are from a wild-type NiV-F or a biologically active variant thereof. In some of any of the provided embodiments, the extracellular domain and transmembrane domain of the variant NiV-F comprise the sequence set forth in SEQ ID NO:2, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:2. In some of any of the provided embodiments, the extracellular domain and transmembrane domain of the variant NiV-F comprise the sequence set forth in SEQ ID NO:2.

In some of any of the provided embodiments, the modified cytoplasmic tail comprises a heterologous cytoplasmic tail set forth in any one of SEQ ID NOS: 80, 133, 147 and 150. In some of any of the provided embodiments, the encoded variant NiV-F comprises the sequence of amino acids set forth in any one of SEQ ID NOS: 307, 308, 309, and 310. In some of any of the provided embodiments, the modified cytoplasmic tail is a truncated NiV-F cytoplasmic tail set forth in any one of SEQ ID NOS: 27. In some of any of the provided embodiments, the encoded variant NiV-F comprises the sequence of amino acids set forth in any one of SEQ ID NOS: 306.

Provided herein is a polynucleotide comprising a nucleic acid molecule encoding a variant Nipah virus F glycoprotein (NiV-F) comprising a modified ectodomain, wherein the modified ectodomain comprises a modified cleavage site selected from: (i) replacement of the cleavage sequence NNTHDLVGDVRLAGV (SEQ ID NO:311) with a modified furin cleavage sequence; or (ii) replacement of the cleavage sequence VGDVR (SEQ ID NO:339) with a modified cleavage sequence from another paramyxovirus, optionally wherein the cleavage sequence is a cathepsin L cleavage site; wherein the replacement is in the sequence of amino acids set forth in SEQ ID NO: 1 or SEQ ID NO:320, or a mature form thereof lacking the signal peptide (amino acids 1-26).

In some of any of the provided embodiments, the furin cleavage sequence is set forth in any one of SEQ ID NOS: 313-327. In some of any of the provided embodiments, the encoded variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26). In some of any of the provided embodiments, the modified cleavage site from another paramyxovirus is set forth in any one of SEQ ID NOS: 329-338. In some of any of the provided embodiments, the encoded variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26). In some of any of the provided embodiments, the encoded variant NiV-F comprises the sequence of amino acids set forth in SEQ ID NO:239, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:239, or is a mature form thereof lacking the signal peptide (e.g. amino acids 1-26).

In some of any of the provided embodiments, the encoded variant NiV-F comprises the sequence of amino acids set forth in SEQ ID NO: 239.

Provided herein is a polynucleotide comprising a nucleic acid molecule encoding a variant Nipah virus F glycoprotein (NiV-F) comprising a heterologous signal sequence compared to the wild-type signal sequence of NiV-F.

In some of any of the provided embodiments, the heterologous signal sequence is from another virus or is a mammalian signal sequence. In some of any of the provided embodiments, the other virus is a paramyxovirus, optionally a henipavirus. In some of any of the provided embodiments, the heterologous signal sequence is a HevF signal sequence, a CevF signal sequence, a HPIV2 signal sequence, a MevF signal sequence, a NDV-F signal sequence, or a SevF signal sequence. In some of any of the provided embodiments, the heterologous signal sequence is set forth in any one of SEQ ID NOS: 340-345. In some of any of the provided embodiments, the other virus is Parainfluenza virus and the heterologous signal sequence is the Human Parainfluenza Virus 2 (HPIV2) signal sequence, optionally set forth in SEQ ID NO: 342. In some of any of the provided embodiments, the encoded variant NiV-F comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251. In some of any of the provided embodiments, the encoded variant NiV-F comprises the sequence of amino acids set forth in SEQ ID NO: 251.

In some of any of the provided embodiments, the other virus is HIV-1, Baboon endogenous retrovirus, Cocal virus, Ebola virus, Gibon ape leukemia virus, Murine leukemia virus, or Vesicular stomatitis virus. In some of any of the provided embodiments, the heterologous signal sequence is a HIV1-Env signal sequence, BaEV signal sequence, Cocal signal sequence, EboV signal sequence, GaLV signal sequence, MLV-A signal sequence, or VSVG signal sequence. In some of any of the provided embodiments, the heterologous signal sequence is set forth in any one of SEQ ID NOS: 346-352. In some of any of the provided embodiments, the other virus is Vesicular stomatitis virus (VSV) and the heterologous signal sequence is the VSV G glycoprotein (VSV-G) signal sequence, optionally set forth in SEQ ID NO: 352. In some of any of the provided embodiments, the encoded variant NiV-F comprises the sequence of amino acids set forth in SEQ ID NO:261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261. In some of any of the provided embodiments, the encoded variant NiV-F comprises the sequence of amino acids set forth in SEQ ID NO: 261.

In some of any of the provided embodiments, the mammalian signal sequence is a signal sequence from a human protein. In some of any of the provided embodiments, the heterologous signal sequence is a Prolactin signal sequence, IgGk-L signal sequence, Albumin signal sequence, CD5 signal sequence, Trypsinogen signal sequence orIL2 signal sequence. In some of any of the provided embodiments, the heterologous signal sequence is set forth in any one of SEQ ID NOS: 353-358.

Provided herein is a polynucleotide comprising a nucleic acid molecule encoding a variant Nipah virus F glycoprotein (NiV-F) comprising a heterologous or modified transmembrane domain compared to wild-type NiV-F.

In some of any of the provided embodiments, the transmembrane domain is a heterologous transmembrane domain from Hendra virus, optionally wherein the transmembrane domain is set forth in SEQ ID NO: 361. In some of any of the provided embodiments, the transmembrane domain is a modified transmembrane domain containing the amino acid replacement S490A, Y498A, S504A or I516V, corresponding to numbering of positions set forth in SEQ ID NO:1, optionally wherein the transmembrane domain is set forth in any one of SEQ ID NOS: 359, 360, 362 or 363. In some of any of the provided embodiments, the encoded variant NiV-F comprises the sequence of amino acids set forth in any one of SEQ ID NO: 268-272 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 268-272.

Provided herein is a polynucleotide comprising a nucleic acid molecule encoding a variant Nipah virus F glycoprotein (NiV-F) comprising a a hyperfusogenic mutation selected from: a mutation that is introduced at a N-glycosylation site with respect to amino acid positions 64, 67, and/or 99 of SEQ ID NO. 1; and/or a mutation that is introduced at a hexamer stability site with respect to amino acid positions 393 and/or 109 of SEQ ID NO. 1.

In some of any of the provided embodiments, the encoded variant NiV-F comprises the sequence of amino acids set forth in SEQ ID NO:247, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:247. In some of any of the provided embodiments, the variant NiV-F comprises the sequence of amino acids set forth in SEQ ID NO: 247. In some of any of the provided embodiments, the encoded variant NiV-F comprises the sequence of amino acids set forth in SEQ ID NO:292, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:292. In some of any of the provided embodiments, the encoded variant NiV-F comprises the sequence of amino acids set forth in SEQ ID NO: 292.

In some of any of the provided embodiments, the nucleic acid sequence is a first nucleic acid sequence and the polynucleotide further comprises a second nucleic acid sequence encoding a paramyxovirus fusion (G) protein molecule or a biologically active portion thereof or functionally active variant thereof. In some of any of the provided embodiments, the G protein or the biologically active portion thereof is a wild-type Nipah virus G (NiV-G) protein or a Hendra virus G protein or is a functionally active variant or biologically active portion thereof. In some of any of the provided embodiments, the G protein or the biologically active portion thereof is a wild-type NiV-G protein or a functionally active variant or a biologically active portion thereof. In some of any of the provided embodiments, the G protein is a biologically active portion of wild-type NiV-G that is truncated in which the G protein lacks up to 34 contiguous amino acids at or near the N-terminus of the wild-type NiV-G set forth in SEQ ID NO:305.

In some of any of the provided embodiments, the polynucleotide exhibits reduced binding to Ephrin B2 or Ephrin B3. In some of any of the provided embodiments, the NiV-G functionally active variant or a biologically active portion thereof protein comprises: one or more amino acid substitutions corresponding to amino acid substitutions selected from the group consisting of E501A, W504A, Q530A and E533A with reference to numbering set forth in SEQ ID NO:305. In some of any of the provided embodiments, the NiV-G functionally active variant or a biologically active portion thereof comprises amino acid substitutions E501A, W504A, Q530A and E533A with reference to numbering set forth in SEQ ID NO:305. In some of any of the provided embodiments, the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301. In some of any of the provided embodiments, the NiV-G functionally active variant or a biologically active portion thereof comprises the amino acid sequence set forth in SEQ ID NO: 301.

In some of any of the provided embodiments, the G protein is linked to a binding domain that binds to a target cell surface molecule on a target cell. In some of any of the provided embodiments, the binding domain is attached to the C-terminus of the G protein. In some of any of the provided embodiments, the cell surface molecule is a protein, glycan, lipid or low molecular weight molecule. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells. In some of any of the provided embodiments, the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell. In some of any of the provided embodiments, the target cell is a hepatocyte. In some of any of the provided embodiments, the binding domain binds to a cell surface molecule selected from the group consisting of ASGR1, ASGR2 and TM4SF. In some of any of the provided embodiments, the target cell is a T cell. In some of any of the provided embodiments, the binding domain binds to a cell surface molecule selected from the group consisting of CD3, CD4 or CD8. In some of any of the provided embodiments, the binding domain is an antibody or antigen-binding fragment, a single domain antibody or a DARPin. In some of any of the provided embodiments, the binding domain is a single domain antibody that is a VHH or is a single chain variable fragment (scFv).

In some of any of the provided embodiments, the binding domain is attached to the G protein via a linker. In some of any of the provided embodiments, the linker is a peptide linker. In some of any of the provided embodiments, the peptide linker is 2 to 65 amino acids in length. In some of any of the provided embodiments, the peptide linker is a flexible linker that comprises GS, GGS, GGGGS (SEQ ID NO:287), GGGGGS (SEQ ID NO:288) or combinations thereof. In some of any of the provided embodiments, the peptide linker is selected from: (GGS)n, wherein n is 1 to 10; (GGGGS)n (SEQ ID NO:289), wherein n is 1 to 10; or (GGGGGS)n (SEQ ID NO:290), wherein n is 1 to 6. In some of any of the provided embodiments, the polynucleotide comprises an IRES or a sequence encoding a linking peptide between the first and second nucleic acid sequences, optionally, wherein the linking peptide is a self-cleaving peptide or a peptide that causes ribosome skipping, optionally a T2A peptide. In some of any of the provided embodiments, the polynucleotides further comprise at least one promoter that is operatively linked to control expression of the nucleic acid, optionally expression of the first nucleic acid sequence and the second nucleic acid sequence.

Provided herein is a vector comprising any of the provided polynucleotides.

In some of any of the provided embodiments, the vector is a mammalian vector, viral vector or artificial chromosome, optionally wherein the artificial chromosome is a bacterial artificial chromosome (BAC).

Provided herein is a plasmid comprising any of the provided polynucleotides.

In some of any of the provided embodiments, the plasmid further comprises one or more nucleic acids encoding proteins for lentivirus production.

Provided herein is a cell comprising any of the provided nucleotides, or vectors, or plasmids.

Provided herein is a method of making a lipid particle comprising a variant Nipah virus F protein and, optionally a paramyxovirus G protein, comprising a) providing a cell that comprises the polynucleotide of any of the provided embodiments, or the vector of any of the provided embodiments, or the plasmid of any of the provided embodiments; b) culturing the cell under conditions that allow for production of a lipid particle, and c) separating, enriching, or purifying the lipid particle from the cell, thereby making the lipid particle.

Provided herein is a method of making a pseudotyped lentiviral vector, comprising: a) providing a producer cell that comprises a lentiviral viral nucleic acid(s), and the polynucleotide of any of the provided embodiments, or the vector of any of the provided embodiments, or the plasmid of any of the provided embodiments; b) culturing the cell under conditions that allow for production of the lentiviral vector, and c) separating, enriching, or purifying the lentiviral vector from the cell, thereby making the pseudotyped lentiviral vector.

In some of any of the provided embodiments, prior to step (b) the method further comprises providing the cell a polynucleotide encoding a henipavirus F protein molecule or biologically active portion thereof. In some of any of the provided embodiments, the cell is a mammalian cell. In some of any of the provided embodiments, the cell is a producer cell comprising viral nucleic acid, optionally retroviral nucleic acid or lentiviral nucleic acid, and the targeted lipid particle is a viral particle or a viral-like particle, optionally a retroviral particle or a retroviral-like particle, optionally a lentiviral particle or lentiviral-like particle.

Provided herein is a producer cell comprising any of the provided polynucleotides, vectors, or plasmids. In some of any of the provided embodiments, the producer cell further comprises a paramyxovirus G protein or a biologically active portion thereof. In some of any of the provided embodiments, the G protein or the biologically active portion thereof is a wild-type Nipah virus G (NiV-G) protein or a Hendra virus G protein or is a functionally active variant or biologically active portion thereof. In some of any of the provided embodiments, the G protein or the biologically active portion thereof is a wild-type NiV-G protein or a functionally active variant or a biologically active portion thereof. In some of any of the provided embodiments, the G protein is a biologically active portion of wild-type NiV-G that is truncated in which the G protein lacks up to 34 contiguous amino acids at or near the N-terminus of the wild-type NiV-G set forth in SEQ ID NO:305. In some of any of the provided embodiments, the producer cell exhibits reduced binding to Ephrin B2 or Ephrin B3. In some of any of the provided embodiments, the NiV-G is a functionally active variant or a biologically active portion thereof protein comprising: one or more amino acid substitutions corresponding to amino acid substitutions selected from the group consisting of E501A, W504A, Q530A and E533A with reference to numbering set forth in SEQ ID NO:305. In some of any of the provided embodiments, the NiV-G functionally active variant or a biologically active portion thereof comprises amino acid substitutions E501A, W504A, Q530A and E533A with reference to numbering set forth in SEQ ID NO:305. In some of any of the provided embodiments, the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301. In some of any of the provided embodiments, the NiV-G functionally active variant or a biologically active portion thereof comprises the amino acid sequence set forth in SEQ ID NO: 301.

In some of any of the provided embodiments, the G protein is linked to a binding domain that binds to a target cell surface molecule on a target cell. In some of any of the provided embodiments, the cell further comprises a viral nucleic acid, optionally wherein the viral nucleic acid is a lentiviral nucleic acid.

Provided herein is a lipid particle or pseudotyped lentiviral vector produced by any of the provided methods or producer cells.

Provided herein is a composition comprising a plurality of any of the provided lipid particles or a plurality of any of the provided lentiviral vectors

In some of any of the provided embodiments, the composition further comprises a pharmaceutically acceptable carrier.

Provided herein is a method of transducing a cell comprising transducing a cell with any of the provided lentiviral vectors or any of the provided composition comprising any of the provided lentiviral vector or any of the provided plurality of lentiviral vectors.

Provided herein is a method of delivering an exogenous agent to a subject (e.g., a human subject), the method comprising administering to the subject any of the provided lipid particle or lentiviral vector or any of the provided composition comprising any of the provided lipid particle or any of the provided lentiviral vector or any of the provided composition comprising a lentiviral vector or plurality of lentiviral vectors, wherein lipid particle or lentiviral vector comprise the exogenous agent.

Provided herein is method of delivering an exogenous agent to a target cell, the method contacting a target cell with any of the provided lipid particle or lentiviral vector or any of the provided composition or plurality of lentiviral vectors, wherein the lipid particle or lentiviral vector comprise the exogenous agent. In some of any of the provided embodiments, the contacting transduces the cell with lentiviral vector or the lipid particle. In some of any of the provided embodiments, the contacting is in vivo in a subject.

Provided herein is a method of treating a disease or disorder in a subject (e.g., a human subject), the method comprising administering to the subject any of the provided lipid particle or any of the provided lentiviral vector or any of the provided compositions.

Provided herein is a method of fusing a mammalian cell to a lipid particle, the method comprising administering to the subject any of the provided lipid particle or the lentiviral vector or any of the provided compositions. In some of any of the provided embodiments, the fusing of the mammalian cell to the lipid particle delivers an exogenous agent to a subject (e.g., a human subject).

### BRIEF DESCRIPTION OF THE DRAWINGS

FIG. 1 depicts a Nipah virus F (NiV-F) glycoprotein with extracellular, transmembrane (TM) and cytoplasmic domains annotated.

FIG. 2 depicts titer for lentiviral lipid particles pseudotyped with exemplary chimeric NiV-F constructs containing a truncated cytoplasmic tail derived from another Paramyxovirus.

### DETAILED DESCRIPTION

Provided herein are variant Nipah virus F glycoproteins (NiV-F). In some embodiments, any of the provided lipid particles contains a variant viral fusion (F) protein molecule or a biologically active portion thereof embedded in the lipid bilayer. In some embodiments, the variant F protein is from a Paramyxovirus, a Hendra (HeV) or a Nipah (NiV) virus, or is a biologically active portion thereof or is a variant or mutant thereof. In particular embodiments, the variant F protein is from a Nipah (NiV) virus. Naturally, the fusion (F) and attachment glycoproteins (G) mediate cellular entry of paramyxovirus, such as Nipah virus. In some embodiments, the combination of a variant F protein, such as a variant NiV-F protein, and NiV-G protein as provided herein is able to mediate cellular entry of a provided lipid particle (e.g. lentiviral vector).

The F protein, such as Nipah Virus F protein, also known as NiV-F, is a class I fusion protein that has structural and functional features in common with fusion proteins of many families (e.g., HIV-1 gp41 or influenza virus hemagglutinin [HA]), such as an ectodomain with a hydrophobic fusion peptide and two heptad repeat regions (White J M et al. 2008. Crit Rev Biochem Mol Biol 43:189-219). F proteins are synthesized as inactive precursors F₀ and are activated by proteolytic cleavage into the two disulfide-linked subunits F₁ and F₂ (Moll M. et al. 2004. J. Virol. 78(18): 9705-9712).

G proteins are attachment proteins of henipavirus (e.g. Nipah virus or Hendra virus) that are type II transmembrane glycoproteins containing an N-terminal cytoplasmic tail, a transmembrane domain, an extracellular stalk, and a globular head (Liu, Q. et al. 2015. Journal of Virology, 89(3):1838-1850). The attachment protein, NiV-G, recognizes the receptors EphrinB2 and EphrinB3. Binding of the receptor to NiV-G triggers a series of conformational changes that eventually lead to the triggering of NiV-F, which exposes the fusion peptide of NiV-F, allowing another series of conformational changes that lead to virus-cell membrane fusion (Stone J. A. et al. 2016. J Virol. 90(23): 10762-10773). EphrinB2 was previously identified as the primary NiV receptor (Negrete et al., 2005), as well as ephrinB3 as an alternate receptor (Negrete et al., 2006). Wild-type NiV-G has a high affinity for ephrinB2 and B3, with affinity binding constants (Kd) in the picomolar range (Negrete et al., 2006) (Kd=0.06 nM and 0.58 nM for cell surface expressed ephrinB2 and B3, respectively).

In aspects of the provided embodiments, a lipid particle provided herein can be engineered to express a variant F protein molecule or biologically active portion thereof; and a NiV-G protein, in which both the variant F protein and the NiV-G protein are embedded in the lipid bilayer of the lipid particle. In some embodiments, the lipid particles can be a virus-like particle, a virus, or a viral vector, such as a lentiviral vector. In some embodiments, provided herein is a lentiviral vector pseudotyped with the combination of a variant NiV-F protein, such as any of the provided variant NiV-F proteins, and an G protein.

In some embodiments, the NiV-G protein may be further linked to a targeting moiety, such as an antigen binding domain, to facilitate specific targeting of the lipid particle to a target molecule for fusion with a desired target cell. In some embodiments, a binding domain is any domain that binds to a cell surface molecule on a target cell. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

Thus, the provided embodiments include embodiments in which the NiV-G protein may be re-targeted to any desired cell type for specific targeting of a lipid particle (e.g. lentiviral vector) and, in some cases, specific delivery to a target cell of a transgene or heterologous protein contained therein.

In provided embodiments, the lipid particles contain a lipid bilayer enclosing a lumen or cavity and a variant NiV-F protein containing the modified cytoplasmic tail domain, a stalk domain, a head domain, wherein at least a portion of the cytoplasmic tail is not that of the Nipah F glycoprotein.

The efficiency of transduction of lipid particles can be improved by engineering mutations in one or both of NiV-F and NiV-G. Several such mutations have been previously described (see, e.g., Lee at al, 2011, Trends in Microbiology). This could be useful, for example, for maintaining the specificity and picomolar affinity of NiV-G for ephrinB2 and/or B3. Additionally, mutations in NiV-G that completely abrogate ephrinB2 and B3 binding, but that do not impact the association of this NiV-G with NiV-F, have been identified (Aguilar, et al. J Biol Chem. 2009; 284(3):1628-1635.; Weise et al. J Virol. 2010; 84(15):7634-764; Negrete et al., J Virol. 2007; 81(19):10804-10814; Negrete et al. PLoS Pathog. 2006; Guillaume et al., J. Virol 2006, 80 (15) 7546-7554). In some cases, methods to improve targeting of lipid particles can be achieved by fusion of a binding molecule with a G protein (e.g. Niv-G, including a Niv-G with mutations to abrogate Ephrin B2 and Ephrin B3 binding). This allows for altered G protein tropism allowing for targeting of other desired cell types that are not ephrinB2+ through the addition of the binding molecule directed against a different cell surface molecule. Thus, in provided aspects, a NiV-G protein may further contain a mutation to reduce or abrogate binding to Ephrin B2 and/B3. In some embodiments, the mutations can include one or more of mutations E501A, W504A, Q530A and E533A, with reference to numbering of wild-type NiV-G set forth in SEQ ID NO:5.

The provided lipid particles, such as lentiviral vectors, containing a variant NiV-F exhibit advantages over available envelope-pseudotyped particles. For instance, VSV-G is the most common envelope glycoprotein used for pseudotyping but its broad tropism is often not ideal or desirable for specific target cell delivery, such as is desired for gene therapy or exogenous protein delivery. Further, although alternative envelope proteins may exhibit reduced tropism or may be amenable to linkage to a binding domain for redirected targeting to a desired target cells, the titer of a preparation of lentiviral vectors containing such envelope proteins may be too low to allow for efficient transduction. Thus, alternative approaches are needed.

It is found herein that certain variant NiV-F proteins when pseudotyped on a lentiviral vector provide lentiviral vectors that exhibit high titers. Further, in provided aspects, the titers are superior to other shorter NiV-F cytoplasmic domain truncations, including those that have been previously identified as being able to provide lentiviral vectors with high titers (Bender et al. 2016, 12:e1005641). For instance, while certain cytoplasmic truncations of NiV-F have been found to increase titer of a lentiviral preparation, the findings herein demonstrate the superiority of particular longer cytoplasmic tail truncations and/or the superiority of certain variant NiV-F that are a chimeric NiV-F with a cytoplasmic tail composed in all or part by a cytoplasmic tail of a heterologous virus or protein. In particular, it is found herein that certain variant NiV-F proteins as provided herein result in improved titer of a lentiviral preparation. Finally, combining the variant NiV-F proteins with a G protein (e.g. a NiV-G or a biologically active portion or variant thereof) retains high fusion activity of the lentiviral vector, and in some cases also delivery of a transgene or other exogenous agent, to a target cell.

Also provided are lipid particles, such as targeted lipid particles, additionally containing one or more exogenous agents, such as for delivery of a diagnostic or therapeutic agent to cells, including following in vivo administration to a subject. Also provided herein are methods and uses of the targeted lipid particles, such in diagnostic and therapeutic methods. Also provided are polynucleotides, methods for engineering, preparing, and producing the targeted lipid non-cell particles, compositions containing the particles, and kits and devices containing and for using, producing and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. In some embodiments, the fusosome is derived from a source cell. A lipid particle also may include an exogenous agent or a nucleic acid encoding an exogenous agent, which may be present in the lumen of the lipid particle.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome. in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G proteins such as those from Nipah Virus (NiV) and biologically active portions or variants thereof including any as described.

As used herein, a “re-targeted fusogen,” such as a re-targeted G protein, refers to a fusogen that comprises a targeting moiety having a sequence that is not part of the naturally-occurring form of the fusogen in which the targeting moiety targets or binds a molecule on a desired cell type. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In some such embodiments, the attachment of the targeting moiety to a fusogen (e.g. G protein) may be directly or indirectly via a linker, such as a peptide linker. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity can include 10%-150% or more of the activity of a full-length or wild-type F protein or G protein. Examples of biologically active portions of F and G proteins include proteins with truncations of the cytoplasmic domain, such as any of the described variant NiV-F with a truncated cytoplasmic tail.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved binding.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle, such as a viral vector, refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### II. VARIANT NIPAH F GLYCOPROTEINS

Provided herein are variant Nipah virus fusion (NiV-F) proteins, and lipid particles (e.g. lentiviral vectors) containing the same. In some embodiments, the Niv-F proteins are modified by one or more of amino acid replacement (substitution), insertion or deletion of amino acid residues compared to a wild-type NiV-F set forth in SEQ ID NO:1 or a mature form thereof lacking the signal peptide (i.e. corresponding to amino acid residues 1-26 of SEQ ID NO:1). In some embodiments, the variant NiV-F protein contains a swap of contiguous amino acid residues from another virus, such as another Paramyxovirus. In some embodiments, the cytoplasmic tail, transmembrane domain and/or ectodomain is modified (e.g. such as by one or more amino acid substitution, insertion or deletion of amino acids). In some embodiments, the ectodomain is modified to alter (e.g. improve) protease cleavage and/or to increase fusogenicity.

Also provided herein are viral particles or viral-like particles, such as lentiviral particles or lentiviral-like particles, that are pseudotyped with any of the provided variant NiV-F proteins. Also provided herein are polynucleotides encoding the variant NiV-F proteins, which, in some aspects, can be used in connection with methods of producing a lipid particle (e.g. lentiviral particle) containing a variant NiV-F as described embedded in its lipid bilayer. In some embodiments, the variant NiV-F is exposed on the surface of the lipid bilayer, such as exposed on the surface of a lentiviral particle. In some embodiments, any of the provided lipid particles (e.g. lentiviral vectors) may also contain an G protein, such as a NiV-G protein, such as a full-length NiV-G protein or a biologically active portion thereof or a variant thereof. For instance, also provided herein are viral particles or viral-like particles, such as lentiviral particles or lentiviral-like particles, that are pseudotyped with any of the provided variant NiV-F proteins and a NiV-G protein, such as a full-length NiV-G protein or a biologically active portion or a variant thereof. Exemplary NiV-G proteins are further described in Section III.

In some embodiments, the variant NiV-F protein is disposed in the lipid bilayer. In some embodiments, the membrane is a plasma cell membrane. In some embodiments, the lipid particle is a retroviral vector, such as a lentiviral vector. In some embodiments, the viral vector, such as retroviral vector (e.g. lentiviral vector) is pseudotyped with the variant NiV-F protein.

In some embodiments, the variant NiV-F protein exhibits fusogenic activity. In some embodiments, the variant NiV-F facilitates the fusion of the lipid particle (e.g. lentiviral vector) to a membrane. F proteins of henipaviruses, including NiV-F and variant NiV-F proteins provided herein are encoded as F0 precursors containing a signal peptide (e.g. for native NiV-F the signal peptide corresponds to amino acid residues 1-26 of SEQ ID NO:1). Following cleavage of the signal peptide, the mature F0 (e.g. SEQ ID NO:1 lacking the signal peptide) is transported to the cell surface, then endocytosed and cleaved by cathepsin L (e.g. between amino acids corresponding to amino acids between amino acids 109-110 of SEQ ID NO:1) into the mature fusogenic subunits F1 (e.g. corresponding to amino acids 110-546 of SEQ ID NO:1) and F2 (e.g. corresponding to amino acid residues 27-109 of SEQ ID NO:1). The F1 and F2 subunits are associated by a disulfide bond and recycled back to the cell surface. The F1 subunit contains the fusion peptide domain located at the N terminus of the F1 subunit (e.g. for native NiV-F corresponding to amino acids 110-129 of SEQ ID NO:1) where it is able to insert into a cell membrane to drive fusion. In particular cases, fusion activity is blocked by association of the F protein with G protein (e.g. a NiV-G as provided herein), until G engages with a target molecule resulting in its disassociation from F and exposure of the fusion peptide to mediate membrane fusion. For wild-type NiV-F, the F protein has the sequence set forth in SEQ ID NO:364. In some examples, the wild-type F protein is cleaved into an F1 subunit comprising the sequence set forth in SEQ ID NO:366 and an F2 subunit comprising the sequence set forth in SEQ ID NO: 365. In some embodiments, the variant NiV-F of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:1) to produce two chains that can be linked by disulfide bond.

In some embodiments, the variant NiV-F protein is a variant F₀ precursor or is a proteolytically cleaved version thereof containing the F1 and F2 subunit linked by a disulfide bond. Hence, it is understood that reference to a particular sequence (SEQ ID NO) of a variant NiV-F herein is typically with reference to the F₀ precursor sequence but also is understood to include the proteolytically cleaved form or sequence thereof containing the two cleaved chains, F1 and F2. In some embodiments, the variant NiV-F contains a cathepsin L cleavage site (e.g. VGDVR (SEQ ID NO:339), such as NNTHDLVGDVRLAGV (SEQ ID NO:311)) and the proteolytically cleaved form is a cathepsin L cleavage product. In some embodiments, the cathepsin L cleavage product includes the two chains (F1 and F2) formed by cleavage that occurs by cleavage after an Arginine or Lysine residue, such as at a position corresponding to position 109 of NiV-F protein with reference to SEQ ID NO:1. In some embodiments, the variant NiV-F contains a modified cleavage sequence as described herein, such as a modified furin cleavage sequence, and the proteolytically cleaved from is a furin cleavage product. The encoding nucleic acid also can encode a signal peptide sequence that has the sequence MVVILDKRCY CNLLILILMI SECSVG (e.g. SEQ ID NO:3).

In some embodiments, the variant NiV-F proteins provided herein retain the cleavage site cleaved by cathepsin L (e.g. corresponding to the cleavage site between amino acids 109-110 of SEQ ID NO:1). In some embodiments, the variant NiV-F proteins provided herein is modified at or near the native cathepsin L cleavage site (e.g. corresponding to the cleavage site between amino acids 109-110 of SEQ ID NO:1) in order to improve cleavage of NiV-F and its expression on the surface of the lipid particle.

In some embodiments, a variant NiV-F protein provided herein comprises an F1 subunit or a fusogenic portion thereof. In some embodiments, the F1 subunit is a proteolytically cleaved portion of the F0 precursor. In some embodiments, the F0 precursor is inactive. In some embodiments, the cleavage of the F0 precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at an Arginine or Lysine residue corresponding to position 109 of NiV-F protein with reference to SEQ ID NO:1 (see also NiV-F cleavage site sequence set forth in SEQ ID NO: 311). In some embodiments, the variant NiV-F proteins provided herein contains an N-terminal hydrophobic fusion peptide domain that is exposed on the outside of lipid bilayer

In some embodiments, the lipid particle further contains a G protein from a paramyxovirus, such as a Nipah glycoprotein (NiV-G) to facilitate fusogenic activity of the variant NiV-F protein. Exemplary G proteins are described in Section III.B below. In some embodiments, the variant NiV-F protein and the G protein (e.g. a NiV-G or variant or biologically active portion thereof) are both disposed in the lipid bilayer. It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g. lentiviral vector), the F protein is heterologous to the G protein, i.e. the F and G protein or biologically active portions are from different henipavirus species. For example, the G protein is from Hendra virus and the variant F protein is a variant NiV-F as described. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus.

In aspects of the description provided herein, F protein sequences may be disclosed herein as expressed sequences including an N-terminal signal sequence. It is understood that as such N-terminal signal sequences are commonly cleaved co- or post-translationally, the mature protein sequences for all F protein sequences disclosed herein are also contemplated as lacking the N-terminal signal sequence.

In some embodiments, titer of the lipid particles following introduction into target cells, such as by transduction (e.g. transduced cells), is increased compared to titer into the same target cells of a reference lipid particle preparation (e.g. reference lentiviral vector). In some embodiments, the reference lipid particles (e.g. reference lentiviral vector) is a similar preparation except that the lipid particles incorporate a full-length native NiV-F wild-type sequence (e.g. set forth in SEQ ID NO:1). In some embodiments, the reference lipid particles (e.g. reference lentiviral vector) is a similar preparation except that the lipid particles incorporate a truncated NiV-F with a cytoplasmic tail truncation of 22 N-terminal amino acids compared to the full-length NiV-F, such as a truncated NiV-F set forth in SEQ ID NO: 302 or the mature form thereof lacking a signal peptide set forth in SEQ ID NO:303. In some embodiments, the reference lipid particles (e.g. reference lentiviral vector) and a provided lipid particle also incorporate a NiV-G protein, which is the same (e.g. any as described in Section III). In some embodiments, the NiV-G is a full-length native NiV-G wild-type sequence (e.g. set forth in SEQ ID NO:304). In some embodiments, the NiV-G is a full-length NiV-G sequence that contains amino acid substitutions E501 A, W504A, Q530A and/or E533A to reduce binding to Ephrin B2 and B3 (e.g. set forth in SEQ ID NO:305). In some embodiments, the NiV-G is a truncated NiV-G with a cytoplasmic tail truncation of 34 C-terminal amino acid residues and contains amino acid substitutions E501 A, W504A, Q530A, E533A compared to the full-length NiV-G, such as a truncated NiV-G set forth in SEQ ID NO:301. In some embodiments, the titer is increased compared to a reference lentiviral particle preparation by at or greater than 5%, 10%, 20%, 30%, 40%, 50%, 60%, 70%, 80%, 90%, 100%, 125%, 150%, 200%, 300%, 400%, 500% or more, compared to titer of the reference lipid particles (e.g. reference lentiviral vector). In some examples, the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, compared to the titer of the reference lipid particles (e.g. reference lentiviral vectors). In some embodiments, the titer of the lipid particles in target cells (e.g. transduced cells) is greater than at or about 1×10⁶ transduction units (TU)/mL. For example, the titer of the lipid particles in target cells (e.g. transduced cells) is greater than at or about 2×10⁶ TU/mL, greater than at or about 3×10⁶ TU/mL, greater than at or about 4×10⁶ TU/mL, greater than at or about 5×10⁶ TU/mL, greater than at or about 6×10⁶ TU/mL, greater than at or about 7×10⁶ TU/mL, greater than at or about 8×10⁶ TU/mL, greater than at or about 9×10⁶ TU/mL, or greater than at or about 1×10⁷ TU/mL.

Any techniques for assessing or quantifying titer may be employed. Non-limiting examples of available techniques for quantifying titer include viral particle number determination and titer by plaque assay. For example, the number of viral-based particles can be determined by measuring the absorbance at A260. Similarly, titer of infectious units (i.e., viral-based particles) can also be determined by quantitative immunofluorescence of particle specific proteins using monoclonal antibodies or by plaque assay. In some embodiments, methods that calculate the titer include the plaque assay, in which titrations of the viral-based particles are grown on cell monolayers and the number of plaques is counted after several days to several weeks. In some embodiments, titer can be determined using an endpoint dilution (TCIDso) method, which determines the dilution of virus at which 50% of the cell cultures are infected/transduced and hence, generally, can determine the titer within a certain range, such as one log.

Subsections below provided description of exemplary variant NiV-F proteins provided herein.

### A. Variant NiV-F Cytoplasmic Tails

In some embodiments, the variant NiV-F comprises a modified NiV-F cytoplasmic tail in which is contained a truncated NiV-F cytoplasmic tail, one or more modifications that regulates Niv-F endocytic trafficking and exposure to a cathepsin (e.g. cathepsin L), or a swap of all or a portion of the cytoplasmic tail of NiV-F with (i) a cytoplasmic tail from a related paramyxovirus F protein or a truncated portion thereof, (ii) a cytoplasmic tail from a related G/H protein or a truncated portion thereof, (iii) a cytoplasmic tail from other viral fusogens, or (iv) a cytoplasmic tail from HIV-1 envelope (gp41) or other retroviral-associated cellular protein. In some embodiments, any of the provided modifications in the cytoplasmic tail of a variant NiV-F described herein can be combined with a modification of the ectodomain, transmembrane domain or signal sequence as described herein.

### 1. Truncated Nipah F Protein Cytoplasmic Tails

In some embodiments, the variant NiV-F comprises a modified cytoplasmic tail which comprises a truncated cytoplasmic tail compared to full-length NiV-F. For instance, the variant NiV-F protein herein is truncated in its cytoplasmic tail compared to the native cytoplasmic tail of wild-type NiV-F set forth in SEQ ID NO:1 (in which the native cytoplasmic tail is set forth as amino acids 519-546 of SEQ ID NO:1). In some embodiments, the modified cytoplasmic tail is a truncated NiV-F cytoplasmic tail that has deletion of one amino acid or more than one contiguous amino acid residues at or near the C-terminus of the wild-type NiV-F cytoplasmic tail set forth in SEQ ID NO:4.

In some embodiments, the variant NiV-F contains a modified cytoplasmic tail in which at least a part of the native cytoplasmic tail (e.g. amino acids 519-546 of SEQ ID NO:1, as set forth in SEQ ID NO:4) is truncated. In some embodiments, the cytoplasmic tail is a truncated portion thereof that is at least 1 amino acid in length. In some embodiments, the cytoplasmic tail is a truncated portion thereof that is at least 5 amino acids in length. In some embodiments, the truncated portion thereof is from or from about 1-5, from or from about 5-20, from or from about 5-10, from or from about 1-15, from or from about 1-10, from or from about 1-20, from or from about 1-25, from or from about 1-26, from or from about 1-27 amino acids in length. In some embodiments, the truncated portion thereof is 1, 2, 3, 4, 5, 6, 7, 8, 9, 10, 11, 12, 13, 14, 15, 16, 17, 18, 19, 20, 21, 22, 23, 24, 25, 26, 27, or 28 amino acids in length. In some embodiments, the variant NiV-F is a sequence composed of the modified cytoplasmic tail (truncated cytoplasmic tail) sequence directly linked to the C terminus of the sequence set forth in SEQ ID NO: 2 (hereinafter also called NiV-F backbone ΔCT, containing the ectodomain and transmembrane domain of wild-type NiV-G).

In some embodiments, the variant NiV-F has a cytoplasmic tail that is a truncated NiV-cytoplasmic tail. In some embodiments, the truncated NiV-cytoplasmic tail has a deletion of up to 28, up to 27, up to 26, up to 25, up to 24, up to 23, up to 22, up to 21, up to 20, up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, or up to 1 contiguous amino acid residues at or near the C-terminus of the wild-type NiV-F cytoplasmic tail set forth in SEQ ID NO: 4. In some embodiments, the variant NiV-F is composed of a sequence in which the truncated cytoplasmic tail is directly linked to the C-terminus of the sequence set forth in SEQ ID NO: 2. In some embodiments, the cytoplasmic tail is the truncated Nipah virus cytoplasmic tail set forth in any one of SEQ ID NOS: 5-31. In some embodiments, the variant NiV-F is composed of a sequence in which the truncated cytoplasmic tail set forth in any one of SEQ ID NOS: 5-31 is directly linked to the C-terminus of the sequence set forth in SEQ ID NO: 2.

In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in any one of SEQ ID NOS: 5-31 is directly linked to the C-terminus of the sequence set forth in SEQ ID NO:383 (together the truncated F1 subunit), and (2) the F2 subunit is set forth as SEQ ID NO:365.

In some embodiments, the modified cytoplasmic tail is a truncated NiV-F cytoplasmic tail that has a deletion of from 23 to 27 contiguous amino acid residues at or near the C-terminus of the wild-type Nipah virus F protein cytoplasmic tail set forth in SEQ ID NO: 4. In some embodiments, the truncated portion has a deletion of 23, 24, 25, 26 or 27 amino acid residues at or near the C-terminus of the wild-type Nipah virus F protein cytoplasmic tail set forth in SEQ ID NO: 4. In some embodiment, the modified cytoplasmic tail has a deletion of at or about 23 amino acid residues at or near the C-terminus of the wild-type Nipah virus F protein cytoplasmic tail set forth in SEQ ID NO: 4. In some embodiment, the modified cytoplasmic tail has a deletion of at or about 24 amino acid residues at or near the C-terminus of the wild-type Nipah virus F protein cytoplasmic tail set forth in SEQ ID NO: 4. In some embodiment, the modified cytoplasmic tail has a deletion of at or about 25 amino acid residues at or near the C-terminus of the wild-type Nipah virus F protein cytoplasmic tail set forth in SEQ ID NO: 4. In some embodiment, the modified cytoplasmic tail has a deletion of at or about 26 amino acid residues at or near the C-terminus of the wild-type Nipah virus F protein cytoplasmic tail set forth in SEQ ID NO: 4. In some embodiment, the modified cytoplasmic tail has a deletion of at or about 27 amino acid residues at or near the C-terminus of the wild-type Nipah virus F protein cytoplasmic tail set forth in SEQ ID NO: 4. In some embodiments, the truncated cytoplasmic tail is directly linked to the C-terminus of the sequence set forth in SEQ ID NO: 2.

In some embodiments, the variant NiV-F cytoplasmic tail has a deletion that is not a deletion of at or about 27 amino acid residues at or near the C-terminus of the wild-type Nipah virus F protein cytoplasmic tail set forth in SEQ ID NO: 4. In some embodiments, the variant NiV-F cytoplasmic tail has a deletion that is not a deletion of at or about 25 amino acid residues at or near the C-terminus of the wild-type Nipah virus F protein cytoplasmic tail set forth in SEQ ID NO: 4. the variant NiV-F cytoplasmic tail has a deletion that is not a deletion of at or about 22 amino acid residues at or near the C-terminus of the wild-type Nipah virus F protein cytoplasmic tail set forth in SEQ ID NO: 4. In some embodiments, the variant NiV-F cytoplasmic tail has a deletion that is not a deletion of at or about 18 amino acid residues at or near the C-terminus of the wild-type Nipah virus F protein cytoplasmic tail set forth in SEQ ID NO: 4. the variant NiV-F cytoplasmic tail has a deletion that is not a deletion of at or about 15 amino acid residues at or near the C-terminus of the wild-type Nipah virus F protein cytoplasmic tail set forth in SEQ ID NO: 4.

In some embodiments, the variant NiV-F has a cytoplasmic tail deletion of 1 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:5. In some embodiments, the truncated cytoplasmic tail set forth in SEQ ID NO:5 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:5 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 2 amino acids at or near the C-terminus of the wild-type Nipah virus F protein cytoplasmic tail set forth in SEQ ID NO:4. In some embodiments, the truncated NiV-F cytoplasmic tail is set forth in SEQ ID NO:6. In some embodiments, the truncated cytoplasmic tail set forth in SEQ ID NO:6 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:6 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 3 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:7. In some embodiments, the truncated cytoplasmic tail set forth in SEQ ID NO:7 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:7 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 4 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:8. In some embodiments, the truncated cytoplasmic tail set forth in SEQ ID NO:8 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:8 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 5 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:9. In some embodiments, the truncated cytoplasmic tail set forth in SEQ ID NO:9 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:9 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 6 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:10. In some embodiments, the truncated cytoplasmic tail set forth in SEQ ID NO:10 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:10 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 7 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:11. In some embodiments, the truncated cytoplasmic tail set forth in SEQ ID NO:11 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:11 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 8 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:12. In some embodiments, the truncated cytoplasmic tail set forth in SEQ ID NO:12 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:12 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 9 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:13. In some embodiments, the truncated cytoplasmic tail set forth in SEQ ID NO:13 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:13 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 10 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:14. In some embodiments, the truncated cytoplasmic tail set forth in SEQ ID NO:14 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:14 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 11 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:15. In some embodiments, the truncated cytoplasmic tail set forth in SEQ ID NO:15 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:15 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 12 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:16. In some embodiments, the truncated cytoplasmic tail set forth in SEQ ID NO:16 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:16 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 13 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:17. In some embodiments, the truncated cytoplasmic tail set forth in SEQ ID NO:17 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:17 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 14 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:18. In some embodiments, the truncated cytoplasmic tail set forth in SEQ ID NO:18 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:18 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 15 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:19. In some embodiments, the truncated cytoplasmic tail set forth in SEQ ID NO:19 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:19 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 16 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:20. In some embodiments, the truncated cytoplasmic tail set forth in SEQ ID NO:20 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:20 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 17 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:21. In some embodiments, the truncated cytoplasmic tail set forth in SEQ ID NO:21 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:21 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 18 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:22. In some embodiments, the truncated cytoplasmic tail set forth in SEQ ID NO:22 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:22 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 19 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:23. In some embodiments, the truncated cytoplasmic tail set forth in SEQ ID NO:23 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:23 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 20 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:24. In some embodiments, the truncated cytoplasmic tail set forth in SEQ ID NO:24 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:24 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 21 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:25. In some embodiments, the truncated cytoplasmic tail set forth in SEQ ID NO:25 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:25 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 22 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:26. In some embodiments, the truncated cytoplasmic tail set forth in SEQ ID NO:26 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:26 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 23 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:27. In some embodiments, the truncated cytoplasmic tail set forth in SEQ ID NO:27 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:27 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 24 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:28. In some embodiments, the truncated cytoplasmic tail set forth in SEQ ID NO:28 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:28 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 25 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:29. In some embodiments, the truncated cytoplasmic tail set forth in SEQ ID NO:29 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:29 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 26 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:30. In some embodiments, the truncated cytoplasmic tail set forth in SEQ ID NO:30 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:30 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail deletion of 27 amino acid at or near the C-terminus of the wild-type Nipah virus F protein cytoplasmic tail set forth in SEQ ID NO:4. In some embodiments, the truncated NiV-F cytoplasmic tail is set forth in SEQ ID NO:31. In some embodiments, the truncated cytoplasmic tail set forth in SEQ ID NO:31 is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:31 is directly linked to the C-terminus of the sequence set forth in SEQ ID NO:383 (together the truncated F1 subunit), and (2) the F2 subunit is set forth as SEQ ID NO:365.

In some embodiments, the variant NiV-F has a cytoplasmic tail that is set forth in SEQ ID NO:27. In some embodiments, the truncated portion thereof is directly linked to the C-terminus of the sequence set forth in SEQ ID NO: 2. In some embodiments, the variant Niv-F protein proteins comprises a truncated F1 subunit containing the truncated cytoplasmic tail and an F2 subunit in which (1) the F1 subunit is a modified F1 composed of a sequence in which the truncated cytoplasmic tail set forth in SEQ ID NO:27 is directly linked to the C-terminus of the sequence set forth in SEQ ID NO:383 (together the truncated F1 subunit), and (2) the F2 subunit is set forth as SEQ ID NO:365.

In some embodiments, the variant NiV-F comprises the sequence of amino acids set forth in SEQ ID NO:306, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:306. In some embodiments, the variant NiV-F comprises the sequence of amino acids set forth in SEQ ID NO: 306.

In some embodiments, the variant Niv-F protein proteins comprises a truncated F1 subunit containing the truncated cytoplasmic tail and an F2 subunit in which (1) the F1 subunit is set forth as amino acids 84-497 of SEQ ID NO:306, and (2) the F2 subunit is set forth as amino acids 1-83 of SEQ ID NO:306.

In some embodiments, the variant NiV-F containing a truncated cytoplasmic tail may further contain one or more modifications (e.g. amino acid substitution/replacement, insertion or deletion) described herein. In some embodiments, the variant NiV-F containing a truncated cytoplasmic tail may further contain any of the provided modifications in the ectodomain, transmembrane domain or signal sequence as described herein.

### 2. Modified Nipah F Protein Endocytosis Motifs

In some embodiments, the variant NiV-F comprises a modified cytoplasmic tail which comprises a mutated cytoplasmic tail from a glycoprotein from the same Nipah virus.

In some embodiments, the variant NiV-F contains a modified cytoplasmic tail in which at least a part of the native cytoplasmic tail (e.g. SEQ ID 3) is a mutated portion thereof from a glycoprotein from Nipah Virus. In some embodiments, the cytoplasmic tail is a mutated portion thereof that is at least 6 amino acids in length. In some embodiments, the mutated portion thereof is 5, 6, 7, 8, 9, 10, 11, 12, 13, 14, 15, 16, 17, 18, 19, 20, 21, 22, 23, 24, 25, 26, 27, 28, 29, or 30 amino acids in length. In some embodiments, the mutated portion thereof is directly linked to the C-terminus of the sequence set forth in SEQ ID NO: 1 or SEQ ID NO:2.

In some embodiments, the cytoplasmic tail is a mutated Nipah virus cytoplasmic tail set forth in any one of SEQ ID NOS: 35-51. In some embodiments, the cytoplasmic tail set forth in any one of SEQ ID NOS: 35-51 is directly linked to the C-terminus of the sequence set forth in SEQ ID NO: 1 or SEQ ID NO:2. In some embodiments, the variant Niv-F protein proteins comprises a modified F1 subunit containing the mutated cytoplasmic tail and an F2 subunit in which (1) the F1 subunit is a modified F1 composed of a sequence in which the mutated cytoplasmic tail set forth in any one of SEQ ID NOS: 35-51 is directly linked to the C-terminus of the sequence set forth in SEQ ID NO:383 (together the modified F1 subunit), and (2) the F2 subunit is set forth as SEQ ID NO:365.

In some embodiments, the variant NiV-F containing a mutated cytoplasmic tail may further contain one or more modifications (e.g. amino acid substitution/replacement, insertion or deletion) described herein. In some embodiments, the variant NiV-F containing a mutated cytoplasmic tail may further contain any of the provided modifications in the ectodomain, transmembrane domain or signal sequence as described herein.

### 3. Chimeric Nipah F Protein Cytoplasmic Tails

In some embodiments, the variant NiV-F comprises a modified cytoplasmic tail which comprises a heterologous cytoplasmic tail or a truncated portion thereof from a glycoprotein from another virus. In some embodiments, the other virus is a member of the Kingdom Orthornavirae. In some embodiments, the other virus is a member of the family Paramyxoviridae, Rhabdoviridae, Arenaviridae, or Retroviridae. In some embodiments, the other virus is a member of the family Paramyxoviridae.

In some embodiments, the variant NiV-F contains a modified cytoplasmic tail in which at least a part of the native cytoplasmic tail (e.g. corresponding to amino acids 519-546 of SEQ ID NO:1; set forth in SEQ ID NO. 4) is replaced by a heterologous cytoplasmic tail or a truncated portion thereof from a glycoprotein from another virus from another virus or viral-associated protein. In some embodiments, the replaced cytoplasmic tail is a heterologous cytoplasmic tail or a truncated portion thereof that is at least 5 amino acids in length. In some embodiments, the replaced heterologous cytoplasmic tail or a truncated portion thereof is from or from about 5-180 amino acids in length, such as from or from about 5-150, from or from about 5-100, from or from about 5-75, from or from about 5-50, from or from about 5-40, from or from about 5-30, from or from about 5-20, from or from about 5-10, from or from about 10-150, from or from about 10-100, from or from about 10-75, from or from about 10-50, from or from about 10-40, from or from about 10-30, from or from about 10-20, from or from about 20-150, from or from about 20-100, from or from about 20-75, from or from about 20-50, from or from about 20-40, from or from about 20-30, from or from about 30-150, from or from about 30-100, from or from about 30-75, from or from about 30-50, from or from about 30-40, from or from about 40-150, from or from about 40-100, from or from about 40-75, from or from about 40-50, from or from about 50-150, from or from about 50-100, from or from about 50-75, from or from about 75-150, from or from about 75-100 or from or from about 100-150 amino acids in length. In some embodiments, the replaced heterologous cytoplasmic tail or a truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length.

In some embodiments, the heterologous cytoplasmic tail or the truncated portion thereof is directly linked to the C-terminus of the sequence set forth in SEQ ID NO: 2.

In some embodiments, the heterologous cytoplasmic tail is a cytoplasmic tail or a truncated portion thereof from a glycoprotein from another virus, such as a paramyxovirus, a retrovirus, a filovirus, a rhabdovirus or an arenavirus.

In some embodiments, the virus is a paramyxovirus other than a Nipah virus. For instance, the virus is a measles virus, Bat paramyxovirus, Cedar Virus, Canine Distemper Virus, Sendai virus, Hendra virus, Human Parainfluenza virus, or Newcastle Disease virus. In some embodiments, the replaced heterologous cytoplasmic tail is the native cytoplasmic tail or a truncated portion of the native cytoplasmic tail of another virus. In some embodiments, the replaced heterologous tail is set forth in any one of SEQ ID NOS: 58-169. In some embodiments, the heterologous cytoplasmic tail set forth in any one of SEQ ID NOS: 58-169 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 58-169 is directly linked to the C-terminus of the sequence set forth in SEQ ID NO:383 (together the modified F1 subunit), and (2) the F2 subunit is set forth as SEQ ID NO:365.

In some embodiments, the virus is a retrovirus. For instance, the virus may be a baboon envelope virus (BaEV), Gibbon Ape Leukemia virus (GaLV), murine leukemia virus, or human immunodeficiency virus 1 (HIV-1). In some embodiments, the virus is a filovirus. For instance, the virus may be an Ebola virus (EboV). In some embodiments, the virus is a rhabdovirus. For instance, the virus may be Cocal vesiculovirus (Cocal) or vesicular stomatitis virus (VSV). In some embodiments, the virus is an arenavirus. For instance, the virus may be Lymphocytic choriomeningitis virus (LCMV). In some embodiments, the replaced heterologous cytoplasmic tail is the native cytoplasmic tail or a truncated portion of the native cytoplasmic tail of another virus, such as set forth in any one of SEQ ID NOS: 170-193. In some embodiments, the heterologous cytoplasmic tail set forth in any one of SEQ ID NOS: 170-193 is directly linked to the C-terminus of the sequence set forth in SEQ ID NO:2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 170-193 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a cytoplasmic tail or a truncated portion thereof from a protein associated with viral infection, such a retroviral associated cellular protein. In some embodiments, the protein is CD63. In some embodiments, the replaced heterologous cytoplasmic tail is the native cytoplasmic tail or a truncated portion of the native cytoplasmic tail of a viral associated protein, such as set forth in SEQ ID NOS: 194-201. In some embodiments, the heterologous cytoplasmic tail set forth in SEQ ID NOS: 194-201 is directly linked to the N-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 194-201 is directly linked to the C-terminus of the sequence set forth in SEQ ID NO:383 (together the modified F1 subunit), and (2) the F2 subunit is set forth as SEQ ID NO:365.

In some embodiments, the variant NiV-F containing a modified cytoplasmic tail that includes all or a portion of a cytoplasmic tail from another virus protein or viral-associated protein may further contain one or more modifications (e.g. amino acid substitution/replacement, insertion or deletion) described herein. In some embodiments, the variant NiV-F containing a truncated cytoplasmic tail may further contain any of the provided modifications in the ectodomain, transmembrane domain or signal sequence as described herein.

a. Paramyxovirus

Paramyxoviral fusion proteins (F) are found in the viral lipid envelope, along with the attachment viral glycoprotein noted as any of HN, H, or G depending on the species. The F glycoprotein is a class I fusion protein having three identical subunits in the complete trimer, wherein cleavage of each of these subunits is required for function. Upon binding at a target surface via the attachment viral glycoprotein, proteolytically processed F glycoprotein undergoes an irreversible conformational change which facilitates the fusion of the viral and target membranes. Fusion via the F glycoprotein results in a pore which allows delivery of the viral nucleic acids into the target lumen (i.e., into a target cell).

In some embodiments, the heterologous cytoplasmic tail, or truncated portion thereof, is from a paramyxovirus. In some embodiments, the paramyxovirus is Hendra virus, Cedar virus, Canine distemper virus, Human Parainfluenza Virus 1, Human Parainfluenza Virus 2, Measles virus, Newcastle virus, or Sendai virus.

### 1) Hendra Virus

In some embodiments, the paramyxovirus attachment glycoprotein is a Hendra virus F protein (HeV-F). F proteins of henipaviruses, i.e., Hendra and Nipah viruses along with Cedar virus (CedV), Kumasi virus (KV), and Mòjiāing virus (MojV), are encoded as F₀ precursors containing a signal peptide (e.g. corresponding to amino acid residues 1-26 of SEQ ID NO:1). Following cleavage of the signal peptide, the mature F₀ is transported to the cell surface, then endocytosed and cleaved by cathepsin L (e.g. between amino acids 109-110 of SEQ ID NO:1) into the mature fusogenic subunits F1 (e.g. corresponding to amino acids 110-546 of SEQ ID NO:1) and F2 (e.g. corresponding to amino acid residues 27-109 of SEQ ID NO:1). The F1 and F2 subunits are associated by a disulfide bond and recycled back to the cell surface. The F1 subunit contains the fusion peptide domain located at the N terminus of the F1 subunit (e.g. g. corresponding to amino acids 110-129 of SEQ ID NO:1) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19).

In some embodiments, the variant NiV-F contains a heterologous cytoplasmic tail that is a cytoplasmic tail or a truncated portion thereof from a glycoprotein from Hendra virus. In some embodiments, the heterologous cytoplasmic tail replaces at least a part of the native cytoplasmic tail of NiV-F (e.g. SEQ ID NO:4). In some embodiments, the heterologous tail is a contiguous sequence of 5, 6, 7, 8, 9, 10, 11, 12, 13, 14, 15, 16, 17, 18, 19, 20, 21, 22, 23, 24, 25, 26, 27, or 28 amino acids of the native cytoplasmic tail of Hendra virus. In some embodiments, the native cytoplasmic tail of Hendra virus is set forth in SEQ ID NO:58.

In some embodiments, the heterologous cytoplasmic tail is a truncated portion of the Hendra virus cytoplasmic tail set forth in any one of SEQ ID NOS: 59-81. In some embodiments, the heterologous cytoplasmic tail set forth in any one of SEQ ID NOS: 59-81 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 59-81 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is a truncated HeV-F cytoplasmic tail. In some embodiments, the truncated HeV-F cytoplasmic tail has a deletion of up to 23, up to 22, up to 21, up to 20, up to 19, up to 18, up to 17, up to 16, up to 15, up to 14, up to 13, up to 12, up to 11, up to 10, up to 9, up to 8, up to 7, up to 6, up to 5 contiguous amino acid residues at or near the C-terminus of the wild-type HeV-F cytoplasmic tail set forth in SEQ ID NO: 58. In some embodiments, the truncated Hev-G cytoplasmic tail has a deletion of at or about or up to 22 contiguous amino acid residues at or near the N-terminus of the wild-type HeV-F cytoplasmic tail set forth in SEQ ID NO: 58.

In some embodiments, the variant NiV-F has a heterologous cytoplasmic tail that is set forth in SEQ ID NO:80. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:80 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail comprising the sequence of amino acids set forth in SEQ ID NO:80, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:80. In some embodiments, the variant NiV-F has a heterologous cytoplasmic tail comprising the sequence of amino acids set forth in SEQ ID NO: 80.

In some embodiments, the variant NiV-F comprises the sequence of amino acids set forth in SEQ ID NO:310, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:310. In some embodiments, the variant NiV-F comprises the sequence of amino acids set forth in SEQ ID NO: 310.

In some embodiments, the variant NiV-F comprises the sequence of amino acids set forth in SEQ ID NO:384, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:384. In some embodiments, the variant NiV-F comprises the sequence of amino acids set forth in SEQ ID NO: 384.

In some embodiments, the variant Niv-F protein proteins comprises a truncated F1 subunit containing the truncated cytoplasmic tail and an F2 subunit in which (1) the F1 subunit is set forth as amino acids 84-498 of SEQ ID NO:384, and (2) the F2 subunit is set forth as amino acids 1-83 of SEQ ID NO:384.

### 2) Measles Virus

In some embodiments, the paramyxovirus attachment glycoprotein is a Measles F protein (MV-F). As described above, Measles F protein (i.e., a paramyxoviral F protein) is a trimeric class I fusion protein with three conformational states corresponding to pre-fusion, intermediate, and post-fusion hairpin structures. The formation of the hairpin has been observed to result in apposition and therefore fusion of the viral and target membranes. The MV-F protein is encoded as a pre-cursor F₀ containing a signal peptide (e.g. corresponding to amino acid residues 1-23 of SEQ ID NO: 137). The mature protein is transported to the surface and cleaved (e.g., between amino acids 112 and 113 of SEQ ID NO:137) to yield the mature fusogenic subunits F1 (e.g. corresponding to amino acid residues 113-550 of SEQ ID NO: 137) and F2 (e.g. corresponding to amino acid residues 24-112 of SEQ ID NO: 137).

In some embodiments, the variant NiV-F contains a heterologous cytoplasmic tail that is a cytoplasmic tail or a truncated portion thereof from a glycoprotein from measles virus. In some embodiments, the heterologous cytoplasmic tail replaces at least a part of the native cytoplasmic tail of NiV-F (e.g. SEQ ID NO: 4). In some embodiments, the heterologous tail is a contiguous sequence of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids of the native cytoplasmic tail of measles virus. In some embodiments, the native cytoplasmic tail of measles virus is set forth in SEQ ID NO:125.

In some embodiments, the heterologous cytoplasmic tail is a truncated measles virus cytoplasmic tail set forth in any one of SEQ ID NOS: 126-140. In some embodiments, the heterologous cytoplasmic tail set forth in any one of SEQ ID NOS: 126-140 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 126-140 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 126. In some embodiments, the heterologous cytoplasmic tail set forth in SEQ ID NO: 126 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 126 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 127. In some embodiments, the heterologous cytoplasmic tail set forth in SEQ ID NO: 127 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 127 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 128. In some embodiments, the heterologous cytoplasmic tail set forth in SEQ ID NO: 128 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 128 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 129. In some embodiments, the heterologous cytoplasmic tail set forth in SEQ ID NO: 129 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 129 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 130. In some embodiments, the heterologous cytoplasmic tail set forth in SEQ ID NO: 130 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 130 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 131. In some embodiments, the heterologous cytoplasmic tail set forth in SEQ ID NO: 131 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 131 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 132. In some embodiments, the heterologous cytoplasmic tail set forth in SEQ ID NO: 132 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 132 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated measles virus cytoplasmic tail set forth in SEQ ID NO: 133. In some embodiments, the heterologous cytoplasmic tail set forth in SEQ ID NO: 133 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 133 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is a truncated MvF cytoplasmic tail. In some embodiments, the truncated MvF cytoplasmic tail has a deletion of up to 32, up to 31, up to 30, up to 29, up to 28, up to 27, up to 26, up to 25, up to 24, up to 23, up to 22, up to 21, up to 20, up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, or up to 1 contiguous amino acid residues at or near the C-terminus of the wild-type MvF cytoplasmic tail set forth in SEQ ID NO: 125. In some embodiments, the truncated MvF cytoplasmic tail has a deletion of at or about or up to 26 contiguous amino acid residues at or near the C-terminus of the wild-type MvF cytoplasmic tail set forth in SEQ ID NO: 125.

In some embodiments, the variant NiV-F has a heterologous cytoplasmic tail that is set forth in SEQ ID NO:133. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:133 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:126. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:126 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:127. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:127 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:128. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:128 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:129. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:129 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:130. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:130 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:131. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:131 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:132. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:132 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is set forth in SEQ ID NO:133. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:133 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail comprising the sequence of amino acids set forth in SEQ ID NO:126, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:126. In some embodiments, the variant NiV-F has a heterologous cytoplasmic tail comprising the sequence of amino acids set forth in SEQ ID NO: 126.

In some embodiments, the variant NiV-F has a heterologous cytoplasmic tail comprising the sequence of amino acids set forth in SEQ ID NO:127, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:127. In some embodiments, the variant NiV-F has a heterologous cytoplasmic tail comprising the sequence of amino acids set forth in SEQ ID NO: 127.

In some embodiments, the variant NiV-F has a heterologous cytoplasmic tail comprising the sequence of amino acids set forth in SEQ ID NO:128, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:128. In some embodiments, the variant NiV-F has a heterologous cytoplasmic tail comprising the sequence of amino acids set forth in SEQ ID NO: 128.

In some embodiments, the variant NiV-F has a heterologous cytoplasmic tail comprising the sequence of amino acids set forth in SEQ ID NO:129, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:129. In some embodiments, the variant NiV-F has a heterologous cytoplasmic tail comprising the sequence of amino acids set forth in SEQ ID NO: 129.

In some embodiments, the variant NiV-F has a heterologous cytoplasmic tail comprising the sequence of amino acids set forth in SEQ ID NO:130, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:130. In some embodiments, the variant NiV-F has a heterologous cytoplasmic tail comprising the sequence of amino acids set forth in SEQ ID NO: 130.

In some embodiments, the variant NiV-F has a heterologous cytoplasmic tail comprising the sequence of amino acids set forth in SEQ ID NO:131, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:131. In some embodiments, the variant NiV-F has a heterologous cytoplasmic tail comprising the sequence of amino acids set forth in SEQ ID NO: 131.

In some embodiments, the variant NiV-F has a heterologous cytoplasmic tail comprising the sequence of amino acids set forth in SEQ ID NO: 132, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 132. In some embodiments, the variant NiV-F has a heterologous cytoplasmic tail comprising the sequence of amino acids set forth in SEQ ID NO: 132.

In some embodiments, the variant NiV-F has a heterologous cytoplasmic tail comprising the sequence of amino acids set forth in SEQ ID NO: 133, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 133. In some embodiments, the variant NiV-F has a heterologous cytoplasmic tail comprising the sequence of amino acids set forth in SEQ ID NO: 133.

In some embodiments, the variant NiV-F comprises the sequence of amino acids set forth in SEQ ID NO:307, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:307. In some embodiments, the variant NiV-F comprises the sequence of amino acids set forth in SEQ ID NO: 307.

In some embodiments, the variant Niv-F protein proteins comprises a truncated F1 subunit containing the truncated cytoplasmic tail and an F2 subunit in which (1) the F1 subunit is set forth as amino acids 84-499 of SEQ ID NO:307, and (2) the F2 subunit is set forth as amino acids 1-83 of SEQ ID NO:307.

### 3) Newcastle Disease Virus

In some embodiments, the paramyxovirus attachment glycoprotein is a Newcastle Disease Virus F protein (NDV-F). As described above, Newcastle Disease Virus F protein (i.e., a paramyxoviral F protein) is a trimeric class I fusion protein with three conformational states corresponding to pre-fusion, intermediate, and post-fusion hairpin structures. The formation of the hairpin has been observed to result in apposition and therefore fusion of the viral and target membranes. The NDV-F protein is encoded as a pre-cursor F0 containing a signal peptide (e.g. corresponding to amino acid residues 1-31 of SEQ ID NO: 253. The mature protein is transported to the surface and cleaved (e.g., between amino acids 116 and 117 of SEQ ID NO:253) to yield the mature fusogenic subunits F1 (e.g. corresponding to amino acid residues 117-553 of SEQ ID NO: 253) and F2 (e.g. corresponding to amino acid residues 32-116 of SEQ ID NO: 253).

In some embodiments, the variant NiV-F contains a heterologous cytoplasmic tail that is a cytoplasmic tail or a truncated portion thereof from a glycoprotein from Newcastle Disease Virus. In some embodiments, the heterologous cytoplasmic tail replaces at least a part of the native cytoplasmic tail of NiV-F (e.g. SEQ ID NO: 4). In some embodiments, the heterologous tail is a contiguous sequence of 1, 2, 3, 4, 5, 6, 7, 8, 9, 10, 11, 12, 13, 14, 15, 16, 17, 18, 19, 20, 21, 22, 23, 24, 25, or 26 amino acids of the native cytoplasmic tail of Newcastle Disease Virus. In some embodiments, the native cytoplasmic tail of Newcastle Disease Virus is set forth in SEQ ID NO:141.

In some embodiments, the heterologous cytoplasmic tail is a truncated Newcastle Disease Virus cytoplasmic tail set forth in any one of SEQ ID NOS: 140-155. In some embodiments, the heterologous cytoplasmic tail set forth in any one of SEQ ID NOS: 140-155 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 140-155 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated Newcastle Disease Virus cytoplasmic tail set forth in SEQ ID NO: 147. In some embodiments, the heterologous cytoplasmic tail set forth in SEQ ID NO: 147 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 147 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated Newcastle Disease Virus cytoplasmic tail set forth in SEQ ID NO: 148. In some embodiments, the heterologous cytoplasmic tail set forth in SEQ ID NO: 148 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 148 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated Newcastle Disease Virus cytoplasmic tail set forth in SEQ ID NO: 149. In some embodiments, the heterologous cytoplasmic tail set forth in SEQ ID NO: 149 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 149 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated Newcastle Disease Virus cytoplasmic tail set forth in SEQ ID NO: 150. In some embodiments, the heterologous cytoplasmic tail set forth in SEQ ID NO: 150 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 150 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated Newcastle Disease Virus cytoplasmic tail set forth in SEQ ID NO: 151. In some embodiments, the heterologous cytoplasmic tail set forth in SEQ ID NO: 151 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 151 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is a truncated NDV-F cytoplasmic tail. In some embodiments, the truncated NDV-F cytoplasmic tail has a deletion of up to 25, up to 24, up to 23, up to 22, up to 21, up to 20, up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, or up to 1 contiguous amino acid residues at or near the C-terminus of the wild-type NDV-F cytoplasmic tail set forth in SEQ ID NO: 141. In some embodiments, the truncated NDV-F cytoplasmic tail has a deletion of at or about or up to 20 contiguous amino acid residues at or near the C-terminus of the wild-type NDV-F cytoplasmic tail set forth in SEQ ID NO: 141. In some embodiments, the truncated NDV-F cytoplasmic tail has a deletion of at or about or up to 17 contiguous amino acid residues at or near the C-terminus of the wild-type NDV-F cytoplasmic tail set forth in SEQ ID NO: 141.

In some embodiments, the variant NiV-F has a heterologous cytoplasmic tail that is set forth in SEQ ID NO:147. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:147 is directly linked to the C-terminus of the sequence set forth in SEQ ID NO:383 (together the modified F1 subunit), and (2) the F2 subunit is set forth as SEQ ID NO:365.

In some embodiments, the variant NiV-F comprises the sequence of amino acids set forth in SEQ ID NO:308, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:308. In some embodiments, the variant NiV-F comprises the sequence of amino acids set forth in SEQ ID NO: 308.

In some embodiments, the variant Niv-F protein proteins comprises a truncated F1 subunit containing the truncated cytoplasmic tail and an F2 subunit in which (1) the F1 subunit is set forth as amino acids 84-501 of SEQ ID NO:308, and (2) the F2 subunit is set forth as amino acids 1-83 of SEQ ID NO:308.

In some embodiments, the variant NiV-F has a heterologous cytoplasmic tail that is set forth in SEQ ID NO:150. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:150 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail comprising the sequence of amino acids set forth in SEQ ID NO:147, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 147. In some embodiments, the variant NiV-F has a heterologous cytoplasmic tail comprising the sequence of amino acids set forth in SEQ ID NO: 147.

In some embodiments, the variant NiV-F has a heterologous cytoplasmic tail comprising the sequence of amino acids set forth in SEQ ID NO:148, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 148. In some embodiments, the variant NiV-F has a heterologous cytoplasmic tail comprising the sequence of amino acids set forth in SEQ ID NO: 148.

In some embodiments, the variant NiV-F has a heterologous cytoplasmic tail comprising the sequence of amino acids set forth in SEQ ID NO:149, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 149. In some embodiments, the variant NiV-F has a heterologous cytoplasmic tail comprising the sequence of amino acids set forth in SEQ ID NO: 149.

In some embodiments, the variant NiV-F has a heterologous cytoplasmic tail comprising the sequence of amino acids set forth in SEQ ID NO:150, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 150. In some embodiments, the variant NiV-F has a heterologous cytoplasmic tail comprising the sequence of amino acids set forth in SEQ ID NO: 150.

In some embodiments, the variant NiV-F has a heterologous cytoplasmic tail comprising the sequence of amino acids set forth in SEQ ID NO:151, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 151. In some embodiments, the variant NiV-F has a heterologous cytoplasmic tail comprising the sequence of amino acids set forth in SEQ ID NO: 151.

In some embodiments, the variant NiV-F comprises the sequence of amino acids set forth in SEQ ID NO: 309, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 309. In some embodiments, the variant NiV-F comprises the sequence of amino acids set forth in SEQ ID NO: 309.

In some embodiments, the variant Niv-F protein proteins comprises a truncated F1 subunit containing the truncated cytoplasmic tail and an F2 subunit in which (1) the F1 subunit is set forth as amino acids 84-498 of SEQ ID NO:308, and (2) the F2 subunit is set forth as amino acids 1-83 of SEQ ID NO:308.

### 4) Cedar Virus

In some embodiments, the paramyxovirus attachment glycoprotein is a Cedar Virus F protein (CedV-F). In some embodiments, the variant NiV-F contains a heterologous cytoplasmic tail that is a cytoplasmic tail or a truncated portion thereof from a glycoprotein from Cedar Virus In some embodiments, the heterologous cytoplasmic tail replaces at least a part of the native cytoplasmic tail of NiV-F (e.g. SEQ ID NO: 4). In some embodiments, the heterologous tail is a contiguous sequence of 1, 2, 3, 4, 5, 6, 7, 8, 9, 10, 11, 12, 13, 14, 15, 16, 17, 18, 19, 20, 21, 22, 23, 24, 25, or 26 amino acids of the native cytoplasmic tail of Cedar Virus. In some embodiments, the native cytoplasmic tail of Cedar Virus is set forth in SEQ ID NO: 96.

In some embodiments, the heterologous cytoplasmic tail is a truncated Cedar Virus cytoplasmic tail set forth in any one of SEQ ID NOS: 96-110. In some embodiments, the heterologous cytoplasmic tail set forth in any one of SEQ ID NOS: 96-110 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in any one of SEQ ID NOS: 96-110 is directly linked to the C-terminus of the sequence set forth in SEQ ID NO:383 (together the modified F1 subunit), and (2) the F2 subunit is set forth as SEQ ID NO:365.

In some embodiments, the heterologous cytoplasmic tail is a truncated Cedar Virus cytoplasmic tail set forth in SEQ ID NO: 109. In some embodiments, the heterologous cytoplasmic tail set forth in SEQ ID NO: 109 is directly linked to the C-terminus of the sequence set forth in SEQ ID NO: 2. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO: 109 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail that is a truncated CedV-F cytoplasmic tail. In some embodiments, the truncated CedV-F cytoplasmic tail has a deletion of up to 25, up to 24, up to 23, up to 22, up to 21, up to 20, up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, or up to 1 contiguous amino acid residues at or near the C-terminus of the wild-type CedV-F cytoplasmic tail set forth in SEQ ID NO: 96. In some embodiments, the truncated CedV-F cytoplasmic tail has a deletion of at or about or up to 20 contiguous amino acid residues at or near the C-terminus of the wild-type CedV-F cytoplasmic tail set forth in SEQ ID NO: 96. In some embodiments, the truncated CedV-F cytoplasmic tail has a deletion of at or about or up to 17 contiguous amino acid residues at or near the C-terminus of the wild-type CedV-F cytoplasmic tail set forth in SEQ ID NO: 96.

In some embodiments, the variant NiV-F has a heterologous cytoplasmic tail that is set forth in SEQ ID NO:109. In some embodiments, the variant Niv-F protein proteins comprises a modified F1 subunit containing the heterologous cytoplasmic tail and an F2 subunit in which (1) the F1 subunit is a modified F1 composed of a sequence in which the heterologous cytoplasmic tail set forth in SEQ ID NO:109 is directly linked to the C-terminus of the sequence set forth in SEQ ID NO:383 (together the modified F1 subunit), and (2) the F2 subunit is set forth as SEQ ID NO:365.

In some embodiments, the variant NiV-F has a heterologous cytoplasmic tail comprising the sequence of amino acids set forth in SEQ ID NO:109, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:109. In some embodiments, the variant NiV-F has a heterologous cytoplasmic tail comprising the sequence of amino acids set forth in SEQ ID NO: 109.

### B. Variant NiV-F Ectodomains

In some embodiments, the variant NiV-F comprises a modified NiV-F ectodomain in which is contained a N-glycan mutation of NiV-F, a mutation that stabilizes interaction among hexameric NiV-F trimers, and/or mutations that engineer the protease (e.g. cathepsin, such as cathepsin L) cleavage site to improve active F processing. In some embodiments, any of the provided modifications in the ectodomain of a variant NiV-F described herein can be combined with a modification of the cytoplasmic tail, transmembrane domain or heterologous signal sequence as described herein.

In some embodiments, the variant NiV-F contains an engineered ectodomain in which the protease cleave site for processing an active F protein is engineered by one more amino acid substitutions. In some embodiments, the wild-type NiV-F cathepsin L cleavage site NNTHDLVGDVRLAGV (SEQ ID NO:311) is replaced with a modified cleavage site containing a furin consensus cleavage sequence R-X-R/K-R (SEQ ID NO:312), in which X is any amino acid. In some embodiments, the cleavage sequence is one set forth in any of SEQ ID NOS: NNTHDSRRHKR/FAGV (SEQ ID NO:313), NNTHDLVRHKR/FAGV (SEQ ID NO: 314), NNTHDLVRHKR/FAGV (SEQ ID NO: 315), NNTHDLVRHRR/FAGV (SEQ ID NO: 316), NNTHDLVRHRR/LAGV (SEQ ID NO: 317), NNGHDSRRHKR/FAGV (SEQ ID NO: 318), NNGHDLVRHKR/FAGV (SEQ ID NO: 319), NNGHDLVRHKR/LAGV (SEQ ID NO: 320), NNGHDLVRHRR/FAGV (SEQ ID NO: 321), NNGHDLVRHRR/LAGV (SEQ ID NO: 322), QNTHDSRRHKR/FAGV (SEQ ID NO: 323), QNTHDLVRHKR/FAGV (SEQ ID NO: 324), QNTHDLVRHKR/LAGV (SEQ ID NO: 325), QNTHDLVRHRR/FAGV (SEQ ID NO: 326), QNTHDLVRHRR/LAGV (SEQ ID NO:327), wherein the “/” mark is used to indicate the site of cleavage within the sequence. In embodiments in which the variant NiV-F contains a modified cleavage site, the mature variant F0 sequence containing the modified cleavage site is able to be cleaved by furin at the cleavage site to produce the mature fusogenic subunits F1 (e.g. sequence beginning with residues “FAGV . . . ” or “LAGV . . . ” after the cleavage site until the C-terminal residue of the cytoplasmic tail of the mature variant F0 sequence) and F2 (e.g. sequence corresponding to the mature N-terminal residue after cleavage of the signal sequence to the sequence QNTHDLVR-X-R/K-R of the mature variant F0 sequence). In some embodiments, the F1 and F2 subunits are associated by a disulfide bond and expressed on the surface of the lipid particle, e.g. lentiviral vector.

In some embodiments, the variant NiV-F comprises the sequence of amino acids set forth in SEQ ID NO:1 in which the amino acid residues NNTHDLVGDVRLAGV (corresponding to residues 99-113 of SEQ ID NO:1) are replaced with a modified cleavage site composed of amino acid residues set forth in any one of SEQ ID NOS: 313-327, or is a mature form thereof lacking the signal peptide (e.g. lacking amino acid residues 1-26 of SEQ ID NO:1). In embodiments in which the variant NiV-F contains a modified cleavage site, the mature variant F0 sequence containing the modified cleavage site is able to be cleaved by furin at the cleavage site to produce the mature fusogenic subunits F1 (e.g. corresponding to amino acid residues 110-546 of SEQ ID NO:1 in which amino acids 110-113 are amino acids FAGV or LAGV as set forth in any one of SEQ ID NOS: 313-327) and F2 (e.g. sequence corresponding to amino acids 27-109 of SEQ ID NO: 1 in which amino acid residues 10⁶-109 are replaced with amino acid residues R-X-R/K-R, such as set forth in any one of SEQ ID NOS: 313-327). In some embodiments, the F1 and F2 subunits are associated by a disulfide bond and expressed on the surface of the lipid particle, e.g. lentiviral vector.

In some embodiments, the variant NiV-F comprises the sequence of amino acids set forth in SEQ ID NO:302 in which the amino acid residues NNTHDLVGDVRLAGV (corresponding to residues 99-113 of SEQ ID NO:302) are replaced with amino acid residues set forth in any one of SEQ ID NOS: 313-327, or is a mature form thereof lacking the signal peptide (e.g. lacking amino acid residues 1-26 of SEQ ID NO:302). In some embodiments, the variant NiV-F comprises the sequence of amino acids set forth in SEQ ID NO:303 in which the amino acid residues NNTHDLVGDVRLAGV (corresponding to residues 73-87 of SEQ ID NO:303) are replaced with amino acid residues set forth in any one of SEQ ID NOS: 313-327. In embodiments in which the variant NiV-F contains a modified cleavage site, the mature variant F0 sequence containing the modified cleavage site is able to be cleaved by furin at the cleavage site to produce the mature fusogenic subunits F1 (e.g. corresponding to amino acid residues 110-524 of SEQ ID NO:302 in which amino acids 110-113 are amino acids FAGV or LAGV as set forth in any one of SEQ ID NOS: 313-327) and F2 (e.g. sequence corresponding to amino acids 27-109 of SEQ ID NO: 302 in which amino acid residues 106-109 are replaced with amino acid residues R-X-R/K-R, such as set forth in any one of SEQ ID NOS: 313-327). In some embodiments, the F1 and F2 subunits are associated by a disulfide bond and expressed on the surface of the lipid particle, e.g. lentiviral vector.

In some embodiments, the variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26). In some embodiments, the variant NiV-F comprises the sequence of amino acids set forth in any of SEQ ID NOS: 224-238, or is a mature form thereof lacking the signal peptide (e.g. lacking amino acids 1-26). In embodiments in which the variant NiV-F contains a modified cleavage site, the mature variant F0 sequence containing the modified cleavage site is able to be cleaved by furin at the cleavage site to produce the mature fusogenic subunits F1 (e.g. corresponding to amino acid residues 110-524 of any of SEQ ID NOS:224-238) and F2 (e.g. sequence corresponding to amino acids 27-109 of any one of SEQ ID NOS: 224-238. In some embodiments, the F1 and F2 subunits are associated by a disulfide bond and expressed on the surface of the lipid particle, e.g. lentiviral vector.

In some embodiments, the variant NiV-F comprises the sequence of amino acids set forth in any of SEQ ID NOS: 385-399 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 385-399 and contains the cleavage site set forth in any one of SEQ ID NOS: 385-399, respectively. In some embodiments, the variant NiV-F comprises the sequence of amino acids set forth in any of SEQ ID NOS: 385-399. In embodiments in which the variant NiV-F contains a modified cleavage site, the mature variant F0 sequence containing the modified cleavage site is able to be cleaved by furin at the cleavage site to produce the mature fusogenic subunits F1 (e.g. corresponding to amino acid residues 84-498 of any of SEQ ID NOS:385-399) and F2 (e.g. sequence corresponding to amino acids 1-83 of any one of SEQ ID NOS: 385-399). In some embodiments, the F1 and F2 subunits are associated by a disulfide bond and expressed on the surface of the lipid particle, e.g. lentiviral vector.

In some embodiments, the variant NiV-F contains an engineered ectodomain in which the cathepsin L cleavage site for processing active F protein is engineered by replacement of the protease site VGDVR (SEQ ID NO:329) with a protease site from another virus. In some embodiments, the protease site is from a virus of the Kingdom Orthornavirae. In some embodiments, the other virus is a member of the family Paramyxoviridae, Rhabdoviridae, Arenaviridae, or Retroviridae. In some embodiments, the other virus is a member of the family Paramyxoviridae. In some embodiments, the other virus is a Hendra virus, Cedar virus, Canine distemper virus, Parainfluenza virus, Measles virus, Newcastle disease virus, or Sendai virus.

In some embodiments, the wild-type NiV-F cathepsin L cleavage site comprising the protease site VGDVR (SEQ ID NO:339) is replaced with a modified cleavage site from another virus. In some embodiments, the cleavage sequence is one set forth in any of SEQ ID NOS: VGDVK (SEQ ID NO:329), RNHNR (SEQ ID NO: 330), RRHKR (SEQ ID NO: 331), RRQKR (SEQ ID NO: 332), GRQGR (SEQ ID NO: 333), TRQKR (SEQ ID NO: 334), EIQSR (SEQ ID NO: 335), VPQSR (SEQ ID NO: 336), NPQSR (SEQ ID NO: 337), PRTKR (SEQ ID NO: 338).

In some embodiments, the variant NiV-F comprises the sequence of amino acids set forth in SEQ ID NO:1 in which the amino acid residues VGDVR (corresponding to residues 105-109 of SEQ ID NO:1) are replaced with a modified cleavage site composed of amino acid residues set forth in any one of SEQ ID NOS: 329-338, or is a mature form thereof lacking the signal peptide (e.g. amino acid residues 1-26 of SEQ ID NO:1). In embodiments in which the variant NiV-F contains a modified cleavage site, the mature variant F0 sequence containing the modified cleavage site is able to be cleaved by a protease for cleaving the cleavage site, e.g. cathepsin L, to produce the mature fusogenic subunits F1 (e.g. corresponding to amino acid residues 110-546 of SEQ ID NO:1) and F2 (e.g. sequence corresponding to amino acids 27-109 of SEQ ID NO: 1 in which amino acid residues 105-109 are replaced with amino acid residues set forth in any one of SEQ ID NOS: 329-338). In some embodiments, the F1 and F2 subunits are associated by a disulfide bond and expressed on the surface of the lipid particle, e.g. lentiviral vector.

In some embodiments, the variant NiV-F comprises the sequence of amino acids set forth in SEQ ID NO:302 in which the amino acid residues VGDVR (corresponding to residues 105-109 of SEQ ID NO:302) are replaced with amino acid residues set forth in any one of SEQ ID NOS: 329-338, or is a mature form thereof lacking the signal peptide (e.g. lacking amino acid residues 1-26 of SEQ ID NO: 302). In some embodiments, the variant NiV-F comprises the sequence of amino acids set forth in SEQ ID NO:303 in which the amino acid residues VGDVR (corresponding to residues 79-83 of SEQ ID NO:303) are replaced with amino acid residues set forth in any one of SEQ ID NOS: 329-338. In embodiments in which the variant NiV-F contains a modified cleavage site, the mature variant F0 sequence containing the modified cleavage site is able to be cleaved by a protease for cleaving the cleavage site, e.g. cathepsin L, to produce the mature fusogenic subunits F1 (e.g. corresponding to amino acid residues 110-524 of SEQ ID NO:302) and F2 (e.g. sequence corresponding to amino acids 27-109 of SEQ ID NO: 302 in which amino acid residues 105-109 are replaced with amino acid residues set forth in any one of SEQ ID NOS: 329-338). In some embodiments, the F1 and F2 subunits are associated by a disulfide bond and expressed on the surface of the lipid particle, e.g. lentiviral vector.

In some embodiments, the variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26). In some embodiments, the variant NiV-F comprises the sequence of amino acids set forth in any of SEQ ID NOS: 239-248, or is a mature form thereof lacking the signal peptide (e.g. lacking amino acids 1-26). In embodiments in which the variant NiV-F contains a modified cleavage site, the mature variant F0 sequence containing the modified cleavage site is able to be cleaved by a protease for cleaving the cleavage site, e.g. cathepsin L, to produce the mature fusogenic subunits F1 (e.g. corresponding to amino acid residues 110-524 of any of SEQ ID NOS:239-248) and F2 (e.g. sequence corresponding to amino acids 27-109 of any one of SEQ ID NOS: 239-248). In some embodiments, the F1 and F2 subunits are associated by a disulfide bond and expressed on the surface of the lipid particle, e.g. lentiviral vector.

In some embodiments, the variant NiV-F comprises the sequence of amino acids set forth in any of SEQ ID NOS: 400-409 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 400-409 and contains the cleavage site set forth in any one of SEQ ID NOS: 2385-399, respectively. In some embodiments, the variant NiV-F comprises the sequence of amino acids set forth in any of SEQ ID NOS: 400-409. In embodiments in which the variant NiV-F contains a modified cleavage site, the mature variant F0 sequence containing the modified cleavage site is able to be cleaved by a protease for cleaving the cleavage site, e.g. cathepsin L, to produce the mature fusogenic subunits F1 (e.g. corresponding to amino acid residues 84-498 of any of SEQ ID NOS:400-409) and F2 (e.g. sequence corresponding to amino acids 1-83 of any one of SEQ ID NOS: 400-409). In some embodiments, the F1 and F2 subunits are associated by a disulfide bond and expressed on the surface of the lipid particle, e.g. lentiviral vector.

In some embodiments, the variant NiV-F comprises the cleavage sequence VGDVK (SEQ ID NO:329). In some embodiments, the variant NiV-F comprises the sequence of amino acids set forth in SEQ ID NO:1 in which the amino acid residues VGDVR (corresponding to residues 105-109 of SEQ ID NO:1) are replaced with amino acid residues VGDVK (SEQ ID NO:329), or is a mature form thereof lacking the signal peptide (e.g. amino acid residues 1-26). In some embodiments, the variant NiV-F comprises the sequence of amino acids set forth in SEQ ID NO:302 in which the amino acid residues VGDVR (corresponding to residues 105-109 of SEQ ID NO:302) are replaced with amino acid residues VGDVK (SEQ ID NO:329), or is a mature form thereof lacking the signal peptide (e.g. lacking amino acid residues 1-26). In some embodiments, the variant NiV-F comprises the sequence of amino acids set forth in SEQ ID NO:303 in which the amino acid residues VGDVR (corresponding to residues 79-83 of SEQ ID NO:302) are replaced with amino acid residues VGDVK (SEQ ID NO:329).

In some embodiments, the variant NiV-F comprises the sequence of amino acids set forth in SEQ ID NOS: 239 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 239 and contains the cleavage site VGDVK, or is a mature form thereof lacking the signal peptide (e.g. lacking amino acids 1-26). In some embodiments, the variant NiV-F comprises the sequence of amino acids set forth in SEQ ID NO: 239, or is a mature form thereof lacking the signal peptide (e.g. lacking amino acids 1-26). In embodiments in which the variant NiV-F contains a modified cleavage site, the mature variant F0 sequence containing the modified cleavage site is able to be cleaved by a protease for cleaving the cleavage site, e.g. cathepsin L, to produce the mature fusogenic subunits F1 set forth as amino acid residues 110-524 of SEQ ID NO:239 and F2 set forth as amino acids 27-109 of SEQ ID NO: 239. In some embodiments, the F1 and F2 subunits are associated by a disulfide bond and expressed on the surface of the lipid particle, e.g. lentiviral vector.

In some embodiments, the variant NiV-F comprises the sequence of amino acids set forth in SEQ ID NOS: 400 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 400 and contains the cleavage site VGDVK. In some embodiments, the variant NiV-F comprises the sequence of amino acids set forth in SEQ ID NO: 400. In embodiments in which the variant NiV-F contains a modified cleavage site, the mature variant F0 sequence containing the modified cleavage site is able to be cleaved by a protease for cleaving the cleavage site, e.g. cathepsin L, to produce the mature fusogenic subunits F1 set forth as amino acid residues 84-498 of SEQ ID NO: 400 and F2 set forth as amino acids 1-83 of SEQ ID NO:400. In some embodiments, the F1 and F2 subunits are associated by a disulfide bond and expressed on the surface of the lipid particle, e.g. lentiviral vector.

### C. Hyperfusogenic Modifications

In some embodiments, the variant NiV-F comprises a hyperfusogenic mutation, such that lipid particles with the variant NiV-F display a hyperfusogenic phenotype. For example, characteristic mutations resulting in hyperfusogenic MeV are known to be implicated in infection of the central nervous system (Angius et al. J Virol. 2019; 93(4).) Fusogenic activity includes the activity of the F protein in conjunction with a G protein as described in Section III.B. to promote or facilitate fusion of two membrane lumens, such as the lumen of the lipid particle having embedded in its lipid bilayer a variant Nipah F glycoprotein, and a cytoplasm of a target cell, e.g. a cell that contains a surface receptor or molecule that is recognized or bound by the variant glycoprotein. In some embodiments, the F protein and G protein are from the same Paramyxovirus species (e.g. NiV-G and NiV-F). In some embodiments, the F protein and G protein are from different Paramyxovirus species (e.g. NiV-G and HeV-F). In some embodiments, the hyperfusogenic mutation is in the cytoplasmic domain of the F protein. In some embodiments, the native or wild-type NiV-F cytoplasmic domain set forth in SEQ ID NO:4 is replaced with a hyperfusogenic variant from a viral protein. In some embodiments, the native or wild-type NiV-F cytoplasmic domain set forth in SEQ ID NO:4 is modified with a hyperfusogenic mutation. Without wishing to be bound by theory, it is observed that some specific residues within the cytoplasmic tail can modulate the fusogenic activity of NiV-F. In some aspects, mutations of polybasic residues KRR (e.g., corresponding to residues 521-523 of SEQ ID NO: 1) increases cell-to-cell fusion by as much as 5.5 fold (Lee et al, J Virol 81(9), 2007). In some embodiments, the cytoplasmic domain contains a KlA mutation such that a Lysine (e.g., corresponding to position 521 of SEQ ID NO: 1) is substituted for Alanine.

In some embodiments, the hyperfusogenic cytoplasmic domain is set forth in SEQ ID NO:53. In some embodiments, the hyperfusogenic cytoplasmic domain is set forth in SEQ ID NO:54. In some embodiments, the hyperfusogenic cytoplasmic domain is set forth in SEQ ID NO:55. In some embodiments, the hyperfusogenic cytoplasmic domain is set forth in SEQ ID NO:56.

In some embodiments, the hyperfusogenic cytoplasmic domain set forth in any one of SEQ ID NOS: 53-56 is directly linked to the C-terminus of the sequence set forth in SEQ ID NO: 2. In some embodiments, the variant Niv-F protein proteins comprises a modified F1 subunit containing the hyperfusogenic cytoplasmic domain and an F2 subunit in which (1) the F1 subunit is a modified F1 composed of a sequence in which the hyperfusogenic cytoplasmic tail set forth in any one of SEQ ID NOS: 53-56 is directly linked to the C-terminus of the sequence set forth in SEQ ID NO:383 (together the modified F1 subunit), and (2) the F2 subunit is set forth as SEQ ID NO:365.

In some embodiments, the hyperfusogenic mutation is to a residue of the F protein which is glycosylated. In some embodiments, the native or wild-type NiV-F set forth in SEQ ID NO:1 or 2, or a functional variant or biologically active portion thereof (e.g. SEQ ID NO: 302 or SEQ ID NO:303) is modified with a hyperfusogenic mutation to an amino acid which is decorated with an N-glycan. Without wishing to be bound by theory, it is observed that the modulation of N-linked glycosylated residues of Paramyxovirus F proteins results in conformational changes of the F protein that may lead to hyperfusogenicity. In some embodiments, the variant NiV-F comprises a mutation at one or more positions 64, 67, 99, 414, and/or 464, with reference to numbering of SEQ ID NO:1, to glutamine (Q) or a conservative amino acid thereof. In some embodiments, the amino acid substitution is to Serine (S), Threonine (T), Asparagine (R) or glutamine (Q).

In some embodiments, the mutation is one of more of N64Q, N67Q, N99Q, N414Q and/or N464Q, with reference to numbering set forth in SEQ ID NO:1. In some embodiments, the mutations are N64Q, N67Q, N99Q, N414Q, N464Q, N67Q/N99Q, N67Q/N414Q, N67Q/N464Q, N99Q/N414Q, N99Q/N464Q, N414Q/N464Q, N67Q/N99Q/N414Q, N67Q/N414Q/N464Q, N99Q/N414Q/N464Q, or N67Q/N99Q/N414Q/N464Q. In some embodiments, the mutations are N64Q/N99Q, N64Q/N99Q/N464Q, N64Q/N67Q/N99Q, or N64Q/N67Q/N99Q/N464Q. In some embodiments, the mutations are in the sequence set forth in SEQ ID NO: 1, SEQ ID NO:2, SEQ ID NO: 302 or SEQ ID NO:303.

In some embodiments, the mutation is one of more of N67Q, N99Q, N414Q and/or N464Q, with reference to numbering set forth in SEQ ID NO:1. In some embodiments, the mutations are N67Q, N99Q, N414Q, N464Q, N67Q/N99Q, N67Q/N414Q, N67Q/N464Q, N99Q/N414Q, N99Q/N464Q, N414Q/N464Q, N67Q/N99Q/N414Q, N67Q/N414Q/N464Q, N99Q/N414Q/N464Q, OR N67Q/N99Q/N414Q/N464Q. In some embodiments, the mutations are in the sequence set forth in SEQ ID NO: 1, SEQ ID NO:2, SEQ ID NO: 302 or SEQ ID NO:303.

In some embodiments, the hyperfusogenic variant NiV-F is encoded by a polynucleotide that encodes the sequence set forth in any one of SEQ ID NOS: 273-280. In some embodiments, the variant NiV-F comprises the sequence of amino acids set forth in any one of SEQ ID NO: 273-280, or a mature form thereof lacking the signal peptide (amino acids 1-26). In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 273-280 and that contains the hyperfusogenic mutations, or a mature form thereof lacking the signal peptide (amino acids 1-26). In embodiments, the variant NiV-F contains a F1 subunit corresponding to amino acid residues 110-524 of any of SEQ ID NOS:273-280) and an F2 subunit corresponding to amino acids 27-109 of any one of SEQ ID NOS: 273-280. In some embodiments, the F1 and F2 subunits are associated by a disulfide bond and expressed on the surface of the lipid particle, e.g. lentiviral vector.

In some embodiments, the hyperfusogenic variant NiV-F is encoded by a polynucleotide that encodes the sequence set forth in SEQ ID NO: 274. In some embodiments, the variant NiV-F comprises the sequence of amino acids set forth in SEQ ID NO: 274, or a mature form thereof lacking the signal peptide (amino acids 1-26). In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 274 and that contains the hyperfusogenic mutations, or a mature form thereof lacking the signal peptide (amino acids 1-26). In embodiments, the variant NiV-F contains a F1 subunit corresponding to amino acid residues 110-524 of any of SEQ ID NO: 274) and an F2 subunit corresponding to amino acids 27-109 of SEQ ID NO: 274. In some embodiments, the F1 and F2 subunits are associated by a disulfide bond and expressed on the surface of the lipid particle, e.g. lentiviral vector.

In some embodiments, the hyperfusogenic variant NiV is encoded by a polynucleotide that encodes the sequence set forth in any one of SEQ ID NOS: 457-464. In some embodiments, the variant NiV-F comprises the sequence of amino acids set forth in any one of SEQ ID NO: 457-464, or a mature form thereof lacking the signal peptide (amino acids 1-26). In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 457-464 and that contains the hyperfusogenic mutations, or a mature form thereof lacking the signal peptide (amino acids 1-26). In embodiments, the variant NiV-F contains a F1 subunit corresponding to amino acid residues 110-524 of any of SEQ ID NOS: 457-464) and an F2 subunit corresponding to amino acids 27-109 of any one of SEQ ID NOS: 457-464. In some embodiments, the F1 and F2 subunits are associated by a disulfide bond and expressed on the surface of the lipid particle, e.g. lentiviral vector.

In some embodiments, the hyperfusogenic variant NiV is encoded by a polynucleotide that encodes the sequence set forth in SEQ ID NO: 290. In some embodiments, the variant NiV-F comprises the sequence of amino acids set forth in SEQ ID NO: 290, or a mature form thereof lacking the signal peptide (amino acids 1-26). In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 290 and that contains the hyperfusogenic mutations, or a mature form thereof lacking the signal peptide (amino acids 1-26). In embodiments, the variant NiV-F contains a F1 subunit corresponding to amino acid residues 110-524 of SEQ ID NO: 290) and an F2 subunit corresponding to amino acids 27-109 of SEQ ID NO: 290. In some embodiments, the F1 and F2 subunits are associated by a disulfide bond and expressed on the surface of the lipid particle, e.g. lentiviral vector.

In some embodiments, the hyperfusogenic variant NiV-F is set forth in SEQ ID NO:273 or a mature form thereof lacking the signal peptide (amino acids 1-26). In some embodiments, the hyperfusogenic variant NiV-is set forth in SEQ ID NO:274 or a mature form thereof lacking the signal peptide (amino acids 1-26). In some embodiments, the hyperfusogenic variant NiV-is set forth in SEQ ID NO:275 or a mature form thereof lacking the signal peptide (amino acids 1-26). In some embodiments, the hyperfusogenic variant NiV-is set forth in SEQ ID NO:276 or a mature form thereof lacking the signal peptide (amino acids 1-26). In some embodiments, the hyperfusogenic variant NiV-is set forth in SEQ ID NO:277, or a mature form thereof lacking the signal peptide (amino acids 1-26). In some embodiments, the hyperfusogenic variant NiV-is set forth in SEQ ID NO:278 or a mature form thereof lacking the signal peptide (amino acids 1-26). In some embodiments, the hyperfusogenic variant NiV-is set forth in SEQ ID NO:279 or a mature form thereof lacking the signal peptide (amino acids 1-26). In some embodiments, the hyperfusogenic variant NiV-is set forth in SEQ ID NO:280 or a mature form thereof lacking the signal peptide (amino acids 1-26). In some of any embodiments, the variant NiV-F is composed of an F1 and F2 subunit of any one of SEQ ID NOS: 273-280.

In some embodiments, the variant NiV-F comprises the sequence of amino acids set forth in any one of SEQ ID NO: 410-416. In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 410-416 and that contains the hyperfusogenic mutations. In embodiments, the variant NiV-F contains an F1 subunit corresponding to amino acid residues 84-498 of any of SEQ ID NOS:410-416) and an F2 subunit corresponding to amino acids 1-83 of any one of SEQ ID NOS: 410-416. In some embodiments, the F1 and F2 subunits are associated by a disulfide bond and expressed on the surface of the lipid particle, e.g. lentiviral vector.

In some embodiments, the hyperfusogenic mutation is to a residue of the F protein which functions in maintaining stability of the hexamer. In some embodiments, the native or wild-type NiV-F set forth in SEQ ID NO: 1 or 2 or a functional variant or biologically active portion thereof (e.g. SEQ ID NO: 302 or SEQ ID NO:303) is modified with a hyperfusogenic mutation to an amino acid which is involved in hexamer stability. Without wishing to be bound by theory, it is observed that the modulation of subunit stability of Paramyxovirus F proteins results in conformational changes or temporal changes to the F protein that may lead to hyperfusogenicity. In some embodiments, the variant NiV-F comprises a mutation at one or more of positions 109 and/or 303, with reference to numbering of SEQ ID NO:1, to leucine (L) or a conservative amino acid thereof. In some embodiments, the amino acid substitution is to leucine (L), isoleucine (I), valine (V), phenylalanine (F), glutamine (Q), threonine (T) or tyrosine (Y). In some embodiments, the mutation is R109L, Q393L or R109L and Q393L, with reference to numbering set forth in SEQ ID NO:1. In some embodiments, the mutations are in the sequence set forth in SEQ ID NO: 1, SEQ ID NO:2, SEQ ID NO: 302 or SEQ ID NO:303.

In some embodiments, the hyperfusogenic variant NiV-F is encoded by a polynucleotide that encodes a sequence set forth in any one of SEQ ID NOS: 291-293.

In some embodiments, the variant NiV-F comprises the sequence of amino acids set forth in any one of SEQ ID NO: 291-293, or a mature form thereof lacking the signal peptide (amino acids 1-26). In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 291-293 and that contains the hyperfusogenic mutations, or a mature form thereof lacking the signal peptide (amino acids 1-26). In embodiments, the variant NiV-F contains an F1 subunit corresponding to amino acid residues 110-524 of any of SEQ ID NOS:291-293 and an F2 subunit corresponding to amino acids 27-109 of any one of SEQ ID NOS: 291-293. In some embodiments, the F1 and F2 subunits are associated by a disulfide bond and expressed on the surface of the lipid particle, e.g. lentiviral vector.

In some embodiments, the hyperfusogenic variant NiV-F is set forth in SEQ ID NO:291 or a mature form thereof lacking the signal peptide (amino acids 1-26). In some embodiments, the hyperfusogenic variant NiV-is set forth in SEQ ID NO:292 or a mature form thereof lacking the signal peptide (amino acids 1-26). In some embodiments, the hyperfusogenic variant NiV-F is set forth in SEQ ID NO:293 or a mature form thereof lacking the signal peptide (amino acids 1-26). In some of any embodiments, the variant NiV-F is composed of an F1 and F2 subunit of any one of SEQ ID NOS: 291-293.

In some embodiments, the variant NiV-F comprises the sequence of amino acids set forth in SEQ ID NO: 292. In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 292.

In some embodiments, the variant NiV-F comprises the sequence of amino acids set forth in any one of SEQ ID NO: 417-419. In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 417-419 and that contains the hyperfusogenic mutations. In embodiments, the variant NiV-F contains and F1 corresponding to amino acid residues 84-498 of any one of SEQ ID NOS:417-419 and an F2 subunit corresponding to amino acids 1-83 of any one of SEQ ID NOS: 417-419. In some embodiments, the F1 and F2 subunits are associated by a disulfide bond and expressed on the surface of the lipid particle, e.g. lentiviral vector.

In some embodiments, the native or wild-type NiV-F set forth in SEQ ID NO:1 or SEQ ID NO:2 contains a hyperfusogenic variant from a viral protein. In some embodiments, the native or wild-type NiV-F set forth in SEQ ID NO:1 or SEQ ID NO:2 contains a hyperfusogenic variant from a Hendra F protein.

In some embodiments, the variant NiV-F is encoded by a polynucleotide that encodes a protein that is set forth in any one of SEQ ID NOS: 281-283, or any one of SEQ ID NOS: 294-296. In some embodiments, the variant NiV-F comprises the sequence of amino acids set forth in any one of SEQ ID NO: 281-283 or 294-296, or a mature form thereof lacking the signal peptide (amino acids 1-26). In some embodiments, the variant NiV-F comprises a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 283-285, 294-296 and that contains the hyperfusognic variant, or a mature form thereof lacking the signal peptide (amino acids 1-26).

### D. Heterologous Signal Peptide

In some embodiments, the variant NiV-F polypeptide is encoded by a nucleotide sequence comprising a sequence encoding a heterologous signal peptide sequence. In some embodiments, the encoded variant NiV-F polypeptide contains a heterologous signal peptide. Thus, in some embodiments, a provided variant NiV-F polypeptide herein may be disclosed as an expressed or encoded sequence including a heterologous N-terminal signal sequence. It is understood that as such a heterologous N-terminal signal sequences are commonly cleaved co- or post-translationally, the mature protein sequences for the F protein sequences disclosed herein are also contemplated as lacking the N-terminal signal sequence. In some embodiments, any of the provided variant NiV-F proteins described herein, including any containing modification in the ectodomain, cytoplasmic tail, and/or transmembrane domain, may include a heterologous signal sequence as described herein.

In some embodiments, the native or wild-type NiV-F signal peptide set forth in SEQ ID NO:3 in a sequence encoding a NiV-F or a variant NiV-F such as any as described, is replaced with a heterologous signal sequence from a viral protein or a mammalian protein. In some embodiments, the signal sequences are from other virus fusogens or other cell proteins that are associated with lentiviral particle budding. In some embodiments, modification of a variant NiV-F with a heterologous signal sequence or peptide may result in improved expression at the cell surface at sites of viral budding.

In some embodiments, the heterologous signal sequence is a signal sequence from a Paramyxovirus, such as a henipavirus. In some embodiments, the heterologous signal sequence is from a Hendra virus, such as the HeVF signal sequence set forth in SEQ ID NO:340. In some embodiments, the heterologous signal sequence is from a Cedar virus, such as the CeVF signal sequence set forth in SEQ ID NO:341. In some embodiments, the heterologous signal sequence is from a Human Parainfluenza Virus 2 (HPIV2), such as the HPIV-2 signal sequence set forth in SEQ ID NO:342. In some embodiments, the heterologous signal sequence is from a Measles virus, such as the MevF signal sequence set forth in SEQ ID NO:343. In some embodiments, the heterologous signal sequence is from a Sendai virus, such as the SevF signal sequence set forth in SEQ ID NO:345.

In some embodiments, the heterologous signal sequence is a signal sequence from a virus that is not a Parmyxovirus. In some embodiments, the heterologous signal sequence is a signal sequence from HIV-1, Baboon endogenous retrovirus, Cocal virus, Ebola virus, Gibon ape leukemia virus, Murine leukemia virus, or Vesicular stomatitis virus. In some embodiments, the heterologous signal sequence is a HIV1-Env signal sequence, BaEV signal sequence, Cocal signal sequence, EboV signal sequence, GaLV signal sequence, MLV-A signal sequence, or VSVG signal sequence. In some embodiments, the heterologous signal sequence is set forth in any one of SEQ ID NOS: 346-352.

In some embodiments, the heterologous signal sequence is a signal sequence from a mammalian protein. In some embodiments, the mammalian protein is a human protein. In some embodiments, the heterologous signal sequence is a Prolactin signal sequence, IgGk-L signal sequence, Albumin signal sequence, CD5 signal sequence, Trypsinogen signal sequence orIL2 signal sequence. In some embodiments, the heterologous signal sequence is set forth in any one of SEQ ID NOS: 353-358.

In some embodiments, the variant NiV-F comprises, or is encoded by a nucleotide sequence encoding, the sequence of amino acids set forth in SEQ ID NO:1 in which the signal sequence (amino acids 1-26) is replaced with a heterologous signal sequence set forth in any one of SEQ ID NOS: 340-358. In some embodiments, the variant NiV-F comprises, or is encoded by a nucleotide sequence encoding, the sequence of amino acids set forth in SEQ ID NO:302 in which the signal sequence (amino acids 1-26) is replaced with a heterologous signal set forth in any one of SEQ ID NOS: 340-358.

In some embodiments, the variant NiV-F comprises the sequence of amino acids set forth in any one of SEQ ID NO:249-267, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 249-267.

In some embodiments, the variant NiV-F comprises the sequence of amino acids set forth in SEQ ID NO: 249, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 249.

In some embodiments, the variant NiV-F comprises the sequence of amino acids set forth in SEQ ID NO: 261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 261.

In some embodiments, the variant NiV-F is encoded by a nucleotide sequence that encodes a variant NiV-F that comprises the sequence of amino acids set forth in any one of SEQ ID NO:249-267, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO:249-267. In some embodiments, the encoded NiV-F, when processed, contains an F1 and an F2 subunit, in which the F1 subunit corresponds to amino acid residues 110-524 of any of SEQ ID NOS:249-267 and F2 subunit corresponds to amino acids 27-109 of any one of SEQ ID NOS: 249-267. In some embodiments, the F1 and F2 subunits are associated by a disulfide bond and expressed on the surface of the lipid particle, e.g. lentiviral vector.

In some embodiments, the variant NiV-F is encoded by a nucleotide sequence that encodes a variant NiV-F that comprises the sequence of amino acids set forth in SEQ ID NO 249, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO 249. In some embodiments, the encoded NiV-F, when processed, contains an F1 and an F2 subunit, in which the F1 subunit corresponds to amino acid residues 110-524 of SEQ ID NO 249 and F2 subunit corresponds to amino acids 27-109 of any one of SEQ ID NO 249. In some embodiments, the F1 and F2 subunits are associated by a disulfide bond and expressed on the surface of the lipid particle, e.g. lentiviral vector.

In some embodiments, the variant NiV-F is encoded by a nucleotide sequence that encodes a variant NiV-F that comprises the sequence of amino acids set forth in SEQ ID NO 261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO 261. In some embodiments, the encoded NiV-F, when processed, contains an F1 and an F2 subunit, in which the F1 subunit corresponds to amino acid residues 110-524 of SEQ ID NO 261 and F2 subunit corresponds to amino acids 27-109 of any one of SEQ ID NO 261. In some embodiments, the F1 and F2 subunits are associated by a disulfide bond and expressed on the surface of the lipid particle, e.g. lentiviral vector.

### E. Heterologous or Modified Transmembrane Domain

In some embodiments, the variant NiV-F polypeptide comprises a heterologous or modified transmembrane domain. In some embodiments, the native or wild-type transmembrane of NiV-F (e.g. corresponding to amino acids 488-518 of SEQ ID NO:1) is replaced with a modified or heterologous transmembrane domain.

In some embodiments, the heterologous transmembrane domain is a transmembrane from another Paramyxovirus, such as a henipavirus. In some embodiments, the heterologous transmembrane domain is from a Hendra virus. In some embodiments, the replaced transmembrane domain is a heterologous transmembrane domain that has the sequence set forth in SEQ ID NO:361.

In some embodiments, the transmembrane domain is a modified transmembrane domain that contains one or more amino acid replacements (substitutions) from S490A, Y498A, S504A or I516V corresponding to numbering of positions set forth in SEQ ID NO:1 or SEQ ID NO:302. In some embodiments, the replaced transmembrane domain is a modified transmembrane domain that has the sequence set forth in SEQ ID NO:359, SEQ ID NO:360, SEQ ID NO:362 or SEQ ID NO:363.

In some embodiments, the variant NiV-F comprises the sequence of amino acids set forth in SEQ ID NO:1 in which the transmembrane domain (amino acids 488-518) is replaced with a heterologous or modified transmembrane domain set forth in any one of SEQ ID NOS: 359-363. In some embodiments, the variant NiV-F comprises the sequence of amino acids set forth in SEQ ID NO:302 in which the transmembrane domain (amino acids 488-518) is replaced with a heterologous or modified transmembrane domain set forth in any one of SEQ ID NOS: 359-363. In some embodiments, the variant NiV-F comprises the sequence of amino acids set forth in SEQ ID NO:303 in which the transmembrane domain (amino acids 462-492) is replaced with a heterologous or modified transmembrane domain set forth in any one of SEQ ID NOS: 359-363.

In some embodiments, the variant NiV-F comprises the sequence of amino acids set forth in any one of SEQ ID NO:268-272, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 268-272. In embodiments, the variant NiV-F contains a F1 subunit corresponding to amino acid residues 110-524 of any of SEQ ID NOS:268-272 and an F2 subunit corresponding to amino acids 27-109 of any one of SEQ ID NOS: 268-272. In some embodiments, the F1 and F2 subunits are associated by a disulfide bond and expressed on the surface of the lipid particle, e.g. lentiviral vector.

In some embodiments, the variant NiV-F comprises the sequence of amino acids set forth in SEQ ID NO: 270, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 270. In embodiments, the variant NiV-F contains a F1 subunit corresponding to amino acid residues 110-524 of SEQ ID NO: 270 and an F2 subunit corresponding to amino acids 27-109 of SEQ ID NO: 270. In some embodiments, the F1 and F2 subunits are associated by a disulfide bond and expressed on the surface of the lipid particle, e.g. lentiviral vector.

In some embodiments, the variant NiV-F comprises the sequence of amino acids set forth in any one of SEQ ID NOs: 277, 284, 285, 420 or 421, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 277, 284, 285, 420 or 421. In embodiments, the variant NiV-F contains an F1 subunit corresponding to amino acid residues 84-498 of any of SEQ ID NOS: 277, 284, 285, 420 or 421 and an F2 subunit corresponding to amino acids 1-83 of any one of SEQ ID NOS: 277, 284, 285, 420 or 421. In some embodiments, the F1 and F2 subunits are associated by a disulfide bond and expressed on the surface of the lipid particle, e.g. lentiviral vector.

### F. Combinatorial Variant Nipah F Glycoproteins

In some embodiments, the variant NiV-F comprises a combination of the various modification strategies described herein, including in Section II.A-E. In some embodiments, modification of an individual F variant (e.g., truncated, modified, or chimeric NiV-F tail, modified ectodomain, hyperfusogenic modification including glycosylation modification, signal sequence modification, and/or heterologous or modified transmembrane domain) is combined with another modification or with other modifications within the NiV-F protein. In some embodiments, modification of an individual F variant is combined with another modification or with other modifications to a different loci within the NiV-F protein.

In some embodiments, an individual NiV-F cytoplasmic tail variant further comprises a modification in any of the ectodomain or transmembrane domain. In some embodiments, an individual NiV-F cytoplasmic tail variant further comprises a heterologous signal peptide. In some embodiments, an individual NiV-F cytoplasmic tail variant further comprises a hyperfusogenic modification, such as a glycosylation mutation or hexamer stabilizing mutation.

In some embodiments, combining modifications (e.g, such as modification or truncations) can result in an additive effect on resultant pseudotyped particle titer.

In some embodiments, the variant NiV-F comprises the sequence of amino acids set forth in any one of SEQ ID NOs: 57, 58, 297-300, 367-378 or 423-456, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 57, 58, 297-300, 367-378 or 423-456. In some embodiments, the variant NiV-F has the sequence set forth in any one of SEQ ID NOs: 57, 58, 297-300, 367-378 or 423-456. In some embodiments, the encoded NiV-F, when processed, contains an F1 and an F2 subunit. In some embodiments, the F1 and F2 subunits are associated by a disulfide bond and expressed on the surface of the lipid particle, e.g. lentiviral vector.

### G. Polynucleotides Encoding Variant NiV-F

Provided herein are polynucleotides comprising a nucleic acid sequence encoding a variant NiV-F protein described herein. The polynucleotides may include a sequence of nucleotides encoding any of the variant NiV-F proteins described above. In some embodiments, the polynucleotide can be a synthetic nucleic acid. Also provided are expression vectors containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the variant NiV-F protein. For expression of the variant NiV-F protein, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-la). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. In some embodiments, a suitable promoter is CAG promoter, optionally wherein said CAG promoter comprises (C) the cytomegalovirus (CMV) early enhancer element, (A) the promoter, the first exon and the first intron of chicken beta-actin gene, and (G) the splice acceptor of the rabbit beta-globin gene

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the variant NiV-F protein. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the variant NiV-F protein is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the variant NiV-F proteins, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### III. LIPID PARTICLES CONTAINING VARIANT NIV-F GLYCOPROTEINS AND METHODS OF PRODUCTION

Provided herein is a lipid particle comprising a lipid bilayer, a lumen surrounded by the lipid bilayer and a variant NiV-F protein, such as any as described, in which the variant NiV-F is embedded within the lipid bilayer. In some embodiments, the lipid particle may additionally contain an exogenous agent (e.g. therapeutic agent) for delivery to a cell. In some embodiments, a lipid particle is introduced to a cell in the subject. Also provided are methods of delivering any of the provided lipid particles to a cell.

In some embodiments, the provided lipid particles exhibit fusogenic activity, which is mediated by the variant NiV-F along with any of the provided G proteins that facilitates merger or fusion of the two lumens of the lipid particle and the target cell membranes. Thus, among provided lipid particles are fusosomes. In some embodiments, the fusosome comprises a naturally derived bilayer of amphipathic lipids with the variant NiV-F as a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the variant NiV-F disposed in the lipid bilayer. In some embodiments, the fusosome comprises several different types of lipids, e.g., amphipathic lipids, such as phospholipids

In some embodiments, the lipid particle includes a naturally derived bilayer of amphipathic lipids that encloses lumen or cavity. In some embodiments, the lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. Exemplary methods for producing lipid-containing particles are provided in Section III.A.3. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the microvesicle is produced, e.g., solutes, proteins, nucleic acids, etc., but not all of the components of a cell, e.g., they lack a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins as described herein such as a variant NiV-F protein and, in some aspects, also a protein such as a NiV-G protein.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a variant NiV-F protein and a G protein (e.g. NiV-G), such as any described including any that are exogenous or overexpressed relative to the source cell, is disposed in the lipid bilayer. In provided embodiments, the variant NiV-F protein and G protein (e.g. NiV-G protein) are exposed on the outside surface of the lipid bilayer of the lipid particle (e.g. lentiviral vector).

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer may be comprised of one or more lipids of the same or different type. In some embodiments, the source cell comprises a cell selected from CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell based particle.

In particular embodiments, the lipid particle is virally derived. In some embodiments, the lipid particle can be a viral-based particle, such as a viral vector particle (e.g. lentiviral vector particle) or a virus-like particle (e.g. a lentiviral-like particle). In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In particular embodiments, the lipid particle is not virally derived. In some embodiments, the lipid particle can be a nanoparticle, a vesicle, an exosome, a dendrimer, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In particular embodiments, an exogenous agent, such as a polynucleotide or polypeptide, is encapsulated within the lumen of a lipid particle. Embodiments of provided lipid particles may have various properties that facilitate delivery of a payload, such as, e.g., a desired transgene or exogenous agent, to a target cell. The exogenous agent may be a polynucleotide or a polypeptide. In some embodiments, a lipid particle provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences (polynucleotide) encoding an exogenous agent or a polypeptide exogenous agent for treating the disease or condition.

The lipid particles can include spherical particles or can include particles of elongated or irregular shape.

In some embodiments, a composition of particles can be assessed for one or more features related to their size, including diameter, range of variation thereof above and below an average (mean) or median value of the diameter, coefficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the provided lipid particle has a diameter of, or the average (mean) diameter of particles in a composition is, less than about 3 μm, less than about 2 μm, less than about 1 μm, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the lipid particle has a diameter of, or the average (mean) diameter of particles in a composition is, less than about 400 nm. In another embodiment, the lipid particle has a diameter of, or the average (mean) diameter of particles in a composition is, less than about 150 nm. In some embodiments, the lipid particle has a diameter of, or the average (mean) diameter of particles in a composition is, between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the lipid particles in a composition fall within 50% of the median diameter of the lipid particles. In some embodiments, 90% of the lipid particles in a composition fall within 25% of the median diameter of the lipid particles. In some embodiments, 90% of the lipid particles in a composition fall within 20% of the median diameter. In some embodiments, 90% of the lipid particles in a composition fall within 15% of the median diameter of lipid particles. In some embodiments, 90% of the lipid particles in a composition fall within 10% of the median diameter of the lipid particles.

In some embodiments, 75% of the lipid particles in a composition fall within +/−2 or +/−1 St Dev standard deviations (St Dev) of the mean diameter of lipid particles. In some embodiments, 80% of the lipid particles in a composition fall within +/−2 St Dev or +/−1 St Dev of the mean diameter of lipid particles. In some embodiments, 85% of the lipid particles in a composition fall within +/−2 St Dev or +/−1 St Dev of the mean diameter of lipid particles. In some embodiments, 90% of the lipid particles in a composition fall within +/−2 St Dev or +/−1 St Dev of the mean diameter of lipid particles. In some embodiments, 95% of the lipid particles in a composition fall within +/−2 St Dev or +/−1 St Dev of the mean diameter of lipid particles.

In some embodiments, the lipid particles have an average hydrodynamic radius, e.g. as determined by dynamic light scattering (DLS), of about 100 nm to about two microns. In some embodiments, the lipid particles have an average hydrodynamic radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the lipid particles have an average geometric radius, e.g. as determined by a multi-angle light scattering, of about 100 nm to about two microns. In some embodiments, the lipid particles have an average geometric radius between at or about 2 μm and at or about 1 sm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of lipid particles is less than at or about 30%, less than at or about 25%, less than at or about 20%, less than at or about 15%, less than at or about 10% or less than at or about 5%.

In some embodiment, provided compositions of lipid particles are characterized by their polydispersity index, which is a measure of the size distribution of the particles wherein values between 1 (maximum dispersion) and 0 (identical size of all of the particles) are possible. In some embodiments, compositions of lipid particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7. Various lipid particles are known, any of which can be generated in accord with the provided embodiments. Non-limiting examples of lipid particles include any as described in, or contain features as described in, International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

Features of the provided lipid particles are described in the following subsections.

### A. Viral-Based Particles

Provided herein are viral-based particles derived from a virus, including those derived from retroviruses or lentiviruses, containing a variant NiV-F, such as described in Section II, and in some cases also an G protein (e.g. NiV-G) as described. In some embodiments, the lipid particle's bilayer of amphipathic lipids is or comprises the viral envelope. In some embodiments, the lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the lipid particle further comprises one or more viral non-structural proteins, e.g., in its cavity or lumen. In some embodiments, the viral-based particle is or comprises a virus-like particle (VLP). In some embodiments, the VLP does not comprise any viral genetic material. In some embodiments, the viral-based particle does not contain any virally derived nucleic acids or viral proteins, such as viral structural proteins.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with viral proteins, including viral glycoproteins, including the variant NiV-F.

In some embodiments, one or more transducing units of viral particles or virus-like particles, e.g. retroviral particles or retroviral-like particles, are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴ transducing units per kg are administered to the subject. In some embodiments at least 1, 10, 100, 1000, 10⁴, 10⁵, 10⁶, 10⁷, 10⁸, 10⁹, 10¹⁰, 10¹¹, 10¹², 10¹³, or 10¹⁴ transducing units per target cell or per target cell per ml of blood are administered to the subject.

### 1. Viral Vector Particles

In some embodiments, the lipid particle is or comprises a virus or a viral vector, e.g., a retrovirus or retroviral vector, e.g., a lentivirus or lentiviral vector. In some embodiments, the virus or viral vector is recombinant. For instance, the viral particle may be referred to as a recombinant virus and/or a recombinant viral vector, which are used interchangeably. In some embodiments, the lipid particle is a recombinant lentivirus vector particle.

In some embodiments, a lipid particle comprises a lipid bilayer comprising a retroviral vector comprising an envelope. For instance, in some embodiments, the bilayer of amphipathic lipids is or comprises the viral envelope. The viral envelope may comprise a fusogen, e.g., a variant NiV-F such as described in Section II, and in some cases also a G protein (e.g. NiV-G) such as described in Section III.B, that is endogenous to the virus or is a pseudotyped fusogen. In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen. In some embodiments, the virus based vector particles are lentivirus. In some embodiments, the lentiviral vector particle is Human Immunodeficiency Virus-1 (HIV-1).

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle, such as retroviral vector particle, comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid. In some embodiments, the lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments, the lipid particle nucleic acid further comprises a retroviral cis-acting RNA packaging element, and a cPPT/CTS element. In some embodiments the lipid particle nucleic acid further comprises one or more insulator element. In some embodiments, the recognition sites are situated between the poly A tail sequence and the WPRE.

In some embodiments, the lipid particle comprises supramolecular complexes formed by viral proteins that self-assemble into capsids. In some embodiments, the lipid particle is a viral particle derived from viral capsids. In some embodiments, the lipid particle is a viral particle derived from viral nucleocapsids. In some embodiments, the lipid particle comprises nucleocapsid-derived that retain the property of packaging nucleic acids.

In some embodiments, the lipid particle packages nucleic acids from host cells carrying one or more viral nucleic acids (e.g. retroviral nucleic acids) during the expression process. In some embodiments, the nucleic acids do not encode any genes involved in virus replication. In particular embodiments, the lipid particle is a virus-based particle, e.g. retrovirus particle such as a lentivirus particle, that is replication defective.

In some cases, the lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or US which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and US). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV-based vector backbones (i.e., HIV cis-acting sequence elements) are used.

A viral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. nucleic acid encoding an exogenous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and US is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and US in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. US and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an exogenous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide (nt) 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and US regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The US region is typically the sequence between the primer binding site and the R region and can contain the polyadenylation sequence. The R (repeat) region can be flanked by the U3 and US regions. The LTR is typically composed of U3, R and US regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Y] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the US region is removed, altered, or replaced, for example, with an exogenous poly(A) sequence. The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Virus-Like Particles

In some embodiments, the viral-based particles are viral-like lipid particles (VLPs) that are derived from virus. In some embodiments, the viral envelope may comprise a fusogen, e.g., a fusogen that is endogenous to the virus or a pseudotyped fusogen, e.g., a variant NiV-F as described in Section II, and in some cases also a G protein (e.g. NiV-G) such as described below in Section III.B. The VLPs include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, the lipid particle's bilayer of amphipathic lipids is or comprises lipids derived from a cell. In some embodiments, a VLP contains at least one type of structural protein from a virus. In most cases this protein will form a proteinaceous capsid. In some cases, the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the vector vehicle particle comprises supramolecular complexes formed by viral proteins that self-assemble into capsids. In some embodiments, the vector vehicle particle is a virus-like particle derived from viral capsid proteins. In some embodiments, the vector vehicle particle is a virus-like particle derived from viral nucleocapsid proteins. In some embodiments, the vector vehicle particle comprises nucleocapsid-derived proteins that retain the property of packaging nucleic acids. In some embodiments, the viral-based particles, such as virus-like particles comprise only viral structural glycoproteins among proteins from the viral genome. In some embodiments, the vector vehicle particle does not contain a viral genome.

In some embodiments, the vector vehicle particle packages nucleic acids from host cells during the expression process, such as a nucleic acid encoding an exogenous agent. In some embodiments, the nucleic acids do not encode any genes involved in virus replication. In particular embodiments, the vector vehicle particle is a virus-like particle, e.g. retrovirus-like particle such as a lentivirus-like particle, that is replication defective.

In some embodiments, the vector vehicle particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

### 3. Methods of Generating Viral-Based Particles

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vector particles may be produced in multiple cell culture systems including bacteria, mammalian cell lines, insect cell lines, yeast and plant cells. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modem retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A virus-like particle (VLP) which comprises a sequence that is devoid of or lacking viral RNA, such as described in Section 111.2, may be the result of removing or eliminating the viral RNA from the sequence. Similar to the viral vector particles, such as described in Section 111.1, VLPs contain a viral outer envelope made from the host cell (i.e., producer cell or source cell) lipid-bilayer as well as at least one viral structural protein. In some embodiments, a viral structural protein refers to any viral protein or fragment thereof which contributes to the structure of the viral core or capsid.

Generally, for lentiviral vector particles, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U1 small nuclear ribonucleoprotein particle, a Nova protein, a TF111 A protein, a TIS 11 protein, a trp RNA-binding attenuation protein (TRAP) or a pseudouridine synthase.

In some embodiments, the gag protein is a polyprotein. In some embodiments, the gag protein is a polyprotein comprising a MA polypeptide, a CA polypeptide, and an NC polypeptide; ii) one or more exogenous polypeptides (such as is described in Section II.C.2); and iii) one or more heterologous protease cleavage sites, wherein at least one of the one or more heterologous protease cleavage sites is between the gag polyprotein and the one or more exogenous polypeptides.

In some embodiments, the MA, CA, and NC portions of the gag polyprotein can be of any retrovirus known in the art. For example in some embodiments, the gag polyprotein is a gag polyprotein of an alpha retrovirus, a beta retrovirus, a gamma retrovirus, a delta retrovirus, an epsilon retrovirus, or a spumavirus. In some embodiments, the gag polyprotein is a gag polyprotein of a human immunodeficiency virus.

In some embodiments, the gag polyprotein is a human immunodeficiency virus (HIV) gag polyprotein comprising a MA polypeptide, a CA polypeptide, a p2 polypeptide, an NC polypeptide, a pi polypeptide, and a p6 polypeptide. In some embodiments, the gag polyprotein comprises one or more heterologous protease cleavage sites between one or more of: i) the MA polypeptide and the CA polypeptide; ii) the CA polypeptide and the p2 polypeptide; iii) the p2 polypeptide and the NC polypeptide; iv) the NC polypeptide and the pi polypeptide; and v) the pi polypeptide and the p6 polypeptide. In some embodiments, a gag polyprotein can comprise: MA-heterologous protease cleavage site-CA-heterologous protease cleavage site-p2-heterologous protease cleavage site-NC-p1-p6. In some embodiments, the heterologous protease cleavage site is a TEV protease cleavage site: ENLYFQS, where cleavage occurs between the Gin and the Ser.

In some embodiments, the assembly of a viral based vector particle (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle, such as described above, can be detected by any suitable technique known in the art. Examples of such techniques include, e.g., electron microscopy, dynamic light scattering, selective chromatographic separation and/or density gradient centrifugation.

### B. G Proteins

In some embodiments, the lipid particle includes an envelope protein exposed on the surface of the targeted lipid particle. In some embodiments, the envelope protein contains a henipavirus envelope attachment glycoprotein G (G protein) or a biologically active portion thereof. In some embodiments, the G protein may be retargeted by linkage to a targeting moiety, such as a binding molecule (e.g. antibody or antigen-binding fragment, e.g. sdAb or scFv) that binds to a target cell. In some embodiments, the G protein and the variant NiV-F protein provided herein together exhibit fusogenic activity to a target cell, such as to deliver an exogenous agent or nucleic acid exogenous agent to the target cell.

The attachment G proteins are type II transmembrane glycoproteins containing an N-terminal cytoplasmic tail (e.g. corresponding to amino acids 1-49 of SEQ ID NO:304), a transmembrane domain (e.g. corresponding to amino acids 50-70 of SEQ ID NO:304), and an extracellular domain containing an extracellular stalk (e.g. corresponding to amino acids 71-187 of SEQ ID NO:304), and a globular head (corresponding to amino acids 188-602 of SEQ ID NO:304). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 304) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described, a lipid particle can contain heterologous G and F proteins from different species. In particular embodiments, the F protein or the functionally active variant or biologically active portion thereof retains fusogenic activity in conjunction with a G protein as provided, such as any set forth below. Fusogenic activity includes the activity of the variant F protein in conjunction with a G protein to promote or facilitate fusion of two membrane lumens, such as the lumen of the lipid particle provided herein (e.g. having embedded in its lipid bilayer, such as exposed on its surface, a G protein and a variant F protein), and a cytoplasm of a target cell, e.g. a cell that contains a surface receptor or molecule that is recognized or bound by the G protein.

Exemplary Henipavirus protein G sequences are provided in Table 2

TABLE 2
Henipavirus protein G sequence clusters. Column 1, Genbank ID includes the Genbank ID
of the whole genome sequence of the virus that is the centroid sequence of the cluster.
Column 2, nucleotides of CDS provides the nucleotides corresponding to the CDS of the
gene in the whole genome. Column 3, Full Gene Name, provides the full name of the gene
including Genbank ID, virus species, strain, and protein name. Column 4, Sequence,
provides the amino acid sequence of the gene. Column 5, #Sequences/Cluster, provides
the number of sequences that cluster with this centroid sequence. Column 6 provides
the SEQ ID numbers for the described sequences.

| Genbank | Nucleotides | Full | | SEQ ID |
|---|---|---|---|---|
| ID | of CDS | sequence ID | Sequence | NO |
| AF017149 | 8913- | gb: AF017149\| | MMADSKLVSLNNNLSGKIKDQGKVIKN | 379 |
| | 10727 | Organism: | YYGTMDIKKINDGLLDSKILGAFNTVIAL | |
| | | Hendra virus\| | LGSIIIIVMNIMIIQNYTRTTDNQALIKESL | |
| | | Strain Name: | QSVQQQIKALTDKIGTEIGPKVSLIDTSST | |
| | | UNKNOWN- | ITIPANIGLLGSKISQSTSSINENVNDKCKF | |
| | | AF017149\| | TLPPLKIHECNISCPNPLPFREYRPISQGVS | |
| | | Protein | DLVGLPNQICLQKTTSTILKPRLISYTLPI | |
| | | Name: | NTREGVCITDPLLAVDNGFFAYSHLEKIG | |
| | | glycoprotein\| | SCTRGIAKQRIIGVGEVLDRGDKVPSMF | |
| | | Gene Symbol: | MTNVWTPPNPSTIHHCSSTYHEDFYYTL | |
| | | G | CAVSHVGDPILNSTSWTESLSLIRLAVRP | |
| | | | KSDSGDYNQKYIAITKVERGKYDKVMP | |
| | | | YGPSGIKQGDTLYFPAVGFLPRTEFQYN | |
| | | | DSNCPIIHCKYSKAENCRLSMGVNSKSH | |
| | | | YILRSGLLKYNLSLGGDIILQFIEIADNRL | |
| | | | TIGSPSKIYNSLGQPVFYQASYSWDTMIK | |
| | | | LGDVDTVDPLRVQWRNNSVISRPGQSQC | |
| | | | PRFNVCPEVCWEGTYNDAFLIDRLNWVS | |
| | | | AGVYLNSNQTAENPVFAVFKDNEILYQV | |
| | | | PLAEDDTNAQKTITDCFLLENVIWCISLV | |
| | | | EIYDTGDSVIRPKLFAVKIPAQCSES | |
| AF212302 | 8943- | gb: AF212302\| | MPAENKKVRFENTTSDKGKIPSKVIKSY | 304 |
| | 10751 | Organism: | YGTMDIKKINEGLLDSKILSAFNTVIALL | |
| | | Nipah virus\| | GSIVIIVMNIMIIQNYTRSTDNQAVIKDAL | |
| | | Strain Name: | QGIQQQIKGLADKIGTEIGPKVSLIDTSSTI | |
| | | UNKNOWN- | TIPANIGLLGSKISQSTASINENVNEKCKF | |
| | | AF212302\| | TLPPLKIHECNISCPNPLPFREYRPQTEGV | |
| | | Protein | SNLVGLPNNICLQKTSNQILKPKLISYTLP | |
| | | Name: | VVGQSGTCITDPLLAMDEGYFAYSHLER | |
| | | attachment | IGSCSRGVSKQRIIGVGEVLDRGDEVPSL | |
| | | glycoprotein\| | FMTNVWTPPNPNTVYHCSAVYNNEFYY | |
| | | Gene | VLCAVSTVGDPILNSTYWSGSLMMTRLA | |
| | | Symbol: G | VKPKSNGGGYNQHQLALRSIEKGRYDK | |
| | | | VMPYGPSGIKQGDTLYFPAVGFLVRTEF | |
| | | | KYNDSNCPITKCQYSKPENCRLSMGIRPN | |
| | | | SHYILRSGLLKYNLSDGENPKVVFIEISD | |
| | | | QRLSIGSPSKIYDSLGQPVFYQASFSWDT | |
| | | | MIKFGDVLTVNPLVVNWRNNTVISRPGQ | |
| | | | SQCPRFNTCPEICWEGVYNDAFLIDRINW | |
| | | | ISAGVFLDSNQTAENPVFTVFKDNEILYR | |
| | | | AQLASEDTNAQKTITNCFLLKNKIWCISL | |
| | | | VEIYDTGDNVIRPKLFAVKIPEQCT | |
| JQ001776 | 8170- | gb: JQ001776: | MLSQLQKNYLDNSNQQGDKMNNPDKK | 380 |
| | 10275 | 8170-10275\| | LSVNFNPLELDKGQKDLNKSYYVKNKN | |
| | | Organism: | YNVSNLLNESLHDIKFCIYCIFSLLIIITIINI | |
| | | Cedar | ITISIVITRLKVHEENNGMESPNLQSIQDS | |
| | | virus\|Strain | LSSLTNMINTEITPRIGILVTATSVTLSSSI | |
| | | Name: CG1a\| | NYVGTKTNQLVNELKDYITKSCGFKVPE | |
| | | Protein | LKLHECNISCADPKISKSAMYSTNAYAEL | |
| | | Name: | AGPPKIFCKSVSKDPDFRLKQIDYVIPVQ | |
| | | attachment | QDRSICMNNPLLDISDGFFTYIHYEGINSC | |
| | | glycoprotein\| | KKSDSFKVLLSHGEIVDRGDYRPSLYLLS | |
| | | Gene | SHYHPYSMQVINCVPVTCNQSSFVFCHIS | |
| | | Symbol: G | NNTKTLDNSDYSSDEYYITYFNGIDRPKT | |
| | | | KKIPINNMTADNRYIHFTFSGGGGVCLGE | |
| | | | EFIIPVTTVINTDVFTHDYCESFNCSVQTG | |
| | | | KSLKEICSESLRSPTNSSRYNLNGIMIISQ | |
| | | | NNMTDFKIQLNGITYNKLSFGSPGRLSKT | |
| | | | LGQVLYYQSSMSWDTYLKAGFVEKWKP | |
| | | | FTPNWMNNTVISRPNQGNCPRYHKCPEI | |
| | | | CYGGTYNDIAPLDLGKDMYVSVILDSDQ | |
| | | | LAENPEITVENSTTILYKERVSKDELNTRS | |
| | | | TTTSCFLFLDEPWCISVLETNRFNGKSIRP | |
| | | | EIYSYKIPKYC | |
| NC_ | 9117- | gb: NC_025256: | MPQKTVEFINMNSPLERGVSTLSDKKTL | 381 |
| 025256 | 11015 | 9117-11015\| | NQSKITKQGYFGLGSHSERNWKKQKNQ | |
| | | Organism: | NDHYMTVSTMILEILVVLGIMENLIVLTM | |
| | | Bat | VYYQNDNINQRMAELTSNITVLNLNLNQ | |
| | | Paramyxovirus | LINKIQREIIPRITLIDTATTITIPSAITYILA | |
| | | Eid_hel/GH- | TLTTRISELLPSINQKCEFKTPTLVLNDCR | |
| | | M74a/GHA/ | INCTPPLNPSDGVKMSSLATNLVAHGPSP | |
| | | 2009\|Strain | CRNFSSVPTIYYYRIPGLYNRTALDERCIL | |
| | | Name: | NPRLTISSTKFAYVHSEYDKNCTRGFKY | |
| | | BatP | YELMTFGEILEGPEKEPRMFSRSFYSPTN | |
| | | V/Eid_hel/ | AVNYHSCTPIVTVNEGYFLCLECTSSDPL | |
| | | GH- | YKANLSNSTFHLVILRHNKDEKIVSMPSF | |
| | | M74a/GHA/ | NLSTDQEYVQIIPAEGGGTAESGNLYFPC | |
| | | 2009\|Protein | IGRLLHKRVTHPLCKKSNCSRTDDESCL | |
| | | Name: | KSYYNQGSPQHQVVNCLIRIRNAQRDNP | |
| | | glycoprotein\| | TWDVITVDLTNTYPGSRSRIFGSFSKPML | |
| | | Gene Symbol: G | YQSSVSWHTLLQVAEITDLDKYQLDWL | |
| | | | DTPYISRPGGSECPFGNYCPTVCWEGTY | |
| | | | NDVYSLTPNNDLFVTVYLKSEQVAENPY | |
| | | | FAIFSRDQILKEFPLDAWISSARTTTISCF | |
| | | | MFNNEIWCIAALEITRLNDDIIRPIYYSFW | |
| | | | LPTDCRTPYPHTGKMTRVPLRSTYNY | |
| NC_ | 8716- | gb: NC_025352: | MATNRDNTITSAEVSQEDKVKKYYGVE | 382 |
| 025352 | 11257 | 8716-11257\| | TAEKVADSISGNKVFILMNTLLILTGAIITI | |
| | | Organism: | TLNITNLTAAKSQQNMLKIIQDDVNAKL | |
| | | Mojiang | EMFVNLDQLVKGEIKPKVSLINTAVSVSI | |
| | | virus\|Strain | PGQISNLQTKFLQKYVYLEESITKQCTCN | |
| | | Name: | PLSGIFPTSGPTYPPTDKPDDDTTDDDKV | |
| | | Tongguan 1\| | DTTIKPIEYPKPDGCNRTGDHFTMEPGAN | |
| | | Protein | FYTVPNLGPASSNSDECYTNPSFSIGSSIY | |
| | | Name: | MFSQEIRKTDCTAGEILSIQIVLGRIVDKG | |
| | | attachment | QQGPQASPLLVWAVPNPKIINSCAVAAG | |
| | | glycoprotein\| | DEMGWVLCSVTLTAASGEPIPHMFDGF | |
| | | Gene | WLYKLEPDTEVVSYRITGYAYLLDKQY | |
| | | Symbol: G | DSVFIGKGGGIQKGNDLYFQMYGLSRNR | |
| | | | QSFKALCEHGSCLGTGGGGYQVLCDRA | |
| | | | VMSFGSEESLITNAYLKVNDLASGKPVII | |
| | | | GQTFPPSDSYKGSNGRMYTIGDKYGLYL | |
| | | | APSSWNRYLRFGITPDISVRSTTWLKSQD | |
| | | | PIMKILSTCTNTDRDMCPEICNTRGYQDI | |
| | | | FPLSEDSEYYTYIGITPNNGGTKNFVAVR | |
| | | | DSDGHIASIDILQNYYSITSATISCFMYKD | |
| | | | EIWCIAITEGKKQKDNPQRIYAHSYKIRQ | |
| | | | MCYNMKSATVTVGNAKNITIRRY | |

In some embodiments, the G protein has a sequence set forth in any of SEQ ID NOS: 304, 379, 380, 381 or 382 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 304, 379, 380, 381 or 382.

In particular embodiments, the G protein or functionally active variant or biologically active portion is a protein that retains fusogenic activity in conjunction with a Henipavirus F protein, such as a variant NiV-F protein described herein. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F).

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 304, 379, 380, 381 or 382.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOS: 304, 379, 380, 381 or 382. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:304, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:304.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein, such as compared to wild-type NiV-G set forth in SEQ ID NO: 304. In some embodiments, the mutant NiV-G protein is truncated and lacks up to 10 contiguous amino acid residues at or near the N-terminus of the wild-type NiV-G protein, such as compared to wild-type NiV-G set forth in SEQ ID NO: 304. In some embodiments, the mutant NiV-G protein is truncated and lacks up to 15 contiguous amino acid residues at or near the N-terminus of the wild-type NiV-G protein, such as compared to wild-type NiV-G set forth in SEQ ID NO: 304. n some embodiments, the mutant NiV-G protein is truncated and lacks up to 20 contiguous amino acid residues at or near the N-terminus of the wild-type NiV-G protein, such as compared to wild-type NiV-G set forth in SEQ ID NO: 304. In some embodiments, the mutant NiV-G protein is truncated and lacks up to 25 contiguous amino acid residues at or near the N-terminus of the wild-type NiV-G protein, such as compared to wild-type NiV-G set forth in SEQ ID NO: 304. In some embodiments, the mutant NiV-G protein is truncated and lacks up to 30 contiguous amino acid residues at or near the N-terminus of the wild-type NiV-G protein, such as compared to wild-type NiV-G set forth in SEQ ID NO: 304. In some embodiments, the mutant NiV-G protein is truncated and lacks up to 35 contiguous amino acid residues at or near the N-terminus of the wild-type NiV-G protein, such as compared to wild-type NiV-G set forth in SEQ ID NO: 304. In some embodiments, the mutant NiV-G protein (also called variant NiV-G) contains an N-terminal methionine.

In some embodiments, the mutant NiV-G protein is truncated and lacks up to amino acid 34 at or near the N-terminus of the wild-type NiV-G protein, such as compared to wild-type NiV-G set forth in SEQ ID NO: 304. In some embodiments, the mutant NiV-G protein (also called variant NiV-G) contains an N-terminal methionine. In some embodiments, the mutant NiV-G protein lacks amino acids 2-34 as compared to wild-type NiV-G set forth in SEQ ID NO:304.

In some embodiments, the G protein or the functionally active variant or biologically active portion thereof binds to Ephrin B2 or Ephrin B3. In some embodiments, the G protein is a mutant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 304, 379, 380, 381 or 382, such as at least 50%, at least 60%, at least 70%, at least 80% or at least 90% of the binding of the wild-type G protein.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:304. In some embodiments, the G protein is a mutant G protein containing one or more amino acid substitutions selected from the group consisting of E501A, W504A, Q530A and E533A with reference to numbering set forth in SEQ ID NO:304. In some embodiments, the G protein is a mutant G protein that contains one or more amino acid substitutions elected from the group consisting of E501A, W504A, Q530A and E533A with reference to SEQ ID NO:304 and is a biologically active portion thereof containing an N-terminal truncation.

In particular embodiments, the G protein has the sequence of amino acids set forth in SEQ ID NO: 301, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 301 and retains fusogenic activity in conjunction with a variant NiV-F protein as described.

Reference to retaining fusogenic activity includes activity of a lipid particle (e.g. lentiviral vector) containing a variant NiV-F protein as described or biologically active portion or functionally active variant of the F protein (in conjunction with a G protein, such as a NiV-G protein as described) that is between at or about 10% and at or about 150% or more of the level or degree of binding of a reference lipid particle (e.g. lentiviral vector) that is similar, such as contains the same variant NiV-F, but that contains the corresponding wild-type G protein, such as set forth in SEQ ID NO: 304. For instance, a lipid particle (e.g. lentiviral vector) that retains fusogenic activity has at least or at least about 10% of the level or degree of fusogenic activity of the reference lipid particle that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

### 1. Re-Targeted G Proteins

In some embodiments, a G protein (such as NiV-G) is further attached or linked to a binding domain that binds to a target molecule, such as a cell surface marker. For instance, provided in some aspects is a targeted lipid particle (e.g. targeted lentiviral vector) that includes a re-targeted G protein containing any of the provided G proteins attached to a binding domain, in which the re-targeted G protein is exposed on the surface of the targeted lipid particle (e.g. targeted lentiviral vector).

In some embodiments, the targeted envelope protein contains a G protein provided herein.

In some embodiments the G protein is any of those provided in Section III.B, including NiV-G proteins with cytoplasmic domain modifications, truncated NiV-G cytoplasmic tails, or modified NiV-G cytoplasmic tails.

In some embodiments, the binding domain can be any agent that binds to a cell surface molecule on a target cells. In some embodiments, the binding domain can be an antibody or an antibody portion or fragment. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

The binding domain may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence (s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

A G protein provided herein and a binding domain that binds to a cell surface molecule on a target cell. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cardiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLCIA3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the binding domain (e.g. sdAb) variable domain binds a cell surface molecule or antigen. In some embodiments, the cell surface molecule is ASGR1, ASGR2, TM4SF5, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD8. In some embodiments, the cell surface molecule is CD4. In some embodiments, the cell surface molecule is LDL-R.

In some embodiments, the G protein or functionally active variant or biologically active portion thereof is linked directly to the binding domain and/or variable domain thereof. In some embodiments, the targeted envelope protein is a fusion protein that has the following structure: (N′-single domain antibody-C′)-(C′-G protein-N′).

In some embodiments, the G protein or functionally active variant or biologically active portion thereof is linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:287), GGGGGS (SEQ ID NO:288) or combinations thereof. In some embodiments, the polypeptide linker has the sequence (GGS)n, wherein n is 1 to 10. In some embodiments, the polypeptide linker has the sequence (GGGGS)n, (SEQ ID NO:289) wherein n is 1 to 10. In some embodiments, the polypeptide linker has the sequence (GGGGGS)n (SEQ ID NO:290), wherein n is 1 to 6.

### C. Exogenous Agent

In some embodiments, the lipid particle as described herein or pharmaceutical composition comprising same described contains an exogenous agent. In some embodiments, the lipid particle or pharmaceutical composition comprising same described herein contains a nucleic acid that encodes an exogenous agent. In some embodiments, the lipid particle contains the exogenous agent. In some embodiments, the lipid particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the lipid particle.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule.

In some embodiments, the lipid particle or pharmaceutical composition delivers to a target cell at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle. In some embodiments, the lipid particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particles, e.g., fusosomes, that contact, e.g., fuse, with the target cell(s). In some embodiments, the lipid particle composition delivers to a target tissue at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle compositions.

In some embodiments, the exogenous agent is not expressed naturally in the cell from which the lipid particle is derived. In some embodiments, the exogenous agent is expressed naturally in the cell from which the lipid particle is derived. In some embodiments, the exogenous agent is loaded into the lipid particle via expression in the cell from which the lipid particle is derived (e.g. expression from DNA or mRNA introduced via transfection, transduction, or electroporation). In some embodiments, the exogenous is expressed from DNA integrated into the genome or maintained episomally. In some embodiments, expression of the exogenous agent is constitutive. In some embodiments, expression of the exogenous agent is induced. In some embodiments, expression of the exogenous agent is induced immediately prior to generating the lipid particle. In some embodiments, expression of the exogenous agent is induced at the same time as expression of the fusogen.

In some embodiments, the exogenous agent is loaded into the lipid particle via electroporation into the lipid particle itself or into the cell from which the lipid particle is derived. In some embodiments, the exogenous agent is loaded into the lipid particle via transfection (e.g., of a DNA or mRNA encoding the exogenous agent) into the lipid particle itself or into the cell from which the lipid particle is derived.

In some embodiments, the exogenous agent may include one or more nucleic acid sequences, one or more polypeptides, a combination of nucleic acid sequences and/or polypeptides, one or more organelles, and any combination thereof. In some embodiments, the exogenous agent may include one or more cellular components. In some embodiments, the exogenous agent includes one or more cytosolic and/or nuclear components.

In some embodiments, the lipid particle contains an exogenous agent that is a nucleic acid or contains a nucleic acid encoding the exogenous agent. In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidt et a, Blood. 2014 Apr. 24; 123(17):e68-78., herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the lipid particle contains an exogenous agent that is a nucleic acid or contains a nucleic acid encoding the exogenous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the lipid particle in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR31, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### 1. Nucleic Acids

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells

In some embodiments, a lipid particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the exogenous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H 1 RNA promoter and the human tRNA-val promoter, or a strong constitutive pol II promoter.

### 2. Polypeptides

In some embodiments, the lipid particle contains a nucleic acid that encodes a protein exogenous agent (also referred to as a “payload gene encoding an exogenous agent.”). In some embodiments, a lipid particle described herein comprises an exogenous agent which is or comprises a protein.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein exogenous agents are described in the following subsections. In some embodiments, a lipid particle provided herein can include any of such exogenous agents. In particular embodiments, a lipid particle contains a nucleic acid encoding any of such exogenous agents.

a. Cytosolic Proteins

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

b. Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

### 1) Chimeric Antigen Receptors (CARs)

In some embodiments, a payload gene described herein encodes a chimeric antigen receptor (CAR) comprising an antigen binding domain. In some embodiments, an exogenous agent described herein comprises a chimeric antigen receptor (CAR) comprising an antigen binding domain. In some embodiments, the payload is or comprises a chimeric antigen receptor (CAR) comprising an antigen binding domain. In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, the antigen binding domain targets an antigen characteristic of a cell type. In some embodiments, the antigen binding domain targets an antigen characteristic of a neoplastic cell. In some embodiments, the antigen characteristic of a neoplastic cell is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (SIPIR), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Tr1; Th3; Treg17; TREG; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGFiR, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LiCAM, LeY, MSLN, IL13Rα1, Li-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACI, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, ETV6-AML, sperm protein 17, XAGEi, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the antigen binding domain targets an antigen characteristic of a T cell. In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain targets an antigen characteristic of a disorder. In some embodiments, the antigen binding domain targets an antigen characteristic of an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds to a ligand expressed on B cells, plasma cells, plasmablasts, CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Eptstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TINFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In some embodiments the exogenous agent is or comprises a CAR, e.g., a first generation CAR or a nucleic acid encoding a first generation CAR. In some embodiments, a first generation CAR comprises an antigen binding domain, a transmembrane domain, and signaling domain. In some embodiments a signaling domain mediates downstream signaling during T cell activation.

In some embodiments the exogenous agent is or comprises a second generation CAR or a nucleic acid encoding a second generation CAR. In some embodiments a second generation CAR comprises an antigen binding domain, a transmembrane domain, and two signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments the exogenous agent is or comprises a third generation CAR or a nucleic acid encoding a third generation CAR. In some embodiments, a third generation CAR comprises an antigen binding domain, a transmembrane domain, and at least three signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments the exogenous is or comprises a fourth generation CAR or a nucleic acid encoding a fourth generation CAR. In some embodiments a fourth generation CAR comprises an antigen binding domain, a transmembrane domain, and at least two, three, or four signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, or IFN-gamma, or functional fragment thereof. In some embodiments a domain which upon successful signaling of the CAR induces expression of a cytokine gene is or comprises a transcription factor or functional domain or fragment thereof. In some embodiments a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, an antigen binding domain binds to a cell surface antigen of a cell. In some embodiments, a cell surface antigen is characteristic of one type of cell. In some embodiments, a cell surface antigen is characteristic of more than one type of cell.

In some embodiments a CAR antigen binding domain binds a cell surface antigen characteristic of a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen characteristic of a T cell may be a T cell receptor. In some embodiments, a T cell receptor may be AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments a CAR comprises a signaling domain which is a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain).

In some embodiments, the CAR contains one or more domains that combine an antigen- or ligand-binding domain (e.g. antibody or antibody fragment) that provides specificity for a desired antigen (e.g., tumor antigen) with intracellular signaling domains. In some embodiments, the intracellular signaling domain is a stimulating or an activating intracellular domain portion, such as a T cell stimulating or activating domain, providing a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in WO200014257, WO2013126726, WO2012/129514, WO2014031687, WO2013/166321, WO2013/071154, WO2013/123061, U.S. patent app. Pub. Nos. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent app. No. EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in WO/2014055668. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., (2013) Nature Reviews Clinical Oncology, 10, 267-276; Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, 8,389,282. The recombinant receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the antigen binding domain of the CAR molecule comprises an antibody, an antibody fragment, an scFv, a Fv, a Fab, a (Fab′)₂, a single domain antibody (SdAb), a VH or VL domain, or a camelid VHH domain.

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a CD19 antibody; CD22 antibody; T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD20 antibody; CD21 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-BB, CD134/OX40, CD3, CD4W, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

Examples of exemplary components of a CAR are described in Table 3. In provided aspects, the sequences of each component in a CAR can include any combination listed in Table 3.

TABLE 3
CAR components and Exemplary Sequences

| | | |
|---|---|---|
| Component | Sequence | SEQ ID NO |

Extracellular binding domain

| | | |
|---|---|---|
| Anti-CD19 scFv (FMC63) | DIQMTQTTSSLSASLGDRVTISCRASQDISKYLNWYQ | 239 |
| | QKPDGTVKLLIYHTSRLHSGVPSRFSGSGSGTDYSLTI | |
| | SNLEQEDIATYFCQQGNTLPYTFGGGTKLEITGSTSGS | |
| | GKPGSGEGSTKGEVKLQESGPGLVAPSQSLSVTCTVS | |
| | GVSLPDYGVSWIRQPPRKGLEWLGVIWGSETTYYNS | |
| | ALKSRLTIIKDNSKSQVFLKMNSLQTDDTAIYYCAKH | |
| | YYYGGSYAMDYWGQGTSVTVSS | |
| Anti-CD19 scFv (FMC63) | DIQMTQTTSSLSASLGDRVTISCRASQDISKYLNWYQ | 240 |
| | QKPDGTVKLLIYHTSRLHSGVPSRFSGSGSGTDYSLTI | |
| | SNLEQEDIATYFCQQGNTLPYTFGGGTKLEITGGGGS | |
| | GGGGSGGGGSEVKLQESGPGLVAPSQSLSVTCTVSG | |
| | VSLPDYGVSWIRQPPRKGLEWLGVIWGSETTYYNSA | |
| | LKSRLTIIKDNSKSQVFLKMNSLQTDDTAIYYCAKHY | |
| | YYGGSYAMDYWGQGTSVTVSS | |

Spacer (e.g. hinge)

| | | |
|---|---|---|
| IgG4 Hinge | ESKYGPPCPPCP | 241 |
| CD8 Hinge | TTTPAPRPPTPAPTIASQPLSLRPE | 242 |
| CD28 | IEVMYPPPYLDNEKSNGTIIHVKGKHLCPSPLFPGPSK | 243 |
| | P | |

Transmembrane

| | | |
|---|---|---|
| CD8 | ACRPAAGGAVHTRGLDFACDIYIWAPLAGTCGVLLL | 244 |
| | SLVITLYC | |
| CD28 | FWVLVVVGGVLACYSLLVTVAFIIFWV | 245 |
| CD28 | FWVLVVVGGVLACYSLLVTVAFIIFWV | 246 |

Costimulatory domain

| | | |
|---|---|---|
| CD28 | RSKRSRLLHSDYMNMTPRRPGPTRKHYQPYAPPRDF | 247 |
| | AAYRS | |
| 4-1BB | KRGRKKLLYIFKQPFMRPVQTTQEEDGCSCRFPEEEE | 248 |
| | GGCEL | |

Primary Signaling Domain

| | | |
|---|---|---|
| CD3zeta | RVKFSRSADAPAYQQGQNQLYNELNLGRREEYDVLD | 249 |
| | KRRGRDPEMGGKPRRKNPQEGLYNELQKDKMAEAY | |
| | SEIGMKGERRRGKGHDGLYQGLSTATKDTYDALHM | |
| | QALPPR | |
| CD3zeta | RVKFSRSADAPAYKQGQNQLYNELNLGRREEYDVLD | 250 |
| | KRRGRDPEMGGKPRRKNPQEGLYNELQKDKMAEAY | |
| | SEIGMKGERRRGKGHDGLYQGLSTATKDTYDALHM | |
| | QALPPR | |

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen targeted by the receptor includes antigens associated with a B cell malignancy, such as any of a number of known B cell markers. In some embodiments, the antigen targeted by the receptor is CD20, CD19, CD22, ROR1, CD45, CD47, CD21, CD5, CD33, Igkappa, Iglambda, CD79a, CD79b or CD30.

In some embodiments, the CAR binds to CD19. In some embodiments, the CAR binds to CD22. In some embodiments, the CAR binds to CD19 and CD22. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antigen targeted by the antigen-binding domain is CD19. In some aspects, the antigen-binding domain of the recombinant receptor, e.g., CAR, and the antigen-binding domain binds, such as specifically binds or specifically recognizes, a CD19, such as a human CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the antigen is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgGl antibody raised against Naim-1 and -16 cells expressing CD19 of human origin (Fing, N. R., et al. (1987). Leucocyte typing III. 302).

In some embodiments, the antibody portion of the recombinant receptor, e.g., CAR, further includes spacer between the transmembrane domain and extracellular antigen binding domain. In some embodiments, the spacer includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgGl. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, WO2014031687, U.S. Pat. No. 8,822,647 or published app. No. US 2014/0271635. In some embodiments, the constant region or portion is of a human IgG, such as IgG4 or IgGl.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an IT AM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40IJCD154, VEGFR2, FAS, and FGFR2B, or functional variant thereof. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or IT AMs. Examples of IT AM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the intracellular component is or includes a CD3-zeta intracellular signaling domain. In some embodiments, the intracellular component is or includes a signaling domain from Fc receptor gamma chain. In some embodiments, the receptor, e.g., CAR, includes the intracellular signaling domain and further includes a portion, such as a transmembrane domain and/or hinge portion, of one or more additional molecules such as CD8, CD4, CD25, or CD16. For example, in some aspects, the CAR or other chimeric receptor is a chimeric molecule of CD3-zeta (CD3-z) or Fc receptor and a portion of one of CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB. In some aspects, the T cell costimulatory molecule is 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, a CD19 specific CAR includes an anti-CD19 single-chain antibody fragment (scFv), a transmembrane domain such as one derived from human CD8α, a 4-1BB (CD137) co-stimulatory signaling domain, and a CD3 signaling domain. In some embodiments, a CD22 specific CAR includes an anti-CD22 scFv, a transmembrane domain such as one derived from human CD8α, a 4-1BB (CD137) co-stimulatory signaling domain, and a CD3ζ signaling domain. In some embodiments, a CD19/CD22-bispecific CAR includes an anti-CD19 scFv, an anti-CD22 scFv, a transmembrane domain such as one derived from human CD8α, a 4-1BB (CD137) co-stimulatory signaling domain, and a CD3ζ signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, AUTO4 from Autolus Limited, UCARTCS from Cellectis, PBCAR19B and PBCAR269A from Precision Biosciences, FT819 from Fate Therapeutics, and CYAD-211 from Clyad Oncology.

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture syndrome, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, e.g., US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gpl20, or CD4-induced epitope on HIV-1 Env.

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain).

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD 137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-IBB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some aspects, the spacer contains only a hinge region of an IgG, such as only a hinge of IgG4 or IgGlIn other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

In some embodiments a lipid particle comprising a CAR or a nucleic acid encoding a CAR (e.g., a DNA, a gDNA, a cDNA, an RNA, a pre-MRNA, an mRNA, an miRNA, an siRNA, etc.) is delivered to a target cell. In some embodiments the target cell is an effector cell, e.g., a cell of the immune system that expresses one or more Fc receptors and mediates one or more effector functions. In some embodiments, a target cell may include, but may not be limited to, one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including but not limited to humans, mice, rats, rabbits, and monkeys.

In certain embodiments, the exogenous agent is a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The provided particles may be used to express one or more CARs in a host cell (e.g., a T cell) for use in cell-based therapies against various target antigens. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 4 below.

TABLE 4
Exemplary sequences of signal peptides

| SEQ ID | | |
|---|---|---|
| NO: | Sequence | Description |
| 465 | MALPVTALLLPLALLLHAARP | CD8α signal peptide |
| 466 | METDTLLLWVLLLWVPGSTG | IgK signal peptide |
| 167 | MLLLVTSLLLCELPHPAFLLIP | GMCSFR-α (CSF2RA) signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 5 below.

TABLE 5
Exemplary sequences of hinge domains

| SEQ ID | | |
|---|---|---|
| NO: | Sequence | Description |
| 468 | TTTPAPRPPTPAPTIASQPLSLRPEACRPAAGGAVH | CD8α hinge domain |
| | TRGLDFACD | |
| 469 | IEVMYPPPYLDNEKSNGTIIHVKGKHLCPSPLFPGP | CD28 hinge domain |
| | SKP | |
| 470 | AAAIEVMYPPPYLDNEKSNGTIIHVKGKHLCPSPLF | CD28 hinge domain |
| | PGPSKP | |
| 471 | ESKYGPPCPPCP | IgG4 hinge domain |
| 472 | ESKYGPPCPSCP | IgG4 hinge domain |
| 473 | ESKYGPPCPPCPAPEFLGGPSVFLFPPKPKDTLMISR | IgG4 hinge-CH2-CH3 |
| | TPEVTCVVVDVSQEDPEVQFNWYVDGVEVHNAK | domain |
| | TKPREEQFNSTYRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCSV | |
| | MHEALHNHYTQKSLSLSLGK | |

In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD37, CD64, CD8, CD86, CD134, CD137, CD154, or a functional variant thereof, including the human versions of each of these sequences. In other embodiments, the transmembrane domain may comprise a transmembrane region of CD8α, CD80β, 4-1BB/CD137, CD28, CD34, CD4, FcεRIγ, CD16, OX40/CD134, CD3ζ, CD3ε, CD3γ, CD3δ, TCRα, TCRPβ, TCRζ, CD32, CD364, CD364, CD45, CD5, CD9, CD22, CD37, CD80, CD86, CD40, CD40UJCD154, VEGFR2, FAS, and FGFR2B, or a functional variant thereof, including the human versions of each of these sequences. Table 6 provides the amino acid sequences of a few exemplary transmembrane domains.

TABLE 6
Exemplary sequences of transmembrane domains

| SEQ ID | | |
|---|---|---|
| NO: | Sequence | Description |
| 473 | IYIWAPLAGTCGVLLLSLVITLYC | CD8α transmembrane domain |
| 474 | FWVLVVVGGVLACYSLLVTVAFIIFWV | CD28 transmembrane domain |
| 475 | MFWVLVVVGGVLACYSLLVTVAFIIFWV | CD28 transmembrane domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H12, B7-H13, B7-H14, B7-1H6, B7-H17, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD3WMITFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/ITNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Ihy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 7 provides the amino acid sequences of a few exemplary intracellular costimulatory and/or signaling domains. In certain embodiments, as in the case of tisagenlecleucel as described below, the CD3ζ signaling domain of SEQ ID NO:99 may have a mutation, e.g., a glutamine (Q) to lysine (K) mutation, at amino acid position 518 (see SEQ ID NO:478).

TABLE 7
Exemplary sequences of intracellular costimulatory and/or signaling domains

| SEQ ID | | |
|---|---|---|
| NO: | Sequence | Description |
| 476 | KRGRKKLLYIFKQPFMRPVQTTQEEDGCSCR | 4-1BB costimulatory domain |
| | FPEEEEGGCEL | |
| 477 | RSKRSRLLHSDYMNMTPRRPGPTRKHYQPY | CD28 costimulatory domain |
| | APPRDFAAYRS | |
| 478 | RVKFSRSADAPAYQQGQNQLYNELNLGRRE | CD3ζ signaling domain |
| | EYDVLDKRRGRDPEMGGKPRRKNPQEGLYN | |
| | ELQKDKMAEAYSEIGMKGERRRGKGHDGLY | |
| | QGLSTATKDTYDALHMQALPPR | |
| 479 | RVKFSRSADAPAYKQGQNQLYNELNLGRRE | CD3ζ signaling domain (with Q |
| | EYDVLDKRRGRDPEMGGKPRRKNPQEGLYN | to K mutation at position 14) |
| | ELQKDKMAEAYSEIGMKGERRRGKGHDGLY | |
| | QGLSTATKDTYDALHMQALPPR | |

### A) CD19 CAR

In some embodiments, the CAR is a CD19 CAR (“CD19-CAR”). In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:465 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:465. In some embodiments, the signal peptide comprises an IgK signal peptide. In some embodiments, the IgK signal peptide comprises or consists of an amino acid sequence set forth in SEQ ID NO:466 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:466. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:467 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:467.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 10 below. In some embodiments, the CD19-specific scFv comprises or consists of an amino acid sequence set forth in SEQ ID NO:479, 480, or 486, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 479, 480, or 486. In some embodiments, the CD19-specific scFv may comprise one or more CDRs having amino acid sequences set forth in SEQ ID NOs: 482-483 and 487-489. In some embodiments, the CD19-specific scFv may comprise a light chain with one or more CDRs having amino acid sequences set forth in SEQ ID NOs: 482-484. In some embodiments, the CD19-specific scFv may comprise a heavy chain with one or more CDRs having amino acid sequences set forth in SEQ ID NOs: 487-489 In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linkers having an amino acid sequence set forth in SEQ ID NO:485. In some embodiments, the Whitlow linker may be replaced by a different linker, for example, a 3×G₄S linker having an amino acid sequence set forth in SEQ ID NO:491, which gives rise to a different FMC63-derived scFv having an amino acid sequence set forth in SEQ ID NO:490. In certain of these embodiments, the CD19-specific scFv comprises or consists of an amino acid sequence set forth in SEQ ID NO:490 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ

TABLE 8
Exemplary sequences of anti-CD19 scFv and components

| SEQ ID NO: | Amino Acid Sequence | Description |
|---|---|---|
| 480 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 481 | DIQMTQTTSSLSASLGDRVTISCRASQDI | Anti-CD19 FMC63 scFv |
| | SKYLNWYQQKPDGTVKLLIYHTSRLHS | light chain variable region |
| | GVPSRFSGSGSGTDYSLTISNLEQEDIAT | |
| | YFCQQGNTLPYTFGGGTKLEIT | |
| 482 | QDISKY | Anti-CD19 FMC63 scFv |
| | | light chain CDR1 |
| 483 | HTS | Anti-CD19 FMC63 scFv |
| | | light chain CDR2 |
| 484 | QQGNTLPYT | Anti-CD19 FMC63 scFv |
| | | light chain CDR3 |
| 485 | GSTSGSGKPGSGEGSTKG | Whitlow linker |
| 486 | EVKLQESGPGLVAPSQSLSVTCTVSGVS | Anti-CD19 FMC63 scFv |
| | LPDYGVSWIRQPPRKGLEWLGVIWGSE | heavy chain variable region |
| | TTYYNSALKSRLTIIKDNSKSQVFLKMN | |
| | SLQTDDTAIYYCAKHYYYGGSYAMDY | |
| | WGQGTSVTVSS | |
| 487 | GVSLPDYG | Anti-CD19 FMC63 scFv |
| | | heavy chain CDR1 |
| 488 | IWGSETT | Anti-CD19 FMC63 scFv |
| | | heavy chain CDR2 |
| 489 | AKHYYYGGSYAMDY | Anti-CD19 FMC63 scFv |
| | | heavy chain CDR3 |
| 490 | DIQMTQTTSSLSASLGDRVTISCRASQDI | Anti-CD19 FMC63 scFv |
| | SKYLNWYQQKPDGTVKLLIYHTSRLHS | entire sequence, with 3xG₄S |
| | GVPSRFSGSGSGTDYSLTISNLEQEDIAT | linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 491 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G37 (Meeker et al., Hybridoma 3:305-320 (1984)), B43 (Bejcek (1995)), BLY3 (Bejcek (1995)), B4 (Freedman et al., 70:418-427 (1987)), B4 HB 12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU 12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the binge domain of the CD19 CAR comprises a CD8α hinge domain, for example, a human CD8α hinge domain. In some embodiments, the CD8α hinge domain comprises or consists of an amino acid sequence set forth in SEQ ID NO:468 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:468. In some embodiments, the hinge domain comprises a CD28 hinge domain, for example, a human CD28 hinge domain. In some embodiments, the CD28 hinge domain comprises or consists of an amino acid sequence set forth in SEQ ID NO:469 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:469. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:471 or SEQ ID NO:472, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:471 or SEQ ID NO:472. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:472 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:472.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:473 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:473. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:474 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:474.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:476 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:476. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:477 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:477. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3 zeta associates with T cell receptors (TCRs) to produce a signal and contains immunoreceptor tyrosine-based activation motifs (ITAMs). The CD3 zeta signaling domain refers to amino acid residues from the cytoplasmic domain of the zeta chain that are sufficient to functionally transmit an initial signal necessary for T cell activation. In some embodiments, the CD3 zeta signaling domain is human. In some embodiments, the CD3 zeta signaling domain comprises or consists of an amino acid sequence set forth in SEQ ID NO:478 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:478.

In some embodiments, the payload agent is a CD19 CAR, including, for example, a CD19 CAR comprising the CD19-specific scFv having sequences set forth in SEQ ID NO:63 or SEQ ID NO:490, the CD8α hinge domain of SEQ ID NO:468, the CD8α transmembrane domain of SEQ ID NO:473, the 4-1BB costimulatory domain of SEQ ID NO:476, the CD3 signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the payload agent is a CD19 CAR, including, for example, a CD19 CAR comprising the CD19-specific scFv having sequences set forth in SEQ ID NO:274 or SEQ ID NO:490, the IgG4 hinge domain of SEQ ID NO:471 or SEQ ID NO:472, the CD28 transmembrane domain of SEQ ID NO:474, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the payload agent is a CD19 CAR, including, for example, a CD19 CAR comprising the CD19-specific scFv having sequences set forth in SEQ ID NO:480 or SEQ ID NO:490, the CD28 hinge domain of SEQ ID NO:469, the CD28 transmembrane domain of SEQ ID NO:474, the CD28 costimulatory domain of SEQ ID NO:477, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the payload agent is a CD19 CAR as encoded by the sequence set forth in SEQ ID NO:492 or a sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:492 (see Table 9). The encoded CD19 CAR has a corresponding amino acid sequence set forth in SEQ ID NO:493 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:493, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the payload agent is a commercially available embodiment of a CD19 CAR. Non-limiting examples of commercially available embodiments of CD19 CARs include tisagenlecleucel, lisocabtagene maraleucel, axicabtagene ciloleucel, and brexucabtagene autoleucel.

In some embodiments, the CAR is tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 9, with annotations of the sequences provided in Table 10.

In some embodiments, the CAR is lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 7, with annotations of the sequences provided in Table 11.

In some embodiments, the CAR is axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 9, with annotations of the sequences provided in Table 12.

In some embodiments, the CAR is brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the CAR is encoded by the sequence set forth in SEQ ID NO: 494, 496, or 498, or a sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 494, 496, or 498. The encoded CD19 CAR has a corresponding amino acid sequence set forth in SEQ ID NO: 495, 497, or 499, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 495, 497, or 499, respectively. Table 9. Exemplary sequences of CD19 CARs

TABLE 9
Exemplary sequences of CD19 CARs

| SEQ ID NO: | Sequence | Description |
|---|---|---|
| 492 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattcgcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaccggcgcccaccategcgtcgcagc | |
| | ccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctcgc | |
| 493 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 494 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaccggcgcccaccatcgcgtcgcagcccctgtccctgc | |
| | gcccagaggcgtgccggccagcggcggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 495 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 496 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcagcggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagegaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggggggcaagggccacgacggcctgtatc | |
| | agggcctgtccaccgccaccaaggatacctacgacgccctgcacatgca | |
| | ggccctgcccccaagg | |
| 497 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 498 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 499 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 10
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3 × G₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 11
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 12
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3 zeta signaling domain | 1132-1467 | 378-489 |

In some embodiments, the CAR is encoded by the sequence set forth in SEQ ID NO: 494, 496, 498, or a sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 494, 496, 498. The encoded CD19 CAR has a corresponding amino acid sequence set forth in SEQ ID NO: 495, 497, 499, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 495, 497, 499, respectively.

### B) CD20 CAR

In some embodiments, the CAR is a CD20 CAR (“CD20-CAR”). CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:465 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:465. In some embodiments, the signal peptide comprises an IgK signal peptide. In some embodiments, the IgK signal peptide comprises or consists of an amino acid sequence set forth in SEQ ID NO:466 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:466. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:467 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:467.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 13 below. In some embodiments, the CD20-specific scFv comprises or consists of an amino acid sequence set forth in SEQ ID NO:500, 504, or 505, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 500, 504, or 505. In some embodiments, the CD20-specific scFv may comprise one or more CDRs having amino acid sequences set forth in SEQ ID NOs: 502-504, 506, 507, and 508. In some embodiments, the CD20-specific scFv may comprise a light chain with one or more CDRs having amino acid sequences set forth in SEQ ID NOs: 502-504. In some embodiments, the CD20-specific scFv may comprise a heavy chain with one or more CDRs having amino acid sequences set forth in SEQ ID NOs: 506, 507, and 508. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 13
Exemplary sequences of anti-CD20 scFv and components

| SEQ ID NO: | Amino Acid Sequence | Description |
|---|---|---|
| 500 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 501 | DIVLTQSPAILSASPGEKVTMTCRASSS | Anti-CD20 Leu16 scFv |
| | VNYMDWYQKKPGSSPKPWIYATSNLA | light chain variable region |
| | SGVPARFSGSGSGTSYSLTISRVEAEDA | |
| | ATYYCQQWSFNPPTFGGGTKLEIK | |
| 502 | RASSSVNYMD | Anti-CD20 Leu16 scFv |
| | | light chain CDR1 |
| 503 | ATSNLAS | Anti-CD20 Leu16 scFv |
| | | light chain CDR2 |
| 504 | QQWSFNPPT | Anti-CD20 Leu16 scFv |
| | | light chain CDR3 |
| 505 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 506 | SYNMH | Anti-CD20 Leu16 scFv |
| | | heavy chain CDR1 |
| 507 | AIYPGNGDTSYNQKFKG | Anti-CD20 Leu16 scFv |
| | | heavy chain CDR2 |
| 508 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:468 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:468. In some embodiments, the hinge domain comprises a CD28 hinge domain, for example, a human CD28 hinge domain. In some embodiments, the CD28 hinge domain comprises or consists of an amino acid sequence set forth in SEQ ID NO:469 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:469. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:471 or SEQ ID NO:472, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:471 or SEQ ID NO:472. In some embodiments, the hinge domain comprises a IgG4 hinge-Ch2-Ch3 domain, for example, a human IgG4 hinge-Ch2-Ch3 domain.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:473 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:473. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:474 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:474.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:476 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:476. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:477 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:477.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:478 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:478.

In some embodiments, the CAR is a CD20 CAR, including, for example, a CD20 CAR comprising the CD20-specific scFv having sequences set forth in SEQ ID NO: 500, the CD8α hinge domain of SEQ ID NO:468, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:500, the CD28 hinge domain of SEQ ID NO:469, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:500, the IgG4 hinge domain of SEQ ID NO:471 or SEQ ID NO:472, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:500, the CD8α hinge domain of SEQ ID NO:468, the CD28 transmembrane domain of SEQ ID NO:474, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:500, the CD28 hinge domain of SEQ ID NO:469, the CD28 transmembrane domain of SEQ ID NO:474, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:500, the IgG4 hinge domain of SEQ ID NO:471 or SEQ ID NO:472, the CD28 transmembrane domain of SEQ ID NO:474, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### C) CD22 CAR

In some embodiments, the CAR is a CD22 CAR (“CD22-CAR”). CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:465 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:465. In some embodiments, the signal peptide comprises an IgK signal peptide. In some embodiments, the IgK signal peptide comprises or consists of an amino acid sequence set forth in SEQ ID NO:466 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:466. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:467 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:467.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 14 below. In some embodiments, the CD22-specific scFv comprises or consists of an amino acid sequence set forth in SEQ ID NO:509, 510, or 514, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 509, 510, or 514. In some embodiments, the CD22-specific scFv may comprise one or more CDRs having amino acid sequences set forth in SEQ ID NOs: 511-513 and 515-517. In some embodiments, the CD22-specific scFv may comprise a heavy chain with one or more CDRs having amino acid sequences set forth in SEQ ID NOs: 511-513. In some embodiments, the CD22-specific scFv may comprise a light chain with one or more CDRs having amino acid sequences set forth in SEQ ID NOs: 515-517. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO:518, 519, or 523, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 518, 519, or 523. In some embodiments, the CD22-specific scFv may comprise one or more CDRs having amino acid sequences set forth in SEQ ID NOs: 520-522 and 524-526. In some embodiments, the CD22-specific scFv may comprise a heavy chain with one or morV CDRs having amino acid sequences set forth in SEQ ID NOs: 520-522. In some embodiments, the CD22-specific scFv may comprise a light chain with one or more CDRs having amino acid sequences set forth in SEQ ID NOs: 524-526. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 14
Exemplary sequences of anti-CD22 scFv and components

| SEQ ID NO: | Amino Acid Sequence | Description |
|---|---|---|
| 509 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG₄S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 510 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 511 | GDSVSSNSAA | Anti-CD22 m971 scFv |
| | | heavy chain CDR1 |
| 512 | TYYRSKWYN | Anti-CD22 m971 scFv |
| | | heavy chain CDR2 |
| 513 | AREVTGDLEDAFDI | Anti-CD22 m971 scFv |
| | | heavy chain CDR3 |
| 514 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 515 | QTIWSY | Anti-CD22 m971 scFv |
| | | light chain CDR1 |
| 516 | AAS | Anti-CD22 m971 scFv |
| | | light chain CDR2 |
| 517 | QQSYSIPQT | Anti-CD22 m971 scFv |
| | | light chain CDR3 |
| 518 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG₄S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 519 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 520 | GDSVSSNSVA | Anti-CD22 m971-L7 scFv |
| | | heavy chain CDR1 |
| 521 | TYYRSTWYN | Anti-CD22 m971-L7 scFv |
| | | heavy chain CDR2 |
| 522 | AREVTGDLEDAFDI | Anti-CD22 m971-L7 scFv |
| | | heavy chain CDR3 |
| 523 | DIQMIQSPSSLSASVGDRVTITCRASQTI | Anti-CD22 m971-L7 scFv |
| | WSYLNWYRQRPGEAPNLLIYAASSLQS | light chain variable region |
| | GVPSRFSGRGSGTDFTLTISSLQAEDFAT | |
| | YYCQQSYSIPQTFGQGTKLEIK | |
| 524 | QTIWSY | Anti-CD22 m971-L7 scFv |
| | | light chain CDR1 |
| 525 | AAS | Anti-CD22 m971-L7 scFv |
| | | light chain CDR2 |
| 526 | QQSYSIPQT | Anti-CD22 m971-L7 scFv |
| | | light chain CDR3 |

In some embodiments, the extracellular binding domain of the CD22 CAR comprises imnmunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:468 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:468. In some embodiments, the hinge domain comprises a CD28 hinge domain, for example, a human CD28 hinge domain. In some embodiments, the CD28 hinge domain comprises or consists of an amino acid sequence set forth in SEQ ID NO:469 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:469. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:471 or SEQ ID NO:472, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:471 or SEQ ID NO:472. In some embodiments, the hinge domain comprises a IgG4 hinge-Ch2-Ch3 domain, for example, a human IgG4 hinge-Ch2-Ch3 domain.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:473 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:473. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:474 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:474.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:476 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:476. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:477 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:477.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:478 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:478.

In some embodiments, the CAR is a CD22 CAR, including, for example, a CD22 CAR comprising the CD22-specific scFv having sequences set forth in SEQ ID NO:509 or SEQ ID NO:518, the CD8α hinge domain of SEQ ID NO:473, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:509 or SEQ ID NO:518, the CD28 hinge domain of SEQ ID NO:469, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:509 or SEQ ID NO:518, the IgG4 hinge domain of SEQ ID NO:471 or SEQ ID NO:472, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:509 or SEQ ID NO:518, the CD8α hinge domain of SEQ ID NO:468, the CD28 transmembrane domain of SEQ ID NO:474, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:509 or SEQ ID NO:518, the CD28 hinge domain of SEQ ID NO:469, the CD28 transmembrane domain of SEQ ID NO:474, the 4-1BB costimulatory domain of SEQ ID NO:270, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:509 or SEQ ID NO:518, the IgG4 hinge domain of SEQ ID NO:471 or SEQ ID NO:472, the CD28 transmembrane domain of SEQ ID NO:474, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### D) BCMA CAR

In some embodiments, the CAR is a BCMA CAR (“BCMA-CAR”). BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:465 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:465. In some embodiments, the signal peptide comprises an IgK signal peptide. In some embodiments, the IgK signal peptide comprises or consists of an amino acid sequence set forth in SEQ ID NO:466 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:466. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:467 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:467.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 15 below. In some embodiments, the BCMA-specific extracellular binding domain comprises or consists of an amino acid sequence set forth in SEQ ID NO:527, 528, or 532, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 527, 528, or 532. In some embodiments, the BCMA-specific extracellular binding domain may comprise one or more CDRs having amino acid sequences set forth in SEQ ID NOs: 529-531 and 533-535. In some embodiments, the BCMA-specific extracellular binding domain may comprise a light chain with one or more CDRs having amino acid sequences set forth in SEQ ID NOs: 529-531. In some embodiments, the BCMA-specific extracellular binding domain may comprise a heavy chain with one or more CDRs having amino acid sequences set forth in SEQ ID NOs: 533-535. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 15 below. In some embodiments, the BCMA-specific extracellular binding domain comprises or consists of an amino acid sequence set forth in SEQ ID NO:536, 537, or 541, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 536, 537, or 541. In some embodiments, the BCMA-specific extracellular binding domain may comprise one or more CDRs having amino acid sequences set forth in SEQ ID NOs: 529-530 and 542-544. In some embodiments, the BCMA-specific extracellular binding domain may comprise a light chain with one or more CDRs having amino acid sequences set forth in SEQ ID NOs: 529-530. In some embodiments, the BCMA-specific extracellular binding domain may comprise a heavy chain with one or more CDRs having amino acid sequences set forth in SEQ ID NOs: 542-544. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 15 below. In some embodiments, the BCMA-specific extracellular binding domain comprises or consists of an amino acid sequence set forth in SEQ ID NO:545 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:545. In some embodiments, the BCMA-specific extracellular binding domain may comprise one or more CDRs having amino acid sequences set forth in SEQ ID NOs: 546-548. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 17 below. In some embodiments, the BCMA-specific extracellular binding domain comprises or consists of an amino acid sequence set forth in SEQ ID NO:549, 550, or 554, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 549, 550, or 554. In some embodiments, the BCMA-specific extracellular binding domain may comprise one or more CDRs having amino acid sequences set forth in SEQ ID NOs: 551-553 and 555-557. In some embodiments, the BCMA-specific extracellular binding domain may comprise a light chain with one or more CDRs having amino acid sequences set forth in SEQ ID NOs: 551-553. In some embodiments, the BCMA-specific extracellular binding domain may comprise a heavy chain with one or more CDRs having amino acid sequences set forth in SEQ ID NOs: 555-557. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 15
Exemplary sequences of anti-BCMA binder and components

| SEQ ID NO: | Amino Acid Sequence | Description |
|---|---|---|
| 527 | DIVLTQSPASLAMSLGKRATISCRASES | Anti-BCMA C11D5.3 |
| | VSVIGAHLIHWYQQKPGQPPKLLIYLAS | scFv entire sequence, with |
| | NLETGVPARFSGSGSGTDFTLTIDPVEE | Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 528 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 529 | RASESVSVIGAHLIH | Anti-BCMA C11D5.3 |
| | | scFv light chain CDR1 |
| 530 | LASNLET | Anti-BCMA C11D5.3 |
| | | scFv light chain CDR2 |
| 531 | LQSRIFPRT | Anti-BCMA C11D5.3 |
| | | scFv light chain CDR3 |
| 532 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 533 | DYSIN | Anti-BCMA C11D5.3 |
| | | scFv heavy chain CDR1 |
| 534 | WINTETREPAYAYDFRG | Anti-BCMA C11D5.3 |
| | | scFv heavy chain CDR2 |
| 535 | DYSYAMDY | Anti-BCMA C11D5.3 |
| | | scFv heavy chain CDR3 |
| 536 | DIVLTQSPPSLAMSLGKRATISCRASESV | Anti-BCMA C12A3.2 |
| | TILGSHLIYWYQQKPGQPPTLLIQLASN | scFv entire sequence, with |
| | VQTGVPARFSGSGSRTDFTLTIDPVEED | Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 537 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 538 | RASESVTILGSHLIY | Anti-BCMA C12A3.2 |
| | | scFv light chain CDR1 |
| 539 | LASNVQT | Anti-BCMA C12A3.2 |
| | | scFv light chain CDR2 |
| 540 | LQSRTIPRT | Anti-BCMA C12A3.2 |
| | | scFv light chain CDR3 |
| 541 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 542 | HYSMN | Anti-BCMA C12A3.2 |
| | | scFv heavy chain CDR1 |
| 543 | RINTESGVPIYADDFKG | Anti-BCMA C12A3.2 |
| | | scFv heavy chain CDR2 |
| 544 | DYLYSLDF | Anti-BCMA C12A3.2 |
| | | scFv heavy chain CDR3 |
| 545 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 546 | GFTFSSYA | Anti-BCMA FHVH33 |
| | | CDR1 |
| 547 | ISGSGDYI | Anti-BCMA FHVH33 |
| | | CDR2 |
| 548 | AKEGTGANSSLADY | Anti-BCMA FHVH33 |
| | | CDR3 |
| 549 | DIQMTQSPSSLSASVGDRVTITCRASQSI | Anti-BCMA CT103A |
| | SSYLNWYQQKPGKAPKLLIYAASSLQS | scFv entire sequence, with |
| | GVPSRFSGSGSGTDFTLTISSLQPEDFAT | Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 550 | DIQMTQSPSSLSASVGDRVTITCRASQSI | Anti-BCMA CT103A |
| | SSYLNWYQQKPGKAPKLLIYAASSLQS | scFv light chain variable |
| | GVPSRFSGSGSGTDFTLTISSLQPEDFAT | region |
| | YYCQQKYDLLTFGGGTKVEIK | |
| 551 | QSISSY | Anti-BCMA CT103A |
| | | scFv light chain CDR1 |
| 552 | AAS | Anti-BCMA CT103A |
| | | scFv light chain CDR2 |
| 553 | QQKYDLLT | Anti-BCMA CT103A |
| | | scFv light chain CDR3 |
| 554 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 555 | GGSISSSSYY | Anti-BCMA CT103A |
| | | scFv heavy chain CDR1 |
| 556 | ISYSGST | Anti-BCMA CT103A |
| | | scFv heavy chain CDR2 |
| 557 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:468 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:468. In some embodiments, the hinge domain comprises a CD28 hinge domain, for example, a human CD28 hinge domain. In some embodiments, the CD28 hinge domain comprises or consists of an amino acid sequence set forth in SEQ ID NO:469 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:469. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:471 or SEQ ID NO:472, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:471 or SEQ ID NO:472. In some embodiments, the hinge domain comprises a IgG4 hinge-Ch2-Ch3 domain, for example, a human IgG4 hinge-Ch2-Ch3 domain.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:473 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:473. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:474 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:474.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:476 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:476. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:477 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:477.

In some embodiments, the intracellular signaling domain of the BCMA CAR comprises a CD3 zeta (Q signaling domain, for example, a human CD3ζ signaling domain. In some embodiments, the CD3ζ signaling domain comprises or consists of an amino acid sequence set forth in SEQ ID NO:478 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:478.

In some embodiments, the CAR is a BCMA CAR, including, for example, a BCMA CAR comprising any of the BCMA-specific extracellular binding domains as described, the CD8α hinge domain of SEQ ID NO:468, the CD8α transmembrane domain of SEQ ID NO:473, the 4-1BB costimulatory domain of SEQ ID NO:476, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the CAR is a BCMA CAR, including, for example, a BCMA CAR comprising any of the BCMA-specific extracellular binding domains as described, the CD8α hinge domain of SEQ ID NO:468, the CD8α transmembrane domain of SEQ ID NO:473, the CD28 costimulatory domain of SEQ ID NO:477, the CD3ζ signaling domain of SEQ ID NO:478, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the CAR is a BCMA CAR as set forth in SEQ ID NO:558 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:558 (see Table 16). The encoded BCMA CAR has a corresponding amino acid sequence set forth in SEQ ID NO:559 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:559, with the following components: CD8α signal peptide, CT103A scFv (V_{L}-Whitlow linker-V), CD8α hinge domain, CD8α transmembrane domain, 4-1BB costimulatory domain, and CD3ζ signaling domain.

In some embodiments, the CAR is a commercially available embodiment of BCMA CAR, including, for example, idecabtagene vicleucel (ide-cel, also called bb2121). In some embodiments, the CAR is idecabtagene vicleucel or portions thereof. Idecabtagene vicleucel comprises a BCMA CAR with the following components: the BB2121 binder, CD8α binge domain, CD8α transmembrane domain, 4-1BB costimulatory domain, and CD3′ signaling domain.

TABLE 16
Exemplary sequences of BCMA CARs

| SEQ ID NO: | Sequence | Description |
|---|---|---|
| 558 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaategccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggcggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 559 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### 3. Genome Modifying Enzymes

In some embodiments, the exogenous agent is or comprises a genome editing technology. In some embodiments, the exogenous agent is or comprises a heterologous protein that is associated with a genome editing technology. Any of a variety of agents associated with gene editing technologies can be included as the exogenous agent and/or heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the exogenous agent and/or heterologous protein mediates single-strand breaks (SSB). In some embodiments, the exogenous agent and/or heterologous protein mediates double-strand breaks (DSB), including in connection with non-homologous end-joining (NHEJ) or homology-directed repair (HDR). In some embodiments, the exogenous agent and/or heterologous protein does not mediate SSB. In some embodiments, the exogenous agent and/or heterologous protein does not mediate DSB. In some embodiments, the exogenous agent and/or heterologous protein can be used for DNA base editing or prime-editing. In some embodiments, the exogenous agent and/or heterologous protein can be used for Programmable Addition via Site-specific Targeting Elements (PASTE).

In some embodiments, the exogenous agent is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is Cas9 from Streptococcus pyogenes. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). In some embodiments, the Cas is a Cas12a (also known as cpf1) from a Prevotella or Francisella bacteria, or the Cas is a Cas12b, e.g., from a Bacillus, optionally Bacillus hisashii. In some embodiments, the Cas is a Cas12a (also known as cpf1) from a Prevotella, Francisella novicida, Acidaminococcus sp., Lachnospiraceae bacterium, or Francisella bacteria. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020). The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety.

In some embodiments, delivery of the nuclease is by a provided vector encoding the nuclease (e.g. Cas).

In some embodiments, the provided viral vector particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided viral vector particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110: 19860-19865; Gong et al., 2005, Nat. Struct Mol Biol, Vol. 12: 304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol. 337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein. In some embodiments, the Cas protein comprises one or more mutations such that the Cas protein is converted into a nickase that is able to cleave only one strand of a double stranded DNA molecule (e.g., a SSB). In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A.

In some embodiments, the Cas protein is selected from the group consisting of Cas3, Cas9, Cas10, Cas12, and Cas13. In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T, S409I, M694I, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., one or more exogenous agent and/or heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., one or more exogenous agent and/or heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, the provided lipid particles can be for use in a method to deliver an exogenous agent which involves introducing, into a cell, one or more agent(s) (e.g., one or more exogenous agent and/or heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., one or more exogenous agent and/or heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the one or more exogenous agent and/or heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the the PAM sequence on the sense strand and the PAM sequence on the antisense strand comprise the same nucleic acid sequence, which can be any PAM sequence disclosed herein. In some embodiments, the the PAM sequence on the sense strand and the PAM sequence on the antisense strand each comprise a different nucleic acid sequence, each of which can be any of the PAM sequences disclosed herein.

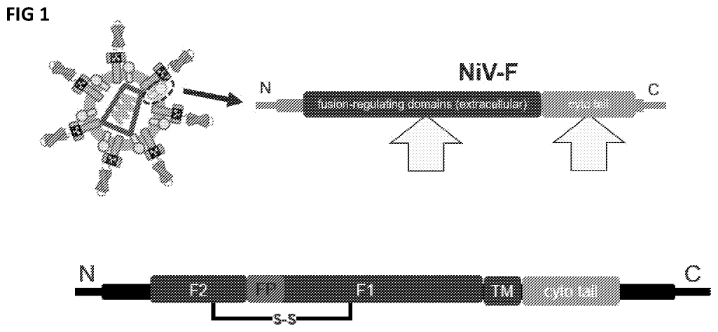

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C•G to T•A and adenine base editors (e.g., ABE7.10) that convert target A•T to G•C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the exogenous agent and/or heterologous protein is or encodes a base editor (e.g., a nucleobase editor). In some embodiments, the exogenous agent and/or heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the exogenous agent and/or heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the hheterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the exogenous agent and/or heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the exogenous agent and/or heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events.

In some embodiments, the exogenous agent and/or heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g. Cas).

In some embodiments, the provided lipid particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided lipid particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g. Cas protein, such as Cas9). In some embodiments, the fusion protein is a cleavable fusion protein between (i) a viral matrix (MA) protein and (ii) a nuclease protein (e.g. Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, a dCas9 version of the CRISPR/Cas9 system can be used to target protein domains for transcriptional regulation, epigenetic modification, and microscopic visualization of specific genome loci.

In some embodiments, the provided virus particles (e.g. lentiviral particles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing viral particles. In some embodiments, the provided virus particles (e.g. lentiviral particles) further comprises, or is further complexed with a targeting nucleic acid.

### 4. Small Molecules

In some embodiments, the exogenous agent includes a small molecule, e.g., ions (e.g. Ca²⁺, Cl—, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

### IV. PHARMACEUTICAL COMPOSITIONS

Also provided are compositions containing the lipid particles herein containing a variant NiV-F or polynucleotides encoding the variant NiV-F, including pharmaceutical compositions and formulations. The pharmaceutical compositions can include any of the described variant NiV-F-containing lipid particles.

The present disclosure also provides, in some aspects, a pharmaceutical composition comprising the composition described herein and pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular lipid particle and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle containing the variant NiV-F is a viral vector or virus-like particle (e.g., Section III). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10⁹ to about 10¹² GC units, inclusive. In some embodiments, the dosage of administration of a viral vector or virus-like particle is from about 10¹² to about 10¹⁴ GC units, inclusive. In some embodiments, the dosage of administration is about 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive. In some embodiments, the dosage of administration of a viral vector or virus-like particle is from about 10⁵ to about 10⁹ infectious units, inclusive. In some embodiments, the dosage of administration of a viral vector or virus-like particle is from about 10⁶ to about 10⁹ infectious units, inclusive. In some embodiments, the dosage of administration of a viral vector or virus-like particle is from about 10⁹ to about 10¹² infectious units, inclusive. In some embodiments, the dosage of administration of a viral vector or virus-like particle is from about 10¹² to about 10¹⁴ infectious units, inclusive. In some embodiments, the dosage of administration is about 1.0×10⁹ infectious units, 5.0×10¹¹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10⁴ infectious units, 5.0×10⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID50) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹¹ pfu, inclusive. In some embodiments, the dosage of administration of a viral vector or virus-like particle is from about 10⁵ to about 10⁹ pfu, inclusive. In some embodiments, the dosage of administration of a viral vector or virus-like particle is from about 10⁶ to about 10⁹ pfu, inclusive. In some embodiments, the dosage of administration of a viral vector or virus-like particle is from about 10⁹ to about 10¹² pfu, inclusive. In some embodiments, the dosage of administration of a viral vector or virus-like particle is from about 10¹² to about 10¹⁴ pfu, inclusive. In some embodiments, the dosage of administration is about 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cyroprotectants are combined with vector vehicles in order to stabilize them during freezing. In some aspects, Frozen storage of RNA between −20° C. and −80° C. may be advantageous for long term (e.g. 36 months) stability of polynucleotide. In some embodiments, the RNA species is mRNA. In some embodiments, cyroprotectants are included in vehicle formulations to stabilize polynucleotide through freeze/thaw cycles and under frozen storage conditions. Cyroprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### V. METHODS OF USE

In some embodiments, the lipid particles (e.g. pseudotyped lentiviral vectors) provided herein or pharmaceutical compositions containing same can be administered to a subject, e.g. a mammal, e.g. a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle, such as a targeted lipid particle, contains nucleic acid sequences encoding an exogenous agent for treating the disease or condition in the subject. For example, the exogenous agent is one that targets or is specific for a protein of a neoplastic cells and the lipid particle is administered to a subject for treating a tumor or cancer in the subject. In another example, the exogenous agent is an inflammatory mediator or immune molecule, such as a cytokine, and lipid particle is administered to a subject for treating any condition in which it is desired to modulate (e.g. increase) the immune response, such as a cancer or infectious disease. In some embodiments, the lipid particle is administered in an effective amount or dose to effect treatment of the disease, condition or disorder. Provided herein are uses of any of the provided lipid particles in such methods and treatments, and in the preparation of a medicament in order to carry out such therapeutic methods. In some embodiments, the methods are carried out by administering the lipid particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intranodal, intracavity, and subcutaneous) administration. In some embodiments, the lipid particle may be administered alone or formulated as a pharmaceutical composition. In some embodiments, the lipid particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the lipid particles may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, the lipid particle composition comprising an exogenous agent or cargo, may be used to deliver such exogenous agent or cargo to a cell tissue or subject. In some embodiments, delivery of a cargo by administration of a lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of lipid particles are from the same subject that is administered a lipid particle composition. In other embodiments, they are different. In some embodiments, the source of lipid particles and recipient tissue may be autologous (from the same subject) or heterologous (from different subjects). In some embodiments, the donor tissue for lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the lipid particle particles is co-administered with an inhibitor of sypressyn, e.g., a siRNA or inhibitory antibody.

### VI. EXEMPLARY EMBODIMENTS

Among the provided embodiments are:

1. A lipid particle, comprising:
(a) a lipid bilayer;
(b) a paramyxovirus glycoprotein (G protein) or a biologically active portion thereof; and
(c) a variant Nipah virus F glycoprotein (NiV-F) comprising a modified cytoplasmic tail, wherein the modified cytoplasmic tail comprises:
   (i) a heterologous cytoplasmic tail or a truncated portion thereof from a glycoprotein from another virus or a virus-associated protein, wherein the variant NiV-F is a chimeric protein;
   (ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or
   (iii) a modified NiV-F cytoplasmic that comprises a modified endocytosis motif,
wherein the G protein or the biologically active portion thereof and the variant NiV-F protein are exposed on the outside of the lipid bilayer.
   2. The lipid particle of embodiment 1, wherein the lipid bilayer is derived from a membrane of a host cell used for producing a retroviral vector or retrovirus-like particle, optionally a lentiviral vector or lentiviral-like particle.
   3. The lipid particle of embodiment 2, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
   4. A pseudotyped lentiviral particle, comprising:
(a) a paramyxovirus glycoprotein (G) or a biologically active portion thereof; and
(b) a variant Nipah virus F glycoprotein (NiV-F) comprising a modified cytoplasmic tail, wherein the modified cytoplasmic tail comprises:
   (i) a heterologous cytoplasmic tail or a truncated portion thereof from a glycoprotein from another virus or a virus-associated protein, wherein the variant NiV-F is a chimeric protein;
   (ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or
   (iii) a modified NiV-F cytoplasmic that comprises a modified endocytosis motif,
wherein the G protein or the biologically active portion thereof and the variant NiV-F protein are exposed on the outside of the lipid bilayer.
   5. The lipid particle or pseudotyped lentiviral particle of any of embodiments 1-4, wherein the variant NiV-F protein exhibits fusogenic activity with a target cell upon binding of the G protein to a target molecule on the target cell.
   6. The lipid particle or pseudotyped lentiviral particle of any of embodiments 1-5, wherein the variant NiV-F protein comprises an F0 precursor or is a proteolytically cleaved form thereof comprising F1 and F2 subunits.
   7. The lipid particle or pseudotyped lentiviral particle of embodiment 6, wherein the proteolytically cleaved form is a cathepsin L cleavage product.
   8. The lipid particle or pseudotyped lentiviral particle of any of embodiments 1-7, wherein the variant Niv-F protein comprises a modified F1 subunit containing the modified cytoplasmic tail and an F2 subunit in which (1) the F1 subunit is a modified F1 composed of the sequence of the modified cytoplasmic tail directly linked to the C-terminus of the sequence set forth in SEQ ID NO:383, and (2) the F2 subunit is set forth as SEQ ID NO:365.
   9. The lipid particle or pseudotyped lentiviral particle of any of embodiments 1-8, wherein the variant NiV-F comprises in order from N-terminus to C-terminus an extracellular domain, a transmembrane domain and the modified cytoplasmic tail.
   10. The lipid particle or pseudotyped lentiviral particle of any of embodiments 1-6, wherein the extracellular domain and transmembrane domain of the variant NiV-F are from a wild-type NiV-F or a biologically active variant thereof.
   11. The lipid particle or pseduotyped lentiviral particle of any of embodiments 1-7, wherein the extracellular domain and transmembrane domain of the variant NiV-F comprise the sequence set forth in SEQ ID NO:2, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:2.
   12. The lipid particle or pseudotyped lentiviral particle of any of embodiments 1-11, wherein the extracellular domain and transmembrane domain of the variant NiV-F comprise the sequence set forth in SEQ ID NO:2.
   13. The lipid particle or pseudotyped lentiviral particle of any of embodiments 1-12, wherein the modified cytoplasmic tail is a truncated NiV-F cytoplasmic tail that has a deletion of from 23 to 27 contiguous amino acid residues at or near the C-terminus of the wild-type Nipah virus F protein cytoplasmic tail set forth in SEQ ID NO:4.
   14. The lipid particle or pseduotyped lentiviral particle of any of embodiments 1-13, the modified cytoplasmic tail is a truncated NiV-G cytoplasmic tail that has a deletion of at or about 23 amino acid residues at or near the C-terminus of the wild-type Nipah virus cytoplasmic tail set forth in SEQ ID NO: 4.
   15. The lipid particle or pseudotyped lentiviral particle of any of embodiments 1-14, wherein the modified cytoplasmic tail is a truncated NiV-F cytoplasmic tail set forth in SEQ ID NO:27.
   16. The lipid particle or pseudotyped lentiviral particle of any of embodiments 1-14, wherein the variant NiV-F comprises the sequence set forth in SEQ ID NO:306, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:306.
   17. The lipid particle or pseudotyped lentiviral particle of any of embodiments 1-16, wherein the variant NiV-F comprises the sequence set forth in SEQ ID NO:306.
   18. The lipid particle or pseudotyped lentiviral particle of any of embodiments 1-12, wherein the variant NiV-F is a chimeric protein and the modified cytoplasmic tail comprises a heterologous cytoplasmic tail or a truncated portion thereof from a glycoprotein from another virus.
   19. The lipid particle or pseudotyped lentiviral particle of any of embodiments 1-12 and 18, wherein the other virus is a member of the Kingdom Orthornavirae.
   20. The lipid particle or pseudotyped lentiviral particle of any of embodiments 1-12, 18 and
   19, wherein the other virus is a member of the family Paramyxoviridae, Rhabdoviridae, Arenaviridae, or Retroviridae.
   21. The lipid particle or pseudotyped lentiviral particle of any of embodiments 1-12 and 18-20, wherein the other virus is a member of the family Paramyxoviridae.
   22. The lipid particle or pseudotyped lentiviral particle of embodiment 21, wherein the other virus is a Hendra virus, Cedar virus, Canine distemper virus, Parainfluenza virus, Measles virus, Newcastle disease virus, or Sendai virus.
   23. The lipid particle or pseudotyped lentiviral particle of any of embodiments 1-12 and 18-22, wherein the other virus is Measles virus and the glycoprotein is a Measles virus fusion (F) protein (MvF).
   24. The lipid particle or pseudotyped lentiviral particle of any of embodiments 1-12 and 18-23, wherein the heterologous cytoplasmic tail is a truncated MvF cytoplasmic tail that has a deletion of up to 32 contiguous amino acid residues at or near the C-terminus of the wild-type MvF cytoplasmic tail set forth in SEQ ID NO: 125.
   25. The lipid particle or pseudotyped lentiviral particle of any of embodiments 1-12 and 18-24, wherein the heterologous cytoplasmic tail is a truncated MvF cytoplasmic tail that has a deletion of at or about or up to 26 contiguous amino acid residues at or near the C-terminus of the wild-type MvF cytoplasmic tail set forth in SEQ ID NO: 125.
   26. The lipid particle or pseudotyped lentiviral particle of any of embodiments 1-12 and 18-25, wherein the modified cytoplasmic tail comprises the heterologous cytoplasmic tail set forth in SEQ ID NO:133.
   27. The lipid particle or pseudotyped lentiviral particle of any of embodiments 1-12 and 18-25, wherein the variant NiV-F comprises the sequence of amino acids set forth in SEQ ID NO:307, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:307.
   28. The lipid particle or pseudotyped lentiviral particle of any of embodiments 1-12 and 18-27, wherein the variant NiV-F comprises the sequence of amino acids set forth in SEQ ID NO: 307.
   29. The lipid particle or pseduotyped lentiviral particle of any of embodiments 1-12 and 18-22, wherein the other virus is Newcastle Disease Virus (NDV) and the glycoprotein is a NDV F protein.
   30. The lipid particle or pseudotyped lentiviral particle of any of embodiments 1-12, 18-22 and 29, wherein the heterologous cytoplasmic tail is a truncated NDV F protein cytoplasmic tail that has a deletion of up to 25 contiguous amino acid residues at or near the C-terminus of the wild-type NDV F protein cytoplasmic tail set forth in SEQ ID NO: 141.
   31. The lipid particle or pseudotyped lentiviral particle of any of embodiments 1-12, 18-22, 29 and 30, wherein the heterologous cytoplasmic tail is a truncated NDV F protein cytoplasmic tail that has a deletion of at or about or up to 17 contiguous amino acid residues at or near the C-terminus of the wild-type NDV F protein cytoplasmic tail set forth in SEQ ID NO: 141.
   32. The lipid particle or pseudotyped lentiviral particle of any of embodiments 1-12 and 18-22 and 29-31, wherein the modified cytoplasmic tail comprises the heterologous cytoplasmic tail set forth in SEQ ID NO:147.
   33. The lipid particle or pseudotyped lentiviral particle of any of embodiments 1-12, 18-22 and 29-32, wherein the variant NiV-F comprises the sequence of amino acids set forth in SEQ ID NO:308, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:308.
   34. The lipid particle or pseudotyped lentiviral particle of any of embodiments 1-12, 18-22 and 29-33, wherein the variant NiV-F comprises the sequence of amino acids set forth in SEQ ID NO: 308.
   35. The lipid particle or pseudotyped lentiviral particle of any of embodiments 1-12, 18-22, 29 and 30, wherein the heterologous cytoplasmic tail is a truncated NDV F protein cytoplasmic tail that has a deletion of at or about or up to 20 contiguous amino acid residues at or near the C-terminus of the wild-type NDV F protein cytoplasmic tail set forth in SEQ ID NO: 141.
   36. The lipid particle or pseudotyped lentiviral particle of any of embodiments 1-12 and 18-22 and 29, 30 and 35, wherein the modified cytoplasmic tail comprises the heterologous cytoplasmic tail set forth in SEQ ID NO:150.
   37. The lipid particle or pseudotyped lentiviral particle of any of embodiments 1-12, 18-22, 29, 30, 35 and 36, wherein the variant NiV-F comprises the sequence of amino acids set forth in SEQ ID NO:309, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:309.
   38. The lipid particle or pseudotyped lentiviral particle of any of embodiments 1-12, 18-22, 29, 30 and 35-37, wherein the variant NiV-G comprises the sequence of amino acids set forth in SEQ ID NO:309.
   39. The lipid particle or pseudotyped lentiviral particle of any of embodiments 1-12 and 18-22, wherein the other virus is Hendra virus (HeV) and the glycoprotein is a HeV F protein.
   40. The lipid particle or pseudotyped lentiviral particle of any of embodiments 1-12, 18-22 and 39, wherein the heterologous cytoplasmic tail is a truncated HeV F cytoplasmic tail that has a deletion of up to 26 contiguous amino acid residues at or near the C-terminus of the wild-type HeV F cytoplasmic tail set forth in SEQ ID NO: 58.
   41. The lipid particle or pseudotyped lentiviral particle of any of embodiments 1-12, 18-22, 39 and 40, wherein the heterologous cytoplasmic tail is a truncated HeV F cytoplasmic tail that has a deletion of at or about or up to 22 contiguous amino acid residues at or near the C-terminus of the wild-type HeV F cytoplasmic tail set forth in SEQ ID NO: 58.
   42. The lipid particle or pseudotyped lentiviral particle of any of embodiments 1-12, 18-22 and 39-41, wherein the modified cytoplasmic tail comprises the heterologous cytoplasmic tail set forth in SEQ ID NO:80.
   43. The lipid particle or pseudotyped lentiviral particle of any of embodiments 1-12, 18-22 and 39-41, wherein the variant NiV-F comprises the sequence of amino acids set forth in SEQ ID NO:310, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:310.
   44. The lipid particle or pseudotped lentiviral particle of any of embodiments 1-12, 18-22 and 39-43, wherein the variant NiV-F comprises the sequence of amino acids set forth in SEQ ID NO: 310.
   45. The lipid particle or pseudotyped lentiviral particle of any one of embodiments 1-12 and 18-22, wherein the modified cytoplasmic tail comprises replacement of the attiachment motif of the NiV-F cytoplasmic tail with a cytoplasmic tail or a truncated portion thereof form the attachment protein of another paramyxovirus.
   46. The lipid particle or pseudotyped lentiviral particle of embodiment 45, wherein the paramyxovirus is a Nipah virus, Hendra virus, or Measles virus.
   47. The lipid particle or pseudotyped lentiviral particle of embodiment 45 or embodiment 46, wherein the attachment protein is a G protein, H protein or HN protein.
   48. The lipid particle or pseudotyped lentiviral particle of any of embodiments 45-47, wherein the modified cytoplasmic tail comprises the heterologous cytoplasmic tail set forth in any one of SEQ ID NOS: 210-222.
   49. The lipid particle or pseudotyped lentiviral particle of any of embodiments 1-48, further comprising a modified ectodomain comprising a modified protease cleavage site.
   50. The lipid particle or pseudotyped lentiviral particle of embodiment 49, wherein the modified cleavage site comprises replacement of the cleavage sequence NNTHDLVGDVRLAGV (SEQ ID NO:311) with a modified furin cleavage sequence set forth in any one of SEQ ID NOS: 313-327.
   51. The lipid particle or pseudotyped lentiviral particle of embodiment 49, wherein the modified cleavage site comprises replacement of the cleavage sequence VGDVR (SEQ ID NO:339) with a modified cleavage sequence from another paramyxovirus, optionally wherein the modified cleavage sequence from another paramyxovirus comprises a cathepsin L cleavage site.
   52. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a modified ectodomain, wherein the modified ectodomain comprises a modified cleavage site selected from:
(i) replacement of the cleavage sequence NNTHDLVGDVRLAGV (SEQ ID NO:311) with a modified furin cleavage sequence; or
(ii) replacement of the cleavage sequence VGDVR (SEQ ID NO:339) with a modified cleavage sequence from another paramyxovirus, optionally wherein the modified cleavage sequence from another paramyxovirus comprises a cathepsin L cleavage site.
   53. The lipid particle of embodiment 52, wherein the lipid bilayer is derived from a membrane of a host cell used for producing a retroviral vector or retrovirus-like particle, optionally a lentiviral vector or lentiviral-like particle.
   54. The lipid particle of embodiment 53, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
   55. A pseudotyped lentiviral particle, comprising:
(a) a paramyxovirus glycoprotein (G) or a biologically active portion thereof; and
(b) a variant Nipah virus F glycoprotein (NiV-F) comprising a modified ectodomain, wherein the modified ectodomain comprises a modified cleavage site selected from:
(i) replacement of the cleavage sequence NNTHDLVGDVRLAGV (SEQ ID NO:311) with a modified furin cleavage sequence; or; or
(ii) replacement of the cleavage sequence VGDVR (SEQ ID NO:329) VGDVR (SEQ ID NO:339) with a modified cleavage sequence from another paramyxovirus, optionally wherein the cleavage sequence is a cathepsin L cleavage site.
   56. The lipid particle or pseudotyped lentiviral particle of any of embodiments 49-55, wherein the variant NiV-F protein comprises an F0 precursor or is a proteolytically cleaved form thereof comprising F1 and F2 subunits.
   57. The lipid particle or pseudotyped lentiviral particle of embodiment 52 or embodiment 56, wherein the replacement cleavage sequence comprises a cathepsin L cleavage site and the proteolytically cleaved form is a cathepsin L cleavage product.
   58. The lipid particle or pseudotyped lentiviral particle of embodiment 52 or embodiment 56, wherein the replacement cleavage sequence comprises a modified furin cleavage site and the proteolytically cleaved from is a furin cleavage product.
   59. The lipid particle or pseudotyped lentiviral vector of any of embodiments 49-58, wherein the replacement is in the sequence of amino acids set forth in SEQ ID NO: 1 or SEQ ID NO:320, or a mature form thereof lacking the signal peptide (amino acids 1-26).
   60. The lipid particle or pseudotyped lentiviral vector of any of embodiments 50, 51 and 53-59, wherein the furin cleavage sequence is set forth in any one of SEQ ID NOS: 313-327.
   61. The lipid particle or psuedotyped lentiviral vector of any of embodiments 50, 51 and 53-60, wherein the variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26).
   62. The lipid particle or pseudotyped lentiviral vector of any of embodiments 50, 52 and 53-60, wherein the modified cleavage site from another paramyxovirus is set forth in any one of SEQ ID NOS: 329-338.
   63. The lipid particle or pseudotyped lentiviral vector of any of embodiments 50, 52, 53-60 and 62, wherein the variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26).
   64. The lipid particle or lentiviral vector of any of embodiments 50, 51, 53-60, 62 and 63, wherein the variant NiV-F comprises the sequence of amino acids set forth in SEQ ID NO:239, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:239, or is a mature form thereof lacking the signal peptide (e.g. amino acids 1-26).
   65. The lipid particle or lentiviral vector of any of embodiments 50, 51, 53-60, 62, 63 and 64, wherein the variant NiV-F comprises the sequence of amino acids set forth in SEQ ID NO: 239.
   66. The lipid particle or lentiviral vector of any of embodiments 1-65, wherein the variant NiV-F comprises a heterologous signal sequence compared to the signal sequence of wild-type NiV-F.
   67. The lipid particle or lentiviral vector of any of embodiments 1-66, wherein the variant NiV-F is encoded by a polynucleotide sequence comprising a sequence that encodes a heterologous signal sequence compared to the encoded signal sequence of wild-type NiV-F.
   68. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a heterologous signal sequence, or encoded by a polynucleotide sequence comprising a sequence that encodes a heterologous signal sequence, compared to the wild-type signal sequence of NiV-F.
   69. The lipid particle of embodiment 68, wherein the lipid bilayer is derived from a membrane of a host cell used for producing a retroviral vector or retrovirus-like particle, optionally a lentiviral vector or lentiviral-like particle.
   70. The lipid particle of embodiment 69, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
   71. A pseudotyped lentiviral particle, comprising:
(a) a paramyxovirus glycoprotein (G) or a biologically active portion thereof; and
(b) a variant Nipah virus F glycoprotein (NiV-F) comprising a heterologous signal sequence, or encoded by a polynucleotide sequence comprising a sequence that encodes a heterologous signal sequence, compared to the wild-type signal sequence of NiV-F.
   72. The lipid particle or pseudotyped lentiviral particle of any of embodiments 68-71, wherein the variant NiV-F protein comprises an F0 precursor or is a proteolytically cleaved form thereof comprising F1 and F2 subunits.
   73. The lipid particle or pseudotyped lentiviral particle of embodiment 72, wherein the proteolytically cleaved form is a cathepsin L cleavage product.
   74. The lipid particle or pseudotyped lentiviral vector of any of embodiments 68-73, wherein the heterologous signal sequence is from another virus or is a mammalian signal sequence.
   75. The lipid particle or pseudotyped lentiviral vector of embodiment 74, wherein the other virus is a paramyxovirus, optionally a henipavirus.
   76. The lipid particle or pseudotyped lentiviral vector of any of embodiments 66-75, wherein the heterologous signal sequence is a HevF signal sequence, a CevF signal sequence, a HPIV2 signal sequence, a MevF signal sequence, a NDV-F signal sequence, or a SevF signal sequence.
   77. The lipid particle or pseudotyped lentiviral vector of any of embodiments 66-76, wherein the heterologous signal sequence is set forth in any one of SEQ ID NOS: 340-345.
   78. The lipid particle or pseudotyped lentiviral vector of any of embodiments 66-77, wherein the other virus is Parainfluenza virus and the heterologous signal sequence is the Human Parainfluenza Virus 2 (HPIV2) signal sequence, optionally set forth in SEQ ID NO: 342.
   79. The lipid particle or pseudotyped lentiviral vector of any of embodiments 66-78, wherein the variant NiV-F comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.
   80. The lipid particle or pseudotyped lentiviral vector of any of embodiments 66-79, wherein the variant NiV-F is encoded by a nucleotide sequence that encodes a variant NiV-F that comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.
   81. The lipid particle or lentiviral vector of any of embodiments 66-80, wherein the variant NiV-F comprises the sequence of amino acids set forth in SEQ ID NO: 251.
   82. The lipid particle or pseudotyped lentiviral vector of any of embodiments 66-74, wherein the other virus is HIV-1, Baboon endogenous retrovirus, Cocal virus, Ebola virus, Gibon ape leukemia virus, Murine leukemia virus, or Vesicular stomatitis virus.
   83. The lipid particle or pseudotyped lentiviral vector of any of embodiments 66-74 and 82, wherein the heterologous signal sequence is a HIV1-Env signal sequence, BaEV signal sequence, Cocal signal sequence, EboV signal sequence, GaLV signal sequence, MLV-A signal sequence, or VSVG signal sequence.
   84. The lipid particle or pseudotyped lentiviral vector of any of embodiments 66-74, 82 and 83, wherein the heterologous signal sequence is set forth in any one of SEQ ID NOS: 346-352.
   85. The lipid particle or pseudotyped lentiviral vector of any of embodiments 66-74, and 82-84, wherein the other virus is Vesicular stomatitis virus (VSV) and the heterologous signal sequence is the VSV G glycoprotein (VSV-G) signal sequence, optionally set forth in SEQ ID NO: 352.
   86. The lipid particle or pseudotyped lentiviral vector of any of embodiments 66-74, and 82-85, wherein the variant NiV-F comprises the sequence of amino acids set forth in SEQ ID NO:261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261.
   87. The lipid particle or pseudotyped lentiviral vector of any of embodiments 66-74, and 82-86, wherein the variant NiV-F is encoded by a nucleotide sequence that encodes a variant NiV-F that comprises the sequence of amino acids set forth in SEQ ID NO:261 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261.
   88. The lipid particle or pseudotyped lentiviral vector of any of embodiments 66-74, and 82-87, wherein the variant NiV-F comprises the sequence of amino acids set forth in SEQ ID NO: 261.
   89. The lipid particle or pseudotyped lentiviral vector of any of embodiments 66-74, wherein the mammalian signal sequence is a signal sequence from a human protein.
   90. The lipid particle or pseudotyped lentiviral vector of any of embodiments 66-74 and 89, wherein the heterologous signal sequence is a Prolactin signal sequence, IgGk-L signal sequence, Albumin signal sequence, CD5 signal sequence, Trypsinogen signal sequence orIL2 signal sequence.
   91. The lipid particle or pseudotyped lentiviral vector of any of embodiments 66-74, 89 and 90, wherein the heterologous signal sequence is set forth in any one of SEQ ID NOS: 353-358.
   92. The lipid particle or lentiviral vector of any of embodiments 1-91, wherein the variant NiV-F comprises a heterologous or modified transmembrane domain compared to the transmembrane domain of wild-type NiV-F.
   93. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a heterologous or modified transmembrane domain compared to wild-type NiV-F.
   94. The lipid particle of embodiment 93, wherein the lipid bilayer is derived from a membrane of a host cell used for producing a retroviral vector or retrovirus-like particle, optionally a lentiviral vector or lentiviral-like particle.
   95. The lipid particle of embodiment 94, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
   96. A pseudotyped lentiviral particle, comprising:
(a) a paramyxovirus glycoprotein (G) or a biologically active portion thereof; and
(b) a variant Nipah virus F glycoprotein (NiV-F) comprising a heterologous or modified transmembrane domain compared to wild-type NiV-F.
   97. The lipid particle or pseudotyped lentiviral particle of any of embodiments 93-96, wherein the variant NiV-F protein comprises an F0 precursor or is a proteolytically cleaved form thereof comprising F1 and F2 subunits.
   98. The lipid particle or pseudotyped lentiviral particle of embodiment 97, wherein the proteolytically cleaved form is a cathepsin L cleavage product.
   99. The lipid particle or pseudotyped lentiviral particle of any of embodiments 93-98, wherein the transmembrane domain is a heterologous transmembrane domain from Hendra virus, optionally wherein the transmembrane domain is set forth in SEQ ID NO: 361.
   100. The lipid particle or pseudotyped lentiviral particle of any of embodiments 93-99, wherein the transmembrane domain is a modified transmembrane domain containing the amino acid replacement S490A, Y498A, S504A or I516V, corresponding to numbering of positions set forth in SEQ ID NO:1, optionally wherein the transmembrane domain is set forth in any one of SEQ ID NOS: 359, 360, 362 or 363.
   101. The lipid particle or pseudotyped lentiviral vector of any of embodiments 93-100, wherein the variant NiV-F comprises the sequence of amino acids set forth in any one of SEQ ID NO: 268-272 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 268-272.
   102. The lipid particle or pseudotyped lentiviral particle of any of embodiments 1-101, which further comprises a hyperfusogenic mutation.
   103. The lipid particle or pseudotyped lentiviral particle of embodiment 102, wherein the hyperfusogenic mutation is introduced at a N-glycosylation site with respect to amino acid positions 64, 67, 99, 414 and/or 464 of SEQ ID NO. 1.
   104. The lipid particle or pseudotyped lentiviral particle of embodiment 102 or 103, wherein the hyperfusogenic mutation is one or more of N64Q, N67Q, N99Q, N414Q and/or N464Q, with reference to numbering set forth in SEQ ID NO:1
   105. The lipid particle or pseudotyped lentiviral particle of embodiment 104, wherein the one or more mutations are selected from the group consisting of N64Q, N67Q, N99Q, N414Q, N464Q, N67Q/N99Q, N67Q/N414Q, N67Q/N464Q, N99Q/N414Q, N99Q/N464Q, N414Q/N464Q, N67Q/N99Q/N414Q, N67Q/N414Q/N464Q, N99Q/N414Q/N464Q, or N67Q/N99Q/N414Q/N464Q, N64Q/N99Q, N64Q/N99Q/N464Q, N64Q/N67Q/N99Q, and N64Q/N67Q/N99Q/N464Q.
   106. The lipid particle or pseudotyped lentiviral particle of embodiment 102, wherein the hyperfusogenic mutation is introduced at a hexamer stability site with respect to amino acid positions 393 and/or 109 of SEQ ID NO. 1.
   107. The lipid particle or pseudotyped lentiviral particle of embodiments 102 or 106, wherein the hyperfusogenic mutation is one or more of R109L, Q393L or R109L and Q393L, with reference to numbering set forth in SEQ ID NO:1.
   108. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a hyperfusogenic mutation selected from:
a mutation that is introduced at a N-glycosylation site with respect to amino acid positions 64, 67, 99, 414 and/or 464 of SEQ ID NO. 1; and/or
a mutation that is introduced at a hexamer stability site with respect to amino acid positions 393 and/or 109 of SEQ ID NO. 1.
   109. The lipid particle of any of embodiments 106, 107 or 108, wherein the hyperfusogenic mutation is introduced at a hexamer stability site with respect to amino acid positions 393 of SEQ ID NO. 1, optionally wherein the mutation is a substitution Q393L.
   110. The lipid particle of embodiment 108 or embodiment 109, wherein the lipid bilayer is derived from a membrane of a host cell used for producing a retroviral vector or retrovirus-like particle, optionally a lentiviral vector or lentiviral-like particle.
   111. The lipid particle of embodiment 110, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
   112. A pseudotyped lentiviral particle, comprising:
(a) a paramyxovirus glycoprotein (G) or a biologically active portion thereof; and
(b) a variant Nipah virus F glycoprotein (NiV-F) comprising a hyperfusogenic mutation selected from:
a mutation that is introduced at a N-glycosylation site with respect to amino acid positions 64, 67, 99, 414, and/or 464 of SEQ ID NO. 1; and/or
a mutation that is introduced at a hexamer stability site with respect to amino acid positions 393 and/or 109 of SEQ ID NO. 1.
   113. The lipid particle or pseudotyped lentiviral particle of any of embodiments 108-112, wherein the variant NiV-F protein comprises an F0 precursor or is a proteolytically cleaved form thereof comprising F1 and F2 subunits.
   114. The lipid particle or pseudotyped lentiviral particle of embodiment 113, wherein the proteolytically cleaved form is a cathepsin L cleavage product.
   115. The lipid particle or pseudotyped lentiviral vector of any of embodiments 102-114, comprising:
a mutation N67Q, N99Q, N414Q or N464Q or any combination thereof; and/or
a mutation R109L or Q393L or a combination thereof.
   116. The lipid particle or pseudotyped lentiviral vector of any of embodiments 102-115, wherein the hyperfusogenic mutation is in the sequence of amino acids set forth in SEQ ID NO:1, SEQ ID NO:2, SEQ ID NO:302 or SEQ ID NO:303.
   117. The lipid particle or pseudotyped lentiviral vector of any of embodiments 1-116, wherein the variant NiV-F comprises the sequence of amino acids set forth in SEQ ID NO:247, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:247.
   118. The lipid particle or pseudotyped lentiviral vector of any of embodiments 1-117, wherein the variant NiV-F comprises the sequence of amino acids set forth in SEQ ID NO: 247.
   119. The lipid particle or pseudotyped lentiviral particle of any of embodiments 1-116, wherein the variant NiV-F comprises the sequence of amino acids set forth in SEQ ID NO:292, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:292.
   120. The lipid particle or pseudotyped lentiviral particle of any of embodiments 1-116 or 119, wherein the variant NiV-F comprises the sequence of amino acids set forth in SEQ ID NO: 292.
   121. The lipid particle or pseudotyped lentiviral particle of any of embodiments 1-120, wherein the G protein or the biologically active portion thereof is a wild-type Nipah virus G (NiV-G) protein or a Hendra virus G protein or is a functionally active variant or biologically active portion thereof.
   122. The lipid particle or pseudotyped lentiviral particle of any of embodiments 1-121, wherein the G protein or the biologically active portion thereof is a wild-type NiV-G protein or a functionally active variant or a biologically active portion thereof.
   123. The lipid particle or pseudotyped lentiviral particle of any of embodiments 1-122, wherein the G protein is a biologically active portion of wild-type NiV-G that is truncated in which the G protein lacks up to 34 contiguous amino acids at or near the N-terminus of the wild-type NiV-G set forth in SEQ ID NO:305.
   124. The lipid particle or pseudotyped lentiviral particle of any of embodiments 1-123, wherein the G-protein is a NiV-G functionally active variant or a biologically active portion thereof that exhibits reduced binding to Ephrin B2 or Ephrin B3.
   125. The lipid particle or pseudotyped lentiviral particle of embodiment 124, wherein the NiV-G functionally active variant or a biologically active portion thereof protein comprises: one or more amino acid substitutions corresponding to amino acid substitutions selected from the group consisting of E501A, W504A, Q530A and E533A with reference to numbering set forth in SEQ ID NO:305.
   126. The lipid particle or pseudotyped lentiviral particle of embodiment 124 or embodiment 125, wherein the NiV-G functionally active variant or a biologically active portion thereof comprises amino acid substitutions E501A, W504A, Q530A and E533A with reference to numbering set forth in SEQ ID NO:305.
   127. The lipid particle or pseudotyped lentiviral particle of any of embodiments 122-126, wherein the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301.
   128. The lipid particle or pseudotyped lentiviral particle of any of embodiments 122-127, wherein the NiV-G functionally active variant or a biologically active portion thereof comprises the amino acid sequence set forth in SEQ ID NO: 301.
   129. The lipid particle or pseudotyped lentiviral particle of any of embodiments 122-128, wherein the G protein is linked to a binding domain that binds to a target cell surface molecule on a target cell.
   130. The lipid particle or pseudotyped lentiviral particle of embodiment 129, wherein the binding domain is attached to the C-terminus of the G protein.
   131. The lipid particle or pseudotyped lentiviral particle of embodiment 129 or embodiment 130, wherein the cell surface molecule is a protein, glycan, lipid or low molecular weight molecule.
   132. The lipid particle or pseudotyped lentiviral particle of any of embodiments 129-131, wherein the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells.
   133. The lipid particle or pseudotyped lentiviral particle of any of embodiments 129-132, wherein the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell.
   134. The lipid particle or pseudotyped lentiviral particle of any of embodiments 129-133 wherein the target cell is a hepatocyte.
   135. The lipid particle or pseudotyped lentiviral particle of any of embodiments 129-133 and 134, wherein the binding domain binds to a cell surface molecule selected from the group consisting of ASGR1, ASGR2 and TM4SF.
   136. The lipid particle or pseudotyped lentiviral particle of any of embodiments 129-133, wherein the target cell is a T cell.
   137. The lipid particle or pseudotyped lentiviral particle of any of embodiments 129-133 and 136, wherein the binding domain binds to a cell surface molecule selected from the group consisting of CD3, CD4 or CD8.
   138. The lipid particle or pseudotyped lentiviral particle of any of embodiments 129-137, wherein the binding domain is an antibody or antigen-binding fragment, a single domain antibody or a DARPin.
   139. The lipid particle or pseudotyped lentiviral particle of any of embodiments 129-138, wherein the binding domain is a single domain antibody that is a VHH or is a single chain variable fragment (scFv).
   140. The lipid particle or pseudotyped lentiviral particle of any of embodiments 129-139, wherein the binding domain is attached to the G protein via a linker.
   141. The lipid particle or pseudotyped lentiviral particle of embodiment 140, wherein the linker is a peptide linker.
   142. The lipid particle or pseudotyped lentiviral particle of embodiment 141, wherein the peptide linker is 2 to 65 amino acids in length.
   143. The lipid particle or pseudotyped lentiviral particle of embodiment 141 or embodiment 142, wherein the peptide linker is a flexible linker that comprises GS, GGS, GGGGS (SEQ ID NO:287), GGGGGS (SEQ ID NO:288) or combinations thereof.
   144. The lipid particle or pseudotyped lentiviral particle of any of embodiments 141-143, wherein the peptide linker is selected from: (GGS)n, wherein n is 1 to 10; (GGGGS)n (SEQ ID NO:289), wherein n is 1 to 10; or (GGGGGS)n (SEQ ID NO:290), wherein n is 1 to 6.
   145. The lipid particle or pseudotyped lentiviral vector of any of embodiments 1-144 that is replication defective.
   146. The lipid particle or pseudotyped lentiviral vector of any of embodiments 1-145 prepared by a method comprising transducing a producer cell with packaging plasmids that encode a Gag-pol, Rev, Tat and the variant NiVG and the F protein.
   147. The lipid particle or pseudotyped lentiviral vector of any of embodiments 1-146, wherein the particle further comprises a viral nucleic acid.
   148. The lipid particle or pseudotyped lentiviral vector of embodiment 147, wherein the viral nucleic acid comprises one or more of (e.g., all of) the following nucleic acid sequences: 5′ LTR (e.g., comprising US and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
   149. The lipid particle or pseudotyped lentiviral vector of any of embodiments 1-147, wherein the particle is devoid of viral genomic DNA.
   150. The lipid particle or pseudotyped lentiviral particle of any of embodiments 1-149, wherein the particle is produced as a preparation with increased titer compared to a reference particle preparation that is similarly produced but with incorporation of the full-length NiV-F protein set forth in SEQ ID NO: 1, optionally wherein the lipid particle or pseudotyped lentiviral particle is a lentivirus vector.
   151. The lipid particle or pseudotyped lentiviral particle of any of embodiments 1-150, wherein the particle is produced as a preparation with increased titer compared to a reference particle preparation that is similarly produced but with incorporation of the truncated NiV-F protein comprising the sequence set forth in SEQ ID NO:303 or encoded by a polynucleotide that encodes the sequence set forth in SEQ ID NO:302, optionally wherein the lipid particle or psuedotyped lentiviral particle is a lentivirus vector.
   152. The lipid particle or pseudotyped lentiviral particle of embodiment 150 or embodiment 151, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.
   153. The lipid particle or pseudotyped lentiviral particle of any of embodiments 150-152, wherein the titer is increased by about or greater than about 2.0-fold.
   154. The lipid particle or pseudotyped lentiviral particle of any of embodiments 150-152, wherein the titer is increased by about or greater than about 3.0-fold.
   155. The lipid particle or pseudotyped lentiviral particle of any of embodiments 150-152, wherein the titer is increased by about or greater than about 4.0-fold.
   156. The lipid particle or pseudotyped lentiviral particle of any of embodiments 150-152, wherein the titer is increased by about or greater than about 5.0-fold.
   157. The lipid particle or pseudotyped lentiviral particle of any of embodiments 1-156, further comprising an exogenous agent for delivery to a target cell.
   158. The lipid particle or pseudotyped lentiviral particle of embodiment 157, wherein the exogenous agent is present in the lumen.
   159. The lipid particle or pseudotyped lentiviral particle of embodiment 157 or embodiment 158, wherein the exogenous agent is a protein or a nucleic acid, optionally wherein the nucleic acid is a DNA or RNA.
   160. The lipid particle or pseudotyped lentiviral particle of any of embodiments 157-159, wherein the exogenous agent is a nucleic acid encoding a cargo for delivery to the target cell.
   161. The lipid particle or lentiviral vector of any of embodiments 157-160, wherein the exogenous agent is or encodes a therapeutic agent or a diagnostic agent.
   162. The lipid particle or pseudotyped lentiviral particle of any of embodiments 157-161, wherein the exogenous agent encodes a membrane protein, optionally wherein the membrane protein is an antigen receptor for targeting cells expressed by or associated with a disease or condition.
   163. The lipid particle or pseudotyped lentiviral particle of embodiment 162, wherein the membrane protein is a chimeric antigen receptor (CAR).
   164. The lipid particle or pseudotyped lentiviral particle of any of embodiments 157-163, wherein the target cell is a T cell.
   165. The lipid particle or lentiviral vector of any of embodiments 157-161, wherein the exogenous agent is a nucleic acid comprising a payload gene for correcting a genetic deficiency, optionally a genetic deficiency in the target cell, optionally wherein the genetic deficiency is associated with a liver cell or a hepatocyte.
   166. The lipid particle or lentiviral vector of any of embodiments 157-165, wherein binding of the G protein to a cell surface molecule on the target cell mediates fusion of the particle with the target cell and delivery of the exogenous agent to the target cell.
   167. The lipid particle or pseudotyped lentiviral particle of any of embodiments 157-166, wherein at or greater than 10%, 20%, 30%, 40%, 50%, 60% of the target cells are delivered the exogenous agent.
   168. The lipid particle or lentiviral vector of any of embodiments 157-167, wherein delivery of the exogenous cell to the target cell is increased compared to a reference particle preparation that is similarly produced but in which the NiV-F protein is the full-length NiV-F protein set forth in SEQ ID NO: 1, optionally wherein the lipid particle or pseudotyped lentiviral particle is a lentivirus vector.
   169. The lipid particle or pseudotyped lentiviral particle of any of embodiments 157-167, wherein delivery of the exogenous cell to the target cell is increased compared to a reference particle preparation that is similarly produced but in which the F protein is the truncated NiV-F comprising the sequence set forth in SEQ ID NO:303 or encoded by a polynucleotide that encodes the sequence set forth in SEQ ID NO:302, optionally wherein the lipid particle or pseudotyped lentiviral particle is a lentivirus vector.
   170. The lipid particle or pseudotyped lentiviral particle of any of embodiments 157-169, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.
   171. A polynucleotide comprising a nucleic acid molecule encoding a variant Nipah virus F glycoprotein (NiV-F) comprising a modified cytoplasmic tail, wherein the modified cytoplasmic tail comprises:
(i) a heterologous cytoplasmic tail or a truncated portion thereof from a glycoprotein from another virus or a virus-associated protein, wherein the variant NiV-F is a chimeric protein;
(ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or
(iii) a modified NiV-F cytoplasmic that comprises a modified endocytosis motif.
   172. The polynucleotide of embodiment 171, wherein the variant NiV-F protein comprises an F0 precursor or is a proteolytically cleaved form thereof comprising F1 and F2 subunits.
   173. The polynucleotide of embodiment 172, wherein the proteolytically cleaved form is a cathepsin L cleavage product.
   174. The polynucleotide of any of embodiments 171-173, wherein the variant Niv-F protein comprises a modified F1 subunit containing the modified cytoplasmic tail and an F2 subunit in which (1) the F1 subunit is a modified F1 composed of the sequence of the modified cytoplasmic tail directly linked to the C-terminus of the sequence set forth in SEQ ID NO:383, and (2) the F2 subunit is set forth as SEQ ID NO:365.
   175. The polynucleotide of any of embodiments 171-174, wherein the variant NiV-F comprises in order from N-terminus to C-terminus the an extracellular domain, a transmembrane domain and the modified cytoplasmic tail.
   176. The polynucleotide of embodiment 175, wherein the extracellular domain and transmembrane domain of the variant NiV-F are from a wild-type NiV-F or a biologically active variant thereof.
   177. The lipid particle or pseduotyped lentiviral particle of embodiment 175 or embodiment 176, wherein the extracellular domain and transmembrane domain of the variant NiV-F comprise the sequence set forth in SEQ ID NO:2, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:2.
   178. The polynucleotide of any of embodiments 175-177, wherein the extracellular domain and transmembrane domain of the variant NiV-F comprise the sequence set forth in SEQ ID NO:2.
   179. The polynucleotide of any of embodiments 174-179, wherein the modified cytoplasmic tail comprises a heterologous cytoplasmic tail set forth in any one of SEQ ID NOS: 80, 133, 147 and 150.
   180. The polynucleotide of any of embodiments 174-179, wherein the encoded variant NiV-F comprises the sequence of amino acids set forth in any one of SEQ ID NOS: 307, 308, 309, and 310.
   181. The polynucleotide of any of embodiments 174-178, wherein the modified cytoplasmic tail is a truncated NiV-F cytoplasmic tail set forth in any one of SEQ ID NOS: 27.
   182. The polynucleotide of any of embodiments 174-178 and 181, wherein the encoded variant NiV-F comprises the sequence of amino acids set forth in any one of SEQ ID NOS: 306.
   183. A polynucleotide comprising a nucleic acid molecule encoding a variant Nipah virus F glycoprotein (NiV-F) comprising a modified ectodomain, wherein the modified ectodomain comprises a modified cleavage site selected from:
(i) replacement of the cleavage sequence NNTHDLVGDVRLAGV (SEQ ID NO:311) with a modified furin cleavage sequence; or
(ii) replacement of the cleavage sequence VGDVR (SEQ ID NO:339) with a modified cleavage sequence from another paramyxovirus, optionally wherein the cleavage sequence is a cathepsin L cleavage site;
   wherein the replacement is in the sequence of amino acids set forth in SEQ ID NO: 1 or SEQ ID NO:320, or a mature form thereof lacking the signal peptide (amino acids 1-26). 184. The polynucleotide of embodiment 183, wherein the furin cleavage sequence is set forth in any one of SEQ ID NOS: 313-327.
   185. The polynucleotide of embodiment 183 or embodiment 184, wherein the encoded variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26).
   186. The polynucleotide of embodiment 183, wherein the modified cleavage site from another paramyxovirus is set forth in any one of SEQ ID NOS: 329-338.
   187. The polynucleotide of embodiment 183 or embodiment 186, wherein the encoded variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26).
   188. The polynucleotide of embodiment 183, embodiment 186 or embodiment 187, wherein the encoded variant NiV-F comprises the sequence of amino acids set forth in SEQ ID NO:239, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:239, or is a mature form thereof lacking the signal peptide (e.g. amino acids 1-26).
   189. The polynucleotide of any of embodiments 183 and 186-188, wherein the encoded variant NiV-F comprises the sequence of amino acids set forth in SEQ ID NO: 239.
   190. A polynucleotide comprising a nucleic acid molecule encoding a variant Nipah virus F glycoprotein (NiV-F) comprising a heterologous signal sequence compared to the wild-type signal sequence of NiV-F.
   191. The polynucleotide of embodiment 190, wherein the heterologous signal sequence is from another virus or is a mammalian signal sequence.
   192. The polynucleotide of embodiment 191, wherein the other virus is a paramyxovirus, optionally a henipavirus.
   193. The polynucleotide of any of embodiments 190-192, wherein the heterologous signal sequence is a HevF signal sequence, a CevF signal sequence, a HPIV2 signal sequence, a MevF signal sequence, a NDV-F signal sequence, or a SevF signal sequence.
   194. The polynucleotide of any of embodiments 190-193, wherein the heterologous signal sequence is set forth in any one of SEQ ID NOS: 340-345.
   195. The polynucleotide of any of embodiments 190-191, wherein the other virus is Parainfluenza virus and the heterologous signal sequence is the Human Parainfluenza Virus 2 (HPIV2) signal sequence, optionally set forth in SEQ ID NO: 342.
   196. The polynucleotide of any of embodiments 190, 191 and 195, wherein the encoded variant NiV-F comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.
   197. The polynucleotide of any of embodiments 190, 191, 195 and 196, wherein the encoded variant NiV-F comprises the sequence of amino acids set forth in SEQ ID NO: 251.
   198. The polynucleotide of any of embodiments 190-191, wherein the other virus is HIV-1, Baboon endogenous retrovirus, Cocal virus, Ebola virus, Gibon ape leukemia virus, Murine leukemia virus, or Vesicular stomatitis virus.
   199. The polynucleotide of any of embodiments 190-191 and 198, wherein the heterologous signal sequence is a HIV1-Env signal sequence, BaEV signal sequence, Cocal signal sequence, EboV signal sequence, GaLV signal sequence, MLV-A signal sequence, or VSVG signal sequence.
   200. The polynucleotide of any of embodiments 190-192, 198 and 199, wherein the heterologous signal sequence is set forth in any one of SEQ ID NOS: 346-352.
   201. The polynucleotide of any of embodiments 190-191 and 198-199, wherein the other virus is Vesicular stomatitis virus (VSV) and the heterologous signal sequence is the VSV G glycoprotein (VSV-G) signal sequence, optionally set forth in SEQ ID NO: 352.
   202. The polynucleotide of any of embodiments 190-191 and 198-201, wherein the encoded variant NiV-F comprises the sequence of amino acids set forth in SEQ ID NO:261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261.
   203. The polynucleotide of any of embodiments 190-191 and 198-202, wherein the encoded variant NiV-F comprises the sequence of amino acids set forth in SEQ ID NO: 261.
   204. The polynucleotide of any of embodiments 190-191, wherein the mammalian signal sequence is a signal sequence from a human protein.
   205. The polynucleotide of any of embodiments 190-191 and 204, wherein the heterologous signal sequence is a Prolactin signal sequence, IgGk-L signal sequence, Albumin signal sequence, CD5 signal sequence, Trypsinogen signal sequence orIL2 signal sequence.
   206. The polynucleotide of any of embodiments 190-192, 204 and 205, wherein the heterologous signal sequence is set forth in any one of SEQ ID NOS: 353-358.
   207. A polynucleotide comprising a nucleic acid molecule encoding a variant Nipah virus F glycoprotein (NiV-F) comprising a heterologous or modified transmembrane domain compared to wild-type NiV-F.
   208. The polynucleotide of embodiment 207, wherein the transmembrane domain is a heterologous transmembrane domain from Hendra virus, optionally wherein the transmembrane domain is set forth in SEQ ID NO: 361.
   209. The polynucleotide of embodiment 207 or embodiment 208, wherein the transmembrane domain is a modified transmembrane domain containing the amino acid replacement S490A, Y498A, S504A or I516V, corresponding to numbering of positions set forth in SEQ ID NO:1, optionally wherein the transmembrane domain is set forth in any one of SEQ ID NOS: 359, 360, 362 or 363.
   210. The polynucleotide of any of embodiments 207-209, wherein the encoded variant NiV-F comprises the sequence of amino acids set forth in any one of SEQ ID NO: 268-272 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 268-272.
   211. A polynucleotide comprising a nucleic acid molecule encoding a variant Nipah virus F glycoprotein (NiV-F) comprising a hyperfusogenic mutation selected from:
a mutation that is introduced at a N-glycosylation site with respect to amino acid positions 64, 67, and/or 99 of SEQ ID NO. 1; and/or
a mutation that is introduced at a hexamer stability site with respect to amino acid positions 393 and/or 109 of SEQ ID NO. 1.
   212. The polynucleotide of embodiment 211, wherein the encoded variant NiV-F comprises the sequence of amino acids set forth in SEQ ID NO:247, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:247.
   213. The polynucleotide of embodiment 211 or embodiment 212, wherein the variant NiV-F comprises the sequence of amino acids set forth in SEQ ID NO: 247.
   214. The polynucleotide of any of embodiments 211-213, wherein the encoded variant NiV-F comprises the sequence of amino acids set forth in SEQ ID NO:292, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:292.
   215. The polynucleotide of any of embodiments 211-214, wherein the encoded variant NiV-F comprises the sequence of amino acids set forth in SEQ ID NO: 292.
   216. The polynucleotide of any of embodiments 171-215, wherein the nucleic acid sequence is a first nucleic acid sequence and the polynucleotide further comprises a second nucleic acid sequence encoding a paramyxovirus fusion (G) protein molecule or a biologically active portion thereof or functionally active variant thereof.
   217. The polynucleotide of embodiment 216, wherein the G protein or the biologically active portion thereof is a wild-type Nipah virus G (NiV-G) protein or a Hendra virus G protein or is a functionally active variant or biologically active portion thereof.
   218. The polynucleotide of embodiment 216 or embodiment 217, wherein the G protein or the biologically active portion thereof is a wild-type NiV-G protein or a functionally active variant or a biologically active portion thereof.
   219. The polynucleotide of any of embodiments 216-218, wherein the G protein is a biologically active portion of wild-type NiV-G that is truncated in which the G protein lacks up to 34 contiguous amino acids at or near the N-terminus of the wild-type NiV-G set forth in SEQ ID NO:305.
   220. The polynucleotide of any of embodiments 216-219, thereof that exhibits reduced binding to Ephrin B2 or Ephrin B3.
   221. The polynucleotide of embodiment 220, wherein the NiV-G functionally active variant or a biologically active portion thereof protein comprises: one or more amino acid substitutions corresponding to amino acid substitutions selected from the group consisting of E501A, W504A, Q530A and E533A with reference to numbering set forth in SEQ ID NO:305.
   222. The polynucleotide of embodiment 220 or embodiment 221, wherein the NiV-G functionally active variant or a biologically active portion thereof comprises amino acid substitutions E501A, W504A, Q530A and E533A with reference to numbering set forth in SEQ ID NO:305.
   223. The polynucleotide of any of embodiments 216-222, wherein the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301.
   224. The polynucleotide of any of embodiments 216-223, wherein the NiV-G functionally active variant or a biologically active portion thereof comprises the amino acid sequence set forth in SEQ ID NO: 301.
   225. The polynucleotide of any of embodiments 216-224, wherein the G protein is linked to a binding domain that binds to a target cell surface molecule on a target cell.
   226. The polynucleotide of embodiment 225, wherein the binding domain is attached to the C-terminus of the G protein.
   227. The polynucleotide of embodiment 225 or embodiment 226, wherein the cell surface molecule is a protein, glycan, lipid or low molecular weight molecule.
   228. The polynucleotide of any of embodiments 225-227, wherein the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells.
   229. The polynucleotide of any of embodiments 225-228, wherein the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell.
   230. The polynucleotide of any of embodiments 225-229, wherein the target cell is a hepatocyte.
   231. The polynucleotide of any of embodiments 225-230, wherein the binding domain binds to a cell surface molecule selected from the group consisting of ASGR1, ASGR2 and TM4SF.
   232. The polynucleotide of any of embodiments 225-229, wherein the target cell is a T cell.
   233. The polynucleotide of any of embodiments 225-229 and 232, wherein the binding domain binds to a cell surface molecule selected from the group consisting of CD3, CD4 or CD8.
   234. The polynucleotide of any of embodiments 225-233, wherein the binding domain is an antibody or antigen-binding fragment, a single domain antibody or a DARPin.
   235. The polynucleotide of any of embodiments 225-234, wherein the binding domain is a single domain antibody that is a VHH or is a single chain variable fragment (scFv).
   236. The polynucleotide of any of embodiments 225-235, wherein the binding domain is attached to the G protein via a linker.
   237. The polynucleotide of embodiment 236, wherein the linker is a peptide linker.
   238. The polynucleotide of embodiment 237, wherein the peptide linker is 2 to 65 amino acids in length.
   239. The polynucleotide of embodiment 237 or embodiment 238, wherein the peptide linker is a flexible linker that comprises GS, GGS, GGGGS (SEQ ID NO:287), GGGGGS (SEQ ID NO:288) or combinations thereof.
   240. The polynucleotide of any of embodiments 237-239, wherein the peptide linker is selected from: (GGS)n, wherein n is 1 to 10; (GGGGS)n (SEQ ID NO:289), wherein n is 1 to 10; or (GGGGGS)n (SEQ ID NO:290), wherein n is 1 to 6.
   241. The polynucleotide of any of embodiments 216-240, wherein the polynucleotide comprises an IRES or a sequence encoding a linking peptide between the first and second nucleic acid sequences, optionally, wherein the linking peptide is a self-cleaving peptide or a peptide that causes ribosome skipping, optionally a T2A peptide.
   242. The polynucleotide of any of embodiments 171-241, further comprising at least one promoter that is operatively linked to control expression of the nucleic acid, optionally expression of the first nucleic acid sequence and the second nucleic acid sequence.
   243. A vector, comprising the polynucleotide of any of embodiments 171-242.
   244. The vector of embodiment 243, wherein the vector is a mammalian vector, viral vector or artificial chromosome, optionally wherein the artificial chromosome is a bacterial artificial chromosome (BAC).
   245. A plasmid, comprising the polynucleotide of any of embodiments 171-242.
   246. The plasmid of embodiment 245, further comprising one or more nucleic acids encoding proteins for lentivirus production.
   247. A cell comprising the polynucleotide of any of embodiments 171-242 or the vector of embodiment 243 or embodiment 244, or the plasmid of embodiment 245 or 246.
   248. A method of making a lipid particle comprising a variant Nipah virus F protein and, optionally a paramyxovirus G protein, comprising:
a) providing a cell that comprises the polynucleotide of any of embodiments 171-242 or the vector of embodiment 243 or embodiment 244, or the plasmid of embodiment 245 or embodiment 246;
b) culturing the cell under conditions that allow for production of a lipid particle, and
c) separating, enriching, or purifying the lipid particle from the cell, thereby making the lipid particle.
   249. A method of making a pseudotyped lentiviral vector, comprising:
a) providing a producer cell that comprises a lentiviral viral nucleic acid(s), and the polynucleotide of any of embodiments 171-242 or the vector of embodiment 243 or embodiment 244, or the plasmid of embodiment 245 or embodiment 246;
b) culturing the cell under conditions that allow for production of the lentiviral vector, and
c) separating, enriching, or purifying the lentiviral vector from the cell, thereby making the pseudotyped lentiviral vector.
   250. The method of embodiment 248 or embodiment 249, wherein prior to step (b) the method further comprises providing the cell a polynucleotide encoding a henipavirus F protein molecule or biologically active portion thereof.
   251. The method of any of embodiments 248-250, wherein the cell is a mammalian cell.
   252. The method of any of embodiments 248-251, wherein the cell is a producer cell comprising viral nucleic acid, optionally retroviral nucleic acid or lentiviral nucleic acid, and the lipid particle is a viral particle or a viral-like particle, optionally a retroviral particle or a retroviral-like particle, optionally a lentiviral particle or lentiviral-like particle.
   253. A producer cell comprising the polynucleotide of any of embodiments 171-242 or the vector of embodiment 243 or embodiment 244, or the plasmid of embodiment 245 or embodiment 246.
   254. The producer cell of embodiment 253, further comprising nucleic acid encoding a paramyxovirus G protein or a biologically active portion thereof.
   255. The producer cell of embodiment 254, wherein the G protein or the biologically active portion thereof is a wild-type Nipah virus G (NiV-G) protein or a Hendra virus G protein or is a functionally active variant or biologically active portion thereof.
   256. The producer cell of embodiment 254 or embodiment 255, wherein the G protein or the biologically active portion thereof is a wild-type NiV-G protein or a functionally active variant or a biologically active portion thereof.
   257. The producer cell of any of embodiments 254-256, wherein the G protein is a biologically active portion of wild-type NiV-G that is truncated in which the G protein lacks up to 34 contiguous amino acids at or near the N-terminus of the wild-type NiV-G set forth in SEQ ID NO:305.
   258. The producer cell of any of embodiments 254-257, thereof that exhibits reduced binding to Ephrin B2 or Ephrin B3.
   259. The producer cell of embodiment 258, wherein the NiV-G is a functionally active variant or a biologically active portion thereof protein comprising: one or more amino acid substitutions corresponding to amino acid substitutions selected from the group consisting of E501A, W504A, Q530A and E533A with reference to numbering set forth in SEQ ID NO:305.
   260. The producer cell of embodiment 258 or embodiment 259, wherein the NiV-G functionally active variant or a biologically active portion thereof comprises amino acid substitutions E501A, W504A, Q530A and E533A with reference to numbering set forth in SEQ ID NO:305.
   261. The producer cell of any of embodiments 253-260, wherein the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301.
   262. The producer cell of any of embodiments 254-261, wherein the NiV-G functionally active variant or a biologically active portion thereof comprises the amino acid sequence set forth in SEQ ID NO: 301.
   263. The producer cell of any of embodiments 254-262, wherein the G protein is linked to a binding domain that binds to a target cell surface molecule on a target cell.
   264. The producer cell of any of embodiments 253-263, wherein the cell further comprises a viral nucleic acid, optionally wherein the viral nucleic acid is a lentiviral nucleic acid.
   265. A lipid particle or pseudotyped lentiviral vector produced by the method of any of embodiments 244-248 or from the producer cell of any of embodiments 253-264.
   266. A composition comprising a plurality of lipid particles or a plurality of lentiviral vectors of any of embodiments 1-170 and 265.
   267. The composition of embodiment 266, further comprising a pharmaceutically acceptable carrier.
   268. A method of transducing a cell comprising transducing a cell with a lentiviral vector of any of embodiments 1-170 and 165 or a composition comprising a lentiviral vector or plurality of lentiviral vectors of embodiment 166 or embodiment 167.
   269. A method of delivering an exogenous agent to a subject (e.g., a human subject), the method comprising administering to the subject the lipid particle or lentiviral vector of any of embodiments 1-170 and 265 or a composition comprising the lipid particle or lentiviral vector of any of embodiments 1-170 and 265 or a composition comprising a lentiviral vector or plurality of lentiviral vectors of embodiment 266 or embodiment 267, wherein lipid particle or lentiviral vector comprise the exogenous agent.
   270. A method of delivering an exogenous agent to a target cell, the method contacting a target cell with the lipid particle or lentiviral vector of any of embodiments 1-170 and 265 or a composition comprising the lipid particle or lentiviral vector of any of embodiments 1-170 and 265 or a composition comprising a lentiviral vector or plurality of lentiviral vectors of embodiment 266 or embodiment 267, wherein the lipid particle or lentiviral vector comprise the exogenous agent.
   271. The method of embodiment 270, wherein the contacting transduces the cell with lentiviral vector or the lipid particle.
   272. The method of embodiment 270 or embodiment 271, wherein the contacting is in vivo in a subject.
   273. A method of treating a disease or disorder in a subject (e.g., a human subject), the method comprising administering to the subject a lipid particle of any of embodiments or the lentiviral vector of any of embodiments 1-170 and 265 or the composition of embodiment 266 or embodiment 267.
   274. A method of fusing a mammalian cell to a lipid particle, the method comprising administering to the subject a lipid particle or the lentiviral vector of any of embodiments 1-170 and 265 or the composition of embodiment 266 or embodiment 267.
   275. The method of embodiment 274, wherein the fusing of the mammalian cell to the lipid particle delivers an exogenous agent to a subject (e.g., a human subject).

### VII. EXAMPLES

The following examples are included for illustrative purposes only and are not intended to limit the scope of the invention.

### Example 1 Optimization of NiV-F Results in Increased Targeted Fusogen Titer

This Example describes generation and assessment of lentiviruses pseudotyped with a modified fusogenic F protein (F protein). For comparison, control NiV-F proteins included a NiV-F containing either (1) a full-length cytoplasmic tail (set forth in SEQ ID NO:4), in which the full sequence of the NiV-F sequence is set forth in SEQ ID NO:1 including the signal peptide or as amino acid residues 27-546 of SEQ ID NO:1 for the mature sequence without the signal peptide; or (2) a truncated cytoplasmic domain lacking 22 contiguous amino acids (set forth in SEQ ID NO:26), in which the full sequence of the control NiV-F sequence is set forth in SEQ ID NO:302 including the signal peptide or in SEQ ID NO:303 for the mature sequence without the signal peptide.

### A. Generation of NiV-F Variants

### 1. NiV-F Cytoplasmic Tail

Exemplary truncated NiV-F protein constructs were generated which contain the transmembrane and ectodomain of NiV-F and a NiV-F cytoplasmic tail which is truncated, or wherein the cytoplasmic tail is derived from a F protein of another Paramyxovirus.

a. NiV-F Truncations and Deletions

Exemplary truncated NiV-F protein constructs were generated in which a series of truncated cytoplasmic tail mutants were generated that lacked up to 27 contiguous C-terminal amino acids of the cytoplasmic tail of NiV-F (see Table E1 “Cytoplasmic Tail Truncations”). In addition, exemplary truncated NiV-F protein constructs were also generated which lacked up to 22 discontinuous intervening amino acids in the membrane-proximal polybasic region, the tyrosine-based endocytic motif, or the highly-charged region (see Table E1 “Domain Deletions”). The extracellular domain (ectodomain) and transmembrane domain of each generated construct was the same and is set forth in SEQ ID NO:2.

The generated truncated NiV-G cytoplasmic tail variants are set forth in Table E1.

TABLE E1
NiV-F Truncations and Deletions

| | | |
|---|---|---|
| SEQ ID | | |
| NO | Name/Description | Cytoplasmic Tail Sequence |

Reference Controls

| | | |
|---|---|---|
| 4 | NivF_CT-28 (full-length) | EKKRNTYSRLEDRRVRPTSSGDLYYIGT |
| 26 | NivF_CT-6 | EKKRNT |

Cytoplasmic Tail Truncations

| | | |
|---|---|---|
| 5 | NivF_CT-27 | EKKRNTYSRLEDRRVRPTSSGDLYYIG |
| 6 | NivF_CT-26 | EKKRNTYSRLEDRRVRPTSSGDLYYI |
| 7 | NivF_CT-25 | EKKRNTYSRLEDRRVRPTSSGDLYY |
| 8 | NivF_CT-24 | EKKRNTYSRLEDRRVRPTSSGDLY |
| 9 | NivF_CT-23 | EKKRNTYSRLEDRRVRPTSSGDL |
| 10 | NivF_CT-22 | EKKRNTYSRLEDRRVRPTSSGD |
| 11 | NivF_CT-21 | EKKRNTYSRLEDRRVRPTSSG |
| 12 | NivF_CT-20 | EKKRNTYSRLEDRRVRPTSS |
| 13 | NivF_CT-19 | EKKRNTYSRLEDRRVRPTS |
| 14 | NivF_CT-18 | EKKRNTYSRLEDRRVRPT |
| 15 | NivF_CT-17 | EKKRNTYSRLEDRRVRP |
| 16 | NivF_CT-16 | EKKRNTYSRLEDRRVR |
| 17 | NivF_CT-15 | EKKRNTYSRLEDRRV |
| 18 | NivF_CT-14 | EKKRNTYSRLEDRR |
| 19 | NivF_CT-13 | EKKRNTYSRLEDR |
| 20 | NivF_CT-12 | EKKRNTYSRLED |
| 21 | NivF_CT-11 | EKKRNTYSRLE |
| 22 | NivF_CT-10 | EKKRNTYSRL |
| 23 | NivF_CT-9 | EKKRNTYSR |
| 24 | NivF_CT-8 | EKKRNTYS |
| 25 | NivF_CT-7 | EKKRNTY |
| 26 | NivF_CT-6 | EKKRNT |
| 27 | NivF_CT-5 | EKKRN |
| 28 | NivF_CT-4 | EKKR |
| 29 | NivF_CT-3 | EKK |
| 30 | NivF_CT-2 | EK |
| 31 | NivF_CT-1 | E |

Domain Deletions

| | | |
|---|---|---|
| 32 | Delete membrane-proximal polybasic | EYSRLEDRRVRPTSSGDLYYIGT |
| | region | |
| 33 | Delete tyrosine-based endocytic | EKKRNTEDRRVRPTSSGDLYYIGT |
| | motif | |
| 34 | Delete highly-charged region | EKKRNTYSRLRVRPTSSGDLYYIGT |

b. NiV-F Endocytosis Motif Variants

Exemplary mutated NiV-F proteins were generated in which amino acid substitutions and deletions were made in the endocytosis motif of the NiV-F cytoplasmic tail, with reference to numbering set forth in SEQ ID NOA4 The extracellular domain (ectodomain) and transmembrane domain of each generated construct was the same as set forth in SEQ ID NO:2.

The generated NiV-F cytoplasmic tail mutants are set forth in Table E2.

TABLE E2
NiV-F Cytoplasmic Tail Mutants

| SEQ | | Tail Sequence |
|---|---|---|
| ID NO | Name | Cytoplasmic |
| 35 | YSRL | EKYSRL |
| 36 | YSRL | EKKYSRL |
| 37 | NIvF_CT-6_AU1 | EKKRNTDTYRYI |
| 38 | NIvF_CT-6_HA | EKKRNTYPYDVPDYA |
| 39 | DKQTLL | EKDKQTLL |
| 40 | DKQTLL | EDKQTLL |
| 41 | DKQTLL | EKKRNTDKQTLL |
| 42 | DKQTLL | EKKRNTDKQTLLEDRRVRPTSSGDLYYI |
| | | GT |
| 43 | NPVY | EKNPVY |
| 44 | FDNPVY | EKFDNPVY |
| 45 | NPVY | EKKRNTNPVY |
| 46 | FDNPVY | EKKRNTFDNPVY |
| 47 | NPVY | EKKRNTNPVYEDRRVRPTSSGDLYYIGT |
| 48 | FDNPVY | EKKRNTFDNPVYEDRRVRPTSSGDLYYI |
| | | GT |
| 49 | YFIPIN | EKYFIPIN |
| 50 | YFIPIN | EKKRNTYFIPIN |
| 51 | YFIPIN | EKKRNTYFIPINEDRRVRPTSSGDLYYI |
| | | GT |

c. Chimeric F Protein Constructs

Exemplary chimeric F protein constructs were generated in which a series of swaps were made to replace the native NiV-F cytoplasmic tail with the cytoplasmic tail, or a truncated cytoplasmic tail thereof, of another virus or protein. The series of chimeric F proteins that were generated contained cytoplasmic tails, or a truncated cytoplasmic tail thereof, from another paramyxovirus, another virus from the Kingdom Orthornavirae, Vesicular Stomatitis Virus (VSV), or another domain of a protein related to a retrovirus, (i.e., HIV-1 gp41 or retroviral associated cellular protein CD29). The extracellular domain (ectodomain) and transmembrane domain of each generated construct was the same as set forth in SEQ ID NO:2.

The generated NiV-F chimeric cytoplasmic tail variants are set forth in Table E3.

TABLE E3
NiV-F Chimeric Cytoplasmic Tail Mutants

| | | |
|---|---|---|
| SEQ | | |
| ID | | |
| NO | Name | Cytoplasmic Tail Sequence |

I. PARAMYXOVIRUS SWAPPING
Hendra virus

| | | |
|---|---|---|
| 58 | HeVF_CT_28_FL | EKKRGNYSRLDDRQVRPVSNGDLYYIGT |
| 59 | HeVF_CT_27 | EKKRGNYSRLDDRQVRPVSNGDLYYIG |
| 60 | HeVF_CT_26 | EKKRGNYSRLDDRQVRPVSNGDLYYI |
| 61 | HeVF_CT_25 | EKKRGNYSRLDDRQVRPVSNGDLYY |
| 62 | HeVF_CT_24 | EKKRGNYSRLDDRQVRPVSNGDLY |
| 63 | HeVF_CT_23 | EKKRGNYSRLDDRQVRPVSNGDL |
| 64 | HeVF_CT_22 | EKKRGNYSRLDDRQVRPVSNGD |
| 65 | HeVF_CT_21 | EKKRGNYSRLDDRQVRPVSNG |
| 66 | HeVF_CT_20 | EKKRGNYSRLDDRQVRPVSN |
| 67 | HeVF_CT_19 | EKKRGNYSRLDDRQVRPVS |
| 68 | HeVF_CT_18 | EKKRGNYSRLDDRQVRPV |
| 69 | HeVF_CT_17 | EKKRGNYSRLDDRQVRP |
| 70 | HeVF_CT_16 | EKKRGNYSRLDDRQVR |
| 71 | HeVF_CT_15 | EKKRGNYSRLDDRQV |
| 72 | HeVF_CT_14 | EKKRGNYSRLDDRQ |
| 73 | HeVF_CT_13 | EKKRGNYSRLDDR |
| 74 | HeVF_CT_12 | EKKRGNYSRLDD |
| 75 | HeVF_CT_11 | EKKRGNYSRLD |
| 76 | HeVF_CT_10 | EKKRGNYSRL |
| 77 | HeVF_CT_9 | EKKRGNYSR |
| 78 | HeVF_CT_8 | EKKRGNYS |
| 79 | HeVF_CT_7 | EKKRGNY |
| 80 | HeVF_CT_6 | EKKRGN |
| 81 | HeVF_CT_5 | EKKRG |

Bat Paramyxovirus

| | | |
|---|---|---|
| 82 | BatPVF_CT_47FL | RRYNKYTPLINSDPSSRRSTIQDVYIIPNPGEHSIRSAARSIDRDRD |
| 83 | BatPVF_CT_14 | RRYNKYTPLINSDP |
| 84 | BatPVF_CT_13 | RRYNKYTPLINSD |
| 85 | BatPVF_CT_12 | RRYNKYTPLINS |
| 86 | BatPVF_CT_11 | RRYNKYTPLIN |
| 87 | BatPVF_CT_10 | RRYNKYTPLI |
| 88 | BatPVF_CT_9 | RRYNKYTPL |
| 89 | BatPVF_CT_8 | RRYNKYTP |
| 90 | BatPVF_CT_7 | RRYNKYT |
| 91 | BatPVF_CT_6 | RRYNKY |
| 92 | BatPVF_CT_5 | RRYNK |
| 93 | BatPVF_CT_4 | RRYN |
| 94 | BatPVF_CT_3 | RRY |
| 95 | BatPVF_CT_2 | RR |

Cedar Virus

| | | |
|---|---|---|
| 96 | CeVF_CT_29_FL | DDPDYYNDYKRERINGKASKSNNIYYVGD |
| 97 | CeVF_CT_14 | DDPDYYNDYKRERI |
| 98 | CeVF_CT_13 | DDPDYYNDYKRER |
| 99 | CeVF_CT_12 | DDPDYYNDYKRE |
| 100 | CeVF_CT_11 | DDPDYYNDYKR |
| 101 | CeVF_CT_10 | DDPDYYNDYK |
| 102 | CeVF_CT_9 | DDPDYYNDY |
| 103 | CeVF_CT_8 | DDPDYYND |
| 104 | CeVF_CT_7 | DDPDYYN |
| 105 | CeVF_CT_6 | DDPDYY |
| 106 | CeVF_CT_5 | DDPDY |
| 107 | CeVF_CT_4 | DDPD |
| 108 | CeVF_CT_3 | DDP |
| 109 | CeVF_CT_2 | DD |
| 110 | CeVF_CT_1 | D |

Human parainfluenza virus

| | | |
|---|---|---|
| 111 | HPIV2F_CT_38FL | KLVSQIHQFRSLAATTMFHRENPAFFSKNNHGNIYGIS |
| 112 | HPIV2F_CT_14 | KLVSQIHQFRSLAA |
| 113 | HPIV2F_CT_13 | KLVSQIHQFRSLA |
| 114 | HPIV2F_CT_12 | KLVSQIHQFRSL |
| 115 | HPIV2F_CT_11 | KLVSQIHQFRS |
| 116 | HPIV2F_CT_10 | KLVSQIHQFR |
| 117 | HPIV2F_CT_9 | KLVSQIHQF |
| 118 | HPIV2F_CT_8 | KLVSQIHQ |
| 119 | HPIV2F_CT_7 | KLVSQIH |
| 120 | HPIV2F_CT_6 | KLVSQI |
| 121 | HPIV2F_CT_5 | KLVSQ |
| 122 | HPIV2F_CT_4 | KLVS |
| 123 | HPIV2F_CT_3 | KLV |
| 124 | HPIV2F_CT_2 | KL |

Measles Virus

| | | |
|---|---|---|
| 125 | MeaslesF_CT_33_FL | RGRCNKKGEQVGMSRPGLKPDLTGTSKSYVRSL |
| 126 | MeaslesF_CT_14 | RGRCNKKGEQVGMS |
| 127 | MeaslesF_CT_13 | RGRCNKKGEQVGM |
| 128 | MeaslesF_CT_12 | RGRCNKKGEQVG |
| 129 | MeaslesF_CT_11 | RGRCNKKGEQV |
| 130 | MeaslesF_CT_10 | RGRCNKKGEQ |
| 131 | MeaslesF_CT_9 | RGRCNKKGE |
| 132 | MeaslesF_CT_8 | RGRCNKKG |
| 133 | MeaslesF_CT_7 | RGRCNKK |
| 134 | MeaslesF_CT_6 | RGRCNK |
| 135 | MeaslesF_CT_5 | RGRCN |
| 136 | MeaslesF_CT_4 | RGRC |
| 138 | MeaslesF_CT_3 | RGR |
| 139 | MeaslesF_CT_2 | RG |
| 140 | MeaslesF_CT_1 | R |

Newcastle Disease Virus

| | | |
|---|---|---|
| 141 | NDVF_CT_26_FL | KQKAQQKTLLWLGNNTLDQMRATTKM |
| 142 | NDVF_CT_14 | KQKAQQKTLLWLGN |
| 143 | NDVF_CT_13 | KQKAQQKTLLWLG |
| 144 | NDVF_CT_12 | KQKAQQKTLLWL |
| 145 | NDVF_CT_11 | KQKAQQKTLLW |
| 146 | NDVF_CT_10 | KQKAQQKTLL |
| 147 | NDVF_CT_9 | KQKAQQKTL |
| 148 | NDVF_CT_8 | KQKAQQKT |
| 149 | NDVF_CT_7 | KQKAQQK |
| 150 | NDVF_CT_6 | KQKAQQ |
| 151 | NDVF_CT_5 | KQKAQ |
| 152 | NDVF_CT_4 | KQKA |
| 153 | NDVF_CT_3 | KQK |
| 154 | NDVF_CT_2 | KQ |
| 155 | NDVF_CT_1 | K |

Sendai Virus

| | | |
|---|---|---|
| 156 | SeVF_CT_42_FL | RLRRSMLMGNPDDRIPRDTYTLEPKIRHMYTNGGFDAMAEKR |
| 157 | SeVF_CT_14 | RLRRSMLMGNPDDR |
| 158 | SeVF_CT_13 | RLRRSMLMGNPDD |
| 159 | SeVF_CT_12 | RLRRSMLMGNPD |
| 160 | SeVF_CT_11 | RLRRSMLMGNP |
| 161 | SeVF_CT_10 | RLRRSMLMGN |
| 162 | SeVF_CT_9 | RLRRSMLMG |
| 163 | SeVF_CT_8 | RLRRSMLM |
| 164 | SeVF_CT_7 | RLRRSML |
| 165 | SeVF_CT_6 | RLRRSM |
| 166 | SeVF_CT_5 | RLRRS |
| 167 | SeVF_CT_4 | RLRR |
| 168 | SeVF_CT_3 | RLR |
| 169 | SeVF_CT_2 | RL |

II. Other Virus

| | | |
|---|---|---|
| 170 | BaEVwt_CT | NRLTAFINDKLNIIHAMVLTQQYQVLRTDEEAQD |
| 171 | Cocal_CT | RYRYQGSNNKRIYNDIEMSRFRK |
| 172 | Ebo -GP_CT | CKFVF |
| 173 | GaLV_CT | KLVQFINDRISAVKILVLRQKYQALENEGNL |
| 174 | GaLV-RLess_CT | KLVQFINDRISAVKIL |
| 175 | LCMV_GP_CT | THRHIKGGSCPKPHRLTNKGICSCGAFKVPGVKTVWKRR |
| 176 | MLV-A- | RLVQFVKDRISVVQAL |
| | RLess_CT | |
| 177 | BaEVRLess_CT | NRLTAFINDKLNIIHA |
| 178 | MLV-A_CT | RLVQFVKDRISVVQALVLTQQYHQLKPIEYEP |
| 179 | VSV-G_CT | RVGIHLCIKLKHTKKRQIYTDIEMNRLGK |

III. HIV

| | | |
|---|---|---|
| 180 | HIV- | NRVRQGYSPLSFQTHLPIPRGPDRPEGIEEEGGERDRDRSIRLVNGS |
| | 1_gp41_LLIR- | LALIWDDLRSLCLFSYHRLRDLLLIVTRIVELLGRRGWEALKYWW |
| | LLP1-LLP3- | NLLQYWSQELKNSAVNLLNATAIAVAEGTDRVIEVLQAAYRAIRHI |
| | LLP2-gp41-CT- | PRRIRQGLERILL |
| | N_FL | |
| 181 | HIV- | NRVRQGYSPLSFQTHLPIPRGPDRPEGIEEEGGERDRDRSIRLVNGS |
| | 1_gp41_LLP1- | LALIWDDLRSLCLFSYHRLRDLLLIVTRIVELLGRRGWEALKYWW |
| | LLP3-LLP2- | NLLQYWSQELKNSAVNLLNATAIAVAEGTDRVIEVLQAAYRAIRHI |
| | gp41-CT-N | PRRIRQGLE |
| 182 | HIV- | NRVRQGYSPLSFQTHLPIPRGPDRPEGIEEEGGERDRDRSIRLVNGS |
| | 1_gp41_LLP3- | LALIWDDLRSLCLFSYHRLRDLLLIVTRIVELLGRRGWEALKYWW |
| | LLP2-gp41-CT-N | NLLQYWSQELKNSAVNLLNATAIAVA |
| 183 | HIV- | NRVRQGYSPLSFQTHLPIPRGPDRPEGIEEEGGERDRDRSIRLVNGS |
| | 1_gp41_LLP2- | LALIWDDLRSLCLFSYHRLRDLLLIVTRIVELL |
| | gp41-CT-N | |
| 184 | HIV- | NRVRQGYSPLSFQTHLPIPRGPDRPEGIEEEGGERDRDRSIRLVNGS |
| | 1_gp41_gp41- | |
| | CT-N | |
| 185 | HIV- | LALIWDDLRSLCLFSYHRLRDLLLIVTRIVELLGRRGWEALKYWW |
| | 1_gp41_LLIR- | NLLQYWSQELKNSAVNLLNATAIAVAEGTDRVIEVLQAAYRAIRHI |
| | LLP1-LLP3- | PRRIRQGLERILL |
| | LLP2 | |
| 186 | HIV- | LALIWDDLRSLCLFSYHRLRDLLLIVTRIVELLGRRGWEALKYWW |
| | 1_gp41_LLP1- | NLLQYWSQELKNSAVNLLNATAIAVAEGTDRVIEVLQAAYRAIRHI |
| | LLP3-LLP2 | PRRIRQGLE |
| 187 | HIV- | LALIWDDLRSLCLFSYHRLRDLLLIVTRIVELLGRRGWEALKYWW |
| | 1_gp41_LLP3- | NLLQYWSQELKNSAVNLLNATAIAVA |
| | LLP2 | |
| 188 | HIV- | LALIWDDLRSLCLFSYHRLRDLLLIVTRIVELL |
| | 1_gp41_LLP2 | |
| 189 | HIV- | WEALKYWWNLLQYWSQELKNSAVNLLNATAIAVAEGTDRVIEVL |
| | 1_gp41_LLIR- | QAAYRAIRHIPRRIRQGLERILL |
| | LLP1-LLP3 | |
| 190 | HIV- | WEALKYWWNLLQYWSQELKNSAVNLLNATAIAVAEGTDRVIEVL |
| | 1_gp41_LLP1- | QAAYRAIRHIPRRIRQGLE |
| | LLP3 | |
| 191 | HIV- | WEALKYWWNLLQYWSQELKNSAVNLLNATAIAVA |
| | 1_gp41_LLP3 | |
| 192 | HIV- | RVIEVLQAAYRAIRHIPRRIRQGLERILL |
| | 1_gp41_LLIR- | |
| | LLP1 | |
| 193 | HIV- | RVIEVLQAAYRAIRHIPRRIRQGLE |
| | 1_gp41_LLP1 | |

IV. Retroviral Associated Cellular Protein

| | | |
|---|---|---|
| 194 | CD29_CT | KLLMIIHDRREFAKFEKEKMNAKWDTGENPIYKSAVTTVVNPKYE |
| | | GK |
| 195 | CD29_CT | EKKRNTKLLMIIHDRREFAKFEKEKMNAKWDTGENPIYKSAVTTV |
| | | VNPKYEGK |
| 196 | CD63_CT_1to11 | AVEGGMKCVK |
| 197 | CD63_CT_73to81 | CGACKENYC |
| 198 | CD63_CT_225to238 | VEYGSRISKVLCC |
| 199 | CD63_CT_1to11 | EKKRNTAVEGGMKCVK |
| 200 | CD63_CT_73to81 | EKKRNTCGACKENYC |
| 201 | CD63_CT_225to238 | EKKRNTVEYGSRISKVLCC |

Additional exemplary chimeric F protein constructs were generated in which the native NiV-F cytoplasmic tail was appended with the cytoplasmic tail, or a truncated cytoplasmic tail thereof, of another virus. The series of chimeric F proteins that were generated contained at least a portion of the cytoplasmic tails, or a truncated cytoplasmic tail thereof, from another paramyxovirus, another virus from the Kingdom Orthornavirae or from Vesicular Stomatitis Virus (VSV), The extracellular domain (ectodomain) and transmembrane domain of each generated construct was the same as set forth in SEQ ID NO:2. As a control, a NiV-F containing a full-length cytoplasmic tail (set forth in SEQ ID NOA4). was generated; the full sequence of the control NiV-F sequence is set forth in SEQ ID NO:1 including the signal peptide or as amino acid residues 27-546 of SEQ ID NO:1 for the mature sequence without the signal peptide.

The generated NiV-F chimeric cytoplasmic tail variants are set forth in Table E4.

TABLE E4
NiV-F Chimeric Cytoplasmic Tail Variants

| SEQ ID | | |
|---|---|---|
| NO | Name | Cytoplasmic Tail Sequence |
| 202 | VSV-G_CT | EKKRNTRVGIHLCIKLKHTKKRQIYTDIEMNRLGK |
| 203 | BaEVRLess_CT | EKKRNTNRLTAFINDKLNIIHA |
| 204 | Cocal_CT | EKKRNTRYRYQGSNNKRIYNDIEMSRFRK |
| 205 | EboV-GP_CT | EKKRNTCKFVF |
| 206 | GaLV_CT | EKKRNTKLVQFINDRISAVKILVLRQKYQALENEGNL |
| 207 | GaLV-RLess_CT | EKKRNTKLVQFINDRISAVKIL |
| 208 | MLV-A_CT | EKKRNTRLVQFVKDRISVVQALVLTQQYHQLKPIEYEP |
| 209 | MLV-A-RLess_CT | EKKRNTRLVQFVKDRISVVQAL |

Exemplary chimeric F protein constructs were generated in which a series of swaps were made to replace the attachment motif of the native NiV-F cytoplasmic tail with the cytoplasmic tail, or truncated cytoplasmic tail, from the attachment protein (i.e., a G, H, or HN protein) of another paramyxovirus. The extracellular domain (ectodomain) and transmembrane domain of each generated construct was the same as set forth in SEQ ID NO:2.

The generated NiV-F chimeric cytoplasmic tail variants are set forth in Table E5.

TABLE E5
NiV-F Chimeric Cytoplasmic Tail Variants

| SEQ | | |
|---|---|---|
| ID NO | Name | Cytoplasmic Tail Sequence |
| 210 | Nipah_virus | KSDLLGENIKKM |
| 211 | Hendra_virus | KSDLLGDNIKKM |
| 212 | Measles_virus | RDIMLHERNIVIRSGM |
| 213 | Nipah_virus | KSD |
| 214 | Measles_virus | RDI |
| 215 | Nipah_virus | KSDLLG |
| 216 | Measles_virus | RDIMLH |
| 217 | Nipah_virus | EKKRKSDLLGENIKKM |
| 218 | Measles_virus | EKKRRDIMLHERNIVIRSGM |
| 219 | Hendra_virus | EKKRNTKSDLLGDNIKKM |
| 220 | Measles_virus | EKKRNTRDIMLHERNIVIRSGM |
| 221 | Hendra_virus | EKKRNTYSRLKSDLLGDNIKKM |
| 222 | Measles_virus | EKKRNTYSRLRDIMLHERNIVIRSGM |

### 2. NiV-F Ectodomain

Exemplary truncated NiV-F protein constructs were generated which contain the transmembrane and cytoplasmic tail of NiV-F and a NiV-F ectodomain which was mutated.

a. Protease Site Modifications

Generally, the ectodomain of the Nipah F protein is subject to cleavage by Cathepsin L to yield the active form (F1+F2). Exemplary chimeric F protein constructs were generated in which this cleavage site NNTHDLVGDVRLAGV (SEQ ID NO:311) was modified to include a recognition site for Furin, a calcium-dependent serine endoprotease which in some instances may be differentially expressed compared to cathepsin L.

Additional exemplary chimeric F proteins were generated in which a series of swaps were made to replace the protease site of the native NiV-F cytoplasmic tail with the protease site from the ectodomain of another virus. The series of chimeric F proteins that were generated contained the protease site, or a truncated motif thereof, from another paramyxovirus.

The variant NiV-F proteins with protease site modifications also were generated to contain a truncated cytoplasmic tail set forth in SEQ ID NO:26 that lacked 22 contiguous C-terminal amino acids of the full-length cytoplasmic tail of wild-type of native NiV-F set forth in SEQ ID NO:4. The cytoplasmic domain and transmembrane domain of each generated construct was the same as set forth in SEQ ID NO:328 and below:

SEQ ID 328: YVLSIASLCIGLITFISFIIVEKKRNT

The generated NiV-F chimeric protease site variants are set forth in Table E6.

TABLE E6
NiV-F Chimeric Protease Site Mutants

| | | |
|---|---|---|
| SEQ ID | | |
| NO | Name | Full Length Sequence |

I. Furin Modifications

| | | |
|---|---|---|
| 224 | NNTHDSRR | MVVILDKRCYCNLLILILMISECSVGILHYEKLSKIGLVKGVTRKY |
| | HKR/FAGV | KIKSNPLTKDIVIKMIPNVSNMSQCTGSVMENYKTRLNGILTPIKG |
| | (SEQ ID NO: | ALEIYKNNTHDSRRHKRFAGVIMAGVAIGIATAAQITAGVALYEA |
| | 313) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 225 | NNTHDLVR | MVVILDKRCYCNLLILILMISECSVGILHYEKLSKIGLVKGVTRKY |
| | HKR/FAGV | KIKSNPLTKDIVIKMIPNVSNMSQCTGSVMENYKTRLNGILTPIKG |
| | (SEQ ID NO: | ALEIYKNNTHDLVRHKRFAGVIMAGVAIGIATAAQITAGVALYEA |
| | 314) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 226 | NNTHDLVR | MVVILDKRCYCNLLILILMISECSVGILHYEKLSKIGLVKGVTRKY |
| | HKR/LAGV | KIKSNPLTKDIVIKMIPNVSNMSQCTGSVMENYKTRLNGILTPIKG |
| | (SEQ ID NO: | ALEIYKNNTHDLVRHKRLAGVIMAGVAIGIATAAQITAGVALYEA |
| | 315) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 227 | NNTHDLVR | MVVILDKRCYCNLLILILMISECSVGILHYEKLSKIGLVKGVTRKY |
| | HRR/FAGV | KIKSNPLTKDIVIKMIPNVSNMSQCTGSVMENYKTRLNGILTPIKG |
| | (SEQ ID NO: | ALEIYKNNTHDLVRHRRFAGVIMAGVAIGIATAAQITAGVALYEA |
| | 316) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 228 | NNTHDLVR | MVVILDKRCYCNLLILILMISECSVGILHYEKLSKIGLVKGVTRKY |
| | HRR/LAGV | KIKSNPLTKDIVIKMIPNVSNMSQCTGSVMENYKTRLNGILTPIKG |
| | (SEQ ID NO: | ALEIYKNNTHDLVRHRRLAGVIMAGVAIGIATAAQITAGVALYEA |
| | 317) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 229 | NNGHDSRR | MVVILDKRCYCNLLILILMISECSVGILHYEKLSKIGLVKGVTRKY |
| | HKR/FAGV | KIKSNPLTKDIVIKMIPNVSNMSQCTGSVMENYKTRLNGILTPIKG |
| | (SEQ ID NO: | ALEIYKNNGHDSRRHKRFAGVIMAGVAIGIATAAQITAGVALYEA |
| | 318) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 230 | NNGHDLVR | MVVILDKRCYCNLLILILMISECSVGILHYEKLSKIGLVKGVTRKY |
| | HKR/FAGV | KIKSNPLTKDIVIKMIPNVSNMSQCTGSVMENYKTRLNGILTPIKG |
| | (SEQ ID NO: | ALEIYKNNGHDLVRHKRFAGVIMAGVAIGIATAAQITAGVALYEA |
| | 319) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 231 | NNGHDLVR | MVVILDKRCYCNLLILILMISECSVGILHYEKLSKIGLVKGVTRKY |
| | HKR/LAGV | KIKSNPLTKDIVIKMIPNVSNMSQCTGSVMENYKTRLNGILTPIKG |
| | (SEQ ID NO: | ALEIYKNNGHDLVRHKRLAGVIMAGVAIGIATAAQITAGVALYEA |
| | 320) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 232 | NNGHDLVR | MVVILDKRCYCNLLILILMISECSVGILHYEKLSKIGLVKGVTRKY |
| | HRR/FAGV | KIKSNPLTKDIVIKMIPNVSNMSQCTGSVMENYKTRLNGILTPIKG |
| | (SEQ ID NO: | ALEIYKNNGHDLVRHRRFAGVIMAGVAIGIATAAQITAGVALYEA |
| | 321) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 233 | NNGHDLVR | MVVILDKRCYCNLLILILMISECSVGILHYEKLSKIGLVKGVTRKY |
| | HRR/LAGV | KIKSNPLTKDIVIKMIPNVSNMSQCTGSVMENYKTRLNGILTPIKG |
| | (SEQ ID NO: | ALEIYKNNGHDLVRHRRLAGVIMAGVAIGIATAAQITAGVALYEA |
| | 322) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 234 | QNTHDSRR | MVVILDKRCYCNLLILILMISECSVGILHYEKLSKIGLVKGVTRKY |
| | HKR/FAGV | KIKSNPLTKDIVIKMIPNVSNMSQCTGSVMENYKTRLNGILTPIKG |
| | (SEQ ID NO: | ALEIYKQNTHDSRRHKRFAGVIMAGVAIGIATAAQITAGVALYEA |
| | 323) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 235 | QNTHDLVR | MVVILDKRCYCNLLILILMISECSVGILHYEKLSKIGLVKGVTRKY |
| | HKR/FAGV | KIKSNPLTKDIVIKMIPNVSNMSQCTGSVMENYKTRLNGILTPIKG |
| | | ALEIYKQNTHDLVRHKRFAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 236 | QNTHDLVR | MVVILDKRCYCNLLILILMISECSVGILHYEKLSKIGLVKGVTRKY |
| | HKR/LAGV | KIKSNPLTKDIVIKMIPNVSNMSQCTGSVMENYKTRLNGILTPIKG |
| | (SEQ ID NO: | ALEIYKQNTHDLVRHKRLAGVIMAGVAIGIATAAQITAGVALYEA |
| | 325) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 237 | QNTHDLVR | MVVILDKRCYCNLLILILMISECSVGILHYEKLSKIGLVKGVTRKY |
| | HRR/FAGV | KIKSNPLTKDIVIKMIPNVSNMSQCTGSVMENYKTRLNGILTPIKG |
| | (SEQ ID NO: | ALEIYKQNTHDLVRHRRFAGVIMAGVAIGIATAAQITAGVALYEA |
| | 326) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 238 | QNTHDLVR | MVVILDKRCYCNLLILILMISECSVGILHYEKLSKIGLVKGVTRKY |
| | HRR/LAGV | KIKSNPLTKDIVIKMIPNVSNMSQCTGSVMENYKTRLNGILTPIKG |
| | (SEQ ID NO: | ALEIYKQNTHDLVRHRRLAGVIMAGVAIGIATAAQITAGVALYEA |
| | 327) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 239 | Hendra virus | MVVILDKRCYCNLLILILMISECSVGILHYEKLSKIGLVKGVTRKY |
| | VGDVK | KIKSNPLTKDIVIKMIPNVSNMSQCTGSVMENYKTRLNGILTPIKG |
| | (SEQ ID | ALEIYKNNTHDLVGDVKLAGVIMAGVAIGIATAAQITAGVALYE |
| | NO: 329 | AMKNADNINKLKSSIESTNEAVVKLQETAEKTVYVLTALQDYINT |
| | | NLVPTIDKISCKQTELSLDLALSKYLSDLLFVFGPNLQDPVSNSMTI |
| | | QAISQAFGGNYETLLRTLGYATEDFDDLLESDSITGQIIYVDLSSYY |
| | | IIVRVYFPILTEIQQAYIQELLPVSFNNDNSEWISIVPNFILVRNTLIS |
| | | NIEIGFCLITKRSVICNQDYATPMTNNMRECLTGSTEKCPRELVVS |
| | | SHVPRFALSNGVLFANCISVTCQCQTTGRAISQSGEQTLLMIDNTT |
| | | CPTAVLGNVIISLGKYLGSVNYNSEGIAIGPPVFTDKVDISSQISSM |
| | | NQSLQQSKDYIKEAQRLLDTVNPSLISMLSMIILYVLSIASLCIGLIT |
| | | FISFIIVEKKRNT |
| 240 | Measles non- | MVVILDKRCYCNLLILILMISECSVGILHYEKLSKIGLVKGVTRKY |
| | cleavable | KIKSNPLTKDIVIKMIPNVSNMSQCTGSVMENYKTRLNGILTPIKG |
| | control site | ALEIYKNNTHDLRNHNRLAGVIMAGVAIGIATAAQITAGVALYEA |
| | RNHNR (SEQ | MKNADNINKLKSSIESTNEAVVKLQETAEKTVYVLTALQDYINTN |
| | ID NO: 330) | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 241 | Measles/Canine | MVVILDKRCYCNLLILILMISECSVGILHYEKLSKIGLVKGVTRKY |
| | Distemper | KIKSNPLTKDIVIKMIPNVSNMSQCTGSVMENYKTRLNGILTPIKG |
| | virus | ALEIYKNNTHDLRRHKRLAGVIMAGVAIGIATAAQITAGVALYEA |
| | RRHKR | MKNADNINKLKSSIESTNEAVVKLQETAEKTVYVLTALQDYINTN |
| | (SEQ ID | LVPTIDKISCKQTELSLDLALSKYLSDLLFVFGPNLQDPVSNSMTIQ |
| | NO: 331) | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 242 | Newcastle | MVVILDKRCYCNLLILILMISECSVGILHYEKLSKIGLVKGVTRKY |
| | Disease virus | KIKSNPLTKDIVIKMIPNVSNMSQCTGSVMENYKTRLNGILTPIKG |
| | (virulent) | ALEIYKNNTHDLRRQKRLAGVIMAGVAIGIATAAQITAGVALYEA |
| | RRQKR (SEQ | MKNADNINKLKSSIESTNEAVVKLQETAEKTVYVLTALQDYINTN |
| | ID NO: 332) | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 243 | Newcastle | MVVILDKRCYCNLLILILMISECSVGILHYEKLSKIGLVKGVTRKY |
| | Disease virus | KIKSNPLTKDIVIKMIPNVSNMSQCTGSVMENYKTRLNGILTPIKG |
| | (avirulent) | ALEIYKNNTHDLGRQGRLAGVIMAGVAIGIATAAQITAGVALYEA |
| | GRQGR (SEQ | MKNADNINKLKSSIESTNEAVVKLQETAEKTVYVLTALQDYINTN |
| | ID NO: 333) | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 244 | PIV2 | MVVILDKRCYCNLLILILMISECSVGILHYEKLSKIGLVKGVTRKY |
| | TRQKR (SEQ | KIKSNPLTKDIVIKMIPNVSNMSQCTGSVMENYKTRLNGILTPIKG |
| | ID NO: 334) | ALEIYKNNTHDLTRQKRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 245 | PIV4 | MVVILDKRCYCNLLILILMISECSVGILHYEKLSKIGLVKGVTRKY |
| | EIQSR (SEQ | KIKSNPLTKDIVIKMIPNVSNMSQCTGSVMENYKTRLNGILTPIKG |
| | ID NO: 335) | ALEIYKNNTHDLEIQSRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 246 | Sendai virus | MVVILDKRCYCNLLILILMISECSVGILHYEKLSKIGLVKGVTRKY |
| | VPQSR (SEQ | KIKSNPLTKDIVIKMIPNVSNMSQCTGSVMENYKTRLNGILTPIKG |
| | ID NO: 336) | ALEIYKNNTHDLVPQSRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 247 | PIV1 | MVVILDKRCYCNLLILILMISECSVGILHYEKLSKIGLVKGVTRKY |
| | NPQSR (SEQ | KIKSNPLTKDIVIKMIPNVSNMSQCTGSVMENYKTRLNGILTPIKG |
| | ID NO: 337) | ALEIYKNNTHDLNPQSRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 248 | PIV3 | MVVILDKRCYCNLLILILMISECSVGILHYEKLSKIGLVKGVTRKY |
| | PRTKR (SEQ | KIKSNPLTKDIVIKMIPNVSNMSQCTGSVMENYKTRLNGILTPIKG |
| | ID NO: 338) | ALEIYKNNTHDLPRTKRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |

b. Signal Sequence Modifications

Exemplary variant NiV-F protein constructs were generated in which the signal sequence was modified to replace the native NiV-F signal sequence with a heterologous signal sequence of another virus or protein. The series of variant NiV-F proteins that were generated contained a signal sequence from a different paramyxovirus, such as Hendra virus, Cedar virus, Human Parainfluenza Virus 2 (HPIV2), Measles virus or Sendai virus; a signal sequence from another virus such as HIV-1, Baboon endogenous retrovirus, Cocal virus, Ebola virus, Gibbon ape leukemia virus, Murine leukemia virus, or Vesicular stomatitis virus; or, or a signal sequence of a human protein.

The variant NiV-F proteins with a heterologous signal sequence also were generated to contain a truncated cytoplasmic tail set forth in SEQ ID NO:26 that lacked 22 contiguous C-terminal amino acids of the full-length cytoplasmic tail of wild-type of native NiV-F set forth in SEQ ID NOA4. The ectodomain, cytoplasmic domain and transmembrane domain of each generated construct was the same as set forth in SEQ ID NO: 303

The generated NiV-F chimeric signal sequence variants are set forth in Table E7.

TABLE E7
NiV-F Chimeric Signal Sequence Mutants

| | | |
|---|---|---|
| SEQ ID NO | Name | Full Length Sequence |

I. Viral Signal Sequences

| | | |
|---|---|---|
| 249 | HevF_signal_ | MATQEVRLKCLLCGIIVLVLSLEGLGILHYEKLSKIGLVKGVTRK |
| | sequence | YKIKSNPLTKDIVIKMIPNVSNMSQCTGSVMENYKTRLNGILTPI |
| | | KGALEIYKNNTHDLVGDVRLAGVIMAGVAIGIATAAQITAGVA |
| | | LYEAMKNADNINKLKSSIESTNEAVVKLQETAEKTVYVLTALQ |
| | | DYINTNLVPTIDKISCKQTELSLDLALSKYLSDLLFVFGPNLQDP |
| | | VSNSMTIQAISQAFGGNYETLLRTLGYATEDFDDLLESDSITGQII |
| | | YVDLSSYYIIVRVYFPILTEIQQAYIQELLPVSFNNDNSEWISIVPN |
| | | FILVRNTLISNIEIGFCLITKRSVICNQDYATPMTNNMRECLTGST |
| | | EKCPRELVVSSHVPRFALSNGVLFANCISVTCQCQTTGRAISQSG |
| | | EQTLLMIDNTTCPTAVLGNVIISLGKYLGSVNYNSEGIAIGPPVFT |
| | | DKVDISSQISSMNQSLQQSKDYIKEAQRLLDTVNPSLISMLSMIIL |
| | | YVLSIASLCIGLITFISFIIVEKKRNT |
| 250 | CevF_signal_ | MSNKRTTVLIIISYTLFYLNNAILHYEKLSKIGLVKGVTRKYKIKS |
| | sequence | NPLTKDIVIKMIPNVSNMSQCTGSVMENYKTRLNGILTPIKGALE |
| | | IYKNNTHDLVGDVRLAGVIMAGVAIGIATAAQITAGVALYEAM |
| | | KNADNINKLKSSIESTNEAVVKLQETAEKTVYVLTALQDYINTN |
| | | LVPTIDKISCKQTELSLDLALSKYLSDLLFVFGPNLQDPVSNSMTI |
| | | QAISQAFGGNYETLLRTLGYATEDFDDLLESDSITGQIIYVDLSS |
| | | YYIIVRVYFPILTEIQQAYIQELLPVSFNNDNSEWISIVPNFILVRN |
| | | TLISNIEIGFCLITKRSVICNQDYATPMTNNMRECLTGSTEKCPRE |
| | | LVVSSHVPRFALSNGVLFANCISVTCQCQTTGRAISQSGEQTLL |
| | | MIDNTTCPTAVLGNVIISLGKYLGSVNYNSEGIAIGPPVFTDKVDI |
| | | SSQISSMNQSLQQSKDYIKEAQRLLDTVNPSLISMLSMIILYVLSI |
| | | ASLCIGLITFISFIIVEKKRNT |
| 251 | HPIV2_signal_ | MHHLHPMIVCIFVMYTGIVGSDAILHYEKLSKIGLVKGVTRKYK |
| | sequence | IKSNPLTKDIVIKMIPNVSNMSQCTGSVMENYKTRLNGILTPIKG |
| | | ALEIYKNNTHDLVGDVRLAGVIMAGVAIGIATAAQITAGVALYE |
| | | AMKNADNINKLKSSIESTNEAVVKLQETAEKTVYVLTALQDYIN |
| | | TNLVPTIDKISCKQTELSLDLALSKYLSDLLFVFGPNLQDPVSNS |
| | | MTIQAISQAFGGNYETLLRTLGYATEDFDDLLESDSITGQIIYVD |
| | | LSSYYIIVRVYFPILTEIQQAYIQELLPVSFNNDNSEWISIVPNFILV |
| | | RNTLISNIEIGFCLITKRSVICNQDYATPMTNNMRECLTGSTEKCP |
| | | RELVVSSHVPRFALSNGVLFANCISVTCQCQTTGRAISQSGEQTL |
| | | LMIDNTTCPTAVLGNVIISLGKYLGSVNYNSEGIAIGPPVFTDKV |
| | | DISSQISSMNQSLQQSKDYIKEAQRLLDTVNPSLISMLSMIILYVL |
| | | SIASLCIGLITFISFIIVEKKRNT |
| 252 | MevF_signal_ | MGLKVNVSAIFMAVLLTLQTPTGQILHYEKLSKIGLVKGVTRKY |
| | sequence | KIKSNPLTKDIVIKMIPNVSNMSQCTGSVMENYKTRLNGILTPIK |
| | | GALEIYKNNTHDLVGDVRLAGVIMAGVAIGIATAAQITAGVAL |
| | | YEAMKNADNINKLKSSIESTNEAVVKLQETAEKTVYVLTALQD |
| | | YINTNLVPTIDKISCKQTELSLDLALSKYLSDLLFVFGPNLQDPVS |
| | | NSMTIQAISQAFGGNYETLLRTLGYATEDFDDLLESDSITGQIIYV |
| | | DLSSYYIIVRVYFPILTEIQQAYIQELLPVSFNNDNSEWISIVPNFIL |
| | | VRNTLISNIEIGFCLITKRSVICNQDYATPMTNNMRECLTGSTEK |
| | | CPRELVVSSHVPRFALSNGVLFANCISVTCQCQTTGRAISQSGEQ |
| | | TLLMIDNTTCPTAVLGNVIISLGKYLGSVNYNSEGIAIGPPVFTD |
| | | KVDISSQISSMNQSLQQSKDYIKEAQRLLDTVNPSLISMLSMIILY |
| | | VLSIASLCIGLITFISFIIVEKKRNT |
| 253 | NDV- | MRSRSSTRIPVPLMLIIRIALTLSCIRLTSSILHYEKLSKIGLVKGV |
| | F_signal_ | TRKYKIKSNPLTKDIVIKMIPNVSNMSQCTGSVMENYKTRLNGI |
| | sequence | LTPIKGALEIYKNNTHDLVGDVRLAGVIMAGVAIGIATAAQITA |
| | | GVALYEAMKNADNINKLKSSIESTNEAVVKLQETAEKTVYVLT |
| | | ALQDYINTNLVPTIDKISCKQTELSLDLALSKYLSDLLFVFGPNL |
| | | QDPVSNSMTIQAISQAFGGNYETLLRTLGYATEDFDDLLESDSIT |
| | | GQIIYVDLSSYYIIVRVYFPILTEIQQAYIQELLPVSFNNDNSEWISI |
| | | VPNFILVRNTLISNIEIGFCLITKRSVICNQDYATPMTNNMRECLT |
| | | GSTEKCPRELVVSSHVPRFALSNGVLFANCISVTCQCQTTGRAIS |
| | | QSGEQTLLMIDNTTCPTAVLGNVIISLGKYLGSVNYNSEGIAIGP |
| | | PVFTDKVDISSQISSMNQSLQQSKDYIKEAQRLLDTVNPSLISML |
| | | SMIILYVLSIASLCIGLITFISFIIVEKKRNT |
| 254 | SevF_signal_ | MTAYIQRSQCISTSLLVVLTTLVSCQIILHYEKLSKIGLVKGVTRK |
| | sequence | YKIKSNPLTKDIVIKMIPNVSNMSQCTGSVMENYKTRLNGILTPI |
| | | KGALEIYKNNTHDLVGDVRLAGVIMAGVAIGIATAAQITAGVA |
| | | LYEAMKNADNINKLKSSIESTNEAVVKLQETAEKTVYVLTALQ |
| | | DYINTNLVPTIDKISCKQTELSLDLALSKYLSDLLFVFGPNLQDP |
| | | VSNSMTIQAISQAFGGNYETLLRTLGYATEDFDDLLESDSITGQII |
| | | YVDLSSYYIIVRVYFPILTEIQQAYIQELLPVSFNNDNSEWISIVPN |
| | | FILVRNTLISNIEIGFCLITKRSVICNQDYATPMTNNMRECLTGST |
| | | EKCPRELVVSSHVPRFALSNGVLFANCISVTCQCQTTGRAISQSG |
| | | EQTLLMIDNTTCPTAVLGNVIISLGKYLGSVNYNSEGIAIGPPVFT |
| | | DKVDISSQISSMNQSLQQSKDYIKEAQRLLDTVNPSLISMLSMIIL |
| | | YVLSIASLCIGLITFISFIIVEKKRNT |
| 255 | HIV1- | MRVKGIRKNYQHLWRWGIMLLGMLMICSAADILHYEKLSKIGL |
| | Env_signal_ | VKGVTRKYKIKSNPLTKDIVIKMIPNVSNMSQCTGSVMENYKTR |
| | sequence | LNGILTPIKGALEIYKNNTHDLVGDVRLAGVIMAGVAIGIATAA |
| | | QITAGVALYEAMKNADNINKLKSSIESTNEAVVKLQETAEKTVY |
| | | VLTALQDYINTNLVPTIDKISCKQTELSLDLALSKYLSDLLFVFG |
| | | PNLQDPVSNSMTIQAISQAFGGNYETLLRTLGYATEDFDDLLES |
| | | DSITGQIIYVDLSSYYIIVRVYFPILTEIQQAYIQELLPVSFNNDNS |
| | | EWISIVPNFILVRNTLISNIEIGFCLITKRSVICNQDYATPMTNNMR |
| | | ECLTGSTEKCPRELVVSSHVPRFALSNGVLFANCISVTCQCQTTG |
| | | RAISQSGEQTLLMIDNTTCPTAVLGNVIISLGKYLGSVNYNSEGI |
| | | AIGPPVFTDKVDISSQISSMNQSLQQSKDYIKEAQRLLDTVNPSLI |
| | | SMLSMIILYVLSIASLCIGLITFISFIIVEKKRNT |
| 256 | BaEV_signal_ | MGFTTKIIFLYNLVLVYAILHYEKLSKIGLVKGVTRKYKIKSNPL |
| | sequence | TKDIVIKMIPNVSNMSQCTGSVMENYKTRLNGILTPIKGALEIYK |
| | | NNTHDLVGDVRLAGVIMAGVAIGIATAAQITAGVALYEAMKN |
| | | ADNINKLKSSIESTNEAVVKLQETAEKTVYVLTALQDYINTNLV |
| | | PTIDKISCKQTELSLDLALSKYLSDLLFVFGPNLQDPVSNSMTIQ |
| | | AISQAFGGNYETLLRTLGYATEDFDDLLESDSITGQIIYVDLSSY |
| | | YIIVRVYFPILTEIQQAYIQELLPVSFNNDNSEWISIVPNFILVRNT |
| | | LISNIEIGFCLITKRSVICNQDYATPMTNNMRECLTGSTEKCPREL |
| | | VVSSHVPRFALSNGVLFANCISVTCQCQTTGRAISQSGEQTLLMI |
| | | DNTTCPTAVLGNVIISLGKYLGSVNYNSEGIAIGPPVFTDKVDISS |
| | | QISSMNQSLQQSKDYIKEAQRLLDTVNPSLISMLSMIILYVLSIAS |
| | | LCIGLITFISFIIVEKKRNT |
| 257 | Cocal_signal_ | MNFLLLTFIVLPLCSHAILHYEKLSKIGLVKGVTRKYKIKSNPLT |
| | sequence | KDIVIKMIPNVSNMSQCTGSVMENYKTRLNGILTPIKGALEIYKN |
| | | NTHDLVGDVRLAGVIMAGVAIGIATAAQITAGVALYEAMKNA |
| | | DNINKLKSSIESTNEAVVKLQETAEKTVYVLTALQDYINTNLVP |
| | | TIDKISCKQTELSLDLALSKYLSDLLFVFGPNLQDPVSNSMTIQAI |
| | | SQAFGGNYETLLRTLGYATEDFDDLLESDSITGQIIYVDLSSYYII |
| | | VRVYFPILTEIQQAYIQELLPVSFNNDNSEWISIVPNFILVRNTLIS |
| | | NIEIGFCLITKRSVICNQDYATPMTNNMRECLTGSTEKCPRELVV |
| | | SSHVPRFALSNGVLFANCISVTCQCQTTGRAISQSGEQTLLMIDN |
| | | TTCPTAVLGNVIISLGKYLGSVNYNSEGIAIGPPVFTDKVDISSQI |
| | | SSMNQSLQQSKDYIKEAQRLLDTVNPSLISMLSMIILYVLSIASLC |
| | | IGLITFISFIIVEKKRNT |
| 258 | EboV_signal_ | MGVTGILQLPRDRFKRTSFFLWVIILFQRTFSIILHYEKLSKIGLV |
| | sequence | KGVTRKYKIKSNPLTKDIVIKMIPNVSNMSQCTGSVMENYKTRL |
| | | NGILTPIKGALEIYKNNTHDLVGDVRLAGVIMAGVAIGIATAAQI |
| | | TAGVALYEAMKNADNINKLKSSIESTNEAVVKLQETAEKTVYV |
| | | LTALQDYINTNLVPTIDKISCKQTELSLDLALSKYLSDLLFVFGP |
| | | NLQDPVSNSMTIQAISQAFGGNYETLLRTLGYATEDFDDLLESD |
| | | SITGQIIYVDLSSYYIIVRVYFPILTEIQQAYIQELLPVSFNNDNSE |
| | | WISIVPNFILVRNTLISNIEIGFCLITKRSVICNQDYATPMTNNMRE |
| | | CLTGSTEKCPRELVVSSHVPRFALSNGVLFANCISVTCQCQTTG |
| | | RAISQSGEQTLLMIDNTTCPTAVLGNVIISLGKYLGSVNYNSEGI |
| | | AIGPPVFTDKVDISSQISSMNQSLQQSKDYIKEAQRLLDTVNPSLI |
| | | SMLSMIILYVLSIASLCIGLITFISFIIVEKKRNT |
| 259 | GaLV_signal_ | MVLLPGSMLLTSNLHHLRHQMSPGSWKRLIILLSCVFGGGGTSI |
| | sequence | LHYEKLSKIGLVKGVTRKYKIKSNPLTKDIVIKMIPNVSNMSQCT |
| | | GSVMENYKTRLNGILTPIKGALEIYKNNTHDLVGDVRLAGVIM |
| | | AGVAIGIATAAQITAGVALYEAMKNADNINKLKSSIESTNEAVV |
| | | KLQETAEKTVYVLTALQDYINTNLVPTIDKISCKQTELSLDLALS |
| | | KYLSDLLFVFGPNLQDPVSNSMTIQAISQAFGGNYETLLRTLGY |
| | | ATEDFDDLLESDSITGQIIYVDLSSYYIIVRVYFPILTEIQQAYIQE |
| | | LLPVSFNNDNSEWISIVPNFILVRNTLISNIEIGFCLITKRSVICNQD |
| | | YATPMTNNMRECLTGSTEKCPRELVVSSHVPRFALSNGVLFAN |
| | | CISVTCQCQTTGRAISQSGEQTLLMIDNTTCPTAVLGNVIISLGK |
| | | YLGSVNYNSEGIAIGPPVFTDKVDISSQISSMNQSLQQSKDYIKE |
| | | AQRLLDTVNPSLISMLSMIILYVLSIASLCIGLITFISFIIVEKKRNT |
| 260 | MLV- | MARSTLSKPLKNKVNPRGPLIPLILLMLRGVSTASPILHYEKLSKI |
| | A_signal_ | GLVKGVTRKYKIKSNPLTKDIVIKMIPNVSNMSQCTGSVMENYK |
| | sequence | TRLNGILTPIKGALEIYKNNTHDLVGDVRLAGVIMAGVAIGIATA |
| | | AQITAGVALYEAMKNADNINKLKSSIESTNEAVVKLQETAEKTV |
| | | YVLTALQDYINTNLVPTIDKISCKQTELSLDLALSKYLSDLLFVF |
| | | GPNLQDPVSNSMTIQAISQAFGGNYETLLRTLGYATEDFDDLLE |
| | | SDSITGQIIYVDLSSYYIIVRVYFPILTEIQQAYIQELLPVSFNNDNS |
| | | EWISIVPNFILVRNTLISNIEIGFCLITKRSVICNQDYATPMTNNMR |
| | | ECLTGSTEKCPRELVVSSHVPRFALSNGVLFANCISVTCQCQTTG |
| | | RAISQSGEQTLLMIDNTTCPTAVLGNVIISLGKYLGSVNYNSEGI |
| | | AIGPPVFTDKVDISSQISSMNQSLQQSKDYIKEAQRLLDTVNPSLI |
| | | SMLSMIILYVLSIASLCIGLITFISFIIVEKKRNT |
| 261 | VSVG_signal_ | MKCLLYLAFLFIGVNCILHYEKLSKIGLVKGVTRKYKIKSNPLTK |
| | sequence | DIVIKMIPNVSNMSQCTGSVMENYKTRLNGILTPIKGALEIYKNN |
| | | THDLVGDVRLAGVIMAGVAIGIATAAQITAGVALYEAMKNAD |
| | | NINKLKSSIESTNEAVVKLQETAEKTVYVLTALQDYINTNLVPTI |
| | | DKISCKQTELSLDLALSKYLSDLLFVFGPNLQDPVSNSMTIQAIS |
| | | QAFGGNYETLLRTLGYATEDFDDLLESDSITGQIIYVDLSSYYIIV |
| | | RVYFPILTEIQQAYIQELLPVSFNNDNSEWISIVPNFILVRNTLISNI |
| | | EIGFCLITKRSVICNQDYATPMTNNMRECLTGSTEKCPRELVVSS |
| | | HVPRFALSNGVLFANCISVTCQCQTTGRAISQSGEQTLLMIDNTT |
| | | CPTAVLGNVIISLGKYLGSVNYNSEGIAIGPPVFTDKVDISSQISS |
| | | MNQSLQQSKDYIKEAQRLLDTVNPSLISMLSMIILYVLSIASLCIG |
| | | LITFISFIIVEKKRNT |
| 262 | Prolactin_ | MNIKGSPWKGSLLLLLVSNLLLCQSVAPILHYEKLSKIGLVKGV |
| | signal_sequence | TRKYKIKSNPLTKDIVIKMIPNVSNMSQCTGSVMENYKTRLNGI |
| | | LTPIKGALEIYKNNTHDLVGDVRLAGVIMAGVAIGIATAAQITA |
| | | GVALYEAMKNADNINKLKSSIESTNEAVVKLQETAEKTVYVLT |
| | | ALQDYINTNLVPTIDKISCKQTELSLDLALSKYLSDLLFVFGPNL |
| | | QDPVSNSMTIQAISQAFGGNYETLLRTLGYATEDFDDLLESDSIT |
| | | GQIIYVDLSSYYIIVRVYFPILTEIQQAYIQELLPVSFNNDNSEWISI |
| | | VPNFILVRNTLISNIEIGFCLITKRSVICNQDYATPMTNNMRECLT |
| | | GSTEKCPRELVVSSHVPRFALSNGVLFANCISVTCQCQTTGRAIS |
| | | QSGEQTLLMIDNTTCPTAVLGNVIISLGKYLGSVNYNSEGIAIGP |
| | | PVFTDKVDISSQISSMNQSLQQSKDYIKEAQRLLDTVNPSLISML |
| | | SMIILYVLSIASLCIGLITFISFIIVEKKRNT |
| 263 | IgGk- | METPAQLLFLLLLWLPDTTGILHYEKLSKIGLVKGVTRKYKIKS |
| | L_signal_ | NPLTKDIVIKMIPNVSNMSQCTGSVMENYKTRLNGILTPIKGALE |
| | sequence | IYKNNTHDLVGDVRLAGVIMAGVAIGIATAAQITAGVALYEAM |
| | | KNADNINKLKSSIESTNEAVVKLQETAEKTVYVLTALQDYINTN |
| | | LVPTIDKISCKQTELSLDLALSKYLSDLLFVFGPNLQDPVSNSMTI |
| | | QAISQAFGGNYETLLRTLGYATEDFDDLLESDSITGQIIYVDLSS |
| | | YYIIVRVYFPILTEIQQAYIQELLPVSFNNDNSEWISIVPNFILVRN |
| | | TLISNIEIGFCLITKRSVICNQDYATPMTNNMRECLTGSTEKCPRE |
| | | LVVSSHVPRFALSNGVLFANCISVTCQCQTTGRAISQSGEQTLL |
| | | MIDNTTCPTAVLGNVIISLGKYLGSVNYNSEGIAIGPPVFTDKVDI |
| | | SSQISSMNQSLQQSKDYIKEAQRLLDTVNPSLISMLSMIILYVLSI |
| | | ASLCIGLITFISFIIVEKKRNT |
| 264 | Albumin_ | MKWVTFISLLFLFSSAYSILHYEKLSKIGLVKGVTRKYKIKSNPL |
| | signal_sequence | TKDIVIKMIPNVSNMSQCTGSVMENYKTRLNGILTPIKGALEIYK |
| | | NNTHDLVGDVRLAGVIMAGVAIGIATAAQITAGVALYEAMKN |
| | | ADNINKLKSSIESTNEAVVKLQETAEKTVYVLTALQDYINTNLV |
| | | PTIDKISCKQTELSLDLALSKYLSDLLFVFGPNLQDPVSNSMTIQ |
| | | AISQAFGGNYETLLRTLGYATEDFDDLLESDSITGQIIYVDLSSY |
| | | YIIVRVYFPILTEIQQAYIQELLPVSFNNDNSEWISIVPNFILVRNT |
| | | LISNIEIGFCLITKRSVICNQDYATPMTNNMRECLTGSTEKCPREL |
| | | VVSSHVPRFALSNGVLFANCISVTCQCQTTGRAISQSGEQTLLMI |
| | | DNTTCPTAVLGNVIISLGKYLGSVNYNSEGIAIGPPVFTDKVDISS |
| | | QISSMNQSLQQSKDYIKEAQRLLDTVNPSLISMLSMIILYVLSIAS |
| | | LCIGLITFISFIIVEKKRNT |
| 265 | CD5_signal_ | MPMGSLQPLATLYLLGMLVASCLGRLILHYEKLSKIGLVKGVTR |
| | sequence | KYKIKSNPLTKDIVIKMIPNVSNMSQCTGSVMENYKTRLNGILTP |
| | | IKGALEIYKNNTHDLVGDVRLAGVIMAGVAIGIATAAQITAGVA |
| | | LYEAMKNADNINKLKSSIESTNEAVVKLQETAEKTVYVLTALQ |
| | | DYINTNLVPTIDKISCKQTELSLDLALSKYLSDLLFVFGPNLQDP |
| | | VSNSMTIQAISQAFGGNYETLLRTLGYATEDFDDLLESDSITGQII |
| | | YVDLSSYYIIVRVYFPILTEIQQAYIQELLPVSFNNDNSEWISIVPN |
| | | FILVRNTLISNIEIGFCLITKRSVICNQDYATPMTNNMRECLTGST |
| | | EKCPRELVVSSHVPRFALSNGVLFANCISVTCQCQTTGRAISQSG |
| | | EQTLLMIDNTTCPTAVLGNVIISLGKYLGSVNYNSEGIAIGPPVFT |
| | | DKVDISSQISSMNQSLQQSKDYIKEAQRLLDTVNPSLISMLSMIIL |
| | | YVLSIASLCIGLITFISFIIVEKKRNT |
| 266 | Trypsinogen_ | MNPLLILTFVAAALAILHYEKLSKIGLVKGVTRKYKIKSNPLTKD |
| | signal_sequence | IVIKMIPNVSNMSQCTGSVMENYKTRLNGILTPIKGALEIYKNNT |
| | | HDLVGDVRLAGVIMAGVAIGIATAAQITAGVALYEAMKNADNI |
| | | NKLKSSIESTNEAVVKLQETAEKTVYVLTALQDYINTNLVPTID |
| | | KISCKQTELSLDLALSKYLSDLLFVFGPNLQDPVSNSMTIQAISQ |
| | | AFGGNYETLLRTLGYATEDFDDLLESDSITGQIIYVDLSSYYIIVR |
| | | VYFPILTEIQQAYIQELLPVSFNNDNSEWISIVPNFILVRNTLISNIE |
| | | IGFCLITKRSVICNQDYATPMTNNMRECLTGSTEKCPRELVVSSH |
| | | VPRFALSNGVLFANCISVTCQCQTTGRAISQSGEQTLLMIDNTTC |
| | | PTAVLGNVIISLGKYLGSVNYNSEGIAIGPPVFTDKVDISSQISSM |
| | | NQSLQQSKDYIKEAQRLLDTVNPSLISMLSMIILYVLSIASLCIGLI |
| | | TFISFIIVEKKRNT |
| 267 | IL2_signal_ | MYRMQLLSCIALSLALVTNSILHYEKLSKIGLVKGVTRKYKIKS |
| | sequence | NPLTKDIVIKMIPNVSNMSQCTGSVMENYKTRLNGILTPIKGALE |
| | | IYKNNTHDLVGDVRLAGVIMAGVAIGIATAAQITAGVALYEAM |
| | | KNADNINKLKSSIESTNEAVVKLQETAEKTVYVLTALQDYINTN |
| | | LVPTIDKISCKQTELSLDLALSKYLSDLLFVFGPNLQDPVSNSMTI |
| | | QAISQAFGGNYETLLRTLGYATEDFDDLLESDSITGQIIYVDLSS |
| | | YYIIVRVYFPILTEIQQAYIQELLPVSFNNDNSEWISIVPNFILVRN |
| | | TLISNIEIGFCLITKRSVICNQDYATPMTNNMRECLTGSTEKCPRE |
| | | LVVSSHVPRFALSNGVLFANCISVTCQCQTTGRAISQSGEQTLL |
| | | MIDNTTCPTAVLGNVIISLGKYLGSVNYNSEGIAIGPPVFTDKVDI |
| | | SSQISSMNQSLQQSKDYIKEAQRLLDTVNPSLISMLSMIILYVLSI |
| | | ASLCIGLITFISFIIVEKKRNT |

### 3. NiV-F Transmembrane Domain

Generally, the transmembrane of the Nipah F protein can participate in regulation of endocytic trafficking and exposure of the cleavage site to available Cathepsin-L proteases. Exemplary variant NiV-F protein constructs were generated in which the transmembrane domain was modified as shown below.

Exemplary variant NiV-F proteins were generated in which a series of swaps were made to replace the transmembrane domain of the native NiV-F (corresponding to amino acids 488-518 of SEQ ID NO:1) with a heterologous transmembrane domain from another virus. In an alternative or additional strategy, variant NiV-F proteins were generated with a modified transmembrane domain containing the amino acid replacement S490A, Y498A, S504A or 1516V corresponding to numbering of positions set forth in SEQ ID NO:1 or SEQ ID NO:302.

The series of variant NiV F proteins that were generated contained the transmembrane domain from another paramyxovirus, such as a henipavirus. The variant NiV-F proteins with a heterologous or modified transmembrane domain also were generated to contain a truncated cytoplasmic tail set forth in SEQ ID NO:26 that lacked 22 contiguous C-terminal amino acids of the full-length cytoplasmic tail of wild-type of native NiV-F set forth in SEQ ID NOA4. Specifically, exemplary variant NiV-F were generated by replacing the transmembrane domain corresponding to amino acid residues 488-518 of SEQ ID NO:302 with the heterologous or modified transmembrane domain

The generated NiV-F chimeric transmembrane domain variants are set forth in Table E8.

TABLE E8
NiV-F Chimeric Transmembrane Domain Mutants

| SEQ ID NO | Name | Full Length Sequence |
|---|---|---|
| 268 | NivF_TM_S490A_ | MVVILDKRCYCNLLILILMISECSVGILHYEKLSKIGLVKGV |
| | dec_fusogenicity | TRKYKIKSNPLTKDIVIKMIPNVSNMSQCTGSVMENYKTRL |
| | | NGILTPIKGALEIYKNNTHDLVGDVRLAGVIMAGVAIGIATA |
| | | AQITAGVALYEAMKNADNINKLKSSIESTNEAVVKLQETAE |
| | | KTVYVLTALQDYINTNLVPTIDKISCKQTELSLDLALSKYLS |
| | | DLLFVFGPNLQDPVSNSMTIQAISQAFGGNYETLLRTLGYA |
| | | TEDFDDLLESDSITGQIIYVDLSSYYIIVRVYFPILTEIQQAYIQ |
| | | ELLPVSFNNDNSEWISIVPNFILVRNTLISNIEIGFCLITKRSVI |
| | | CNQDYATPMTNNMRECLTGSTEKCPRELVVSSHVPRFALS |
| | | NGVLFANCISVTCQCQTTGRAISQSGEQTLLMIDNTTCPTAV |
| | | LGNVIISLGKYLGSVNYNSEGIAIGPPVFTDKVDISSQISSMN |
| | | QSLQQSKDYIKEAQRLLDTVNPSLIAMLSMIILYVLSIASLCI |
| | | GLITFISFIIVEKKRNT |
| 269 | NivF_TM_Y498A_ | MVVILDKRCYCNLLILILMISECSVGILHYEKLSKIGLVKGV |
| | dec_fusogenicity | TRKYKIKSNPLTKDIVIKMIPNVSNMSQCTGSVMENYKTRL |
| | | NGILTPIKGALEIYKNNTHDLVGDVRLAGVIMAGVAIGIATA |
| | | AQITAGVALYEAMKNADNINKLKSSIESTNEAVVKLQETAE |
| | | KTVYVLTALQDYINTNLVPTIDKISCKQTELSLDLALSKYLS |
| | | DLLFVFGPNLQDPVSNSMTIQAISQAFGGNYETLLRTLGYA |
| | | TEDFDDLLESDSITGQIIYVDLSSYYIIVRVYFPILTEIQQAYIQ |
| | | ELLPVSFNNDNSEWISIVPNFILVRNTLISNIEIGFCLITKRSVI |
| | | CNQDYATPMTNNMRECLTGSTEKCPRELVVSSHVPRFALS |
| | | NGVLFANCISVTCQCQTTGRAISQSGEQTLLMIDNTTCPTAV |
| | | LGNVIISLGKYLGSVNYNSEGIAIGPPVFTDKVDISSQISSMN |
| | | QSLQQSKDYIKEAQRLLDTVNPSLISMLSMIILAVLSIASLCI |
| | | GLITFISFIIVEKKRNT |
| 270 | HevF_TM_FL | MVVILDKRCYCNLLILILMISECSVGILHYEKLSKIGLVKGV |
| | | TRKYKIKSNPLTKDIVIKMIPNVSNMSQCTGSVMENYKTRL |
| | | NGILTPIKGALEIYKNNTHDLVGDVRLAGVIMAGVAIGIATA |
| | | AQITAGVALYEAMKNADNINKLKSSIESTNEAVVKLQETAE |
| | | KTVYVLTALQDYINTNLVPTIDKISCKQTELSLDLALSKYLS |
| | | DLLFVFGPNLQDPVSNSMTIQAISQAFGGNYETLLRTLGYA |
| | | TEDFDDLLESDSITGQIIYVDLSSYYIIVRVYFPILTEIQQAYIQ |
| | | ELLPVSFNNDNSEWISIVPNFILVRNTLISNIEIGFCLITKRSVI |
| | | CNQDYATPMTNNMRECLTGSTEKCPRELVVSSHVPRFALS |
| | | NGVLFANCISVTCQCQTTGRAISQSGEQTLLMIDNTTCPTAV |
| | | LGNVIISLGKYLGSVNYNSEGIAIGPPVFTDKVDISSQISSMN |
| | | QSLQQSKDYIKEAQRLLDTVNPSLISMLSMIILYVLSIAALCI |
| | | GLITFISFVIVEKKRNT |
| 271 | NivF_TM_HevF_ | MVVILDKRCYCNLLILILMISECSVGILHYEKLSKIGLVKGV |
| | S504A | TRKYKIKSNPLTKDIVIKMIPNVSNMSQCTGSVMENYKTRL |
| | | NGILTPIKGALEIYKNNTHDLVGDVRLAGVIMAGVAIGIATA |
| | | AQITAGVALYEAMKNADNINKLKSSIESTNEAVVKLQETAE |
| | | KTVYVLTALQDYINTNLVPTIDKISCKQTELSLDLALSKYLS |
| | | DLLFVFGPNLQDPVSNSMTIQAISQAFGGNYETLLRTLGYA |
| | | TEDFDDLLESDSITGQIIYVDLSSYYIIVRVYFPILTEIQQAYIQ |
| | | ELLPVSFNNDNSEWISIVPNFILVRNTLISNIEIGFCLITKRSVI |
| | | CNQDYATPMTNNMRECLTGSTEKCPRELVVSSHVPRFALS |
| | | NGVLFANCISVTCQCQTTGRAISQSGEQTLLMIDNTTCPTAV |
| | | LGNVIISLGKYLGSVNYNSEGIAIGPPVFTDKVDISSQISSMN |
| | | QSLQQSKDYIKEAQRLLDTVNPSLISMLSMIILYVLSIAALCI |
| | | GLITFISFIIVEKKRNT |
| 272 | NivF_TM_HevF_ | MVVILDKRCYCNLLILILMISECSVGILHYEKLSKIGLVKGV |
| | I516V | TRKYKIKSNPLTKDIVIKMIPNVSNMSQCTGSVMENYKTRL |
| | | NGILTPIKGALEIYKNNTHDLVGDVRLAGVIMAGVAIGIATA |
| | | AQITAGVALYEAMKNADNINKLKSSIESTNEAVVKLQETAE |
| | | KTVYVLTALQDYINTNLVPTIDKISCKQTELSLDLALSKYLS |
| | | DLLFVFGPNLQDPVSNSMTIQAISQAFGGNYETLLRTLGYA |
| | | TEDFDDLLESDSITGQIIYVDLSSYYIIVRVYFPILTEIQQAYIQ |
| | | ELLPVSFNNDNSEWISIVPNFILVRNTLISNIEIGFCLITKRSVI |
| | | CNQDYATPMTNNMRECLTGSTEKCPRELVVSSHVPRFALS |
| | | NGVLFANCISVTCQCQTTGRAISQSGEQTLLMIDNTTCPTAV |
| | | LGNVIISLGKYLGSVNYNSEGIAIGPPVFTDKVDISSQISSMN |
| | | QSLQQSKDYIKEAQRLLDTVNPSLISMLSMIILYVLSIASLCI |
| | | GLITFISFVIVEKKRNT |

### 4. Hyperfuspgenic Modifications

Generally, the viral F protein directs fusion of viral and cellular membranes which leads to delivery of the nucleocapsid (and/or its contents) into the cellular cytoplasm. The F protein of Nipah virus is a Class I viral fusion protein with at least three defined structural states: a pre-fusion native state, a pre-hairpin intermediate state, and a post-fusion. hairpin state. Without wishing to be bound by theory, it is thought that F protein interacts with the G protein also at the viral surface such that once the G protein binds to its cognate cellular receptor, the F protein undergoes a conformational change. The hairpin formation of the F protein then allows for the hydrophobic fusion peptide (amino acids 110-134 of SEQ ID NO: 1) to embed in the target cell membrane. Further, F protein expression on infected cells can also lead to the formation of syncytia, as neighboring cells can become targets for cell fusion.

Exemplary chimeric F proteins were generated in which a series of hyperfusogenic mutations were introduced. The series of chimeric F proteins that were generated contained hyperfusogenic mutations in either of the cytoplasmic tail or extracellular domains.

Selected exemplary mutated NiV-F proteins were generated in which truncated NiV-F protein as described above were further mutated around the fusogenic motif of the NiV-F cytoplasmic tail, found at positions 1-10 with reference to numbering set forth in SEQ ID NO:3 The extracellular domain (ectodomain) and transmembrane domain of each generated construct was the same as set forth in SEQ ID NO:2. As a control, a NiV-F containing a full-length cytoplasmic tail (set forth in SEQ ID NO:3). was generated; the full sequence of the control NiV-F sequence is set forth in SEQ ID NO:1

Fusogenic properties of viral F proteins, including the Nipah F protein, can be modulated by a number of structural factors, including location of glycosylation sites. Mutations to amino acids known to be implicated in hexamer stability can also change subsequent fusion potential, as the pre-fusion conformation of the Nipah F protein has been revealed by X-ray crystallography to be a so-called “hexamer-of-timers assembly”. Further exemplary mutated NiV-F proteins were generated in which a native NiV-F protein or variant NiV-F as described above was mutated or further mutated at a N-glycosylation site (with respect to amino acid positions 64, 67, 99 and/or 464 of SEQ ID NO:1) or at positions implicated in stability of F protein hexamers (i.e., Q393L and/or R109L in comparison to SEQ ID NO:1).

Chimeric F proteins were also generated, in which the cytoplasmic tail was derived from a F protein of another Paramyxovirus known to participate in fusion, either at the plasma membrane of an infected target cell or via the formation of syncytia. Specifically, exemplary hyperfsogenic Hendra virus cytoplasmic tails were engineered with hyperfusogenic mutations, such that the extracellular domain (ectodomain) and transmembrane domain of each generated construct was the same and is set forth in SEQ ID NO:2.

The generated NiV-F cytoplasmic tail mutants are set forth in Table E9.

TABLE E9
NiV-F Fusogenic Mutants

| | | |
|---|---|---|
| SEQ ID | | |
| NO | Name | Sequence (Cytoplasmic tail or Full Length) |

Cytoplasmic Tail Mutations

| | | |
|---|---|---|
| 53 | CT-06_K1A | EAKRNT |
| 54 | CT- | EAYSRL |
| | 02_YSRL_K1A | |
| 55 | CT-28_K1A | EAKRNTYSRLEDRRVRPTSSGDLYYIGT |
| | (full length) | |
| 56 | CT-10_K1A | EAKRNTYSRL |

N-glycan Mutations

| | | |
|---|---|---|
| 273 | NivF_hyper- | MVVILDKRCYCNLLILILMISECSVGILHYEKLSKIGLVKGVTRKY |
| | fusogenic_ | KIKSNPLTKDIVIKMIPNVSNMSQCTGSVMENYKTRLNGILTPIKG |
| | mutations_Nglycan | ALEIYKQNTHDLVGDVRLAGVIMAGVAIGIATAAQITAGVALYEA |
| | (F3) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 274 | NivF_hyper- | MVVILDKRCYCNLLILILMISECSVGILHYEKLSKIGLVKGVTRKY |
| | fusogenic_ | KIKSNPLTKDIVIKMIPNVSNMSQCTGSVMENYKTRLNGILTPIKG |
| | mutations_Nglycan | ALEIYKNNTHDLVGDVRLAGVIMAGVAIGIATAAQITAGVALYEA |
| | (F5) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 275 | NivF_hyper- | MVVILDKRCYCNLLILILMISECSVGILHYEKLSKIGLVKGVTRKY |
| | fusogenic_ | KIKSNPLTKDIVIKMIPNVSNMSQCTGSVMENYKTRLNGILTPIKG |
| | mutations_Nglycan | ALEIYKNNTHDLVGDVRLAGVIMAGVAIGIATAAQITAGVALYEA |
| | (F4F5) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDQTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 276 | NivF_hyper- | MVVILDKRCYCNLLILILMISECSVGILHYEKLSKIGLVKGVTRKY |
| | fusogenic_ | KIKSNPLTKDIVIKMIPNVSQMSQCTGSVMENYKTRLNGILTPIKG |
| | mutations_Nglycan | ALEIYKNNTHDLVGDVRLAGVIMAGVAIGIATAAQITAGVALYEA |
| | (F2F4F5) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDQTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 278 | NivF_hyper- | MVVILDKRCYCNLLILILMISECSVGILHYEKLSKIGLVKGVTRKY |
| | fusogenic_ | KIKSNPLTKDIVIKMIPNVSQMSQCTGSVMENYKTRLNGILTPIKG |
| | mutations_Nglycan | ALEIYKQNTHDLVGDVRLAGVIMAGVAIGIATAAQITAGVALYEA |
| | (F2F3) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 279 | NivF_hyper- | MVVILDKRCYCNLLILILMISECSVGILHYEKLSKIGLVKGVTRKY |
| | fusogenic_ | KIKSNPLTKDIVIKMIPNVSQMSQCTGSVMENYKTRLNGILTPIKG |
| | mutations_Nglycan | ALEIYKNNTHDLVGDVRLAGVIMAGVAIGIATAAQITAGVALYEA |
| | (F2F5) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 280 | NivF_hyper- | MVVILDKRCYCNLLILILMISECSVGILHYEKLSKIGLVKGVTRKY |
| | fusogenic_ | KIKSNPLTKDIVIKMIPNVSNMSQCTGSVMENYKTRLNGILTPIKG |
| | mutations_Nglycan | ALEIYKQNTHDLVGDVRLAGVIMAGVAIGIATAAQITAGVALYEA |
| | (F3F5) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 290 | NivF_hyper- | MVVILDKRCYCNLLILILMISECSVGILHYEKLSKIGLVKGVTRKY |
| | fusogenic_ | KIKSNPLTKDIVIKMIPNVSQMSQCTGSVMENYKTRLNGILTPIKG |
| | mutations_Nglycan | ALEIYKQNTHDLVGDVRLAGVIMAGVAIGIATAAQITAGVALYEA |
| | (F2F3F5) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 457 | F1F2_CT06 | MVVILDKRCYCNLLILILMISECSVGILHYEKLSKIGLVKGVTRKY |
| | | KIKSNPLTKDIVIKMIPQVSQMSQCTGSVMENYKTRLNGILTPIKG |
| | | ALEIYKNNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 458 | F1F2F3_CT06 | MVVILDKRCYCNLLILILMISECSVGILHYEKLSKIGLVKGVTRKY |
| | | KIKSNPLTKDIVIKMIPQVSQMSQCTGSVMENYKTRLNGILTPIKG |
| | | ALEIYKQNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 459 | F1F2F3F5_CT06 | MVVILDKRCYCNLLILILMISECSVGILHYEKLSKIGLVKGVTRKY |
| | | KIKSNPLTKDIVIKMIPQVSQMSQCTGSVMENYKTRLNGILTPIKG |
| | | ALEIYKQNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 460 | F1F3_CT06 | MVVILDKRCYCNLLILILMISECSVGILHYEKLSKIGLVKGVTRKY |
| | | KIKSNPLTKDIVIKMIPQVSNMSQCTGSVMENYKTRLNGILTPIKG |
| | | ALEIYKQNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 461 | F1F2F5_CT06 | MVVILDKRCYCNLLILILMISECSVGILHYEKLSKIGLVKGVTRKY |
| | | KIKSNPLTKDIVIKMIPQVSQMSQCTGSVMENYKTRLNGILTPIKG |
| | | ALEIYKNNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 462 | F1F3F5_CT06 | MVVILDKRCYCNLLILILMISECSVGILHYEKLSKIGLVKGVTRKY |
| | | KIKSNPLTKDIVIKMIPQVSNMSQCTGSVMENYKTRLNGILTPIKG |
| | | ALEIYKNNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 463 | F1F5_CT06 | MVVILDKRCYCNLLILILMISECSVGILHYEKLSKIGLVKGVTRKY |
| | | KIKSNPLTKDIVIKMIPQVSNMSQCTGSVMENYKTRLNGILTPIKG |
| | | ALEIYKNNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |
| 464 | F2F3F5_CT06 | MVVILDKRCYCNLLILILMISECSVGILHYEKLSKIGLVKGVTRKY |
| | | KIKSNPLTKDIVIKMIPNVSQMSQCTGSVMENYKTRLNGILTPIKG |
| | | ALEIYKQNTHDLVGDVRLAGVIMAGVAIGIATAAQITAGVALYEA |
| | | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMQQ |
| | | SLQQSKDYIKEAQRLLDTVNPSLISMLSMIILYVLSIASLCIGLITFIS |
| | | FIIVEKKRNT |

Hexamer Stability Mutations

| | | |
|---|---|---|
| 291 | NivF_hyperfuso- | MVVILDKRCYCNLLILILMISECSVGILHYEKLSKIGLVKGVTRKY |
| | genic_mutations_ | KIKSNPLTKDIVIKMIPNVSNMSQCTGSVMENYKTRLNGILTPIKG |
| | Hexamer- | ALEIYKNNTHDLVGDVLLAGVIMAGVAIGIATAAQITAGVALYEA |
| | stabl(R109L) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCQCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 292 | NivF_hyperfus | MVVILDKRCYCNLLILILMISECSVGILHYEKLSKIGLVKGVTRKY |
| | genic_mutations_ | KIKSNPLTKDIVIKMIPNVSNMSQCTGSVMENYKTRLNGILTPIKG |
| | Hexamer- | ALEIYKNNTHDLVGDVRLAGVIMAGVAIGIATAAQITAGVALYEA |
| | stab1(Q393L) | MKNADNINKLKSSIESTNEAVVKLQETAEKTVYVLTALQDYINTN |
| | | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCLCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 293 | NivF_hyperfus | MVVILDKRCYCNLLILILMISECSVGILHYEKLSKIGLVKGVTRKY |
| | genic_mutations_ | KIKSNPLTKDIVIKMIPNVSNMSQCTGSVMENYKTRLNGILTPIKG |
| | Hexamer- | ALEIYKNNTHDLVGDVLLAGVIMAGVAIGIATAAQITAGVALYEA |
| | stab1(R109L_ | MKNADNINKLKSSIESTNEAVVKLQETAEKTVYVLTALQDYINTN |
| | Q393L) | LVPTIDKISCKQTELSLDLALSKYLSDLLFVFGPNLQDPVSNSMTIQ |
| | | AISQAFGGNYETLLRTLGYATEDFDDLLESDSITGQIIYVDLSSYYII |
| | | VRVYFPILTEIQQAYIQELLPVSFNNDNSEWISIVPNFILVRNTLISNI |
| | | EIGFCLITKRSVICNQDYATPMTNNMRECLTGSTEKCPRELVVSSH |
| | | VPRFALSNGVLFANCISVTCLCQTTGRAISQSGEQTLLMIDNTTCP |
| | | TAVLGNVIISLGKYLGSVNYNSEGIAIGPPVFTDKVDISSQISSMNQ |
| | | SLQQSKDYIKEAQRLLDTVNPSLISMLSMIILYVLSIASLCIGLITFIS |
| | | FIIVEKKRNT |
| 294 | NivF_hyperfuso- | MATQEVRLKCLLCGIIVLVLSLEGLGILHYEKLSKIGLVKGITRKY |
| | genic_mutations_ | KIKSNPLTKDIVIKMIPNVSNVSKCTGTVMENYKSRLTGILSPIKGA |
| | HendraF | IELYNNNTHDLVGDVKLAGVVMAGIAIGIATAAQITAGVALYEA |
| | | MKNADNINKLKSSIESTNEAVVKLQETAEKTVYVLTALQDYINTN |
| | | LVPTIDQISCKQTELALDLALSKYLSDLLFVFGPNLQDPVSNSMTI |
| | | QAISQAFGGNYETLLRTLGYATEDFDDLLESDSIAGQIVYVDLSSY |
| | | YIIVRVYFPILTEIQQAYVQELLPVSFNNDNSEWISIVPNFVLIRNTL |
| | | ISNIEVKYCLITKKSVICNQDYATPMTASVRECLTGSTDKCPRELV |
| | | VSSHVPRFALSGGVLFANCISVTCQCQTTGRAISQSGEQTLLMIDN |
| | | TTCTTVVLGNIIISLGKYLGSINYNSESIAVGPPVYTDKVDISSQISS |
| | | MNQSLQQSKDYIKEAQKILDTVNPSLISMLSMIILYVLSIAALCIGLI |
| | | TFISFVIVEKKRGNYSRLDDRQVRPVSNGDLYYIGT |
| 295 | NivF_hyperfuso- | MATQEVRLKCLLCGIIVLVLSLEGLGILHYEKLSKIGLVKGITRKY |
| | genic_mutations_ | KIKSNPLTKDIVIKMIPNVSNVSKCTGTVMENYKSRLTGILSPIKGA |
| | HendraF | IELYNNNTHDLVGDVKLAGVVMAGIAIGIATAAQITAGVALYEA |
| | (HevF_CT-06) | MKNADNINKLKSSIESTNEAVVKLQETAEKTVYVLTALQDYINTN |
| | | LVPTIDQISCKQTELALDLALSKYLSDLLFVFGPNLQDPVSNSMTI |
| | | QAISQAFGGNYETLLRTLGYATEDFDDLLESDSIAGQIVYVDLSSY |
| | | YIIVRVYFPILTEIQQAYVQELLPVSFNNDNSEWISIVPNFVLIRNTL |
| | | ISNIEVKYCLITKKSVICNQDYATPMTASVRECLTGSTDKCPRELV |
| | | VSSHVPRFALSGGVLFANCISVTCQCQTTGRAISQSGEQTLLMIDN |
| | | TTCTTVVLGNIIISLGKYLGSINYNSESIAVGPPVYTDKVDISSQISS |
| | | MNQSLQQSKDYIKEAQKILDTVNPSLISMLSMIILYVLSIAALCIGLI |
| | | TFISFVIVEKKRGN |
| 296 | NivF_hyperfuso- | MATQEVRLKCLLCGIIVLVLSLEGLGILHYEKLSKIGLVKGITRKY |
| | genic_mutations_ | KIKSNPLTKDIVIKMIPNVSNVSKCTGTVMENYKSRLTGILSPIKGA |
| | HendraF | IELYNNNTHDLVGDVKLAGVVMAGIAIGIATAAQITAGVALYEA |
| | (HevF_CT-10) | MKNADNINKLKSSIESTNEAVVKLQETAEKTVYVLTALQDYINTN |
| | | LVPTIDQISCKQTELALDLALSKYLSDLLFVFGPNLQDPVSNSMTI |
| | | QAISQAFGGNYETLLRTLGYATEDFDDLLESDSIAGQIVYVDLSSY |
| | | YIIVRVYFPILTEIQQAYVQELLPVSFNNDNSEWISIVPNFVLIRNTL |
| | | ISNIEVKYCLITKKSVICNQDYATPMTASVRECLTGSTDKCPRELV |
| | | VSSHVPRFALSGGVLFANCISVTCQCQTTGRAISQSGEQTLLMIDN |
| | | TTCTTVVLGNIIISLGKYLGSINYNSESIAVGPPVYTDKVDISSQISS |
| | | MNQSLQQSKDYIKEAQKILDTVNPSLISMLSMIILYVLSIAALCIGLI |
| | | TFISFVIVEKKRGNYSRL |
| 281 | HevF488NivF_ | MATQEVRLKCLLCGIIVLVLSLEGLGILHYEKLSKIGLVKGITRKY |
| | full-length | KIKSNPLTKDIVIKMIPNVSNVSKCTGTVMENYKSRLTGILSPIKGA |
| | | IELYNNNTHDLVGDVKLAGVVMAGIAIGIATAAQITAGVALYEA |
| | | MKNADNINKLKSSIESTNEAVVKLQETAEKTVYVLTALQDYINTN |
| | | LVPTIDQISCKQTELALDLALSKYLSDLLFVFGPNLQDPVSNSMTI |
| | | QAISQAFGGNYETLLRTLGYATEDFDDLLESDSIAGQIVYVDLSSY |
| | | YIIVRVYFPILTEIQQAYVQELLPVSFNNDNSEWISIVPNFVLIRNTL |
| | | ISNIEVKYCLITKKSVICNQDYATPMTASVRECLTGSTDKCPRELV |
| | | VSSHVPRFALSGGVLFANCISVTCQCQTTGRAISQSGEQTLLMIDN |
| | | TTCTTVVLGNIIISLGKYLGSINYNSESIAVGPPVYTDKVDISSQISS |
| | | MNQSLQQSKDYIKEAQKILDTVNPSLISMLSMIILYVLSIASLCIGLI |
| | | TFISFIIVEKKRNTYSRLEDRRVRPTSSGDLYYIGT |
| 282 | HevF488NivF_ | MATQEVRLKCLLCGIIVLVLSLEGLGILHYEKLSKIGLVKGITRKY |
| | CT-06 | KIKSNPLTKDIVIKMIPNVSNVSKCTGTVMENYKSRLTGILSPIKGA |
| | | IELYNNNTHDLVGDVKLAGVVMAGIAIGIATAAQITAGVALYEA |
| | | MKNADNINKLKSSIESTNEAVVKLQETAEKTVYVLTALQDYINTN |
| | | LVPTIDQISCKQTELALDLALSKYLSDLLFVFGPNLQDPVSNSMTI |
| | | QAISQAFGGNYETLLRTLGYATEDFDDLLESDSIAGQIVYVDLSSY |
| | | YIIVRVYFPILTEIQQAYVQELLPVSFNNDNSEWISIVPNFVLIRNTL |
| | | ISNIEVKYCLITKKSVICNQDYATPMTASVRECLTGSTDKCPRELV |
| | | VSSHVPRFALSGGVLFANCISVTCQCQTTGRAISQSGEQTLLMIDN |
| | | TTCTTVVLGNIIISLGKYLGSINYNSESIAVGPPVYTDKVDISSQISS |
| | | MNQSLQQSKDYIKEAQKILDTVNPSLISMLSMIILYVLSIASLCIGLI |
| | | TFISFIIVEKKRNT |
| 283 | HevF488NivF_ | MATQEVRLKCLLCGIIVLVLSLEGLGILHYEKLSKIGLVKGITRKY |
| | CT-10 | KIKSNPLTKDIVIKMIPNVSNVSKCTGTVMENYKSRLTGILSPIKGA |
| | | IELYNNNTHDLVGDVKLAGVVMAGIAIGIATAAQITAGVALYEA |
| | | MKNADNINKLKSSIESTNEAVVKLQETAEKTVYVLTALQDYINTN |
| | | LVPTIDQISCKQTELALDLALSKYLSDLLFVFGPNLQDPVSNSMTI |
| | | QAISQAFGGNYETLLRTLGYATEDFDDLLESDSIAGQIVYVDLSSY |
| | | YIIVRVYFPILTEIQQAYVQELLPVSFNNDNSEWISIVPNFVLIRNTL |
| | | ISNIEVKYCLITKKSVICNQDYATPMTASVRECLTGSTDKCPRELV |
| | | VSSHVPRFALSGGVLFANCISVTCQCQTTGRAISQSGEQTLLMIDN |
| | | TTCTTVVLGNIIISLGKYLGSINYNSESIAVGPPVYTDKVDISSQISS |
| | | MNQSLQQSKDYIKEAQKILDTVNPSLISMLSMIILYVLSIASLCIGLI |
| | | TFISFIIVEKKRNTYSRL |

### 5. Combinatorial Variants

Further exemplary NiV-F protein constructs were generated as combinations of various modification strategies described above. Here, modification of an individual NiV-F variant was combined with other modifications to a different loci within the NiV-F protein. For example, an individual NiV-F cytoplasmic tail variant was generated to also have a signal sequence modification in the ectodomain as described above. In another example, an individual NiV-F cytoplasmic tail variant was generated to be truncated as described above and to also have a modification in any of the ectodomain or transmembrane domain. In some aspects, combining modifications or truncations can result in an additive effect on resultant pseudotyped particle titer.

Additionally, exemplary chimeric F protein constructs containing a cytoplasmic tail or a truncated cytoplasmic tail from another virus, described in Example A.1.C, were generated to further contain combinations of various other modification strategies described above, including in Section II. F. Here, modifications of an individual chimeric F protein construct was combined with other modifications to a different loci within the F protein. For example, an individual chimeric F cytoplasmic tail variant was generated to also have a signal sequence modification in the ectodomain as described above. In another example, an individual chimeric F variant was generated to be truncated as described above and to also have a modification in any of the ectodomain or transmembrane domain. In some aspects, combining modifications or truncations can result in an additive effect on resultant pseudotyped particle titer.

Exemplary generated combinatorial variants are set forth in Table E10 below.

TABLE E10
Combinatorial Variants

| SEQ ID NO | Name/Description | Full Length Sequence |
|---|---|---|
| 58 | F3F5_Q393L_ | MVVILDKRCYCNLLILILMISECSVGILHYEKLSKIGLVKGVT |
| | CT-06_K1A | RKYKIKSNPLTKDIVIKMIPNVSNMSQCTGSVMENYKTRLN |
| | | GILTPIKGALEIYKQNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEAKRNT |
| 57 | F3F5_Q393L_ | MVVILDKRCYCNLLILILMISECSVGILHYEKLSKIGLVKGVT |
| | CT-10_K1A | RKYKIKSNPLTKDIVIKMIPNVSNMSQCTGSVMENYKTRLN |
| | | GILTPIKGALEIYKQNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEAKRNTYSRL |
| 297 | VSVG_signal_ | MKCLLYLAFLFIGVNCILHYEKLSKIGLVKGVTRKYKIKSNP |
| | sequence_ | LTKDIVIKMIPNVSNMSQCTGSVMENYKTRLNGILTPIKGAL |
| | MeaslesF_CT_3 | EIYKNNTHDLVGDVRLAGVIMAGVAIGIATAAQITAGVALY |
| | | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CQCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVRGR |
| 298 | IL2_signal_ | MYRMQLLSCIALSLALVTNSILHYEKLSKIGLVKGVTRKYKI |
| | sequence_MeaslesF_ | KSNPLTKDIVIKMIPNVSNMSQCTGSVMENYKTRLNGILTPI |
| | CT_3 | KGALEIYKNNTHDLVGDVRLAGVIMAGVAIGIATAAQITAG |
| | | VALYEAMKNADNINKLKSSIESTNEAVVKLQETAEKTVYV |
| | | LTALQDYINTNLVPTIDKISCKQTELSLDLALSKYLSDLLFVF |
| | | GPNLQDPVSNSMTIQAISQAFGGNYETLLRTLGYATEDFDD |
| | | LLESDSITGQIIYVDLSSYYIIVRVYFPILTEIQQAYIQELLPVS |
| | | FNNDNSEWISIVPNFILVRNTLISNIEIGFCLITKRSVICNQDY |
| | | ATPMTNNMRECLTGSTEKCPRELVVSSHVPRFALSNGVLFA |
| | | NCISVTCQCQTTGRAISQSGEQTLLMIDNTTCPTAVLGNVIIS |
| | | LGKYLGSVNYNSEGIAIGPPVFTDKVDISSQISSMNQSLQQS |
| | | KDYIKEAQRLLDTVNPSLISMLSMIILYVLSIASLCIGLITFISF |
| | | IIVRGR |
| 299 | F3F5_ | MKCLLYLAFLFIGVNCILHYEKLSKIGLVKGVTRKYKIKSNP |
| | VSVG_signal_ | LTKDIVIKMIPNVSNMSQCTGSVMENYKTRLNGILTPIKGAL |
| | sequence_ | EIYKQNTHDLVGDVRLAGVIMAGVAIGIATAAQITAGVALY |
| | MeaslesF_CT_3 | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CQCQTTGRAISQSGEQTLLMIDNTTCPTAVLGNVIISLGKYL |
| | | GSVNYNSEGIAIGPPVFTDKVDISSQISSMQQSLQQSKDYIKE |
| | | AQRLLDTVNPSLISMLSMIILYVLSIASLCIGLITFISFIIVEKK |
| | | RNT |
| 300 | F3F5_ | MKCLLYLAFLFIGVNCILHYEKLSKIGLVKGITRKYKIKSNP |
| | HevF488NivF_ | LTKDIVIKMIPNVSNVSKCTGTVMENYKSRLTGILSPIKGAIE |
| | CT-06 | LYNNNTHDLVGDVKLAGVVMAGIAIGIATAAQITAGVALY |
| | | EAMKNADNINKLKSSIESTNEAVVKLQETAEKTVYVLTALQ |
| | | DYINTNLVPTIDQISCKQTELALDLALSKYLSDLLFVFGPNL |
| | | QDPVSNSMTIQAISQAFGGNYETLLRTLGYATEDFDDLLES |
| | | DSIAGQIVYVDLSSYYIIVRVYFPILTEIQQAYVQELLPVSFN |
| | | NDNSEWISIVPNFVLIRNTLISNIEVKYCLITKKSVICNQDYA |
| | | TPMTASVRECLTGSTDKCPRELVVSSHVPRFALSGGVLFAN |
| | | CISVTCQCQTTGRAISQSGEQTLLMIDNTTCTTVVLGNIIISL |
| | | GKYLGSINYNSESIAVGPPVYTDKVDISSQISSMNQSLQQSK |
| | | DYIKEAQKILDTVNPSLISMLSMIILYVLSIASLCIGLITFISFII |
| | | VEKKRNT |
| 367 | F5_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEKKRNT |
| 368 | F5_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | VGDVK | GILTPIKGALEIYKNNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEKKRNT |
| 369 | F5_ VGDVK | MVVILDKRCYCNLLILILMISECSVGILHYEKLSKIGLVKGVT |
| | | RKYKIKSNPLTKDIVIKMIPNVSNMSQCTGSVMENYKTRLN |
| | | GILTPIKGALEIYKNNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCQCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEKKRNT |
| 370 | Q393L_ | MVVILDKRCYCNLLILILMISECSVGILHYEKLSKIGLVKGVT |
| | VGDVK | RKYKIKSNPLTKDIVIKMIPNVSNMSQCTGSVMENYKTRLN |
| | | GILTPIKGALEIYKNNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRNT |
| 371 | F5_ | MHHLHPMIVCIFVMYTGIVGSDAILHYEKLSKIGLVKGVTR |
| | Q393L_ | KYKIKSNPLTKDIVIKMIPNVSNMSQCTGSVMENYKTRLNGI |
| | HPIV2_signal_ | LTPIKGALEIYKNNTHDLVGDVRLAGVIMAGVAIGIATAAQI |
| | sequence | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMQQSL |
| | | QQSKDYIKEAQRLLDTVNPSLISMLSMIILYVLSIASLCIGLIT |
| | | FISFIIVEKKRNT |
| 372 | F5_ | MKCLLYLAFLFIGVNCILHYEKLSKIGLVKGVTRKYKIKSNP |
| | Q393L_ | LTKDIVIKMIPNVSNMSQCTGSVMENYKTRLNGILTPIKGAL |
| | VSVG_signal_ | EIYKNNTHDLVGDVRLAGVIMAGVAIGIATAAQITAGVALY |
| | sequence | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSENNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMQQSLQQSKDYIKE |
| | | AQRLLDTVNPSLISMLSMIILYVLSIASLCIGLITFISFIIVEKK |
| | | RNT |
| 373 | F5_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | NDVF_CT_6 | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVKQKAQQ |
| 374 | F5_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | HeVF_CT_6 | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEKKRGN |
| 375 | F5_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | NDVF_CT_6 | GILTPIKGALEIYKNNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVKQKAQQ |
| 376 | F5_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | VGDVK | GILTPIKGALEIYKNNTHDLVGDVKLAGVIMAGVAIGIATAA |
| | HeVF_CT_6 | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVEKKRGN |
| 377 | F5_NDVF_CT_ | MVVILDKRCYCNLLILILMISECSVGILHYEKLSKIGLVKGVT |
| | 6 | RKYKIKSNPLTKDIVIKMIPNVSNMSQCTGSVMENYKTRLN |
| | | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCQCQTTGRAISQSGEQTLLMIDNTTCPTAVLG |
| | | NVIISLGKYLGSVNYNSEGIAIGPPVFTDKVDISSQISSMQQS |
| | | LQQSKDYIKEAQRLLDTVNPSLISMLSMIILYVLSIASLCIGLI |
| | | TFISFIIVKQKAQQ |
| 378 | Q393L_NDVF_ | MVVILDKRCYCNLLILILMISECSVGILHYEKLSKIGLVKGVT |
| | CT_6 | RKYKIKSNPLTKDIVIKMIPNVSNMSQCTGSVMENYKTRLN |
| | | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVKQKAQQ |
| 423 | HPIV2_signal_ | MHHLHPMIVCIFVMYTGIVGSDAILHYEKLSKIGLVKGVTR |
| | sequence_CT_6_ | KYKIKSNPLTKDIVIKMIPNVSNMSQCTGSVMENYKTRLNGI |
| | Q393L | LTPIKGALEIYKNNTHDLVGDVRLAGVIMAGVAIGIATAAQI |
| | | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRNT |
| 424 | VSVG_signal_ | MKCLLYLAFLFIGVNCILHYEKLSKIGLVKGVTRKYKIKSNP |
| | sequence_ | LTKDIVIKMIPNVSNMSQCTGSVMENYKTRLNGILTPIKGAL |
| | CT_6_Q393L | EIYKNNTHDLVGDVRLAGVIMAGVAIGIATAAQITAGVALY |
| | | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RNT |
| 425 | VGDVK | MHHLHPMIVCIFVMYTGIVGSDAILHYEKLSKIGLVKGVTR |
| | (HeV)_HPIV2_ | KYKIKSNPLTKDIVIKMIPNVSNMSQCTGSVMENYKTRLNGI |
| | signal_sequence_ | LTPIKGALEIYKNNTHDLVGDVKLAGVIMAGVAIGIATAAQI |
| | CT_6_Q393L | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRNT |
| 426 | VGDVK | MKCLLYLAFLFIGVNCILHYEKLSKIGLVKGVTRKYKIKSNP |
| | (HeV)_VSVG_ | LTKDIVIKMIPNVSNMSQCTGSVMENYKTRLNGILTPIKGAL |
| | signal_sequence_ | EIYKNNTHDLVGDVKLAGVIMAGVAIGIATAAQITAGVALY |
| | CT_6_Q393L | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RNT |
| 427 | NDVF_CT_6_ | MVVILDKRCYCNLLILILMISECSVGILHYEKLSKIGLVKGVT |
| | Q393L_ | RKYKIKSNPLTKDIVIKMIPNVSNMSQCTGSVMENYKTRLN |
| | | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVKQKAQQ |
| 428 | VGDVK | MVVILDKRCYCNLLILILMISECSVGILHYEKLSKIGLVKGVT |
| | (HeV)_NDVF_ | RKYKIKSNPLTKDIVIKMIPNVSNMSQCTGSVMENYKTRLN |
| | CT_6_Q393L | GILTPIKGALEIYKNNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVKQKAQQ |
| 429 | HPIV2_signal_ | MHHLHPMIVCIFVMYTGIVGSDAILHYEKLSKIGLVKGVTR |
| | sequence- | KYKIKSNPLTKDIVIKMIPNVSNMSQCTGSVMENYKTRLNGI |
| | NDVF_CT_6_ | LTPIKGALEIYKNNTHDLVGDVRLAGVIMAGVAIGIATAAQI |
| | Q393L | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVKQKAQQ |
| 430 | VSVG_signal_ | MKCLLYLAFLFIGVNCILHYEKLSKIGLVKGVTRKYKIKSNP |
| | sequence_NDVF_ | LTKDIVIKMIPNVSNMSQCTGSVMENYKTRLNGILTPIKGAL |
| | CT_6_Q393L | EIYKNNTHDLVGDVRLAGVIMAGVAIGIATAAQITAGVALY |
| | | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVKQK |
| | | AQQ |
| 431 | VGDVK | MHHLHPMIVCIFVMYTGIVGSDAILHYEKLSKIGLVKGVTR |
| | (HeV)_HPIV2_ | KYKIKSNPLTKDIVIKMIPNVSNMSQCTGSVMENYKTRLNGI |
| | signal_sequence_ | LTPIKGALEIYKNNTHDLVGDVKLAGVIMAGVAIGIATAAQI |
| | NDVF_CT_6_ | TAGVALYEAMKNADNINKLKSSIESTNEAVVKLQETAEKT |
| | Q393L_ | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVKQKAQQ |
| 432 | VGDVK | MKCLLYLAFLFIGVNCILHYEKLSKIGLVKGVTRKYKIKSNP |
| | (HeV)_VSVG_ | LTKDIVIKMIPNVSNMSQCTGSVMENYKTRLNGILTPIKGAL |
| | signal_sequence_ | EIYKNNTHDLVGDVKLAGVIMAGVAIGIATAAQITAGVALY |
| | NDVF_CT_6_ | EAMKNADNINKLKSSIESTNEAVVKLQETAEKTVYVLTALQ |
| | Q393L_ | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVKQK |
| | | AQQ |
| 433 | HeVF_CT_6_ | MVVILDKRCYCNLLILILMISECSVGILHYEKLSKIGLVKGVT |
| | Q393L | RKYKIKSNPLTKDIVIKMIPNVSNMSQCTGSVMENYKTRLN |
| | | GILTPIKGALEIYKNNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRGN |
| 434 | VGDVK (HeV)_ | MVVILDKRCYCNLLILILMISECSVGILHYEKLSKIGLVKGVT |
| | HeVF_CT_6_ | RKYKIKSNPLTKDIVIKMIPNVSNMSQCTGSVMENYKTRLN |
| | Q393L | GILTPIKGALEIYKNNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRGN |
| 435 | HPIV2_signal_ | MHHLHPMIVCIFVMYTGIVGSDAILHYEKLSKIGLVKGVTR |
| | sequence_HeVF_ | KYKIKSNPLTKDIVIKMIPNVSNMSQCTGSVMENYKTRLNGI |
| | CT_6_Q393L | LTPIKGALEIYKNNTHDLVGDVRLAGVIMAGVAIGIATAAQI |
| | | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRGN |
| 436 | VSVG_signal_ | MKCLLYLAFLFIGVNCILHYEKLSKIGLVKGVTRKYKIKSNP |
| | sequence_HeVF_ | LTKDIVIKMIPNVSNMSQCTGSVMENYKTRLNGILTPIKGAL |
| | CT_6_Q393L | EIYKNNTHDLVGDVRLAGVIMAGVAIGIATAAQITAGVALY |
| | | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSENNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RGN |
| 437 | VGDVK | MHHLHPMIVCIFVMYTGIVGSDAILHYEKLSKIGLVKGVTR |
| | (HeV)_HPIV2_ | KYKIKSNPLTKDIVIKMIPNVSNMSQCTGSVMENYKTRLNGI |
| | signal_sequence_ | LTPIKGALEIYKNNTHDLVGDVKLAGVIMAGVAIGIATAAQI |
| | HeVF_CT_6_ | TAGVALYEAMKNADNINKLKSSIESTNEAVVKLQETAEKT |
| | Q393L | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRGN |
| 438 | VGDVK | MKCLLYLAFLFIGVNCILHYEKLSKIGLVKGVTRKYKIKSNP |
| | (HeV)_VSVG_ | LTKDIVIKMIPNVSNMSQCTGSVMENYKTRLNGILTPIKGAL |
| | signal_sequence_ | EIYKNNTHDLVGDVKLAGVIMAGVAIGIATAAQITAGVALY |
| | HeVF_CT_6_ | EAMKNADNINKLKSSIESTNEAVVKLQETAEKTVYVLTALQ |
| | Q393L | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RGN |
| 439 | F3_CT06_Q393L | MVVILDKRCYCNLLILILMISECSVGILHYEKLSKIGLVKGVT |
| | | RKYKIKSNPLTKDIVIKMIPNVSNMSQCTGSVMENYKTRLN |
| | | GILTPIKGALEIYKQNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRNT |
| 440 | F3_ | MVVILDKRCYCNLLILILMISECSVGILHYEKLSKIGLVKGVT |
| | VGDVK (HeV)_ | RKYKIKSNPLTKDIVIKMIPNVSNMSQCTGSVMENYKTRLN |
| | CT_6_Q393L | GILTPIKGALEIYKQNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRNT |
| 441 | F3-_HPIV2_signal_ | MHHLHPMIVCIFVMYTGIVGSDAILHYEKLSKIGLVKGVTR |
| | sequence_ | KYKIKSNPLTKDIVIKMIPNVSNMSQCTGSVMENYKTRLNGI |
| | CT_6_Q393L | LTPIKGALEIYKQNTHDLVGDVRLAGVIMAGVAIGIATAAQI |
| | | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRNT |
| 442 | F3- | MKCLLYLAFLFIGVNCILHYEKLSKIGLVKGVTRKYKIKSNP |
| | _VSVG_signal | LTKDIVIKMIPNVSNMSQCTGSVMENYKTRLNGILTPIKGAL |
| | sequence_ | EIYKQNTHDLVGDVRLAGVIMAGVAIGIATAAQITAGVALY |
| | CT_6_Q393L | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSENNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RNT |
| 443 | F3_VGDVK | MHHLHPMIVCIFVMYTGIVGSDAILHYEKLSKIGLVKGVTR |
| | (HeV)_HPIV2_ | KYKIKSNPLTKDIVIKMIPNVSNMSQCTGSVMENYKTRLNGI |
| | signal_sequence | LTPIKGALEIYKQNTHDLVGDVKLAGVIMAGVAIGIATAAQI |
| | CT_6_Q393L | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRNT |
| 444 | F3_VGDVK | MKCLLYLAFLFIGVNCILHYEKLSKIGLVKGVTRKYKIKSNP |
| | (HeV)_VSVG_ | LTKDIVIKMIPNVSNMSQCTGSVMENYKTRLNGILTPIKGAL |
| | signal_sequence_ | EIYKQNTHDLVGDVKLAGVIMAGVAIGIATAAQITAGVALY |
| | CT_6_Q393L | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RNT |
| 445 | F3_NDVF_ | MVVILDKRCYCNLLILILMISECSVGILHYEKLSKIGLVKGVT |
| | CT_6_Q393L | RKYKIKSNPLTKDIVIKMIPNVSNMSQCTGSVMENYKTRLN |
| | | GILTPIKGALEIYKQNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVKQKAQQ |
| 446 | F3_VGDVK | MVVILDKRCYCNLLILILMISECSVGILHYEKLSKIGLVKGVT |
| | (HeV)_NDVF_ | RKYKIKSNPLTKDIVIKMIPNVSNMSQCTGSVMENYKTRLN |
| | CT_6_Q393L | GILTPIKGALEIYKQNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVKQKAQQ |
| 447 | F3_HPIV2_signal_ | MHHLHPMIVCIFVMYTGIVGSDAILHYEKLSKIGLVKGVTR |
| | sequence_NDVF_ | KYKIKSNPLTKDIVIKMIPNVSNMSQCTGSVMENYKTRLNGI |
| | CT_6_Q393L | LTPIKGALEIYKQNTHDLVGDVRLAGVIMAGVAIGIATAAQI |
| | | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVKQKAQQ |
| 448 | F3_VSVG_signal_ | MKCLLYLAFLFIGVNCILHYEKLSKIGLVKGVTRKYKIKSNP |
| | sequence_NDVF_ | LTKDIVIKMIPNVSNMSQCTGSVMENYKTRLNGILTPIKGAL |
| | CT_6_Q393L | EIYKQNTHDLVGDVRLAGVIMAGVAIGIATAAQITAGVALY |
| | | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVKQK |
| | | AQQ |
| 449 | F3_VGDVK | MHHLHPMIVCIFVMYTGIVGSDAILHYEKLSKIGLVKGVTR |
| | (HeV)_HPIV2_ | KYKIKSNPLTKDIVIKMIPNVSNMSQCTGSVMENYKTRLNGI |
| | signal_sequence_ | LTPIKGALEIYKQNTHDLVGDVKLAGVIMAGVAIGIATAAQI |
| | NDVF_CT_6_ | TAGVALYEAMKNADNINKLKSSIESTNEAVVKLQETAEKT |
| | Q393L | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVKQKAQQ |
| 450 | F3_VGDVK | MKCLLYLAFLFIGVNCILHYEKLSKIGLVKGVTRKYKIKSNP |
| | (HeV)_VSVG_ | LTKDIVIKMIPNVSNMSQCTGSVMENYKTRLNGILTPIKGAL |
| | signal_sequence_ | EIYKQNTHDLVGDVKLAGVIMAGVAIGIATAAQITAGVALY |
| | NDVF_CT_6_ | EAMKNADNINKLKSSIESTNEAVVKLQETAEKTVYVLTALQ |
| | Q393L | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSENNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVKQK |
| | | AQQ |
| 451 | F3_HeVF_CT_6_ | MVVILDKRCYCNLLILILMISECSVGILHYEKLSKIGLVKGVT |
| | Q393L | RKYKIKSNPLTKDIVIKMIPNVSNMSQCTGSVMENYKTRLN |
| | | GILTPIKGALEIYKQNTHDLVGDVRLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRGN |
| 452 | F3_VGDVK | MVVILDKRCYCNLLILILMISECSVGILHYEKLSKIGLVKGVT |
| | (HeV)_HeVF_ | RKYKIKSNPLTKDIVIKMIPNVSNMSQCTGSVMENYKTRLN |
| | CT_6_Q393L | GILTPIKGALEIYKQNTHDLVGDVKLAGVIMAGVAIGIATAA |
| | | QITAGVALYEAMKNADNINKLKSSIESTNEAVVKLQETAEK |
| | | TVYVLTALQDYINTNLVPTIDKISCKQTELSLDLALSKYLSD |
| | | LLFVFGPNLQDPVSNSMTIQAISQAFGGNYETLLRTLGYATE |
| | | DFDDLLESDSITGQIIYVDLSSYYIIVRVYFPILTEIQQAYIQEL |
| | | LPVSFNNDNSEWISIVPNFILVRNTLISNIEIGFCLITKRSVICN |
| | | QDYATPMTNNMRECLTGSTEKCPRELVVSSHVPRFALSNG |
| | | VLFANCISVTCLCQTTGRAISQSGEQTLLMIDNTTCPTAVLG |
| | | NVIISLGKYLGSVNYNSEGIAIGPPVFTDKVDISSQISSMNQS |
| | | LQQSKDYIKEAQRLLDTVNPSLISMLSMIILYVLSIASLCIGLI |
| | | TFISFIIVEKKRGN |
| 453 | F3_HPIV2_signal | MHHLHPMIVCIFVMYTGIVGSDAILHYEKLSKIGLVKGVTR |
| | sequence_HeVF_ | KYKIKSNPLTKDIVIKMIPNVSNMSQCTGSVMENYKTRLNGI |
| | CT_6_Q393L | LTPIKGALEIYKQNTHDLVGDVRLAGVIMAGVAIGIATAAQI |
| | | TAGVALYEAMKNADNINKLKSSIESTNEAVVKLQETAEKT |
| | | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRGN |
| 454 | F3- | MKCLLYLAFLFIGVNCILHYEKLSKIGLVKGVTRKYKIKSNP |
| | _VSVG_signal_ | LTKDIVIKMIPNVSNMSQCTGSVMENYKTRLNGILTPIKGAL |
| | sequence_HeVF_ | EIYKQNTHDLVGDVRLAGVIMAGVAIGIATAAQITAGVALY |
| | CT_6_Q393L | EAMKNADNINKLKSSIESTNEAVVKLQETAEKTVYVLTALQ |
| | | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSENNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RGN |
| 455 | F3_VGDVK | MHHLHPMIVCIFVMYTGIVGSDAILHYEKLSKIGLVKGVTR |
| | (HeV)_HPIV2_ | KYKIKSNPLTKDIVIKMIPNVSNMSQCTGSVMENYKTRLNGI |
| | signal_sequence_ | LTPIKGALEIYKQNTHDLVGDVKLAGVIMAGVAIGIATAAQI |
| | HeVF_CT_6_ | TAGVALYEAMKNADNINKLKSSIESTNEAVVKLQETAEKT |
| | Q393L | VYVLTALQDYINTNLVPTIDKISCKQTELSLDLALSKYLSDL |
| | | LFVFGPNLQDPVSNSMTIQAISQAFGGNYETLLRTLGYATED |
| | | FDDLLESDSITGQIIYVDLSSYYIIVRVYFPILTEIQQAYIQELL |
| | | PVSFNNDNSEWISIVPNFILVRNTLISNIEIGFCLITKRSVICNQ |
| | | DYATPMTNNMRECLTGSTEKCPRELVVSSHVPRFALSNGV |
| | | LFANCISVTCLCQTTGRAISQSGEQTLLMIDNTTCPTAVLGN |
| | | VIISLGKYLGSVNYNSEGIAIGPPVFTDKVDISSQISSMNQSL |
| | | QQSKDYIKEAQRLLDTVNPSLISMLSMIILYVLSIASLCIGLIT |
| | | FISFIIVEKKRGN |
| 456 | F3_VGDVK | MKCLLYLAFLFIGVNCILHYEKLSKIGLVKGVTRKYKIKSNP |
| | (HeV)_VSVG_ | LTKDIVIKMIPNVSNMSQCTGSVMENYKTRLNGILTPIKGAL |
| | signal_sequence_ | EIYKQNTHDLVGDVKLAGVIMAGVAIGIATAAQITAGVALY |
| | HeVF_CT_6_ | EAMKNADNINKLKSSIESTNEAVVKLQETAEKTVYVLTALQ |
| | Q393L_ | DYINTNLVPTIDKISCKQTELSLDLALSKYLSDLLFVFGPNLQ |
| | | DPVSNSMTIQAISQAFGGNYETLLRTLGYATEDFDDLLESDS |
| | | ITGQIIYVDLSSYYIIVRVYFPILTEIQQAYIQELLPVSFNNDNS |
| | | EWISIVPNFILVRNTLISNIEIGFCLITKRSVICNQDYATPMTN |
| | | NMRECLTGSTEKCPRELVVSSHVPRFALSNGVLFANCISVT |
| | | CLCQTTGRAISQSGEQTLLMIDNTTCPTAVLGNVIISLGKYL |
| | | GSVNYNSEGIAIGPPVFTDKVDISSQISSMNQSLQQSKDYIKE |
| | | AQRLLDTVNPSLISMLSMIILYVLSIASLCIGLITFISFIIVEKK |
| | | RGN |

### Example 2 Generation of NiV-F Pseudotyped Lentiviral Vectors with a Re-Targeted NiV-G

The variant NiV-F constructs described above were used to pseudotype lentiviral particles along with a re-targeted NiV-G construct that was formatted as a retargeted fusogen containing a binder sequence against a cell surface protein (e.g. scFv against CD8). The exemplary binder sequence was codon optimized and was then subcloned into a pCAGGS vector for fusion of the binding modality to the C-terminus of NiV-G sequence (SEQ ID NO 301).

After subcloning, the re-targeted NiV-G protein construct was transfected into Lenti-X 293 (HEK 293) cells, along with a vector containing a nucleotide sequence encoding one of the exemplary variant NiV-F sequences described in Example 1 above, a packaging plasmid for vector production containing an empty backbone, an HIV-1 pol, HIV-1 gag, HIV-1 Rev, HIV-1 Tat, an AmpR promoter and an SV40 promoter, and a lentiviral vector containing an enhanced green fluorescent protein (eGFP) under the control of a SFFV promoter pLenti-SFFV-eGFP.

Adherent Lenti-X 293 cells were seeded into 24 well plates in 0.5 mL growth media at a density of roughly 0.8×10⁵ cell/mL. 18-24 hours post plating, cells were transfected using polyethlenimine (PET) TransIT®-293 Transfection Reagent (Mirus Bio, Madison, WI USA) according to manufacturer protocol. Briefly, transfection reagent/DNA complexes were prepared by mixing a 3:1 ratio of TransIT®-293 Transfection Reagent:purified plasmid DNA and incubating at room temperature. The transfection reagent/DNA complexes were aliquoted in a single transfer step to the seeded Lenti-X293 cells using ViaFlo equipment (Integra, Konstanz, Germany). 24-72 hours post transfection, crude lentivirus was harvested.

Transduction efficiency of the produced pseudotyped lentivirus preparations for recipient target cells was assessed by monitoring GFP expression by flow cytometry. Transduction efficiency was compared to reference lentivirus pseudotyped as described above but with a NiV-F sequence encoding a NiVF Δ22 (SEQ ID NO:302; Bender et al. 2016 PLoS Pathol 12(6):e1005641).

Crude NiV-G lentivirus (LV) preparations, each pseudotyped with one of the variant NiV-F constructs and a retargeted NiV-G against CD8, were transduced into recipient SupT1 cells (ATCC No. CRL-1942; a human T cell lymphoblast cell line that is positive for both CD4 and CD8, CD4+CD8+) by single point dilutions followed by analysis of transduced cells for GFP expression by flow cytometry. Specific titer was determined by % of cells that were GFP positive (GFP+). In some cases, multiple point dilutions were titered and flow cytometry was carried out on those showing about 20-30% GFP+ cells for the control condition.

Exemplary results are depicted below in Table E11 (Cytoplasmic tail modifications, 0.02 LV dilution) and Table E12 (extracellular or transmembrane domain modifications, 0.2 LV dilution) following transduction of recipient cells with LV pseudotyped with representative variant NiV-F cytoplasmic tail constructs. As shown, it was observed that many of the exemplary variant NiV-F constructs containing a modified cytoplasmic tail resulted in increased titers as determined by GFP+ cells, as compared to lentiviruses pseudotyped with the retargeted control NiV-G construct.

TABLE E11
Cytoplasmic tail modifications

| | Average % GFP | |
|---|---|---|
| | Positive Cells (at | Standard |
| SEQ ID NO | 0.02 LV dilution) | Deviation |
| SEQ ID NO 27 | 35.35938 | 4.211251 |

Hyperfusogenic Mutations

| | | |
|---|---|---|
| SEQ ID NO 32 | 20.75 | 0.353553 |
| SEQ ID NO 33 | 16.5 | 0.282843 |
| SEQ ID NO 34 | 0.7 | 0 |
| SEQ ID NO 53 | 10.05 | 3.416138 |
| SEQ ID NO 54 | 0.275 | 0.095743 |
| SEQ ID NO 55 | 0.5 | 0.216025 |
| SEQ ID NO 56 | 0.5 | 0.141421 |

NiV-F Truncations

| | | |
|---|---|---|
| SEQ ID NO 4 | 0.575 | 0.492443 |
| SEQ ID NO 5 | 1.2 | 0 |
| SEQ ID NO 6 | 1.2 | 0.141421 |
| SEQ ID NO 7 | 0.35 | 0.129099 |
| SEQ ID NO 8 | 3.05 | 0.777817 |
| SEQ ID NO 10 | 2.1 | 0.141421 |
| SEQ ID NO 11 | 2.5 | 0.565685 |
| SEQ ID NO 12 | 1.2 | 0.469042 |
| SEQ ID NO 13 | 2.35 | 0.353553 |
| SEQ ID NO 14 | 2.65 | 0.070711 |
| SEQ ID NO 15 | 2.2 | 0.282843 |
| SEQ ID NO 16 | 2.4 | 0 |
| SEQ ID NO 17 | 2.8 | 2.124461 |
| SEQ ID NO 18 | 1.85 | 1.609348 |
| SEQ ID NO 19 | 2.325 | 1.8246 |
| SEQ ID NO 20 | 2.55 | 1.443376 |
| SEQ ID NO 21 | 2.075 | 1.466004 |
| SEQ ID NO 22 | 0.625 | 0.189297 |
| SEQ ID NO 23 | 25.15 | 3.960219 |
| SEQ ID NO 24 | 24 | 5.238957 |
| SEQ ID NO 25 | 9.875 | 3.637192 |
| SEQ ID NO 26 | 22.775 | 8.461826 |
| SEQ ID NO 28 | 8.925 | 1.86257 |
| SEQ ID NO 29 | 3.65 | 2.456963 |
| SEQ ID NO 30 | 16 | 5.621981 |
| SEQ ID NO 31 | 21.7 | 2.105548 |
| SEQ ID NO 32 | 20.75 | 0.353553 |

HeV-F Truncations

| | | |
|---|---|---|
| SEQ ID NO 58 | 0.9 | 0.282843 |
| SEQ ID NO 59 | n/a | n/a |
| SEQ ID NO 60 | n/a | n/a |
| SEQ ID NO 61 | 0.45 | 0.070711 |
| SEQ ID NO 62 | 3.15 | 1.343503 |
| SEQ ID NO 63 | 1.55 | 0.212132 |
| SEQ ID NO 64 | 0.3 | 0 |
| SEQ ID NO 65 | 0.75 | 0.212132 |
| SEQ ID NO 66 | 0.85 | 0.212132 |
| SEQ ID NO 67 | 0.8 | 0.282843 |
| SEQ ID NO 68 | 0.1 | 0 |
| SEQ ID NO 69 | 1.15 | 0.070711 |
| SEQ ID NO 74 | 3.5 | 3.236253 |
| SEQ ID NO 75 | 1.125 | 0.403113 |
| SEQ ID NO 76 | 0.525 | 0.170783 |
| SEQ ID NO 77 | 26.35 | 7.808756 |
| SEQ ID NO 78 | 27.45 | 7.105632 |
| SEQ ID NO 79 | 10.7 | 4.75114 |
| SEQ ID NO 80 | 30.475 | 5.416872 |
| SEQ ID NO 81 | 19.6 | 6.925797 |

MeV-F Truncations

| | | |
|---|---|---|
| SEQ ID NO 125 | 9.7 | 0.424264 |
| SEQ ID NO 126 | 57.425 | 9.033041 |
| SEQ ID NO 127 | 58.525 | 7.647821 |
| SEQ ID NO 128 | 58.525 | 7.431633 |
| SEQ ID NO 129 | 61.425 | 8.137721 |
| SEQ ID NO 130 | 59.7 | 9.007404 |
| SEQ ID NO 131 | 56.5 | 7.107273 |
| SEQ ID NO 132 | 51.5 | 9.920685 |
| SEQ ID NO 133 | 47.525 | 11.20844 |
| SEQ ID NO 134 | 26.175 | 7.205727 |
| SEQ ID NO 135 | 9.2 | 4.33359 |
| SEQ ID NO 136 | 6.35 | 2.441994 |
| SEQ ID NO 138 | 17.15 | 1.343503 |
| SEQ ID NO 139 | 18.8 | 2.828427 |
| SEQ ID NO 140 | 17.65 | 2.616295 |

CeV-F Truncations

| | | |
|---|---|---|
| SEQ ID NO 96 | 0.65 | 0.353553 |
| SEQ ID NO 97 | 0.375 | 0.275379 |
| SEQ ID NO 98 | 1.75 | 1.744515 |
| SEQ ID NO 99 | 0 | 0 |
| SEQ ID NO 100 | 1.925 | 1.906786 |
| SEQ ID NO 101 | 1.5 | 1.462874 |
| SEQ ID NO 102 | 0.55 | 0.369685 |
| SEQ ID NO 103 | 4.35 | 2.564501 |
| SEQ ID NO 104 | 5.5 | 2.586503 |
| SEQ ID NO 105 | 1.425 | 1.364734 |
| SEQ ID NO 106 | 12.7 | 7.748118 |
| SEQ ID NO 107 | 16 | 6.837641 |
| SEQ ID NO 108 | 28.95 | 4.030509 |
| SEQ ID NO 109 | 40.65 | 3.040559 |
| SEQ ID NO 110 | 31.65 | 4.879037 |

NDV-F Truncations

| | | |
|---|---|---|
| SEQ ID NO 141 | 0.15 | 0.070711 |
| SEQ ID NO 142 | 3.525 | 2.183842 |
| SEQ ID NO 143 | 0.6 | 0.355903 |
| SEQ ID NO 144 | 1.8 | 1.134313 |
| SEQ ID NO 145 | 2.225 | 1.034005 |
| SEQ ID NO 146 | 10 | 3.580503 |
| SEQ ID NO 147 | 59.675 | 6.053856 |
| SEQ ID NO 148 | 62.2 | 6.678822 |
| SEQ ID NO 149 | 63.05 | 9.081299 |
| SEQ ID NO 150 | 64.2 | 9.49807 |
| SEQ ID NO 151 | 50.25 | 10.21943 |
| SEQ ID NO 152 | 11.475 | 2.027108 |
| SEQ ID NO 153 | 12.95 | 0.070711 |
| SEQ ID NO 154 | 21.1 | 1.272792 |
| SEQ ID NO 155 | 16.55 | 4.737615 |

HPIV-2-F Truncations

| | | |
|---|---|---|
| SEQ ID NO 111 | 0 | 0 |
| SEQ ID NO 112 | 0 | 0 |
| SEQ ID NO 113 | 0 | 0 |
| SEQ ID NO 114 | 0 | 0 |
| SEQ ID NO 115 | 0 | 0 |
| SEQ ID NO 116 | 0 | 0 |
| SEQ ID NO 117 | 0 | 0 |
| SEQ ID NO 118 | 0.55 | 0.070711 |
| SEQ ID NO 119 | 0.5 | 0 |
| SEQ ID NO 120 | 0.2 | 0 |
| SEQ ID NO 121 | 2.25 | 0.070711 |
| SEQ ID NO 122 | 2.05 | 0.212132 |
| SEQ ID NO 123 | 10.15 | 1.202082 |
| SEQ ID NO 124 | 11.15 | 2.616295 |

Sendai-F Truncations

| | | |
|---|---|---|
| SEQ ID NO 156 | 0 | 0 |
| SEQ ID NO 157 | 13.9 | 0.848528 |
| SEQ ID NO 158 | 9.25 | 0.212132 |
| SEQ ID NO 159 | 5.9 | 0.565685 |
| SEQ ID NO 160 | 2.75 | 0.212132 |
| SEQ ID NO 161 | 3.8 | 0.141421 |
| SEQ ID NO 162 | 2.85 | 0.070711 |
| SEQ ID NO 163 | 3.75 | 0.070711 |
| SEQ ID NO 164 | 9.75 | 3.606245 |
| SEQ ID NO 165 | 11.15 | 0.353553 |
| SEQ ID NO 166 | 12.65 | 0.212132 |
| SEQ ID NO 167 | 5.15 | 1.767767 |
| SEQ ID NO 168 | 3.6 | 0.282843 |
| SEQ ID NO 169 | 6.7 | 0.707107 |

Bat PV-F Truncations

| | | |
|---|---|---|
| SEQ ID NO 82 | 1.9 | 0 |
| SEQ ID NO 83 | 5.2 | 2.404163 |
| SEQ ID NO 84 | 1.7 | 0 |
| SEQ ID NO 85 | 3.65 | 0.636396 |
| SEQ ID NO 86 | 2.85 | 0.070711 |
| SEQ ID NO 87 | 0.95 | 0.070711 |
| SEQ ID NO 88 | 2.1 | 0 |
| SEQ ID NO 89 | 10.05 | 4.313351 |
| SEQ ID NO 90 | 16.35 | 2.757716 |
| SEQ ID NO 91 | 7 | 0.424264 |
| SEQ ID NO 92 | 28.05 | 9.828784 |
| SEQ ID NO 93 | 7.65 | 1.06066 |
| SEQ ID NO 94 | 6.3 | 1.272792 |
| SEQ ID NO 95 | 11.35 | 1.626346 |

Paramyxo-Attachment Proteins

| | | |
|---|---|---|
| SEQ ID NO 210 | 64.5 | 15.69777 |
| SEQ ID NO 211 | 49.85 | 6.29325 |
| SEQ ID NO 212 | 0.65 | 0.070711 |
| SEQ ID NO 213 | 21.6 | 3.676955 |
| SEQ ID NO 214 | 13.7 | 2.969848 |
| SEQ ID NO 215 | 9.4 | 0.707107 |
| SEQ ID NO 216 | 0.35 | 0.070711 |
| SEQ ID NO 217 | 20.65 | 2.192031 |
| SEQ ID NO 218 | 12.4 | 0 |
| SEQ ID NO 219 | 9.15 | 2.05061 |
| SEQ ID NO 220 | 2.8 | 0.282843 |
| SEQ ID NO 221 | 1.3 | 0.141421 |
| SEQ ID NO 222 | 0 | 0 |

Viral Glycoprotein CT Swaps

| | | |
|---|---|---|
| SEQ ID NO 170 | 0.35 | 0.070711 |
| SEQ ID NO 171 | 0 | 0 |
| SEQ ID NO 172 | 0 | 0 |
| SEQ ID NO 173 | 0 | 0 |
| SEQ ID NO 174 | 0 | 0 |
| SEQ ID NO 175 | 0 | 0 |
| SEQ ID NO 176 | 0 | 0 |
| SEQ ID NO 177 | 0 | 0 |
| SEQ ID NO 178 | 0 | 0 |
| SEQ ID NO 179 | 0 | 0 |
| SEQ ID NO 202 | 0 | 0 |
| SEQ ID NO 203 | 20.45 | 1.06066 |
| SEQ ID NO 204 | 0 | 0 |
| SEQ ID NO 205 | 1.4 | 0.141421 |
| SEQ ID NO 206 | 0.05 | 0.070711 |
| SEQ ID NO 207 | 0 | 0 |
| SEQ ID NO 208 | 0 | 0 |
| SEQ ID NO 209 | 0.75 | 0.070711 |

Retroviral Associate Protein CT Swaps

| | | |
|---|---|---|
| SEQ ID NO 194 | 0 | 0 |
| SEQ ID NO 195 | 0.05 | 0.070711 |
| SEQ ID NO 196 | 0.95 | 0.212132 |
| SEQ ID NO 197 | 7.45 | 0.636396 |
| SEQ ID NO 198 | 0 | 0 |
| SEQ ID NO 199 | 15 | 1.272792 |
| SEQ ID NO 200 | 28.4 | 3.818377 |
| SEQ ID NO 201 | 21.1 | 4.666905 |

HIV-1 gp41 CT domains

| | | |
|---|---|---|
| SEQ ID NO 180 | 0 | 0 |
| SEQ ID NO 185 | 0 | 0 |
| SEQ ID NO 186 | 0 | 0 |
| SEQ ID NO 187 | 0 | 0 |
| SEQ ID NO 188 | 0 | 0 |
| SEQ ID NO 189 | 0 | 0 |
| SEQ ID NO 190 | 0 | 0 |
| SEQ ID NO 191 | 0 | 0 |
| SEQ ID NO 192 | 0 | 0 |
| SEQ ID NO 193 | 0 | 0 |
| SEQ ID NO 286 | 0.25 | 0.070711 |

Endocytosis (YSRL) variants

| | | |
|---|---|---|
| SEQ ID NO 35 | 7.225 | 2.045116 |
| SEQ ID NO 36 | 5.675 | 3.540598 |
| SEQ ID NO 37 | 5.95 | 2.29855 |
| SEQ ID NO 38 | 45.5 | 14.20868 |
| SEQ ID NO 39 | 5.175 | 1.687947 |
| SEQ ID NO 40 | 10.225 | 6.693965 |
| SEQ ID NO 41 | 23.5 | 7.544093 |
| SEQ ID NO 42 | 18.525 | 5.309347 |
| SEQ ID NO 43 | 13.725 | 5.145467 |
| SEQ ID NO 44 | 5.225 | 0.960469 |
| SEQ ID NO 45 | 6.85 | 1.953629 |
| SEQ ID NO 46 | 7.35 | 1.652271 |
| SEQ ID NO 48 | 31.675 | 12.16535 |
| SEQ ID NO 49 | 3.225 | 2.311385 |
| SEQ ID NO 50 | 18.25 | 6.324819 |
| SEQ ID NO 47 | 15.05 | 1.909188 |
| SEQ ID NO 51 | 17.6 | 1.414214 |

TABLE E12
Extracellular or transmembrane domain modifications

| | Average % | |
|---|---|---|
| | GFP Positive | |
| | Cells (at 0.02 | Standard |
| SEQ ID NO | LV dilution) | Deviation |
| SEQ ID NO 27 | 35.35938 | 4.211251 |

N-glycan Mutants

| | | |
|---|---|---|
| SEQ ID NO 273 | 33.875 | 8.974919 |
| SEQ ID NO 274 | 49.35 | 5.303301 |
| SEQ ID NO 275 | 0 | 0 |
| SEQ ID NO 276 | 0 | 0 |
| SEQ ID NO 278 | 16.375 | 10.42221 |
| SEQ ID NO 279 | 34 | 9.701546 |
| SEQ ID NO 280 | 8.05 | 2.369951 |
| SEQ ID NO 290 | 1.35 | 0.602771 |

Hexamer Stabilizing Mutants

| | | |
|---|---|---|
| SEQ ID NO 291 | 9.4 | 3.073543 |
| SEQ ID NO 292 | 33.6 | 10.27975 |
| SEQ ID NO 293 | 13.05 | 10.91253 |

Hendra F Swap

| | | |
|---|---|---|
| SEQ ID NO 294 | 0 | 0 |
| SEQ ID NO 295 | 0 | 0 |
| SEQ ID NO 296 | 0 | 0 |
| SEQ ID NO 281 | 0 | 0 |
| SEQ ID NO 282 | 0.05 | 0.070711 |
| SEQ ID NO 283 | 0 | 0 |

Protease Site Mutations

| | | |
|---|---|---|
| SEQ ID NO 224 | 0 | 0 |
| SEQ ID NO 225 | 26.45 | 1.626346 |
| SEQ ID NO 226 | 13.95 | 1.202082 |
| SEQ ID NO 227 | 16.1 | 1.979899 |
| SEQ ID NO 228 | 17.7 | 0.424264 |
| SEQ ID NO 229 | 0 | 0 |
| SEQ ID NO 230 | 1.15 | 0.070711 |
| SEQ ID NO 231 | 0.6 | 0 |
| SEQ ID NO 232 | 0.7 | 0.141421 |
| SEQ ID NO 233 | 0.5 | 0.282843 |
| SEQ ID NO 234 | 0 | 0 |
| SEQ ID NO 235 | 3.3 | 1.697056 |
| SEQ ID NO 236 | 1.25 | 0.353553 |
| SEQ ID NO 237 | 1.6 | 0.565685 |
| SEQ ID NO 238 | 1.1 | 0 |
| SEQ ID NO 239 | 52.4 | 13.85929 |
| SEQ ID NO 240 | 4.3 | 0.848528 |
| SEQ ID NO 241 | 0.4 | 0.282843 |
| SEQ ID NO 242 | 3.95 | 0.919239 |
| SEQ ID NO 243 | 0.2 | 0.141421 |
| SEQ ID NO 244 | 5.15 | 0.353553 |
| SEQ ID NO 245 | 1.25 | 0.353553 |
| SEQ ID NO 246 | 0 | 0 |
| SEQ ID NO 247 | 0 | 0 |
| SEQ ID NO 248 | 0.25 | 0.070711 |

Signal Sequence Mutants

| | | |
|---|---|---|
| SEQ ID NO 249 | 36.4 | 7.353911 |
| SEQ ID NO 250 | 30.15 | 3.181981 |
| SEQ ID NO 251 | 25.3 | 5.798276 |
| SEQ ID NO252 | 0 | 0 |
| SEQ ID NO 253 | 0 | 0 |
| SEQ ID NO 254 | 0.1 | 0 |
| SEQ ID NO 255 | 4.05 | 0.070711 |
| SEQ ID NO 256 | 0 | 0 |
| SEQ ID NO 257 | 8.95 | 0.212132 |
| SEQ ID NO 258 | 0 | 0 |
| SEQ ID NO 259 | 0 | 0 |
| SEQ ID NO 260 | 0 | 0 |
| SEQ ID NO 261 | 40.05 | 8.555992 |
| SEQ ID NO 262 | 32.2 | 6.646804 |
| SEQ ID NO 263 | 21.8 | 4.384062 |
| SEQ ID NO 264 | 31.3 | 1.838478 |
| SEQ ID NO 265 | 0 | 0 |
| SEQ ID NO 266 | 0 | 0 |
| SEQ ID NO 267 | 21.95 | 3.040559 |

Transmembrane Domain Mutants

| | | |
|---|---|---|
| SEQ ID NO 268 | 0.7 | 0.141421 |
| SEQ ID NO 269 | 0 | 0 |
| SEQ ID NO 270 | 36.95 | 5.020458 |
| SEQ ID NO 271 | 29.5 | 9.899495 |
| SEQ ID NO 272 | 30.55 | 3.040559 |

Combinatorial Variants

| | | |
|---|---|---|
| SEQ ID NO 56 | 0.05 | 0.070711 |
| SEQ ID NO 57 | 0 | 0 |
| SEQ ID NO 297 | 12.7 | 3.394113 |
| SEQ ID NO 298 | 5.4 | 0.707107 |
| SEQ ID NO 299 | 8.5 | 1.414214 |
| SEQ ID NO 300 | 0.05 | 0.070711 |

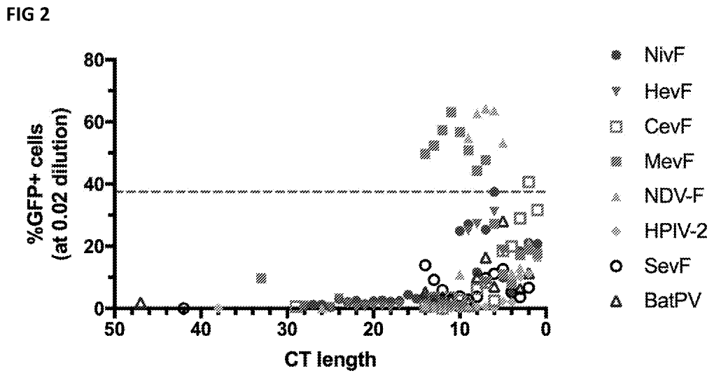

For example, cells transduced with lentivirus pseudotyped with chimeric NiV-F constructs containing exemplary truncated cytoplasmic tail derived from a another Paramyxovirus, such as Measles, resulted in an increase in titer for NiV-CD8^{scFv} affinity binders when compared to exemplary constructs containing a NiV-F truncated tail on human T cells (FIG. 2). Thus, the results indicate that some cytoplasmic tailed modifications may more universally increase titer, while others may be binder/fusogen specific.

Together, the results demonstrated that lentivirus preparations pseudotyped with certain variant NiV-F constructs increased titer in recipient cells.

Modified NiV-F cytoplasmic tail constructs that improved titer compared to the control NiV-F are set forth in Table E13.

TABLE E13
Representative NiV-F constructs that improved titer

| | SEQ ID NO |
|---|---|
| | NiV-G |
| Name/Description | construct |
| NivF_CT-5 | 306 |
| MeaslesF_CT_7 | 307 |
| NDVF_CT_9 | 308 |
| NDVF_CT_6 | 309 |
| HeVF_CT_6 | 310 |
| Nipah_virus | 210 |
| NivF_hyperfusogenic_mutations_Nglycan (F5) | 274 |
| NivF_hyperfusogenic_mutations_Hexamer- | 292 |
| stabl(Q393L) | |
| NivF_protease_site_mod_Other-paramyxo | 239 |
| HPIV2_signal_sequence | 251 |
| VSVG_signal_sequence | 261 |
| F5_VGDVK | 369 |
| F5_Q393L_VSVG_signal_sequence | 372 |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

VIII. SEQUENCES

| SEQ ID | | |
|---|---|---|
| NO | SEQUENCE | DESCRIPTION |
| 1 | MVVILDKRCYCNLLILILMISECSVGILHYEKLSKIGLVKGV | Uniprot Q9IH63 |
| | TRKYKIKSNPLTKDIVIKMIPNVSNMSQCTGSVMENYKTRL | with signal |
| | NGILTPIKGALEIYKNNTHDLVGDVRLAGVIMAGVAIGIAT | sequence |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNTYSRLEDRRVRPTSSGDLYY | |
| | IGT | |
| 2 | ILHYEKLSKIGLVKGVTRKYKIKSNPLTKDIVIKMIPNVSNM | NiV-F backbone |
| | SQCTGSVMENYKTRLNGILTPIKGALEIYKNNTHDLVGDV | ΔCT |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIV | |
| 3 | MVVILDKRCYCNLLILILMISECSVG | Signal Peptide |
| 4 | EKKRNTYSRLEDRRVRPTSSGDLYYIGT | NivF_CT-28 (full- |
| | | length) |
| 5 | EKKRNTYSRLEDRRVRPTSSGDLYYIG | NivF_CT-27 |
| 6 | EKKRNTYSRLEDRRVRPTSSGDLYYI | NivF_CT-26 |
| 7 | EKKRNTYSRLEDRRVRPTSSGDLYY | NivF_CT-25 |
| 8 | EKKRNTYSRLEDRRVRPTSSGDLY | NivF_CT-24 |
| 9 | EKKRNTYSRLEDRRVRPTSSGDL | NivF_CT-23 |
| 10 | EKKRNTYSRLEDRRVRPTSSGD | NivF_CT-22 |
| 11 | EKKRNTYSRLEDRRVRPTSSG | NivF_CT-21 |
| 12 | EKKRNTYSRLEDRRVRPTSS | NivF_CT-20 |
| 13 | EKKRNTYSRLEDRRVRPTS | NivF_CT-19 |
| 14 | EKKRNTYSRLEDRRVRPT | NivF_CT-18 |
| 15 | EKKRNTYSRLEDRRVRP | NivF_CT-17 |
| 16 | EKKRNTYSRLEDRRVR | NivF_CT-16 |
| 17 | EKKRNTYSRLEDRRV | NivF_CT-15 |
| 18 | EKKRNTYSRLEDRR | NivF_CT-14 |
| 19 | EKKRNTYSRLEDR | NivF_CT-13 |
| 20 | EKKRNTYSRLED | NivF_CT-12 |
| 21 | EKKRNTYSRLE | NivF_CT-11 |
| 22 | EKKRNTYSRL | NivF_CT-10 |
| 23 | EKKRNTYSR | NivF_CT-9 |
| 24 | EKKRNTYS | NivF_CT-8 |
| 25 | EKKRNTY | NivF_CT-7 |
| 26 | EKKRNT | NivF_CT-06 |
| 27 | EKKRN | NivF_CT-5 |
| 28 | EKKR | NivF_CT-4 |
| 29 | EKK | NivF_CT-3 |
| 30 | EK | NivF_CT-2 |
| 31 | E | NivF_CT-1 |
| 32 | EYSRLEDRRVRPTSSGDLYYIGT | YSRL |
| 33 | EKKRNTEDRRVRPTSSGDLYYIGT | YSRL |
| 34 | EKKRNTYSRLRVRPTSSGDLYYIGT | YSRL |
| 35 | EKYSRL | YSRL |
| 36 | EKKYSRL | YSRL |
| 37 | EKKRNTDTYRYI | NIvF_CT-6_AU1 |
| 38 | EKKRNTYPYDVPDYA | NIvF_CT-6_HA |
| 39 | EKDKQTLL | DKQTLL |
| 40 | EDKQTLL | DKQTLL |
| 41 | EKKRNTDKQTLL | DKQTLL |
| 42 | EKKRNTDKQTLLEDRRVRPTSSGDLYYIGT | DKQTLL |
| 43 | EKNPVY | NPVY |
| 44 | EKFDNPVY | FDNPVY |
| 45 | EKKRNTNPVY | NPVY |
| 46 | EKKRNTFDNPVY | FDNPVY |
| 47 | EKKRNTNPVYEDRRVRPTSSGDLYYIGT | NPVY |
| 48 | EKKRNTFDNPVYEDRRVRPTSSGDLYYIGT | FDNPVY |
| 49 | EKYFIPIN | YFIPIN |
| 50 | EKKRNTYFIPIN | YFIPIN |
| 51 | EKKRNTYFIPINEDRRVRPTSSGDLYYIGT | YFIPIN |
| 52 | EKKRGNYSRLDDRQVRPVSNGDLYYIGT | HeVF_CT_28_FL |
| 53 | EAKRNT | CT-06_K1A |
| 54 | EAYSRL | CT- |
| | | 02_YSRL_K1A |
| 55 | EAKRNTYSRLEDRRVRPTSSGDLYYIGT | CT-28_K1A |
| 56 | EAKRNTYSRL | CT-10_K1A |
| 57 | MVVILDKRCYCNLLILILMISECSVGILHYEKLSKIGLVKGV | F3F5_Q393L_CT- |
| | TRKYKIKSNPLTKDIVIKMIPNVSNMSQCTGSVMENYKTRL | 10_K1A |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVEAKRNTYSRL | |
| 58 | MVVILDKRCYCNLLILILMISECSVGILHYEKLSKIGLVKGV | F3F5_Q393L_CT- |
| | TRKYKIKSNPLTKDIVIKMIPNVSNMSQCTGSVMENYKTRL | 06_K1A |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVEAKRNT | |
| 59 | EKKRGNYSRLDDRQVRPVSNGDLYYIG | HeVF_CT_27 |
| 60 | EKKRGNYSRLDDRQVRPVSNGDLYYI | HeVF_CT_26 |
| 61 | EKKRGNYSRLDDRQVRPVSNGDLYY | HeVF_CT_25 |
| 62 | EKKRGNYSRLDDRQVRPVSNGDLY | HeVF_CT_24 |
| 63 | EKKRGNYSRLDDRQVRPVSNGDL | HeVF_CT_23 |
| 64 | EKKRGNYSRLDDRQVRPVSNGD | HeVF_CT_22 |
| 65 | EKKRGNYSRLDDRQVRPVSNG | HeVF_CT_21 |
| 66 | EKKRGNYSRLDDRQVRPVSN | HeVF_CT_20 |
| 67 | EKKRGNYSRLDDRQVRPVS | HeVF_CT_19 |
| 68 | EKKRGNYSRLDDRQVRPV | HeVF_CT_18 |
| 69 | EKKRGNYSRLDDRQVRP | HeVF_CT_17 |
| 70 | EKKRGNYSRLDDRQVR | HeVF_CT_16 |
| 71 | EKKRGNYSRLDDRQV | HeVF_CT_15 |
| 72 | EKKRGNYSRLDDRQ | HeVF_CT_14 |
| 73 | EKKRGNYSRLDDR | HeVF_CT_13 |
| 74 | EKKRGNYSRLDD | HeVF_CT_12 |
| 75 | EKKRGNYSRLD | HeVF_CT_11 |
| 76 | EKKRGNYSRL | HeVF_CT_10 |
| 77 | EKKRGNYSR | HeVF_CT_9 |
| 78 | EKKRGNYS | HeVF_CT_8 |
| 79 | EKKRGNY | HeVF_CT_7 |
| 80 | EKKRGN | HeVF_CT_6 |
| 81 | EKKRG | HeVF_CT_5 |
| 82 | RRYNKYTPLINSDPSSRRSTIQDVYIIPNPGEHSIRSAARSID | BatPVF_CT_47_ |
| | RDRD | FL |
| 83 | RRYNKYTPLINSDP | BatPVF_CT_14 |
| 84 | RRYNKYTPLINSD | BatPVF_CT_13 |
| 85 | RRYNKYTPLINS | BatPVF_CT_12 |
| 86 | RRYNKYTPLIN | BatPVF_CT_11 |
| 87 | RRYNKYTPLI | BatPVF_CT_10 |
| 88 | RRYNKYTPL | BatPVF_CT_9 |
| 89 | RRYNKYTP | BatPVF_CT_8 |
| 90 | RRYNKYT | BatPVF_CT_7 |
| 91 | RRYNKY | BatPVF_CT_6 |
| 92 | RRYNK | BatPVF_CT_5 |
| 93 | RRYN | BatPVF_CT_4 |
| 94 | RRY | BatPVF_CT_3 |
| 95 | RR | BatPVF_CT_2 |
| 96 | DDPDYYNDYKRERINGKASKSNNIYYVGD | CeVF_CT_29_FL |
| 97 | DDPDYYNDYKRERI | CeVF_CT_14 |
| 98 | DDPDYYNDYKRER | CeVF_CT_13 |
| 99 | DDPDYYNDYKRE | CeVF_CT_12 |
| 100 | DDPDYYNDYKR | CeVF_CT_11 |
| 101 | DDPDYYNDYK | CeVF_CT_10 |
| 102 | DDPDYYNDY | CeVF_CT_9 |
| 103 | DDPDYYND | CeVF_CT_8 |
| 104 | DDPDYYN | CeVF_CT_7 |
| 105 | DDPDYY | CeVF_CT_6 |
| 106 | DDPDY | CeVF_CT_5 |
| 107 | DDPD | CeVF_CT_4 |
| 108 | DDP | CeVF_CT_3 |
| 109 | DD | CeVF_CT_2 |
| 110 | D | CeVF_CT_1 |
| 111 | KLVSQIHQFRSLAATTMFHRENPAFFSKNNHGNIYGIS | HPIV2F_CT_38_FL |
| 112 | KLVSQIHQFRSLAA | HPIV2F_CT_14 |
| 113 | KLVSQIHQFRSLA | HPIV2F_CT_13 |
| 114 | KLVSQIHQFRSL | HPIV2F_CT_12 |
| 115 | KLVSQIHQFRS | HPIV2F_CT_11 |
| 116 | KLVSQIHQFR | HPIV2F_CT_10 |
| 117 | KLVSQIHQF | HPIV2F_CT_9 |
| 118 | KLVSQIHQ | HPIV2F_CT_8 |
| 119 | KLVSQIH | HPIV2F_CT_7 |
| 120 | KLVSQI | HPIV2F_CT_6 |
| 121 | KLVSQ | HPIV2F_CT 5 |
| 122 | KLVS | HPIV2F_CT_4 |
| 123 | KLV | HPIV2F_CT_3 |
| 124 | KL | HPIV2F_CT_2 |
| 125 | RGRCNKKGEQVGMSRPGLKPDLTGTSKSYVRSL | MeaslesF_CT_33_FL |
| 126 | RGRCNKKGEQVGMS | MeaslesF_CT_14 |
| 127 | RGRCNKKGEQVGM | MeaslesF_CT_13 |
| 128 | RGRCNKKGEQVG | MeaslesF_CT_12 |
| 129 | RGRCNKKGEQV | MeaslesF_CT_11 |
| 130 | RGRCNKKGEQ | MeaslesF_CT_10 |
| 131 | RGRCNKKGE | MeaslesF_CT_9 |
| 132 | RGRCNKKG | MeaslesF_CT_8 |
| 133 | RGRCNKK | MeaslesF_CT_7 |
| 134 | RGRCNK | MeaslesF_CT_6 |
| 135 | RGRCN | MeaslesF_CT_5 |
| 136 | RGRC | MeaslesF_CT_4 |
| 137 | MGLKVNVSAIFMAVLLTLQTPTGQIHWGNLSKIGVVGIGS | Full MV-F |
| | ASYKVMTRSSHQSLVIKLMPNITLLNNCTRVEIAEYRRLLR | |
| | TVLEPIRDALNAMTQNIRPVQSVASSRRHKRFAGVVLAGA | |
| | ALGVATAAQITAGIALHQSMLNSQAIDNLRASLETTNQAIE | |
| | AIRQAGQEMILAVQGVQDYINNELIPSMNQLSCDLIGQKLG | |
| | LKLLRYYTEILSLFGPSLRDPISAEISIQALSYALGGDINKVL | |
| | EKLGYSGGDLLGILESRGIKARITHVDTESYFIVLSIAYPTLS | |
| | EIKGVIVHRLEGVSYNIGSQEWYTTVPKYVATQGYLISNFD | |
| | ESSCTFMPEGTVCSQNALYPMSPLLQECLRGSTKSCARTLV | |
| | SGSFGNRFILSQGNLIANCASILCKCYTTGTIINQDPDKILTY | |
| | IAADHCPVVEVNGVTIQVGSRRYPDAVYLHRIDLGPPISLE | |
| | RLDVGTNLGNAIAKLEDAKELLESSDQILRSMKGLSSTSIV | |
| | YILIAVCLGGLIGIPALICCCRGRCNKKGEQVGMSRPGLKP | |
| | DLTGTSKSYVRSL | |
| 138 | RGR | MeaslesF_CT_3 |
| 139 | RG | MeaslesF_CT_2 |
| 140 | R | MeaslesF_CT_1 |
| 141 | KQKAQQKTLLWLGNNTLDQMRATTKM | NDVF_CT_26_FL |
| 142 | KQKAQQKTLLWLGN | NDVF_CT_14 |
| 143 | KQKAQQKTLLWLG | NDVF_CT_13 |
| 144 | KQKAQQKTLLWL | NDVF_CT 12 |
| 145 | KQKAQQKTLLW | NDVF_CT_11 |
| 146 | KQKAQQKTLL | NDVF_CT_10 |
| 147 | KQKAQQKTL | NDVF_CT_9 |
| 148 | KQKAQQKT | NDVF_CT_8 |
| 149 | KQKAQQK | NDVF_CT_7 |
| 150 | KQKAQQ | NDVF_CT_6 |
| 151 | KQKAQ | NDVF_CT_5 |
| 152 | KQKA | NDVF_CT_4 |
| 153 | KQK | NDVF_CT_3 |
| 154 | KQ | NDVF_CT_2 |
| 155 | K | NDVF_CT_1 |
| 156 | RLRRSMLMGNPDDRIPRDTYTLEPKIRHMYTNGGFDAMAE | SeVF_CT_42_FL |
| | KR | |
| 157 | RLRRSMLMGNPDDR | SeVF_CT_14 |
| 158 | RLRRSMLMGNPDD | SeVF_CT_13 |
| 159 | RLRRSMLMGNPD | SeVF_CT_12 |
| 160 | RLRRSMLMGNP | SeVF_CT_11 |
| 161 | RLRRSMLMGN | SeVF_CT_10 |
| 162 | RLRRSMLMG | SeVF_CT_9 |
| 163 | RLRRSMLM | SeVF_CT_8 |
| 164 | RLRRSML | SeVF_CT_7 |
| 165 | RLRRSM | SeVF_CT_6 |
| 166 | RLRRS | SeVF_CT_5 |
| 167 | RLRR | SeVF_CT_4 |
| 168 | RLR | SeVF_CT_3 |
| 169 | RL | SeVF_CT_2 |
| 170 | NRLTAFINDKLNIIHAMVLTQQYQVLRTDEEAQD | BaEVwt_CT |
| 171 | RYRYQGSNNKRIYNDIEMSRFRK | Coca1_CT |
| 172 | CKFVF | EboV-GP_CT |
| 173 | KLVQFINDRISAVKILVLRQKYQALENEGNL | GaLV_CT |
| 174 | KLVQFINDRISAVKIL | GaLV-RLess_CT |
| 175 | THRHIKGGSCPKPHRLTNKGICSCGAFKVPGVKTVWKRR | LCMV_GP_CT |
| 176 | RLVQFVKDRISVVQAL | MLV-A-RLess_CT |
| 177 | NRLTAFINDKLNIIHA | BaEVRLess_CT |
| 178 | RLVQFVKDRISVVQALVLTQQYHQLKPIEYEP | MLV-A_CT |
| 179 | RVGIHLCIKLKHTKKRQIYTDIEMNRLGK | VSV-G_CT |
| 180 | NRVRQGYSPLSFQTHLPIPRGPDRPEGIEEEGGERDRDRSIR | HIV- |
| | LVNGSLALIWDDLRSLCLFSYHRLRDLLLIVTRIVELLGRR | 1_gp41_LLIR- |
| | GWEALKYWWNLLQYWSQELKNSAVNLLNATAIAVAEGT | LLP1-LLP3-LLP2- |
| | DRVIEVLQAAYRAIRHIPRRIRQGLERILL | gp41-CT-N_FL |
| 181 | NRVRQGYSPLSFQTHLPIPRGPDRPEGIEEEGGERDRDRSIR | HIV- |
| | LVNGSLALIWDDLRSLCLFSYHRLRDLLLIVTRIVELLGRR | 1_gp41_LLP1- |
| | GWEALKYWWNLLQYWSQELKNSAVNLLNATAIAVAEGT | LLP3-LLP2-gp41- |
| | DRVIEVLQAAYRAIRHIPRRIRQGLE | CT-N |
| 182 | NRVRQGYSPLSFQTHLPIPRGPDRPEGIEEEGGERDRDRSIR | HIV- |
| | LVNGSLALIWDDLRSLCLFSYHRLRDLLLIVTRIVELLGRR | 1_gp41_LLP3- |
| | GWEALKYWWNLLQYWSQELKNSAVNLLNATAIAVA | LLP2-gp41-CT-N |
| 183 | NRVRQGYSPLSFQTHLPIPRGPDRPEGIEEEGGERDRDRSIR | HIV- |
| | LVNGSLALIWDDLRSLCLFSYHRLRDLLLIVTRIVELL | 1_gp41_LLP2- |
| | | gp41-CT-N |
| 184 | NRVRQGYSPLSFQTHLPIPRGPDRPEGIEEEGGERDRDRSIR | HIV- |
| | LVNGS | 1_gp41_gp41-CT- |
| | | N |
| 185 | LALIWDDLRSLCLFSYHRLRDLLLIVTRIVELLGRRGWEAL | HIV- |
| | KYWWNLLQYWSQELKNSAVNLLNATAIAVAEGTDRVIEV | 1_gp41_LLIR- |
| | LQAAYRAIRHIPRRIRQGLERILL | LLP1-LLP3-LLP2 |
| 186 | LALIWDDLRSLCLFSYHRLRDLLLIVTRIVELLGRRGWEAL | HIV- |
| | KYWWNLLQYWSQELKNSAVNLLNATAIAVAEGTDRVIEV | 1_gp41_LLP1- |
| | LQAAYRAIRHIPRRIRQGLE | LLP3-LLP2 |
| 187 | LALIWDDLRSLCLFSYHRLRDLLLIVTRIVELLGRRGWEAL | HIV- |
| | KYWWNLLQYWSQELKNSAVNLLNATAIAVA | 1_gp41_LLP3- |
| | | LLP2 |
| 188 | LALIWDDLRSLCLFSYHRLRDLLLIVTRIVELL | HIV-1_gp41_LLP2 |
| 189 | WEALKYWWNLLQYWSQELKNSAVNLLNATAIAVAEGTD | HIV- |
| | RVIEVLQAAYRAIRHIPRRIRQGLERILL | 1_gp41_LLIR- |
| | | LLP1-LLP3 |
| 190 | WEALKYWWNLLQYWSQELKNSAVNLLNATAIAVAEGTD | HIV- |
| | RVIEVLQAAYRAIRHIPRRIRQGLE | 1_gp41_LLP1- |
| | | LLP3 |
| 191 | WEALKYWWNLLQYWSQELKNSAVNLLNATAIAVA | HIV-1_gp41_LLP3 |
| 192 | RVIEVLQAAYRAIRHIPRRIRQGLERILL | HIV- |
| | | 1_gp41_LLIR- |
| | | LLP1 |
| 193 | RVIEVLQAAYRAIRHIPRRIRQGLE | HIV-1_gp41_LLP1 |
| 194 | KLLMIIHDRREFAKFEKEKMNAKWDTGENPIYKSAVTTVV | CD29_CT |
| | NPKYEGK | |
| 195 | EKKRNTKLLMIIHDRREFAKFEKEKMNAKWDTGENPIYKS | CD29_CT |
| | AVTTVVNPKYEGK | |
| 196 | AVEGGMKCVK | CD63_CT_1to11 |
| 197 | CGACKENYC | CD63_CT_73to81 |
| 198 | VEYGSRISKVLCC | CD63_CT_225to238 |
| 199 | EKKRNTAVEGGMKCVK | CD63_CT_1to11 |
| 200 | EKKRNTCGACKENYC | CD63_CT_73to81 |
| 201 | EKKRNTVEYGSRISKVLCC | CD63_CT_225to238 |
| 202 | EKKRNTRVGIHLCIKLKHTKKRQIYTDIEMNRLGK | VSV-G_CT |
| 203 | EKKRNTNRLTAFINDKLNIIHA | BaEVRLess_CT |
| 204 | EKKRNTRYRYQGSNNKRIYNDIEMSRFRK | Coca1_CT |
| 205 | EKKRNTCKFVF | EboV-GP_CT |
| 206 | EKKRNTKLVQFINDRISAVKILVLRQKYQALENEGNL | GaLV_CT |
| 207 | EKKRNTKLVQFINDRISAVKIL | GaLV-RLess_CT |
| 208 | EKKRNTRLVQFVKDRISVVQALVLTQQYHQLKPIEYEP | MLV-A_CT |
| 209 | EKKRNTRLVQFVKDRISVVQAL | MLV-A-RLess_CT |
| 210 | KSDLLGENIKKM | Paramyxo_attachment_ |
| | | CTs |
| 211 | KSDLLGDNIKKM | Paramyxo_attachment_ |
| | | CTs |
| 212 | RDIMLHERNIVIRSGM | Paramyxo_attachment_ |
| | | CTs |
| 213 | KSD | Paramyxo_attachment_ |
| | | CTs |
| 214 | RDI | Paramyxo_attachment_ |
| | | CTs |
| 215 | KSDLLG | Paramyxo_attachment_ |
| | | CTs |
| 216 | RDIMLH | Paramyxo_attachment_ |
| | | CTs |
| 217 | EKKRKSDLLGENIKKM | Paramyxo_attachment_ |
| | | CTs |
| 218 | EKKRRDIMLHERNIVIRSGM | Paramyxo_attachment_ |
| | | CTs |
| 219 | EKKRNTKSDLLGDNIKKM | Paramyxo_attachment_ |
| | | CTs |
| 220 | EKKRNTRDIMLHERNIVIRSGM | Paramyxo_attachment_ |
| | | CTs |
| 221 | EKKRNTYSRLKSDLLGDNIKKM | Paramyxo_attachment_ |
| | | CTs |
| 222 | EKKRNTYSRLRDIMLHERNIVIRSGM | Paramyxo_attachment_ |
| | | CTs |
| 223 | LISMLSMIILYVLSIASLCIGLITFISFIIVEKKRNTYSRLEDRR | NIV CT and TM |
| | VRPTSSGDLYYIGT | |
| 224 | MVVILDKRCYCNLLILILMISECSVGILHYEKLSKIGLVKGV | NNTHDSRRHKR/ |
| | TRKYKIKSNPLTKDIVIKMIPNVSNMSQCTGSVMENYKTRL | FAGV |
| | NGILTPIKGALEIYKNNTHDSRRHKRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 225 | MVVILDKRCYCNLLILILMISECSVGILHYEKLSKIGLVKGV | NNTHDLVRHKR/ |
| | TRKYKIKSNPLTKDIVIKMIPNVSNMSQCTGSVMENYKTRL | FAGV |
| | NGILTPIKGALEIYKNNTHDLVRHKRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 226 | MVVILDKRCYCNLLILILMISECSVGILHYEKLSKIGLVKGV | NNTHDLVRHKR/ |
| | TRKYKIKSNPLTKDIVIKMIPNVSNMSQCTGSVMENYKTRL | LAGV |
| | NGILTPIKGALEIYKNNTHDLVRHKRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 227 | MVVILDKRCYCNLLILILMISECSVGILHYEKLSKIGLVKGV | NNTHDLVRHRR/ |
| | TRKYKIKSNPLTKDIVIKMIPNVSNMSQCTGSVMENYKTRL | FAGV |
| | NGILTPIKGALEIYKNNTHDLVRHRRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 228 | MVVILDKRCYCNLLILILMISECSVGILHYEKLSKIGLVKGV | NNTHDLVRHRR/ |
| | TRKYKIKSNPLTKDIVIKMIPNVSNMSQCTGSVMENYKTRL | LAGV |
| | NGILTPIKGALEIYKNNTHDLVRHRRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 229 | MVVILDKRCYCNLLILILMISECSVGILHYEKLSKIGLVKGV | NNGHDSRRHKR/ |
| | TRKYKIKSNPLTKDIVIKMIPNVSNMSQCTGSVMENYKTRL | FAGV |
| | NGILTPIKGALEIYKNNGHDSRRHKRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 230 | MVVILDKRCYCNLLILILMISECSVGILHYEKLSKIGLVKGV | NNGHDLVRHKR/ |
| | TRKYKIKSNPLTKDIVIKMIPNVSNMSQCTGSVMENYKTRL | FAGV |
| | NGILTPIKGALEIYKNNGHDLVRHKRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 231 | MVVILDKRCYCNLLILILMISECSVGILHYEKLSKIGLVKGV | NNGHDLVRHKR/ |
| | TRKYKIKSNPLTKDIVIKMIPNVSNMSQCTGSVMENYKTRL | LAGV |
| | NGILTPIKGALEIYKNNGHDLVRHKRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 232 | MVVILDKRCYCNLLILILMISECSVGILHYEKLSKIGLVKGV | NNGHDLVRHRR/ |
| | TRKYKIKSNPLTKDIVIKMIPNVSNMSQCTGSVMENYKTRL | FAGV |
| | NGILTPIKGALEIYKNNGHDLVRHRRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 233 | MVVILDKRCYCNLLILILMISECSVGILHYEKLSKIGLVKGV | NNGHDLVRHRR/ |
| | TRKYKIKSNPLTKDIVIKMIPNVSNMSQCTGSVMENYKTRL | LAGV |
| | NGILTPIKGALEIYKNNGHDLVRHRRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 234 | MVVILDKRCYCNLLILILMISECSVGILHYEKLSKIGLVKGV | QNTHDSRRHKR/ |
| | TRKYKIKSNPLTKDIVIKMIPNVSNMSQCTGSVMENYKTRL | FAGV |
| | NGILTPIKGALEIYKQNTHDSRRHKRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 235 | MVVILDKRCYCNLLILILMISECSVGILHYEKLSKIGLVKGV | QNTHDLVRHKR/ |
| | TRKYKIKSNPLTKDIVIKMIPNVSNMSQCTGSVMENYKTRL | FAGV |
| | NGILTPIKGALEIYKQNTHDLVRHKRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 236 | MVVILDKRCYCNLLILILMISECSVGILHYEKLSKIGLVKGV | QNTHDLVRHKR/ |
| | TRKYKIKSNPLTKDIVIKMIPNVSNMSQCTGSVMENYKTRL | LAGV |
| | NGILTPIKGALEIYKQNTHDLVRHKRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 237 | MVVILDKRCYCNLLILILMISECSVGILHYEKLSKIGLVKGV | QNTHDLVRHRR/ |
| | TRKYKIKSNPLTKDIVIKMIPNVSNMSQCTGSVMENYKTRL | FAGV |
| | NGILTPIKGALEIYKQNTHDLVRHRRFAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 238 | MVVILDKRCYCNLLILILMISECSVGILHYEKLSKIGLVKGV | QNTHDLVRHRR/ |
| | TRKYKIKSNPLTKDIVIKMIPNVSNMSQCTGSVMENYKTRL | LAGV |
| | NGILTPIKGALEIYKQNTHDLVRHRRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 239 | MVVILDKRCYCNLLILILMISECSVGILHYEKLSKIGLVKGV | VGDVK |
| | TRKYKIKSNPLTKDIVIKMIPNVSNMSQCTGSVMENYKTRL | |
| | NGILTPIKGALEIYKNNTHDLVGDVKLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 240 | MVVILDKRCYCNLLILILMISECSVGILHYEKLSKIGLVKGV | RNHNR |
| | TRKYKIKSNPLTKDIVIKMIPNVSNMSQCTGSVMENYKTRL | |
| | NGILTPIKGALEIYKNNTHDLRNHNRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 241 | MVVILDKRCYCNLLILILMISECSVGILHYEKLSKIGLVKGV | RRHKR |
| | TRKYKIKSNPLTKDIVIKMIPNVSNMSQCTGSVMENYKTRL | |
| | NGILTPIKGALEIYKNNTHDLRRHKRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 242 | MVVILDKRCYCNLLILILMISECSVGILHYEKLSKIGLVKGV | RRQKR |
| | TRKYKIKSNPLTKDIVIKMIPNVSNMSQCTGSVMENYKTRL | |
| | NGILTPIKGALEIYKNNTHDLRRQKRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 243 | MVVILDKRCYCNLLILILMISECSVGILHYEKLSKIGLVKGV | GRQGR |
| | TRKYKIKSNPLTKDIVIKMIPNVSNMSQCTGSVMENYKTRL | |
| | NGILTPIKGALEIYKNNTHDLGRQGRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 244 | MVVILDKRCYCNLLILILMISECSVGILHYEKLSKIGLVKGV | TRQKR |
| | TRKYKIKSNPLTKDIVIKMIPNVSNMSQCTGSVMENYKTRL | |
| | NGILTPIKGALEIYKNNTHDLTRQKRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 245 | MVVILDKRCYCNLLILILMISECSVGILHYEKLSKIGLVKGV | EIQSR |
| | TRKYKIKSNPLTKDIVIKMIPNVSNMSQCTGSVMENYKTRL | |
| | NGILTPIKGALEIYKNNTHDLEIQSRLAGVIMAGVAIGIATA | |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 246 | MVVILDKRCYCNLLILILMISECSVGILHYEKLSKIGLVKGV | VPQSR |
| | TRKYKIKSNPLTKDIVIKMIPNVSNMSQCTGSVMENYKTRL | |
| | NGILTPIKGALEIYKNNTHDLVPQSRLAGVIMAGVAIGIATA | |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 247 | MVVILDKRCYCNLLILILMISECSVGILHYEKLSKIGLVKGV | NPQSR |
| | TRKYKIKSNPLTKDIVIKMIPNVSNMSQCTGSVMENYKTRL | |
| | NGILTPIKGALEIYKNNTHDLNPQSRLAGVIMAGVAIGIATA | |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 248 | MVVILDKRCYCNLLILILMISECSVGILHYEKLSKIGLVKGV | PRTKR |
| | TRKYKIKSNPLTKDIVIKMIPNVSNMSQCTGSVMENYKTRL | |
| | NGILTPIKGALEIYKNNTHDLPRTKRLAGVIMAGVAIGIATA | |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 249 | MATQEVRLKCLLCGIIVLVLSLEGLGILHYEKLSKIGLVKG | HevF_signal_sequence |
| | VTRKYKIKSNPLTKDIVIKMIPNVSNMSQCTGSVMENYKTR | |
| | LNGILTPIKGALEIYKNNTHDLVGDVRLAGVIMAGVAIGIA | |
| | TAAQITAGVALYEAMKNADNINKLKSSIESTNEAVVKLQE | |
| | TAEKTVYVLTALQDYINTNLVPTIDKISCKQTELSLDLALS | |
| | KYLSDLLFVFGPNLQDPVSNSMTIQAISQAFGGNYETLLRT | |
| | LGYATEDFDDLLESDSITGQIIYVDLSSYYIIVRVYFPILTEIQ | |
| | QAYIQELLPVSFNNDNSEWISIVPNFILVRNTLISNIEIGFCLI | |
| | TKRSVICNQDYATPMTNNMRECLTGSTEKCPRELVVSSHV | |
| | PRFALSNGVLFANCISVTCQCQTTGRAISQSGEQTLLMIDN | |
| | TTCPTAVLGNVIISLGKYLGSVNYNSEGIAIGPPVFTDKVDI | |
| | SSQISSMNQSLQQSKDYIKEAQRLLDTVNPSLISMLSMIILY | |
| | VLSIASLCIGLITFISFIIVEKKRNT | |
| 250 | MSNKRTTVLIIISYTLFYLNNAILHYEKLSKIGLVKGVTRKY | CevF_signal_sequence |
| | KIKSNPLTKDIVIKMIPNVSNMSQCTGSVMENYKTRLNGIL | |
| | TPIKGALEIYKNNTHDLVGDVRLAGVIMAGVAIGIATAAQI | |
| | TAGVALYEAMKNADNINKLKSSIESTNEAVVKLQETAEKT | |
| | VYVLTALQDYINTNLVPTIDKISCKQTELSLDLALSKYLSDL | |
| | LFVFGPNLQDPVSNSMTIQAISQAFGGNYETLLRTLGYATE | |
| | DFDDLLESDSITGQIIYVDLSSYYIIVRVYFPILTEIQQAYIQE | |
| | LLPVSFNNDNSEWISIVPNFILVRNTLISNIEIGFCLITKRSVIC | |
| | NQDYATPMTNNMRECLTGSTEKCPRELVVSSHVPRFALSN | |
| | GVLFANCISVTCQCQTTGRAISQSGEQTLLMIDNTTCPTAV | |
| | LGNVIISLGKYLGSVNYNSEGIAIGPPVFTDKVDISSQISSMN | |
| | QSLQQSKDYIKEAQRLLDTVNPSLISMLSMIILYVLSIASLCI | |
| | GLITFISFIIVEKKRNT | |
| 251 | MHHLHPMIVCIFVMYTGIVGSDAILHYEKLSKIGLVKGVTR | HPIV2_signal_sequence |
| | KYKIKSNPLTKDIVIKMIPNVSNMSQCTGSVMENYKTRLN | |
| | GILTPIKGALEIYKNNTHDLVGDVRLAGVIMAGVAIGIATA | |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 252 | MGLKVNVSAIFMAVLLTLQTPTGQILHYEKLSKIGLVKGV | MevF_signal_sequence |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 253 | MRSRSSTRIPVPLMLIIRIALTLSCIRLTSSILHYEKLSKIGLV | NDV- |
| | KGVTRKYKIKSNPLTKDIVIKMIPNVSNMSQCTGSVMENY | F_signal_sequence |
| | KTRLNGILTPIKGALEIYKNNTHDLVGDVRLAGVIMAGVAI | |
| | GIATAAQITAGVALYEAMKNADNINKLKSSIESTNEAVVKL | |
| | QETAEKTVYVLTALQDYINTNLVPTIDKISCKQTELSLDLA | |
| | LSKYLSDLLFVFGPNLQDPVSNSMTIQAISQAFGGNYETLL | |
| | RTLGYATEDFDDLLESDSITGQIIYVDLSSYYIIVRVYFPILT | |
| | EIQQAYIQELLPVSFNNDNSEWISIVPNFILVRNTLISNIEIGF | |
| | CLITKRSVICNQDYATPMTNNMRECLTGSTEKCPRELVVSS | |
| | HVPRFALSNGVLFANCISVTCQCQTTGRAISQSGEQTLLMI | |
| | DNTTCPTAVLGNVIISLGKYLGSVNYNSEGIAIGPPVFTDKV | |
| | DISSQISSMNQSLQQSKDYIKEAQRLLDTVNPSLISMLSMIIL | |
| | YVLSIASLCIGLITFISFIIVEKKRNT | |
| 254 | MTAYIQRSQCISTSLLVVLTTLVSCQIILHYEKLSKIGLVKG | SevF_signal_sequence |
| | VTRKYKIKSNPLTKDIVIKMIPNVSNMSQCTGSVMENYKTR | |
| | LNGILTPIKGALEIYKNNTHDLVGDVRLAGVIMAGVAIGIA | |
| | TAAQITAGVALYEAMKNADNINKLKSSIESTNEAVVKLQE | |
| | TAEKTVYVLTALQDYINTNLVPTIDKISCKQTELSLDLALS | |
| | KYLSDLLFVFGPNLQDPVSNSMTIQAISQAFGGNYETLLRT | |
| | LGYATEDFDDLLESDSITGQIIYVDLSSYYIIVRVYFPILTEIQ | |
| | QAYIQELLPVSFNNDNSEWISIVPNFILVRNTLISNIEIGFCLI | |
| | TKRSVICNQDYATPMTNNMRECLTGSTEKCPRELVVSSHV | |
| | PRFALSNGVLFANCISVTCQCQTTGRAISQSGEQTLLMIDN | |
| | TTCPTAVLGNVIISLGKYLGSVNYNSEGIAIGPPVFTDKVDI | |
| | SSQISSMNQSLQQSKDYIKEAQRLLDTVNPSLISMLSMIILY | |
| | VLSIASLCIGLITFISFIIVEKKRNT | |
| 255 | MRVKGIRKNYQHLWRWGIMLLGMLMICSAADILHYEKLS | HIV1- |
| | KIGLVKGVTRKYKIKSNPLTKDIVIKMIPNVSNMSQCTGSV | Env_signal_sequence |
| | MENYKTRLNGILTPIKGALEIYKNNTHDLVGDVRLAGVIM | |
| | AGVAIGIATAAQITAGVALYEAMKNADNINKLKSSIESTNE | |
| | AVVKLQETAEKTVYVLTALQDYINTNLVPTIDKISCKQTEL | |
| | SLDLALSKYLSDLLFVFGPNLQDPVSNSMTIQAISQAFGGN | |
| | YETLLRTLGYATEDFDDLLESDSITGQIIYVDLSSYYIIVRVY | |
| | FPILTEIQQAYIQELLPVSFNNDNSEWISIVPNFILVRNTLISN | |
| | IEIGFCLITKRSVICNQDYATPMTNNMRECLTGSTEKCPREL | |
| | VVSSHVPRFALSNGVLFANCISVTCQCQTTGRAISQSGEQT | |
| | LLMIDNTTCPTAVLGNVIISLGKYLGSVNYNSEGIAIGPPVF | |
| | TDKVDISSQISSMNQSLQQSKDYIKEAQRLLDTVNPSLISML | |
| | SMIILYVLSIASLCIGLITFISFIIVEKKRNT | |
| 256 | MGFTTKIIFLYNLVLVYAILHYEKLSKIGLVKGVTRKYKIKS | BaEV_signal_sequence |
| | NPLTKDIVIKMIPNVSNMSQCTGSVMENYKTRLNGILTPIK | |
| | GALEIYKNNTHDLVGDVRLAGVIMAGVAIGIATAAQITAG | |
| | VALYEAMKNADNINKLKSSIESTNEAVVKLQETAEKTVYV | |
| | LTALQDYINTNLVPTIDKISCKQTELSLDLALSKYLSDLLFV | |
| | FGPNLQDPVSNSMTIQAISQAFGGNYETLLRTLGYATEDFD | |
| | DLLESDSITGQIIYVDLSSYYIIVRVYFPILTEIQQAYIQELLP | |
| | VSFNNDNSEWISIVPNFILVRNTLISNIEIGFCLITKRSVICNQ | |
| | DYATPMTNNMRECLTGSTEKCPRELVVSSHVPRFALSNGV | |
| | LFANCISVTCQCQTTGRAISQSGEQTLLMIDNTTCPTAVLG | |
| | NVIISLGKYLGSVNYNSEGIAIGPPVFTDKVDISSQISSMNQS | |
| | LQQSKDYIKEAQRLLDTVNPSLISMLSMIILYVLSIASLCIGL | |
| | ITFISFIIVEKKRNT | |
| 257 | MNFLLLTFIVLPLCSHAILHYEKLSKIGLVKGVTRKYKIKSN | Cocal_signal_sequence |
| | PLTKDIVIKMIPNVSNMSQCTGSVMENYKTRLNGILTPIKG | |
| | ALEIYKNNTHDLVGDVRLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCQCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRNT | |
| 258 | MGVTGILQLPRDRFKRTSFFLWVIILFQRTFSIILHYEKLSKI | EboV_signal_sequence |
| | GLVKGVTRKYKIKSNPLTKDIVIKMIPNVSNMSQCTGSVM | |
| | ENYKTRLNGILTPIKGALEIYKNNTHDLVGDVRLAGVIMA | |
| | GVAIGIATAAQITAGVALYEAMKNADNINKLKSSIESTNEA | |
| | VVKLQETAEKTVYVLTALQDYINTNLVPTIDKISCKQTELS | |
| | LDLALSKYLSDLLFVFGPNLQDPVSNSMTIQAISQAFGGNY | |
| | ETLLRTLGYATEDFDDLLESDSITGQIIYVDLSSYYIIVRVYF | |
| | PILTEIQQAYIQELLPVSFNNDNSEWISIVPNFILVRNTLISNI | |
| | EIGFCLITKRSVICNQDYATPMTNNMRECLTGSTEKCPREL | |
| | VVSSHVPRFALSNGVLFANCISVTCQCQTTGRAISQSGEQT | |
| | LLMIDNTTCPTAVLGNVIISLGKYLGSVNYNSEGIAIGPPVF | |
| | TDKVDISSQISSMNQSLQQSKDYIKEAQRLLDTVNPSLISML | |
| | SMIILYVLSIASLCIGLITFISFIIVEKKRNT | |
| 259 | MVLLPGSMLLTSNLHHLRHQMSPGSWKRLIILLSCVFGGG | GaLV_signal_sequence |
| | GTSILHYEKLSKIGLVKGVTRKYKIKSNPLTKDIVIKMIPNV | |
| | SNMSQCTGSVMENYKTRLNGILTPIKGALEIYKNNTHDLV | |
| | GDVRLAGVIMAGVAIGIATAAQITAGVALYEAMKNADNIN | |
| | KLKSSIESTNEAVVKLQETAEKTVYVLTALQDYINTNLVPT | |
| | IDKISCKQTELSLDLALSKYLSDLLFVFGPNLQDPVSNSMTI | |
| | QAISQAFGGNYETLLRTLGYATEDFDDLLESDSITGQIIYVD | |
| | LSSYYIIVRVYFPILTEIQQAYIQELLPVSFNNDNSEWISIVPN | |
| | FILVRNTLISNIEIGFCLITKRSVICNQDYATPMTNNMRECLT | |
| | GSTEKCPRELVVSSHVPRFALSNGVLFANCISVTCQCQTTG | |
| | RAISQSGEQTLLMIDNTTCPTAVLGNVIISLGKYLGSVNYNS | |
| | EGIAIGPPVFTDKVDISSQISSMNQSLQQSKDYIKEAQRLLD | |
| | TVNPSLISMLSMIILYVLSIASLCIGLITFISFIIVEKKRNT | |
| 260 | MARSTLSKPLKNKVNPRGPLIPLILLMLRGVSTASPILHYEK | MLV- |
| | LSKIGLVKGVTRKYKIKSNPLTKDIVIKMIPNVSNMSQCTG | A_signal_sequence |
| | SVMENYKTRLNGILTPIKGALEIYKNNTHDLVGDVRLAGVI | |
| | MAGVAIGIATAAQITAGVALYEAMKNADNINKLKSSIESTN | |
| | EAVVKLQETAEKTVYVLTALQDYINTNLVPTIDKISCKQTE | |
| | LSLDLALSKYLSDLLFVFGPNLQDPVSNSMTIQAISQAFGG | |
| | NYETLLRTLGYATEDFDDLLESDSITGQIIYVDLSSYYIIVRV | |
| | YFPILTEIQQAYIQELLPVSFNNDNSEWISIVPNFILVRNTLIS | |
| | NIEIGFCLITKRSVICNQDYATPMTNNMRECLTGSTEKCPRE | |
| | LVVSSHVPRFALSNGVLFANCISVTCQCQTTGRAISQSGEQ | |
| | TLLMIDNTTCPTAVLGNVIISLGKYLGSVNYNSEGIAIGPPV | |
| | FTDKVDISSQISSMNQSLQQSKDYIKEAQRLLDTVNPSLISM | |
| | LSMIILYVLSIASLCIGLITFISFIIVEKKRNT | |
| 261 | MKCLLYLAFLFIGVNCILHYEKLSKIGLVKGVTRKYKIKSN | VSVG_signal_sequence |
| | PLTKDIVIKMIPNVSNMSQCTGSVMENYKTRLNGILTPIKG | |
| | ALEIYKNNTHDLVGDVRLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCQCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRNT | |
| 262 | MNIKGSPWKGSLLLLLVSNLLLCQSVAPILHYEKLSKIGLV | Prolactin_signal_ |
| | KGVTRKYKIKSNPLTKDIVIKMIPNVSNMSQCTGSVMENY | sequence |
| | KTRLNGILTPIKGALEIYKNNTHDLVGDVRLAGVIMAGVAI | |
| | GIATAAQITAGVALYEAMKNADNINKLKSSIESTNEAVVKL | |
| | QETAEKTVYVLTALQDYINTNLVPTIDKISCKQTELSLDLA | |
| | LSKYLSDLLFVFGPNLQDPVSNSMTIQAISQAFGGNYETLL | |
| | RTLGYATEDFDDLLESDSITGQIIYVDLSSYYIIVRVYFPILT | |
| | EIQQAYIQELLPVSFNNDNSEWISIVPNFILVRNTLISNIEIGF | |
| | CLITKRSVICNQDYATPMTNNMRECLTGSTEKCPRELVVSS | |
| | HVPRFALSNGVLFANCISVTCQCQTTGRAISQSGEQTLLMI | |
| | DNTTCPTAVLGNVIISLGKYLGSVNYNSEGIAIGPPVFTDKV | |
| | DISSQISSMNQSLQQSKDYIKEAQRLLDTVNPSLISMLSMIIL | |
| | YVLSIASLCIGLITFISFIIVEKKRNT | |
| 263 | METPAQLLFLLLLWLPDTTGILHYEKLSKIGLVKGVTRKYK | IgGk- |
| | IKSNPLTKDIVIKMIPNVSNMSQCTGSVMENYKTRLNGILTP | L_signal_sequence |
| | IKGALEIYKNNTHDLVGDVRLAGVIMAGVAIGIATAAQITA | |
| | GVALYEAMKNADNINKLKSSIESTNEAVVKLQETAEKTVY | |
| | VLTALQDYINTNLVPTIDKISCKQTELSLDLALSKYLSDLLF | |
| | VFGPNLQDPVSNSMTIQAISQAFGGNYETLLRTLGYATEDF | |
| | DDLLESDSITGQIIYVDLSSYYIIVRVYFPILTEIQQAYIQELL | |
| | PVSFNNDNSEWISIVPNFILVRNTLISNIEIGFCLITKRSVICN | |
| | QDYATPMTNNMRECLTGSTEKCPRELVVSSHVPRFALSNG | |
| | VLFANCISVTCQCQTTGRAISQSGEQTLLMIDNTTCPTAVL | |
| | GNVIISLGKYLGSVNYNSEGIAIGPPVFTDKVDISSQISSMN | |
| | QSLQQSKDYIKEAQRLLDTVNPSLISMLSMIILYVLSIASLCI | |
| | GLITFISFIIVEKKRNT | |
| 264 | MKWVTFISLLFLFSSAYSILHYEKLSKIGLVKGVTRKYKIKS | Albumin_signal_ |
| | NPLTKDIVIKMIPNVSNMSQCTGSVMENYKTRLNGILTPIK | sequence |
| | GALEIYKNNTHDLVGDVRLAGVIMAGVAIGIATAAQITAG | |
| | VALYEAMKNADNINKLKSSIESTNEAVVKLQETAEKTVYV | |
| | LTALQDYINTNLVPTIDKISCKQTELSLDLALSKYLSDLLFV | |
| | FGPNLQDPVSNSMTIQAISQAFGGNYETLLRTLGYATEDFD | |
| | DLLESDSITGQIIYVDLSSYYIIVRVYFPILTEIQQAYIQELLP | |
| | VSFNNDNSEWISIVPNFILVRNTLISNIEIGFCLITKRSVICNQ | |
| | DYATPMTNNMRECLTGSTEKCPRELVVSSHVPRFALSNGV | |
| | LFANCISVTCQCQTTGRAISQSGEQTLLMIDNTTCPTAVLG | |
| | NVIISLGKYLGSVNYNSEGIAIGPPVFTDKVDISSQISSMNQS | |
| | LQQSKDYIKEAQRLLDTVNPSLISMLSMIILYVLSIASLCIGL | |
| | ITFISFIIVEKKRNT | |
| 265 | MPMGSLQPLATLYLLGMLVASCLGRLILHYEKLSKIGLVK | CD5_signal_sequence |
| | GVTRKYKIKSNPLTKDIVIKMIPNVSNMSQCTGSVMENYK | |
| | TRLNGILTPIKGALEIYKNNTHDLVGDVRLAGVIMAGVAIG | |
| | IATAAQITAGVALYEAMKNADNINKLKSSIESTNEAVVKLQ | |
| | ETAEKTVYVLTALQDYINTNLVPTIDKISCKQTELSLDLALS | |
| | KYLSDLLFVFGPNLQDPVSNSMTIQAISQAFGGNYETLLRT | |
| | LGYATEDFDDLLESDSITGQIIYVDLSSYYIIVRVYFPILTEIQ | |
| | QAYIQELLPVSFNNDNSEWISIVPNFILVRNTLISNIEIGFCLI | |
| | TKRSVICNQDYATPMTNNMRECLTGSTEKCPRELVVSSHV | |
| | PRFALSNGVLFANCISVTCQCQTTGRAISQSGEQTLLMIDN | |
| | TTCPTAVLGNVIISLGKYLGSVNYNSEGIAIGPPVFTDKVDI | |
| | SSQISSMNQSLQQSKDYIKEAQRLLDTVNPSLISMLSMIILY | |
| | VLSIASLCIGLITFISFIIVEKKRNT | |
| 266 | MNPLLILTFVAAALAILHYEKLSKIGLVKGVTRKYKIKSNP | Trypsinogen_signal_ |
| | LTKDIVIKMIPNVSNMSQCTGSVMENYKTRLNGILTPIKGA | sequence |
| | LEIYKNNTHDLVGDVRLAGVIMAGVAIGIATAAQITAGVA | |
| | LYEAMKNADNINKLKSSIESTNEAVVKLQETAEKTVYVLT | |
| | ALQDYINTNLVPTIDKISCKQTELSLDLALSKYLSDLLFVFG | |
| | PNLQDPVSNSMTIQAISQAFGGNYETLLRTLGYATEDFDDL | |
| | LESDSITGQIIYVDLSSYYIIVRVYFPILTEIQQAYIQELLPVSF | |
| | NNDNSEWISIVPNFILVRNTLISNIEIGFCLITKRSVICNQDYA | |
| | TPMTNNMRECLTGSTEKCPRELVVSSHVPRFALSNGVLFA | |
| | NCISVTCQCQTTGRAISQSGEQTLLMIDNTTCPTAVLGNVII | |
| | SLGKYLGSVNYNSEGIAIGPPVFTDKVDISSQISSMNQSLQQ | |
| | SKDYIKEAQRLLDTVNPSLISMLSMIILYVLSIASLCIGLITFI | |
| | SFIIVEKKRNT | |
| 267 | MYRMQLLSCIALSLALVTNSILHYEKLSKIGLVKGVTRKYK | IL2_signal_sequence |
| | IKSNPLTKDIVIKMIPNVSNMSQCTGSVMENYKTRLNGILTP | |
| | IKGALEIYKNNTHDLVGDVRLAGVIMAGVAIGIATAAQITA | |
| | GVALYEAMKNADNINKLKSSIESTNEAVVKLQETAEKTVY | |
| | VLTALQDYINTNLVPTIDKISCKQTELSLDLALSKYLSDLLF | |
| | VFGPNLQDPVSNSMTIQAISQAFGGNYETLLRTLGYATEDF | |
| | DDLLESDSITGQIIYVDLSSYYIIVRVYFPILTEIQQAYIQELL | |
| | PVSFNNDNSEWISIVPNFILVRNTLISNIEIGFCLITKRSVICN | |
| | QDYATPMTNNMRECLTGSTEKCPRELVVSSHVPRFALSNG | |
| | VLFANCISVTCQCQTTGRAISQSGEQTLLMIDNTTCPTAVL | |
| | GNVIISLGKYLGSVNYNSEGIAIGPPVFTDKVDISSQISSMN | |
| | QSLQQSKDYIKEAQRLLDTVNPSLISMLSMIILYVLSIASLCI | |
| | GLITFISFIIVEKKRNT | |
| 268 | MVVILDKRCYCNLLILILMISECSVGILHYEKLSKIGLVKGV | NivF_TM_S490A_ |
| | TRKYKIKSNPLTKDIVIKMIPNVSNMSQCTGSVMENYKTRL | dec_fusogenicity |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLIAMLSMIILYV | |
| | LSIASLCIGLITFISFIIVEKKRNT | |
| 269 | MVVILDKRCYCNLLILILMISECSVGILHYEKLSKIGLVKGV | NivF_TM_Y498A_ |
| | TRKYKIKSNPLTKDIVIKMIPNVSNMSQCTGSVMENYKTRL | dec_fusogenicity |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILAV | |
| | LSIASLCIGLITFISFIIVEKKRNT | |
| 270 | MVVILDKRCYCNLLILILMISECSVGILHYEKLSKIGLVKGV | HevF_TM_FL |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIAALCIGLITFISFVIVEKKRNT | |
| 271 | MVVILDKRCYCNLLILILMISECSVGILHYEKLSKIGLVKGV | NivF_TM_HevF_S504A |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIAALCIGLITFISFIIVEKKRNT | |
| 272 | MVVILDKRCYCNLLILILMISECSVGILHYEKLSKIGLVKGV | NivF_TM_HevF_I516V |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFVIVEKKRNT | |
| 273 | MVVILDKRCYCNLLILILMISECSVGILHYEKLSKIGLVKGV | F3 |
| | TRKYKIKSNPLTKDIVIKMIPNVSNMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 274 | MVVILDKRCYCNLLILILMISECSVGILHYEKLSKIGLVKGV | F5 |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 275 | MVVILDKRCYCNLLILILMISECSVGILHYEKLSKIGLVKGV | F4F5 |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDQT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 276 | MVVILDKRCYCNLLILILMISECSVGILHYEKLSKIGLVKGV | F2F4F5 |
| | TRKYKIKSNPLTKDIVIKMIPNVSQMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDQT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 277 | ILHYEKLSKIGLVKGVTRKYKIKSNPLTKDIVIKMIPNVSNM | NivF_TM_S490A_ |
| | SQCTGSVMENYKTRLNGILTPIKGALEIYKNNTHDLVGDV | dec_fusogenicity |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLIAMLSMIILYVLSIASLCIGLITFISFIIVEKKRNT | |
| 278 | MVVILDKRCYCNLLILILMISECSVGILHYEKLSKIGLVKGV | F2F3 |
| | TRKYKIKSNPLTKDIVIKMIPNVSQMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 279 | MVVILDKRCYCNLLILILMISECSVGILHYEKLSKIGLVKGV | F2F5 |
| | TRKYKIKSNPLTKDIVIKMIPNVSQMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 280 | MVVILDKRCYCNLLILILMISECSVGILHYEKLSKIGLVKGV | F3F5 |
| | TRKYKIKSNPLTKDIVIKMIPNVSNMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 281 | MATQEVRLKCLLCGIIVLVLSLEGLGILHYEKLSKIGLVKGI | FL017HevF488NivF_ |
| | TRKYKIKSNPLTKDIVIKMIPNVSNVSKCTGTVMENYKSRL | full-length |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILY | |
| | VLSIASLCIGLITFISFIIVEKKRNTYSRLEDRRVRPTSSGDLY | |
| | YIGT | |
| 282 | MATQEVRLKCLLCGIIVLVLSLEGLGILHYEKLSKIGLVKGI | FL018HevF488NivF_ |
| | TRKYKIKSNPLTKDIVIKMIPNVSNVSKCTGTVMENYKSRL | CT-06 |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILY | |
| | VLSIASLCIGLITFISFIIVEKKRNT | |
| 283 | MATQEVRLKCLLCGIIVLVLSLEGLGILHYEKLSKIGLVKGI | FL019HevF488NivF_ |
| | TRKYKIKSNPLTKDIVIKMIPNVSNVSKCTGTVMENYKSRL | CT-10 |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILY | |
| | VLSIASLCIGLITFISFIIVEKKRNTYSRL | |
| 284 | ILHYEKLSKIGLVKGVTRKYKIKSNPLTKDIVIKMIPNVSNM | NivF_TM_Y498A_ |
| | SQCTGSVMENYKTRLNGILTPIKGALEIYKNNTHDLVGDV | _dec_fusogenicity |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILAVLSIASLCIGLITFISFIIVEKKRNT | |
| 285 | ILHYEKLSKIGLVKGVTRKYKIKSNPLTKDIVIKMIPNVSNM | HevF_TM_FL |
| | SQCTGSVMENYKTRLNGILTPIKGALEIYKNNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIAALCIGLITFISFVIVEKKRNT | |
| 286 | GGGGS | Linker |
| 287 | GGGGGS | Linker |
| 288 | (GGGGS)n | Linker |
| 289 | (GGGGGS)n | Linker |
| 290 | MVVILDKRCYCNLLILILMISECSVGILHYEKLSKIGLVKGV | F2F3F5 |
| | TRKYKIKSNPLTKDIVIKMIPNVSQMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 291 | MVVILDKRCYCNLLILILMISECSVGILHYEKLSKIGLVKGV | R109L |
| | TRKYKIKSNPLTKDIVIKMIPNVSNMSQCTGSVMENYKTRL | |
| | NGILTPIKGALEIYKNNTHDLVGDVLLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 292 | MVVILDKRCYCNLLILILMISECSVGILHYEKLSKIGLVKGV | Q393L |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 293 | MVVILDKRCYCNLLILILMISECSVGILHYEKLSKIGLVKGV | R109L_Q393L |
| | TRKYKIKSNPLTKDIVIKMIPNVSNMSQCTGSVMENYKTRL | |
| | NGILTPIKGALEIYKNNTHDLVGDVLLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 294 | MATQEVRLKCLLCGIIVLVLSLEGLGILHYEKLSKIGLVKGI | FL012 |
| | TRKYKIKSNPLTKDIVIKMIPNVSNVSKCTGTVMENYKSRL | HevF_full-length |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILY | |
| | VLSIAALCIGLITFISFVIVEKKRGNYSRLDDRQVRPVSNGD | |
| | LYYIGT | |
| 295 | MATQEVRLKCLLCGIIVLVLSLEGLGILHYEKLSKIGLVKGI | FL_013HevF_CT- |
| | TRKYKIKSNPLTKDIVIKMIPNVSNVSKCTGTVMENYKSRL | 06 |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILY | |
| | VLSIAALCIGLITFISFVIVEKKRGN | |
| 296 | MATQEVRLKCLLCGIIVLVLSLEGLGILHYEKLSKIGLVKGI | FL014 |
| | TRKYKIKSNPLTKDIVIKMIPNVSNVSKCTGTVMENYKSRL | HevF_CT-10 |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILY | |
| | VLSIAALCIGLITFISFVIVEKKRGNYSRL | |
| 297 | MKCLLYLAFLFIGVNCILHYEKLSKIGLVKGVTRKYKIKSN | FL057_CT060 |
| | PLTKDIVIKMIPNVSNMSQCTGSVMENYKTRLNGILTPIKG | |
| | ALEIYKNNTHDLVGDVRLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCQCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVRGR | |
| 298 | MYRMQLLSCIALSLALVTNSILHYEKLSKIGLVKGVTRKYK | FL063_CT060 |
| | IKSNPLTKDIVIKMIPNVSNMSQCTGSVMENYKTRLNGILTP | |
| | IKGALEIYKNNTHDLVGDVRLAGVIMAGVAIGIATAAQITA | |
| | GVALYEAMKNADNINKLKSSIESTNEAVVKLQETAEKTVY | |
| | VLTALQDYINTNLVPTIDKISCKQTELSLDLALSKYLSDLLF | |
| | VFGPNLQDPVSNSMTIQAISQAFGGNYETLLRTLGYATEDF | |
| | DDLLESDSITGQIIYVDLSSYYIIVRVYFPILTEIQQAYIQELL | |
| | PVSFNNDNSEWISIVPNFILVRNTLISNIEIGFCLITKRSVICN | |
| | QDYATPMTNNMRECLTGSTEKCPRELVVSSHVPRFALSNG | |
| | VLFANCISVTCQCQTTGRAISQSGEQTLLMIDNTTCPTAVL | |
| | GNVIISLGKYLGSVNYNSEGIAIGPPVFTDKVDISSQISSMN | |
| | QSLQQSKDYIKEAQRLLDTVNPSLISMLSMIILYVLSIASLCI | |
| | GLITFISFIIVRGR | |
| 299 | MKCLLYLAFLFIGVNCILHYEKLSKIGLVKGVTRKYKIKSN | FL007_FL057_CT |
| | PLTKDIVIKMIPNVSNMSQCTGSVMENYKTRLNGILTPIKG | 060 |
| | ALEIYKQNTHDLVGDVRLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCQCQTTGRAISQSGEQTLLMIDNTTCPTAVLGN | |
| | VIISLGKYLGSVNYNSEGIAIGPPVFTDKVDISSQISSMQQSL | |
| | QQSKDYIKEAQRLLDTVNPSLISMLSMIILYVLSIASLCIGLI | |
| | TFISFIIVEKKRNT | |
| 300 | MKCLLYLAFLFIGVNCILHYEKLSKIGLVKGITRKYKIKSNP | FL007_FL018 |
| | LTKDIVIKMIPNVSNVSKCTGTVMENYKSRLTGILSPIKGAI | |
| | ELYNNNTHDLVGDVKLAGVVMAGIAIGIATAAQITAGVAL | |
| | YEAMKNADNINKLKSSIESTNEAVVKLQETAEKTVYVLTA | |
| | LQDYINTNLVPTIDQISCKQTELALDLALSKYLSDLLFVFGP | |
| | NLQDPVSNSMTIQAISQAFGGNYETLLRTLGYATEDFDDLL | |
| | ESDSIAGQIVYVDLSSYYIIVRVYFPILTEIQQAYVQELLPVS | |
| | FNNDNSEWISIVPNFVLIRNTLISNIEVKYCLITKKSVICNQD | |
| | YATPMTASVRECLTGSTDKCPRELVVSSHVPRFALSGGVLF | |
| | ANCISVTCQCQTTGRAISQSGEQTLLMIDNTTCTTVVLGNII | |
| | ISLGKYLGSINYNSESIAVGPPVYTDKVDISSQISSMNQSLQ | |
| | QSKDYIKEAQKILDTVNPSLISMLSMIILYVLSIASLCIGLITF | |
| | ISFIIVEKKRNT | |
| 301 | MKKINE GLLDSKILSA FNTVIALLGS IVIIVMNIMI | (E501 A, W504A, |
| | IQNYTRSTDN QAVIKDALQG IQQQIKGLAD KIGTEIGPKV | Q530A, E533A) |
| | SLIDTSSTIT IPANIGLLGS KISQSTASIN ENVNEKCKFT | NiV G protein (Gc |
| | LPPLKIHECN ISCPNPLPFR EYRPQTEGVS NLVGLPNNIC | Δ 34) |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST | |
| | VGDPILNSTY WSGSLMMTRL AVKPKSNGGG | |
| | YNQHQLALRS IEKGRYDKVM PYGPSGIKQG | |
| | DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC | |
| | PAICAEGVYN DAFLIDRINW ISAGVFLDSN ATAANPVFTV | |
| | FKDNEILYRA QLASEDTNAQ KTITNCFLLK NKIWCISLVE | |
| | IYDTGDNVIR PKLFAVKIPE QC | |
| 302 | MVVILDKRCY CNLLILILMI SECSVGILHY EKLSKIGLVK | (FcDelta22) at |
| | GVTRKYKIKS NPLTKDIVIK MIPNVSNMSQ | cytoplasmic tail |
| | CTGSVMENYK TRLNGILTPI KGALEIYKNN | (with signal |
| | THDLVGDVRL AGVIMAGVAI GIATAAQITA | sequence) |
| | GVALYEAMKN ADNINKLKSS IESTNEAVVK | |
| | LQETAEKTVY VLTALQDYIN TNLVPTIDKI SCKQTELSLD | |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNT | |
| 303 | ILHY EKLSKIGLVK GVTRKYKIKS NPLTKDIVIK | Truncated mature |
| | MIPNVSNMSQ CTGSVMENYK TRLNGILTPI | NiV fusion |
| | KGALEIYKNN THDLVGDVRL AGVIMAGVAI | glycoprotein |
| | GIATAAQITA GVALYEAMKN ADNINKLKSS | (FcDelta22) at |
| | IESTNEAVVK LQETAEKTVY VLTALQDYIN TNLVPTIDKI | cytoplasmic tail |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 304 | MPAENKKVRFENTTSDKGKIPSKVIKSYYGTMDIKKINEGL | gb: AF212302\| |
| | LDSKILSAFNTVIALLGSIVIIVMNIMIIQNYTRSTDNQAVIK | Organism: Nipah |
| | DALQGIQQQIKGLADKIGTEIGPKVSLIDTSSTITIPANIGLLG | virus\|Strain |
| | SKISQSTASINENVNEKCKFTLPPLKIHECNISCPNPLPFREY | Name: UNKNOWN- |
| | RPQTEGVSNLVGLPNNICLQKTSNQILKPKLISYTLPVVGQS | AF212302\|Protein |
| | GTCITDPLLAMDEGYFAYSHLERIGSCSRGVSKQRIIGVGE | Name: attachment |
| | VLDRGDEVPSLFMTNVWTPPNPNTVYHCSAVYNNEFYYV | glycoprotein\|Gene |
| | LCAVSTVGDPILNSTYWSGSLMMTRLAVKPKSNGGGYNQ | Symbol: G |
| | HQLALRSIEKGRYDKVMPYGPSGIKQGDTLYFPAVGFLVR | Uniprot Q9IH62 |
| | TEFKYNDSNCPITKCQYSKPENCRLSMGIRPNSHYILRSGLL | |
| | KYNLSDGENPKVVFIEISDQRLSIGSPSKIYDSLGQPVFYQA | |
| | SFSWDTMIKFGDVLTVNPLVVNWRNNTVISRPGQSQCPRF | |
| | NTCPEICWEGVYNDAFLIDRINWISAGVFLDSNQTAENPVF | |
| | TVFKDNEILYRAQLASEDTNAQKTITNCFLLKNKIWCISLV | |
| | EIYDTGDNVIRPKLFAVKIPEQCT | |
| 305 | MPAENKKVRFENTTSDKGKIPSKVIKSYYGTMDIKKINEGL | Full-length NiVG |
| | LDSKILSAFNTVIALLGSIVIIVMNIMIIQNYTRSTDNQAVIK | with (E501 A, |
| | DALQGIQQQIKGLADKIGTEIGPKVSLIDTSSTITIPANIGLLG | W504A, Q530A, |
| | SKISQSTASINENVNEKCKFTLPPLKIHECNISCPNPLPFREY | E533A) |
| | RPQTEGVSNLVGLPNNICLQKTSNQILKPKLISYTLPVVGQS | |
| | GTCITDPLLAMDEGYFAYSHLERIGSCSRGVSKQRIIGVGE | |
| | VLDRGDEVPSLFMTNVWTPPNPNTVYHCSAVYNNEFYYV | |
| | LCAVSTVGDPILNSTYWSGSLMMTRLAVKPKSNGGGYNQ | |
| | HQLALRSIEKGRYDKVMPYGPSGIKQGDTLYFPAVGFLVR | |
| | TEFKYNDSNCPITKCQYSKPENCRLSMGIRPNSHYILRSGLL | |
| | KYNLSDGENPKVVFIEISDQRLSIGSPSKIYDSLGQPVFYQA | |
| | SFSWDTMIKFGDVLTVNPLVVNWRNNTVISRPGQSQCPRF | |
| | NTCPAICAEGVYNDAFLIDRINWISAGVFLDSNATAANPVF | |
| | TVFKDNEILYRAQLASEDTNAQKTITNCFLLKNKIWCISLV | |
| | EIYDTGDNVIRPKLFAVKIPEQCT | |
| 306 | ILHY EKLSKIGLVK GVTRKYKIKS NPLTKDIVIK | mature NiV-F |
| | MIPNVSNMSQ CTGSVMENYK TRLNGILTPI | CT_5 |
| | KGALEIYKNN THDLVGDVRL AGVIMAGVAI | |
| | GIATAAQITA GVALYEAMKN ADNINKLKSS | |
| | IESTNEAVVK LQETAEKTVY VLTALQDYIN TNLVPTIDKI | |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRN | |
| 307 | ILHYEKLSKIGLVKGVTRKYKIKSNPLTKDIVIKMIPNVSNM | mature |
| | SQCTGSVMENYKTRLNGILTPIKGALEIYKNNTHDLVGDV | MeaslesF_CT_7 |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVRGRCNKK | |
| 308 | ILHYEKLSKIGLVKGVTRKYKIKSNPLTKDIVIKMIPNVSNM | mature |
| | SQCTGSVMENYKTRLNGILTPIKGALEIYKNNTHDLVGDV | NDVF_CT_9 |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVKQKAQQKTL | |
| 309 | ILHYEKLSKIGLVKGVTRKYKIKSNPLTKDIVIKMIPNVSNM | mature |
| | SQCTGSVMENYKTRLNGILTPIKGALEIYKNNTHDLVGDV | NDVF_CT_6 |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIV KQKAQQ | |
| 310 | MVVILDKRCYCNLLILILMISECSVGILHYEKLSKIGLVKGV | mature |
| | TRKYKIKSNPLTKDIVIKMIPNVSNMSQCTGSVMENYKTRL | HeVF_CT_6 |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRGN | |
| 311 | NNTHDLVGDVRLAGV | NiV-F cleavage |
| | | site |
| 312 | R-X-R/K-R, X is any amino acid | Furin cleavage seq |
| 313 | NNTHDSRRHKR/FAGV | Modified protease |
| | | site |
| 314 | NNTHDLVRHKR/FAGV | Modified protease |
| | | site |
| 315 | NNTHDLVRHKR/FAGV | Modified protease |
| | | site |
| 316 | NNTHDLVRHRR/FAGV | Modified protease |
| | | site |
| 317 | NNTHDLVRHRR/LAGV | Modified protease |
| | | site |
| 318 | NNGHDSRRHKR/FAGV | Modified protease |
| | | site |
| 319 | NNGHDLVRHKR/FAGV | Modified protease |
| | | site |
| 320 | NNGHDLVRHKR/LAGV | Modified protease |
| | | site |
| 321 | NNGHDLVRHRR/FAGV | Modified protease |
| | | site |
| 322 | NNGHDLVRHRR/LAGV | Modified protease |
| | | site |
| 323 | QNTHDSRRHKR/FAGV | Modified protease |
| | | site |
| 324 | QNTHDLVRHKR/FAGV | Modified protease |
| | | site |
| 325 | QNTHDLVRHKR/LAGV | Modified protease |
| | | site |
| 326 | QNTHDLVRHRR/FAGV | Modified protease |
| | | site |
| 327 | QNTHDLVRHRR/LAGV | Modified protease |
| | | site |
| 328 | LISMLSMIILYVLSIASLCIGLITFISFIIVEKKRNT | Modified protease |
| | | site |
| 329 | VGDVK | Modified protease |
| | | site |
| 330 | RNHNR | Modified protease |
| | | site |
| 331 | RRHKR | Modified protease |
| | | site |
| 332 | RRQKR | Modified protease |
| | | site |
| 333 | GRQGR | Modified protease |
| | | site |
| 334 | TRQKR | Modified protease |
| | | site |
| 335 | EIQSR | Modified protease |
| | | site |
| 336 | VPQSR | Modified protease |
| | | site |
| 337 | NPQSR | Modified protease |
| | | site |
| 338 | PRTKR | Modified protease |
| | | site |
| 339 | VGDVR | Modified protease |
| | | site |
| 340 | MATQEVRLKCLLCGIIVLVLSLEGLG | HevF_signal_ |
| | | sequence |
| 341 | MSNKRTTVLIIISYTLFYLNNA | CevF_signal_ |
| | | sequence |
| 342 | MHHLHPMIVCIFVMYTGIVGSDA | HPIV2_signal_ |
| | | sequence |
| 343 | MGLKVNVSAIFMAVLLTLQTPTGQ | MevF_signal_ |
| | | sequence |
| 344 | MRSRSSTRIPVPLMLIIRIALTLSCIRLTSS | NDV- |
| | | F_signal_sequence |
| 345 | MTAYIQRSQCISTSLLVVLTTLVSCQI | SevF_signal_ |
| | | sequence |
| 346 | MRVKGIRKNYQHLWRWGIMLLGMLMICSAAD | HIV1- |
| | | Env_signal_ |
| | | sequence |
| 347 | MGFTTKIIFLYNLVLVYA | BaEV_signal_ |
| | | sequence |
| 348 | MNFLLLTFIVLPLCSHA | Cocal_signal_ |
| | | sequence |
| 349 | MGVTGILQLPRDRFKRTSFFLWVIILFQRTFSI | EboV_signal_ |
| | | sequence |
| 350 | MVLLPGSMLLTSNLHHLRHQMSPGSWKRLIILLSCVFGGG | GaLV_signal_ |
| | GTS | sequence |
| 351 | MARSTLSKPLKNKVNPRGPLIPLILLMLRGVSTASP | MLV- |
| | | A_signal_sequence |
| 352 | MKCLLYLAFLFIGVNCI | VSVG_signal_ |
| | | sequence |
| 353 | MNIKGSPWKGSLLLLLVSNLLLCQSVAP | Prolactin_signal_ |
| | | sequence |
| 354 | METPAQLLFLLLLWLPDTTG | IgGk- |
| | | L_signal_sequence |
| 355 | MKWVTFISLLFLFSSAYS | Albumin_signal_ |
| | | sequence |
| 356 | MPMGSLQPLATLYLLGMLVASCLGRL | CD5_signal_ |
| | | sequence |
| 357 | MNPLLILTFVAAALA | Trypsinogen_signal_ |
| | | sequence |
| 358 | MYRMQLLSCIALSLALVTNS | IL2_signal_ |
| | | sequence |
| 359 | LIAMLSMIILYVLSIASLCIGLITFISFIIV | NivF_TM_S490A |
| 360 | LISMLSMIILAVLSIASLCIGLITFISFIIV | NivF_TM_Y498A |
| 361 | LISMLSMIILYVLSIAALCIGLITFISFVIV | HevF_TM_FL |
| 362 | LISMLSMIILYVLSIAALCIGLITFISFIIV | NivF_TM_HevF_ |
| | | S504A |
| 363 | LISMLSMIILYVLSIASLCIGLITFISFVIV | NivF_TM_HevF_ |
| | | I516V |
| 364 | ILHY EKLSKIGLVK GVTRKYKIKS NPLTKDIVIK | Nipah virus NiV- |
| | MIPNVSNMSQ CTGSVMENYK TRLNGILTPI | FF0 (aa 27-546) |
| | KGALEIYKNN THDLVGDVRL AGVIMAGVAI | |
| | GIATAAQITA GVALYEAMKN ADNINKLKSS | |
| | IESTNEAVVK LQETAEKTVY VLTALQDYIN TNLVPTIDKI | |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTYSRLED RRVRPTSSGD LYYIGT | |
| 365 | ILHYEKLSKIGLVKGVTRKYKIKSNPLTKDIVIKMIPNVSNM | Nipah virus NiV- |
| | SQCTGSVMENYKTRLNGILTPIKGALEIYKNNTHDLVGDV | FF2 (aa 27-109 of |
| | R | SEQ ID NO: 1) |
| 366 | LAGVIMAGVAIGIATAAQITAGVALYEAMKNADNINKLKS | Nipah virus NiV |
| | SIESTNEAVVKLQETAEKTVYVLTALQDYINTNLVPTIDKIS | FF1 (aa 110-546) |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNTYSRLED | |
| | RRVRPTSSGDLYYIGT | |
| 367 | MVVILDKRCYCNLLILILMISECSVGILHYEKLSKIGLVKGV | F3F5_Q393L_CT- |
| | TRKYKIKSNPLTKDIVIKMIPNVSNMSQCTGSVMENYKTRL | 06_K1A |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVEKKRNT | |
| 368 | MVVILDKRCYCNLLILILMISECSVGILHYEKLSKIGLVKGV | F3F5_Q393L_CT- |
| | TRKYKIKSNPLTKDIVIKMIPNVSNMSQCTGSVMENYKTRL | 10_K1A |
| | NGILTPIKGALEIYKNNTHDLVGDVKLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVEKKRNT | |
| 369 | MVVILDKRCYCNLLILILMISECSVGILHYEKLSKIGLVKGV | VSVG_signal_ |
| | TRKYKIKSNPLTKDIVIKMIPNVSNMSQCTGSVMENYKTRL | sequence_ |
| | NGILTPIKGALEIYKNNTHDLVGDVKLAGVIMAGVAIGIAT | MeaslesF_CT_3 |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 370 | MVVILDKRCYCNLLILILMISECSVGILHYEKLSKIGLVKGV | IL2_signal_sequence_ |
| | TRKYKIKSNPLTKDIVIKMIPNVSNMSQCTGSVMENYKTRL | MeaslesF_CT_ |
| | NGILTPIKGALEIYKNNTHDLVGDVKLAGVIMAGVAIGIAT | 3 |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 371 | MHHLHPMIVCIFVMYTGIVGSDAILHYEKLSKIGLVKGVTR | F3F5_ |
| | KYKIKSNPLTKDIVIKMIPNVSNMSQCTGSVMENYKTRLN | VSVG_signal_ |
| | GILTPIKGALEIYKNNTHDLVGDVRLAGVIMAGVAIGIATA | sequence_ |
| | AQITAGVALYEAMKNADNINKLKSSIESTNEAVVKLQETA | MeaslesF_CT_3 |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMQQSLQQSKDYIKEAQRLLDTVNPSLISMLSMIILYVLSI | |
| | ASLCIGLITFISFIIVEKKRNT | |
| 372 | MKCLLYLAFLFIGVNCILHYEKLSKIGLVKGVTRKYKIKSN | F3F5_ |
| | PLTKDIVIKMIPNVSNMSQCTGSVMENYKTRLNGILTPIKG | HevF488NivF_CT- |
| | ALEIYKNNTHDLVGDVRLAGVIMAGVAIGIATAAQITAGV | 06 |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMQQSL | |
| | QQSKDYIKEAQRLLDTVNPSLISMLSMIILYVLSIASLCIGLI | |
| | TFISFIIVEKKRNT | |
| 373 | MVVILDKRCYCNLLILILMISECSVGILHYEKLSKIGLVKGV | F5_ |
| | TRKYKIKSNPLTKDIVIKMIPNVSNMSQCTGSVMENYKTRL | Q393L |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVKQKAQQ | |
| 374 | MVVILDKRCYCNLLILILMISECSVGILHYEKLSKIGLVKGV | F5_ |
| | TRKYKIKSNPLTKDIVIKMIPNVSNMSQCTGSVMENYKTRL | Q393L_ VGDVK |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVEKKRGN | |
| 375 | MVVILDKRCYCNLLILILMISECSVGILHYEKLSKIGLVKGV | F5_VGDVK |
| | TRKYKIKSNPLTKDIVIKMIPNVSNMSQCTGSVMENYKTRL | |
| | NGILTPIKGALEIYKNNTHDLVGDVKLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVKQKAQQ | |
| 376 | MVVILDKRCYCNLLILILMISECSVGILHYEKLSKIGLVKGV | Q393L_ VGDVK |
| | TRKYKIKSNPLTKDIVIKMIPNVSNMSQCTGSVMENYKTRL | |
| | NGILTPIKGALEIYKNNTHDLVGDVKLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMQQSLQQSKDYIKEAQRLLDTVNPSLISMLSMIILYVL | |
| | SIASLCIGLITFISFIIVEKKRGN | |
| 377 | MVVILDKRCYCNLLILILMISECSVGILHYEKLSKIGLVKGV | F5_ |
| | TRKYKIKSNPLTKDIVIKMIPNVSNMSQCTGSVMENYKTRL | Q393L_ |
| | NGILTPIKGALEIYKNNTHDLVGDVRLAGVIMAGVAIGIAT | HPIV2_signal_ |
| | AAQITAGVALYEAMKNADNINKLKSSIESTNEAVVKLQET | sequence |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVKQKAQQ | |
| 378 | MVVILDKRCYCNLLILILMISECSVGILHYEKLSKIGLVKGV | F5_ |
| | TRKYKIKSNPLTKDIVIKMIPNVSNMSQCTGSVMENYKTRL | Q393L_ |
| | NGILTPIKGALEIYKNNTHDLVGDVRLAGVIMAGVAIGIAT | VSVG_signal_ |
| | AAQITAGVALYEAMKNADNINKLKSSIESTNEAVVKLQET | sequence |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVKQKAQQ | |
| 379 | MMADSKLVSLNNNLSGKIKDQGKVIKNYYGTMDIKKIND | Hendra virus G |
| | GLLDSKILGAFNTVIALLGSIIIIVMNIMIIQNYTRTTDNQALI | protein Uniprot |
| | KESLQSVQQQIKALTDKIGTEIGPKVSLIDTSSTITIPANIGLL | O89343 |
| | GSKISQSTSSINENVNDKCKFTLPPLKIHECNISCPNPLPFRE | |
| | YRPISQGVSDLVGLPNQICLQKTTSTILKPRLISYTLPINTRE | |
| | GVCITDPLLAVDNGFFAYSHLEKIGSCTRGIAKQRIIGVGEV | |
| | LDRGDKVPSMFMTNVWTPPNPSTIHHCSSTYHEDFYYTLC | |
| | AVSHVGDPILNSTSWTESLSLIRLAVRPKSDSGDYNQKYIAI | |
| | TKVERGKYDKVMPYGPSGIKQGDTLYFPAVGFLPRTEFQY | |
| | NDSNCPIIHCKYSKAENCRLSMGVNSKSHYILRSGLLKYNL | |
| | SLGGDIILQFIEIADNRLTIGSPSKIYNSLGQPVFYQASYSWD | |
| | TMIKLGDVDTVDPLRVQWRNNSVISRPGQSQCPRFNVCPE | |
| | VCWEGTYNDAFLIDRLNWVSAGVYLNSNQTAENPVFAVF | |
| | KDNEILYQVPLAEDDTNAQKTITDCFLLENVIWCISLVEIYD | |
| | TGDSVIRPKLFAVKIPAQCSES | |
| 380 | MLSQLQKNYLDNSNQQGDKMNNPDKKLSVNFNPLELDKG | gb: JQ001776: 8170- |
| | QKDLNKSYYVKNKNYNVSNLLNESLHDIKFCIYCIFSLLIIIT | 10275\|Organism: |
| | IINIITISIVITRLKVHEENNGMESPNLQSIQDSLSSLTNMINT | Cedar virus\|Strain |
| | EITPRIGILVTATSVTLSSSINYVGTKTNQLVNELKDYITKSC | Name: CG1a\|Protein |
| | GFKVPELKLHECNISCADPKISKSAMYSTNAYAELAGPPKI | Name: attachment |
| | FCKSVSKDPDFRLKQIDYVIPVQQDRSICMNNPLLDISDGFF | glycoprotein\|Gene |
| | TYIHYEGINSCKKSDSFKVLLSHGEIVDRGDYRPSLYLLSSH | Symbol: G |
| | YHPYSMQVINCVPVTCNQSSFVFCHISNNTKTLDNSDYSSD | |
| | EYYITYFNGIDRPKTKKIPINNMTADNRYIHFTFSGGGGVCL | |
| | GEEFIIPVTTVINTDVFTHDYCESFNCSVQTGKSLKEICSESL | |
| | RSPTNSSRYNLNGIMIISQNNMTDFKIQLNGITYNKLSFGSP | |
| | GRLSKTLGQVLYYQSSMSWDTYLKAGFVEKWKPFTPNW | |
| | MNNTVISRPNQGNCPRYHKCPEICYGGTYNDIAPLDLGKD | |
| | MYVSVILDSDQLAENPEITVENSTTILYKERVSKDELNTRST | |
| | TTSCFLFLDEPWCISVLETNRFNGKSIRPEIYSYKIPKYC | |
| 381 | MPQKTVEFINMNSPLERGVSTLSDKKTLNQSKITKQGYFGL | gb:NC_025256: |
| | GSHSERNWKKQKNQNDHYMTVSTMILEILVVLGIMENLIV | 9117- |
| | LTMVYYQNDNINQRMAELTSNITVLNLNLNQLINKIQREII | 11015\|Organism: |
| | PRITLIDTATTITIPSAITYILATLTTRISELLPSINQKCEFKTPT | Bat Paramyxovirus |
| | LVLNDCRINCTPPLNPSDGVKMSSLATNLVAHGPSPCRNFS | Eid_hel/GH- |
| | SVPTIYYYRIPGLYNRTALDERCILNPRLTISSTKFAYVHSE | M74a/GHA/2009\| |
| | YDKNCTRGFKYYELMTFGEILEGPEKEPRMFSRSFYSPTNA | Strain |
| | VNYHSCTPIVTVNEGYFLCLECTSSDPLYKANLSNSTFHLVI | Name: BatPV/Eid_ |
| | LRHNKDEKIVSMPSFNLSTDQEYVQIIPAEGGGTAESGNLY | hel/GH- |
| | FPCIGRLLHKRVTHPLCKKSNCSRTDDESCLKSYYNQGSPQ | M74a/GHA/2009\| |
| | HQVVNCLIRIRNAQRDNPTWDVITVDLTNTYPGSRSRIFGS | Protein |
| | FSKPMLYQSSVSWHTLLQVAEITDLDKYQLDWLDTPYISR | Name: glycoprotein\| |
| | PGGSECPFGNYCPTVCWEGTYNDVYSLTPNNDLFVTVYLK | Gene Symbol: G |
| | SEQVAENPYFAIFSRDQILKEFPLDAWISSARTTTISCFMFN | |
| | NEIWCIAALEITRLNDDIIRPIYYSFWLPTDCRTPYPHTGKM | |
| | TRVPLRSTYNY | |
| 382 | MATNRDNTITSAEVSQEDKVKKYYGVETAEKVADSISGNK | gb:NC_025352: |
| | VFILMNTLLILTGAIITITLNITNLTAAKSQQNMLKIIQDDVN | 8716- |
| | AKLEMFVNLDQLVKGEIKPKVSLINTAVSVSIPGQISNLQT | 11257\|Organism: |
| | KFLQKYVYLEESITKQCTCNPLSGIFPTSGPTYPPTDKPDDD | Mojiang virus\|Strain |
| | TTDDDKVDTTIKPIEYPKPDGCNRTGDHFTMEPGANFYTVP | Name: Tongguan1\| |
| | NLGPASSNSDECYTNPSFSIGSSIYMFSQEIRKTDCTAGEILS | Protein |
| | IQIVLGRIVDKGQQGPQASPLLVWAVPNPKIINSCAVAAGD | Name: attachment |
| | EMGWVLCSVTLTAASGEPIPHMFDGFWLYKLEPDTEVVSY | glycoprotein\|Gene |
| | RITGYAYLLDKQYDSVFIGKGGGIQKGNDLYFQMYGLSRN | Symbol: G |
| | RQSFKALCEHGSCLGTGGGGYQVLCDRAVMSFGSEESLIT | |
| | NAYLKVNDLASGKPVIIGQTFPPSDSYKGSNGRMYTIGDK | |
| | YGLYLAPSSWNRYLRFGITPDISVRSTTWLKSQDPIMKILST | |
| | CTNTDRDMCPEICNTRGYQDIFPLSEDSEYYTYIGITPNNGG | |
| | TKNFVAVRDSDGHIASIDILQNYYSITSATISCFMYKDEIWC | |
| | IAITEGKKQKDNPQRIYAHSYKIRQMCYNMKSATVTVGNA | |
| | KNITIRRY | |
| 383 | LAGVIMAGVAIGIATAAQITAGVALYEAMKNADNINKLKS | Nipah virus NiV F |
| | SIESTNEAVVKLQETAEKTVYVLTALQDYINTNLVPTIDKIS | F1 ΔCT |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIV | |
| 384 | ILHYEKLSKIGLVKGVTRKYKIKSNPLTKDIVIKMIPNVSNM | mature |
| | SQCTGSVMENYKTRLNGILTPIKGALEIYKNNTHDLVGDV | HeVF_CT_6 |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRGN | |
| 385 | ILHYEKLSKIGLVKGVTRKYKIKSNPLTKDIVIKMIPNVSNM | NNTHDSRRHKR/ |
| | SQCTGSVMENYKTRLNGILTPIKGALEIYKNNTHDSRRHKR | FAGV |
| | FAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 386 | ILHYEKLSKIGLVKGVTRKYKIKSNPLTKDIVIKMIPNVSNM | NNTHDLVRHKR/ |
| | SQCTGSVMENYKTRLNGILTPIKGALEIYKNNTHDLVRHK | FAGV |
| | RFAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 387 | ILHYEKLSKIGLVKGVTRKYKIKSNPLTKDIVIKMIPNVSNM | NNTHDLVRHKR/ |
| | SQCTGSVMENYKTRLNGILTPIKGALEIYKNNTHDLVRHK | LAGV |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 388 | ILHYEKLSKIGLVKGVTRKYKIKSNPLTKDIVIKMIPNVSNM | NNTHDLVRHRR/ |
| | SQCTGSVMENYKTRLNGILTPIKGALEIYKNNTHDLVRHRR | FAGV |
| | FAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 389 | ILHYEKLSKIGLVKGVTRKYKIKSNPLTKDIVIKMIPNVSNM | NNTHDLVRHRR/ |
| | SQCTGSVMENYKTRLNGILTPIKGALEIYKNNTHDLVRHRR | LAGV |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 390 | ILHYEKLSKIGLVKGVTRKYKIKSNPLTKDIVIKMIPNVSNM | NNGHDSRRHKR/ |
| | SQCTGSVMENYKTRLNGILTPIKGALEIYKNNGHDSRRHK | FAGV |
| | RFAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 391 | ILHYEKLSKIGLVKGVTRKYKIKSNPLTKDIVIKMIPNVSNM | NNGHDLVRHKR/ |
| | SQCTGSVMENYKTRLNGILTPIKGALEIYKNNGHDLVRHK | FAGV |
| | RFAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 392 | ILHYEKLSKIGLVKGVTRKYKIKSNPLTKDIVIKMIPNVSNM | NNGHDLVRHKR/ |
| | SQCTGSVMENYKTRLNGILTPIKGALEIYKNNGHDLVRHK | LAGV |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 393 | ILHYEKLSKIGLVKGVTRKYKIKSNPLTKDIVIKMIPNVSNM | NNGHDLVRHRR/ |
| | SQCTGSVMENYKTRLNGILTPIKGALEIYKNNGHDLVRHR | FAGV |
| | RFAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 394 | ILHYEKLSKIGLVKGVTRKYKIKSNPLTKDIVIKMIPNVSNM | NNGHDLVRHRR/ |
| | SQCTGSVMENYKTRLNGILTPIKGALEIYKNNGHDLVRHR | LAGV |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 395 | ILHYEKLSKIGLVKGVTRKYKIKSNPLTKDIVIKMIPNVSNM | QNTHDSRRHKR/ |
| | SQCTGSVMENYKTRLNGILTPIKGALEIYKQNTHDSRRHKR | FAGV |
| | FAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 396 | ILHYEKLSKIGLVKGVTRKYKIKSNPLTKDIVIKMIPNVSNM | QNTHDLVRHKR/ |
| | SQCTGSVMENYKTRLNGILTPIKGALEIYKQNTHDLVRHK | FAGV |
| | RFAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 397 | ILHYEKLSKIGLVKGVTRKYKIKSNPLTKDIVIKMIPNVSNM | QNTHDLVRHKR/ |
| | SQCTGSVMENYKTRLNGILTPIKGALEIYKQNTHDLVRHK | LAGV |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 398 | ILHYEKLSKIGLVKGVTRKYKIKSNPLTKDIVIKMIPNVSNM | QNTHDLVRHRR/ |
| | SQCTGSVMENYKTRLNGILTPIKGALEIYKQNTHDLVRHRR | FAGV |
| | FAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 399 | ILHYEKLSKIGLVKGVTRKYKIKSNPLTKDIVIKMIPNVSNM | QNTHDLVRHRR/ |
| | SQCTGSVMENYKTRLNGILTPIKGALEIYKQNTHDLVRHRR | LAGV |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 400 | ILHYEKLSKIGLVKGVTRKYKIKSNPLTKDIVIKMIPNVSNM | VGDVK |
| | SQCTGSVMENYKTRLNGILTPIKGALEIYKNNTHDLVGDV | |
| | KLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 401 | ILHYEKLSKIGLVKGVTRKYKIKSNPLTKDIVIKMIPNVSNM | RNHNR |
| | SQCTGSVMENYKTRLNGILTPIKGALEIYKNNTHDLRNHN | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 402 | ILHYEKLSKIGLVKGVTRKYKIKSNPLTKDIVIKMIPNVSNM | RRHKR |
| | SQCTGSVMENYKTRLNGILTPIKGALEIYKNNTHDLRRHKR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 403 | ILHYEKLSKIGLVKGVTRKYKIKSNPLTKDIVIKMIPNVSNM | RRQKR |
| | SQCTGSVMENYKTRLNGILTPIKGALEIYKNNTHDLRRQKR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 404 | ILHYEKLSKIGLVKGVTRKYKIKSNPLTKDIVIKMIPNVSNM | GRQGR |
| | SQCTGSVMENYKTRLNGILTPIKGALEIYKNNTHDLGRQG | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 405 | ILHYEKLSKIGLVKGVTRKYKIKSNPLTKDIVIKMIPNVSNM | TRQKR |
| | SQCTGSVMENYKTRLNGILTPIKGALEIYKNNTHDLTRQKR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 406 | ILHYEKLSKIGLVKGVTRKYKIKSNPLTKDIVIKMIPNVSNM | EIQSR |
| | SQCTGSVMENYKTRLNGILTPIKGALEIYKNNTHDLEIQSR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 407 | ILHYEKLSKIGLVKGVTRKYKIKSNPLTKDIVIKMIPNVSNM | VPQSR |
| | SQCTGSVMENYKTRLNGILTPIKGALEIYKNNTHDLVPQSR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 408 | ILHYEKLSKIGLVKGVTRKYKIKSNPLTKDIVIKMIPNVSNM | NPQSR |
| | SQCTGSVMENYKTRLNGILTPIKGALEIYKNNTHDLNPQSR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 409 | ILHYEKLSKIGLVKGVTRKYKIKSNPLTKDIVIKMIPNVSNM | PRTKR |
| | SQCTGSVMENYKTRLNGILTPIKGALEIYKNNTHDLPRTKR | |
| | LAGVIMAGVAIGIATAAQITAGVALYEAMKNADNINKLKS | |
| | SIESTNEAVVKLQETAEKTVYVLTALQDYINTNLVPTIDKIS | |
| | CKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 410 | ILHYEKLSKIGLVKGVTRKYKIKSNPLTKDIVIKMIPNVSNM | F3 |
| | SQCTGSVMENYKTRLNGILTPIKGALEIYKQNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 411 | ILHYEKLSKIGLVKGVTRKYKIKSNPLTKDIVIKMIPNVSNM | F5 |
| | SQCTGSVMENYKTRLNGILTPIKGALEIYKNNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMQQSLQQSKDYIKEAQRLLDTVN | |
| | PSLISMLSMIILYVLSIASLCIGLITFISFIIVEKKRNT | |
| 412 | ILHYEKLSKIGLVKGVTRKYKIKSNPLTKDIVIKMIPNVSNM | F4F5 |
| | SQCTGSVMENYKTRLNGILTPIKGALEIYKNNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDQTTCPTAVLGNVIISLGKYLGSVNYNSEGI | |
| | AIGPPVFTDKVDISSQISSMQQSLQQSKDYIKEAQRLLDTVN | |
| | PSLISMLSMIILYVLSIASLCIGLITFISFIIVEKKRNT | |
| 413 | ILHYEKLSKIGLVKGVTRKYKIKSNPLTKDIVIKMIPNVSQM | F2F4F5 |
| | SQCTGSVMENYKTRLNGILTPIKGALEIYKNNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDQTTCPTAVLGNVIISLGKYLGSVNYNSEGI | |
| | AIGPPVFTDKVDISSQISSMQQSLQQSKDYIKEAQRLLDTVN | |
| | PSLISMLSMIILYVLSIASLCIGLITFISFIIVEKKRNT | |
| 414 | ILHYEKLSKIGLVKGVTRKYKIKSNPLTKDIVIKMIPNVSQM | F2F5 |
| | SQCTGSVMENYKTRLNGILTPIKGALEIYKNNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMQQSLQQSKDYIKEAQRLLDTVN | |
| | PSLISMLSMIILYVLSIASLCIGLITFISFIIVEKKRNT | |
| 415 | ILHYEKLSKIGLVKGVTRKYKIKSNPLTKDIVIKMIPNVSNM | F3F5 |
| | SQCTGSVMENYKTRLNGILTPIKGALEIYKQNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMQQSLQQSKDYIKEAQRLLDTVN | |
| | PSLISMLSMIILYVLSIASLCIGLITFISFIIVEKKRNT | |
| 416 | ILHYEKLSKIGLVKGVTRKYKIKSNPLTKDIVIKMIPNVSNM | F3 |
| | SQCTGSVMENYKTRLNGILTPIKGALEIYKQNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 417 | ILHYEKLSKIGLVKGVTRKYKIKSNPLTKDIVIKMIPNVSNM | R109L |
| | SQCTGSVMENYKTRLNGILTPIKGALEIYKNNTHDLVGDV | |
| | LLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 418 | ILHYEKLSKIGLVKGVTRKYKIKSNPLTKDIVIKMIPNVSNM | Q393L |
| | SQCTGSVMENYKTRLNGILTPIKGALEIYKNNTHDLVGDV | |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCLCQTTGRAIS | |
| | QSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 419 | ILHYEKLSKIGLVKGVTRKYKIKSNPLTKDIVIKMIPNVSNM | R109L_Q393L |
| | SQCTGSVMENYKTRLNGILTPIKGALEIYKNNTHDLVGDV | |
| | LLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCLCQTTGRAIS | |
| | QSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 420 | ILHYEKLSKIGLVKGVTRKYKIKSNPLTKDIVIKMIPNVSNM | NivF_TM_HevF_ |
| | SQCTGSVMENYKTRLNGILTPIKGALEIYKNNTHDLVGDV | S504A |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIAALCIGLITFISFIIVEKKRNT | |
| 421 | ILHYEKLSKIGLVKGVTRKYKIKSNPLTKDIVIKMIPNVSNM | NivF_TM_HevF_ |
| | SQCTGSVMENYKTRLNGILTPIKGALEIYKNNTHDLVGDV | I516V |
| | RLAGVIMAGVAIGIATAAQITAGVALYEAMKNADNINKLK | |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFVIVEKKRNT | |
| 422 | ILHYEKLSKIGLVKGVTRKYKIKSNPLTKDIVIKMIPNVSQM | HPIV2_signal_ |
| | SQCTGSVMENYKTRLNGILTPIKGALEIYKQNTHDLVGDV | sequence_CT06_ |
| | RLAGVIMAGVAIGIATAAQITAGVALYEAMKNADNINKLK | Q393L |
| | SSIESTNEAVVKLQETAEKTVYVLTALQDYINTNLVPTIDKI | |
| | SCKQTELSLDLALSKYLSDLLFVFGPNLQDPVSNSMTIQAIS | |
| | QAFGGNYETLLRTLGYATEDFDDLLESDSITGQIIYVDLSSY | |
| | YIIVRVYFPILTEIQQAYIQELLPVSFNNDNSEWISIVPNFILV | |
| | RNTLISNIEIGFCLITKRSVICNQDYATPMTNNMRECLTGST | |
| | EKCPRELVVSSHVPRFALSNGVLFANCISVTCQCQTTGRAI | |
| | SQSGEQTLLMIDNTTCPTAVLGNVIISLGKYLGSVNYNSEGI | |
| | AIGPPVFTDKVDISSQISSMNQSLQQSKDYIKEAQRLLDTVN | |
| | PSLISMLSMIILYVLSIASLCIGLITFISFIIVEKKRNT | |
| 423 | MHHLHPMIVCIFVMYTGIVGSDAILHYEKLSKIGLVKGVTR | VSVG_signal_ |
| | KYKIKSNPLTKDIVIKMIPNVSNMSQCTGSVMENYKTRLN | sequence_CT06_ |
| | GILTPIKGALEIYKNNTHDLVGDVRLAGVIMAGVAIGIATA | Q393L |
| | AQITAGVALYEAMKNADNINKLKSSIESTNEA VVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRNT | |
| 424 | MKCLLYLAFLFIGVNCILHYEKLSKIGLVKGVTRKYKIKSN | VGDVK |
| | PLTKDIVIKMIPNVSNMSQCTGSVMENYKTRLNGILTPIKG | (HeV)_HPIV2_signal_ |
| | ALEIYKNNTHDLVGDVRLAGVIMAGVAIGIATAAQITAGV | sequence_CT06_ |
| | ALYEAMKNADNINKLKSSIESTNEAVVKLQETAEKTVYVL | Q393L |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRNT | |
| 425 | MHHLHPMIVCIFVMYTGIVGSDAILHYEKLSKIGLVKGVTR | VGDVK |
| | KYKIKSNPLTKDIVIKMIPNVSNMSQCTGSVMENYKTRLN | (HeV)_VSVG_signal_ |
| | GILTPIKGALEIYKNNTHDLVGDVKLAGVIMAGVAIGIATA | sequence_CT06_ |
| | AQITAGVALYEAMKNADNINKLKSSIESTNEAVVKLQETA | Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRNT | |
| 426 | MKCLLYLAFLFIGVNCILHYEKLSKIGLVKGVTRKYKIKSN | NDVF_CT_6_ |
| | PLTKDIVIKMIPNVSNMSQCTGSVMENYKTRLNGILTPIKG | Q393L |
| | ALEIYKNNTHDLVGDVKLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRNT | |
| 427 | MVVILDKRCYCNLLILILMISECSVGILHYEKLSKIGLVKGV | VGDVK |
| | TRKYKIKSNPLTKDIVIKMIPNVSNMSQCTGSVMENYKTRL | (HeV)_NDVF_CT_ |
| | NGILTPIKGALEIYKNNTHDLVGDVRLAGVIMAGVAIGIAT | 6_Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVKQKAQQ | |
| 428 | MVVILDKRCYCNLLILILMISECSVGILHYEKLSKIGLVKGV | HPIV2_signal_ |
| | TRKYKIKSNPLTKDIVIKMIPNVSNMSQCTGSVMENYKTRL | sequence- |
| | NGILTPIKGALEIYKNNTHDLVGDVKLAGVIMAGVAIGIAT | NDVF_CT_6_ |
| | AAQITAGVALYEAMKNADNINKLKSSIESTNEAVVKLQET | Q393L |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVKQKAQQ | |
| 429 | MHHLHPMIVCIFVMYTGIVGSDAILHYEKLSKIGLVKGVTR | VSVG_signal_ |
| | KYKIKSNPLTKDIVIKMIPNVSNMSQCTGSVMENYKTRLN | sequence_NDVF_ |
| | GILTPIKGALEIYKNNTHDLVGDVRLAGVIMAGVAIGIATA | CT6_Q393L |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVKQKAQQ | |
| 430 | MKCLLYLAFLFIGVNCILHYEKLSKIGLVKGVTRKYKIKSN | VGDVK |
| | PLTKDIVIKMIPNVSNMSQCTGSVMENYKTRLNGILTPIKG | (HeV)_HPIV2_signal_ |
| | ALEIYKNNTHDLVGDVRLAGVIMAGVAIGIATAAQITAGV | sequence_NDVF_ |
| | ALYEAMKNADNINKLKSSIESTNEAVVKLQETAEKTVYVL | CT_6_Q393L |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVKQKAQQ | |
| 431 | MHHLHPMIVCIFVMYTGIVGSDAILHYEKLSKIGLVKGVTR | VGDVK |
| | KYKIKSNPLTKDIVIKMIPNVSNMSQCTGSVMENYKTRLN | (HeV)_VSVG_signal_ |
| | GILTPIKGALEIYKNNTHDLVGDVKLAGVIMAGVAIGIATA | sequence_NDVF_ |
| | AQITAGVALYEAMKNADNINKLKSSIESTNEAVVKLQETA | CT_6_Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVKQKAQQ | |
| 432 | MKCLLYLAFLFIGVNCILHYEKLSKIGLVKGVTRKYKIKSN | HeVF_CT_6_ |
| | PLTKDIVIKMIPNVSNMSQCTGSVMENYKTRLNGILTPIKG | Q393L |
| | ALEIYKNNTHDLVGDVKLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVKQKAQQ | |
| 433 | MVVILDKRCYCNLLILILMISECSVGILHYEKLSKIGLVKGV | VGDVK (HeV)_ |
| | TRKYKIKSNPLTKDIVIKMIPNVSNMSQCTGSVMENYKTRL | HeVF_CT_6_ |
| | NGILTPIKGALEIYKNNTHDLVGDVRLAGVIMAGVAIGIAT | Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRGN | |
| 434 | MVVILDKRCYCNLLILILMISECSVGILHYEKLSKIGLVKGV | HPIV2_signal_ |
| | TRKYKIKSNPLTKDIVIKMIPNVSNMSQCTGSVMENYKTRL | sequence_HeVF_CT_ |
| | NGILTPIKGALEIYKNNTHDLVGDVKLAGVIMAGVAIGIAT | 6_Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRGN | |
| 435 | MHHLHPMIVCIFVMYTGIVGSDAILHYEKLSKIGLVKGVTR | VSVG_signal_ |
| | KYKIKSNPLTKDIVIKMIPNVSNMSQCTGSVMENYKTRLN | sequence_HeVF_ |
| | GILTPIKGALEIYKNNTHDLVGDVRLAGVIMAGVAIGIATA | CT6_Q393L |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRGN | |
| 436 | MKCLLYLAFLFIGVNCILHYEKLSKIGLVKGVTRKYKIKSN | VGDVK |
| | PLTKDIVIKMIPNVSNMSQCTGSVMENYKTRLNGILTPIKG | (HeV)_HPIV2_signal_ |
| | ALEIYKNNTHDLVGDVRLAGVIMAGVAIGIATAAQITAGV | sequence_HeVF_ |
| | ALYEAMKNADNINKLKSSIESTNEAVVKLQETAEKTVYVL | CT_6_Q393L |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRGN | |
| 437 | MHHLHPMIVCIFVMYTGIVGSDAILHYEKLSKIGLVKGVTR | VGDVK |
| | KYKIKSNPLTKDIVIKMIPNVSNMSQCTGSVMENYKTRLN | (HeV)_VSVG_signal_ |
| | GILTPIKGALEIYKNNTHDLVGDVKLAGVIMAGVAIGIATA | sequence_HeVF_ |
| | AQITAGVALYEAMKNADNINKLKSSIESTNEAVVKLQETA | CT_6_Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRGN | |
| 438 | MKCLLYLAFLFIGVNCILHYEKLSKIGLVKGVTRKYKIKSN | F3_CT06_Q393L |
| | PLTKDIVIKMIPNVSNMSQCTGSVMENYKTRLNGILTPIKG | |
| | ALEIYKNNTHDLVGDVKLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRGN | |
| 439 | MVVILDKRCYCNLLILILMISECSVGILHYEKLSKIGLVKGV | F3_VGDVK |
| | TRKYKIKSNPLTKDIVIKMIPNVSNMSQCTGSVMENYKTRL | (HeV)_CT06_ |
| | NGILTPIKGALEIYKQNTHDLVGDVRLAGVIMAGVAIGIAT | Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 440 | MVVILDKRCYCNLLILILMISECSVGILHYEKLSKIGLVKGV | F3- |
| | TRKYKIKSNPLTKDIVIKMIPNVSNMSQCTGSVMENYKTRL | _HPIV2_signal_ |
| | NGILTPIKGALEIYKQNTHDLVGDVKLAGVIMAGVAIGIAT | sequence_CT06_ |
| | AAQITAGVALYEAMKNADNINKLKSSIESTNEAVVKLQET | Q393L |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNT | |
| 441 | MHHLHPMIVCIFVMYTGIVGSDAILHYEKLSKIGLVKGVTR | F3- |
| | KYKIKSNPLTKDIVIKMIPNVSNMSQCTGSVMENYKTRLN | _VSVG_signal_ |
| | GILTPIKGALEIYKQNTHDLVGDVRLAGVIMAGVAIGIATA | sequence_CT06_ |
| | AQITAGVALYEAMKNADNINKLKSSIESTNEAVVKLQETA | Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRNT | |
| 442 | MKCLLYLAFLFIGVNCILHYEKLSKIGLVKGVTRKYKIKSN | F3_VGDVK |
| | PLTKDIVIKMIPNVSNMSQCTGSVMENYKTRLNGILTPIKG | (HeV)_HPIV2_signal_ |
| | ALEIYKQNTHDLVGDVRLAGVIMAGVAIGIATAAQITAGV | sequence_CT06_ |
| | ALYEAMKNADNINKLKSSIESTNEAVVKLQETAEKTVYVL | Q393L |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRNT | |
| 443 | MHHLHPMIVCIFVMYTGIVGSDAILHYEKLSKIGLVKGVTR | F3_VGDVK |
| | KYKIKSNPLTKDIVIKMIPNVSNMSQCTGSVMENYKTRLN | (HeV)_VSVG_signal_ |
| | GILTPIKGALEIYKQNTHDLVGDVKLAGVIMAGVAIGIATA | sequence_CT06_ |
| | AQITAGVALYEAMKNADNINKLKSSIESTNEAVVKLQETA | Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRNT | |
| 444 | MKCLLYLAFLFIGVNCILHYEKLSKIGLVKGVTRKYKIKSN | F3- |
| | PLTKDIVIKMIPNVSNMSQCTGSVMENYKTRLNGILTPIKG | NDVF_CT_6_ |
| | ALEIYKQNTHDLVGDVKLAGVIMAGVAIGIATAAQITAGV | Q393L |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRNT | |
| 445 | MVVILDKRCYCNLLILILMISECSVGILHYEKLSKIGLVKGV | F3_VGDVK |
| | TRKYKIKSNPLTKDIVIKMIPNVSNMSQCTGSVMENYKTRL | (HeV)_NDVF_ |
| | NGILTPIKGALEIYKQNTHDLVGDVRLAGVIMAGVAIGIAT | CT_6_Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVKQKAQQ | |
| 446 | MVVILDKRCYCNLLILILMISECSVGILHYEKLSKIGLVKGV | F3_HPIV2_signal_ |
| | TRKYKIKSNPLTKDIVIKMIPNVSNMSQCTGSVMENYKTRL | sequence_NDVF_ |
| | NGILTPIKGALEIYKQNTHDLVGDVKLAGVIMAGVAIGIAT | CT_6_Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVKQKAQQ | |
| 447 | MHHLHPMIVCIFVMYTGIVGSDAILHYEKLSKIGLVKGVTR | F3_VSVG_signal |
| | KYKIKSNPLTKDIVIKMIPNVSNMSQCTGSVMENYKTRLN | sequence_NDVF_ |
| | GILTPIKGALEIYKQNTHDLVGDVRLAGVIMAGVAIGIATA | CT_6_Q393L |
| | AQITAGVALYEAMKNADNINKLKSSIESTNEAVVKLQETA | |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVKQKAQQ | |
| 448 | MKCLLYLAFLFIGVNCILHYEKLSKIGLVKGVTRKYKIKSN | F3_VGDVK |
| | PLTKDIVIKMIPNVSNMSQCTGSVMENYKTRLNGILTPIKG | (HeV)_HPIV2_ |
| | ALEIYKQNTHDLVGDVRLAGVIMAGVAIGIATAAQITAGV | signal_sequence_ |
| | ALYEAMKNADNINKLKSSIESTNEAVVKLQETAEKTVYVL | NDVF_CT_6_Q393L |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVKQKAQQ | |
| 449 | MHHLHPMIVCIFVMYTGIVGSDAILHYEKLSKIGLVKGVTR | F3_VGDVK |
| | KYKIKSNPLTKDIVIKMIPNVSNMSQCTGSVMENYKTRLN | (HeV)_VSVG_ |
| | GILTPIKGALEIYKQNTHDLVGDVKLAGVIMAGVAIGIATA | signal_sequence_ |
| | AQITAGVALYEAMKNADNINKLKSSIESTNEAVVKLQETA | NDVF_CT_6_Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVKQKAQQ | |
| 450 | MKCLLYLAFLFIGVNCILHYEKLSKIGLVKGVTRKYKIKSN | F3_HeVF_CT_6_ |
| | PLTKDIVIKMIPNVSNMSQCTGSVMENYKTRLNGILTPIKG | Q393L |
| | ALEIYKQNTHDLVGDVKLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVKQKAQQ | |
| 451 | MVVILDKRCYCNLLILILMISECSVGILHYEKLSKIGLVKGV | F3_VGDVK |
| | TRKYKIKSNPLTKDIVIKMIPNVSNMSQCTGSVMENYKTRL | (HeV)_HeVF_CT_ |
| | NGILTPIKGALEIYKQNTHDLVGDVRLAGVIMAGVAIGIAT | 6_Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRGN | |
| 452 | MVVILDKRCYCNLLILILMISECSVGILHYEKLSKIGLVKGV | F3_HPIV2_signal_ |
| | TRKYKIKSNPLTKDIVIKMIPNVSNMSQCTGSVMENYKTRL | sequence_HeVF_ |
| | NGILTPIKGALEIYKQNTHDLVGDVKLAGVIMAGVAIGIAT | CT_6_Q393L |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCLCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRGN | |
| 453 | MHHLHPMIVCIFVMYTGIVGSDAILHYEKLSKIGLVKGVTR | F3- |
| | KYKIKSNPLTKDIVIKMIPNVSNMSQCTGSVMENYKTRLN | _VSVG_signal_ |
| | GILTPIKGALEIYKQNTHDLVGDVRLAGVIMAGVAIGIATA | sequence_HeVF_CT_ |
| | AQITAGVALYEAMKNADNINKLKSSIESTNEAVVKLQETA | 6_Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRGN | |
| 454 | MKCLLYLAFLFIGVNCILHYEKLSKIGLVKGVTRKYKIKSN | F3_VGDVK |
| | PLTKDIVIKMIPNVSNMSQCTGSVMENYKTRLNGILTPIKG | (HeV)_HPIV2_ |
| | ALEIYKQNTHDLVGDVRLAGVIMAGVAIGIATAAQITAGV | signal_sequence_ |
| | ALYEAMKNADNINKLKSSIESTNEAVVKLQETAEKTVYVL | HeVF_CT_6_Q393L |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRGN | |
| 455 | MHHLHPMIVCIFVMYTGIVGSDAILHYEKLSKIGLVKGVTR | F3_VGDVK |
| | KYKIKSNPLTKDIVIKMIPNVSNMSQCTGSVMENYKTRLN | (HeV)_VSVG_ |
| | GILTPIKGALEIYKQNTHDLVGDVKLAGVIMAGVAIGIATA | signal_sequence_ |
| | AQITAGVALYEAMKNADNINKLKSSIESTNEAVVKLQETA | HeVF_CT_6_Q393L |
| | EKTVYVLTALQDYINTNLVPTIDKISCKQTELSLDLALSKY | |
| | LSDLLFVFGPNLQDPVSNSMTIQAISQAFGGNYETLLRTLG | |
| | YATEDFDDLLESDSITGQIIYVDLSSYYIIVRVYFPILTEIQQA | |
| | YIQELLPVSFNNDNSEWISIVPNFILVRNTLISNIEIGFCLITK | |
| | RSVICNQDYATPMTNNMRECLTGSTEKCPRELVVSSHVPR | |
| | FALSNGVLFANCISVTCLCQTTGRAISQSGEQTLLMIDNTTC | |
| | PTAVLGNVIISLGKYLGSVNYNSEGIAIGPPVFTDKVDISSQI | |
| | SSMNQSLQQSKDYIKEAQRLLDTVNPSLISMLSMIILYVLSI | |
| | ASLCIGLITFISFIIVEKKRGN | |
| 456 | MKCLLYLAFLFIGVNCILHYEKLSKIGLVKGVTRKYKIKSN | HPIV2_signal_ |
| | PLTKDIVIKMIPNVSNMSQCTGSVMENYKTRLNGILTPIKG | sequence_CT06_Q393L |
| | ALEIYKQNTHDLVGDVKLAGVIMAGVAIGIATAAQITAGV | |
| | ALYEAMKNADNINKLKSSIESTNEAVVKLQETAEKTVYVL | |
| | TALQDYINTNLVPTIDKISCKQTELSLDLALSKYLSDLLFVF | |
| | GPNLQDPVSNSMTIQAISQAFGGNYETLLRTLGYATEDFDD | |
| | LLESDSITGQIIYVDLSSYYIIVRVYFPILTEIQQAYIQELLPV | |
| | SFNNDNSEWISIVPNFILVRNTLISNIEIGFCLITKRSVICNQD | |
| | YATPMTNNMRECLTGSTEKCPRELVVSSHVPRFALSNGVL | |
| | FANCISVTCLCQTTGRAISQSGEQTLLMIDNTTCPTAVLGN | |
| | VIISLGKYLGSVNYNSEGIAIGPPVFTDKVDISSQISSMNQSL | |
| | QQSKDYIKEAQRLLDTVNPSLISMLSMIILYVLSIASLCIGLI | |
| | TFISFIIVEKKRGN | |
| 457 | MVVILDKRCYCNLLILILMISECSVGILHYEKLSKIGLVKGV | N1N2_CT06 |
| | TRKYKIKSNPLTKDIVIKMIPQVSQMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 458 | MVVILDKRCYCNLLILILMISECSVGILHYEKLSKIGLVKGV | N1N2N3_CT06 |
| | TRKYKIKSNPLTKDIVIKMIPQVSQMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 459 | MVVILDKRCYCNLLILILMISECSVGILHYEKLSKIGLVKGV | N1N2N3N5_CT06 |
| | TRKYKIKSNPLTKDIVIKMIPQVSQMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 460 | MVVILDKRCYCNLLILILMISECSVGILHYEKLSKIGLVKGV | N1N3_CT06 |
| | TRKYKIKSNPLTKDIVIKMIPQVSNMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMNQSLQQSKDYIKEAQRLLDTVNPSLISMLSMIILYV | |
| | LSIASLCIGLITFISFIIVEKKRNT | |
| 461 | MVVILDKRCYCNLLILILMISECSVGILHYEKLSKIGLVKGV | N1N2N5_CT06 |
| | TRKYKIKSNPLTKDIVIKMIPQVSQMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 462 | MVVILDKRCYCNLLILILMISECSVGILHYEKLSKIGLVKGV | N1N3N5_CT06 |
| | TRKYKIKSNPLTKDIVIKMIPQVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 463 | MVVILDKRCYCNLLILILMISECSVGILHYEKLSKIGLVKGV | N1N5_CT06 |
| | TRKYKIKSNPLTKDIVIKMIPQVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 464 | MVVILDKRCYCNLLILILMISECSVGILHYEKLSKIGLVKGV | N2N3N5_CT06 |
| | TRKYKIKSNPLTKDIVIKMIPNVSQMSQCTGSVMENYKTRL | |
| | NGILTPIKGALEIYKQNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNT | |
| | TCPTAVLGNVIISLGKYLGSVNYNSEGIAIGPPVFTDKVDIS | |
| | SQISSMQQSLQQSKDYIKEAQRLLDTVNPSLISMLSMIILYV | |
| | LSIASLCIGLITFISFIIVEKKRNT | |
| 465 | MALPVTALLLPLALLLHAARP | CD8α signal |
| | | peptide |
| 466 | METDTLLLWVLLLWVPGSTG | IgK signal peptide |
| 467 | MLLLVTSLLLCELPHPAFLLIP | GMCSFR-α |
| | | (CSF2RA) signal |
| | | peptide |
| 468 | TTTPAPRPPTPAPTIASQPLSLRPEACRPAAGGAVHTRGLDF | CD8α hinge |
| | ACD | domain |
| 469 | IEVMYPPPYLDNEKSNGTIIHVKGKHLCPSPLFPGPSKP | CD28 hinge |
| | | domain |
| 470 | AAAIEVMYPPPYLDNEKSNGTIIHVKGKHLCPSPLFPGPSKP | CD28 hinge |
| | | domain |
| 471 | ESKYGPPCPPCP | IgG4 hinge domain |
| 472 | ESKYGPPCPSCP | IgG4 hinge domain |
| 473 | IYIWAPLAGTCGVLLLSLVITLYC | CD8α |
| | | transmembrane |
| | | domain |
| 474 | FWVLVVVGGVLACYSLLVTVAFIIFWV | CD28 |
| | | transmembrane |
| | | domain |
| 475 | MFWVLVVVGGVLACYSLLVTVAFIIFWV | CD28 |
| | | transmembrane |
| | | domain |
| 476 | KRGRKKLLYIFKQPFMRPVQTTQEEDGCSCRFPEEEEGGCE | 4-1BB |
| | L | costimulatory |
| | | domain |
| 477 | RSKRSRLLHSDYMNMTPRRPGPTRKHYQPYAPPRDFAAYR | CD28 |
| | S | costimulatory |
| | | domain |
| 478 | RVKFSRSADAPAYQQGQNQLYNELNLGRREEYDVLDKRR | CD3ζ signaling |
| | GRDPEMGGKPRRKNPQEGLYNELQKDKMAEAYSEIGMKG | domain |
| | ERRRGKGHDGLYQGLSTATKDTYDALHMQALPPR | |
| 479 | RVKFSRSADAPAYKQGQNQLYNELNLGRREEYDVLDKRR | CD3ζ signaling |
| | GRDPEMGGKPRRKNPQEGLYNELQKDKMAEAYSEIGMKG | domain (with Q to |
| | ERRRGKGHDGLYQGLSTATKDTYDALHMQALPPR | K mutation at |
| | | position 14) |
| 480 | DIQMTQTTSSLSASLGDRVTISCRASQDISKYLNWYQQKPD | Anti-CD19 FMC63 |
| | GTVKLLIYHTSRLHSGVPSRFSGSGSGTDYSLTISNLEQEDI | scFv entire |
| | ATYFCQQGNTLPYTFGGGTKLEITGSTSGSGKPGSGEGSTK | sequence, with |
| | GEVKLQESGPGLVAPSQSLSVTCTVSGVSLPDYGVSWIRQP | Whitlow linker |
| | PRKGLEWLGVIWGSETTYYNSALKSRLTIIKDNSKSQVFLK | |
| | MNSLQTDDTAIYYCAKHYYYGGSYAMDYWGQGTSVTVS | |
| | S | |
| 481 | DIQMTQTTSSLSASLGDRVTISCRASQDISKYLNWYQQKPD | Anti-CD19 FMC63 |
| | GTVKLLIYHTSRLHSGVPSRFSGSGSGTDYSLTISNLEQEDI | scFv light chain |
| | ATYFCQQGNTLPYTFGGGTKLEIT | variable region |
| 482 | QDISKY | Anti-CD19 FMC63 |
| | | scFv light chain |
| | | CDR1 |
| 483 | HTS | Anti-CD19 FMC63 |
| | | scFv light chain |
| | | CDR2 |
| 484 | QQGNTLPYT | Anti-CD19 FMC63 |
| | | scFv light chain |
| | | CDR3 |
| 485 | GSTSGSGKPGSGEGSTKG | Whitlow linker |
| 486 | EVKLQESGPGLVAPSQSLSVTCTVSGVSLPDYGVSWIRQPP | Anti-CD19 FMC63 |
| | RKGLEWLGVIWGSETTYYNSALKSRLTIIKDNSKSQVFLK | scFv heavy chain |
| | MNSLQTDDTAIYYCAKHYYYGGSYAMDYWGQGTSVTVS | variable region |
| | S | |
| 487 | GVSLPDYG | Anti-CD19 FMC63 |
| | | scFv heavy chain |
| | | CDR1 |
| 488 | IWGSETT | Anti-CD19 FMC63 |
| | | scFv heavy chain |
| | | CDR2 |
| 489 | AKHYYYGGSYAMDY | Anti-CD19 FMC63 |
| | | scFv heavy chain |
| | | CDR3 |
| 490 | DIQMTQTTSSLSASLGDRVTISCRASQDISKYLNWYQQKPD | Anti-CD19 FMC63 |
| | GTVKLLIYHTSRLHSGVPSRFSGSGSGTDYSLTISNLEQEDI | scFv entire |
| | ATYFCQQGNTLPYTFGGGTKLEITGGGGSGGGGSGGGGSE | sequence, with |
| | VKLQESGPGLVAPSQSLSVTCTVSGVSLPDYGVSWIRQPPR | 3xG₄S linker |
| | KGLEWLGVIWGSETTYYNSALKSRLTIIKDNSKSQVFLKM | |
| | NSLQTDDTAIYYCAKHYYYGGSYAMDYWGQGTSVTVSS | |
| 491 | GGGGSGGGGSGGGGS | 3xG₄S linker |
| 492 | atggccttaccagtgaccgccttgctcctgccgctggccttgctgctccacgccgccaggcc | Exemplary CD19 |
| | ggacatccagatgacacagactacatcctccctgtctgcctctctgggagacagagtcaccat | CAR nucleotide |
| | cagttgcagggcaagtcaggacattagtaaatatttaaattggtatcagcagaaaccagatgg | sequence |
| | aactgttaaactcctgattaccatacatcaagattacactcaggagtcccatcaaggttcagtg | |
| | gcagtgggtctggaacagattattctctcaccattagcaacctggagcaagaagatattgcca | |
| | cttacttttgccaacagggtaatacgcttccgtacacgttcggaggggggaccaagctggaga | |
| | tcacaggctccacctctggatccggcaagcccggatctggcgagggatccaccaagggcg | |
| | aggtgaaactgcaggagtcaggacctggcctggtggcgccctcacagagcctgtccgtcac | |
| | atgcactgtctcaggggtctcattacccgactatggtgtaagctggattcgccagcctccacga | |
| | aagggtctggagtggctgggagtaatatggggtagtgaaaccacatactataattcagctctc | |
| | aaatccagactgaccatcatcaaggacaactccaagagccaagttttcttaaaaatgaacagt | |
| | ctgcaaactgatgacacagccatttactactgtgccaaacattattactacggtggtagctatgc | |
| | tatggactactggggccaaggaacctcagtcaccgtctcctcaaccacgacgccagcgccg | |
| | cgaccaccaacaccggcgcccaccatcgcgtcgcagcccctgtccctgcgcccagaggcg | |
| | tgccggccagcggcggggggcgcagtgcacacgagggggctggacttcgcctgtgatatc | |
| | tacatctgggcgcccttggccgggacttgtggggtccttctcctgtcactggttatcacccttta | |
| | ctgcaaacggggcagaaagaaactcctgtatatattcaaacaaccatttatgagaccagtaca | |
| | aactactcaagaggaagatggctgtagctgccgatttccagaagaagaagaaggaggatgt | |
| | gaactgagagtgaagttcagcaggagcgcagacgcccccgcgtaccagcagggccagaa | |
| | ccagctctataacgagctcaatctaggacgaagagaggagtacgatgttttggacaagagac | |
| | gtggccgggaccctgagatggggggaaagccgagaaggaagaaccctcaggaaggcct | |
| | gtacaatgaactgcagaaagataagatggcggaggcctacagtgagattgggatgaaaggc | |
| | gagcgccggaggggcaaggggcacgatggcctttaccagggtctcagtacagccaccaag | |
| | gacacctacgacgcccttcacatgcaggccctgccccctcgc | |
| 493 | MALPVTALLLPLALLLHAARPDIQMTQTTSSLSASLGDRVT | Exemplary CD19 |
| | ISCRASQDISKYLNWYQQKPDGTVKLLIYHTSRLHSGVPSR | CAR amino acid |
| | FSGSGSGTDYSLTISNLEQEDIATYFCQQGNTLPYTFGGGT | sequence |
| | KLEITGSTSGSGKPGSGEGSTKGEVKLQESGPGLVAPSQSLS | |
| | VTCTVSGVSLPDYGVSWIRQPPRKGLEWLGVIWGSETTYY | |
| | NSALKSRLTIIKDNSKSQVFLKMNSLQTDDTAIYYCAKHYY | |
| | YGGSYAMDYWGQGTSVTVSSTTTPAPRPPTPAPTIASQPLS | |
| | LRPEACRPAAGGAVHTRGLDFACDIYIWAPLAGTCGVLLL | |
| | SLVITLYCKRGRKKLLYIFKQPFMRPVQTTQEEDGCSCRFP | |
| | EEEEGGCELRVKFSRSADAPAYQQGQNQLYNELNLGRREE | |
| | YDVLDKRRGRDPEMGGKPRRKNPQEGLYNELQKDKMAE | |
| | AYSEIGMKGERRRGKGHDGLYQGLSTATKDTYDALHMQA | |
| | LPPR | |
| 494 | atggccttaccagtgaccgccttgctcctgccgctggccttgctgctccacgccgccaggcc | Tisagenlecleucel |
| | ggacatccagatgacacagactacatcctccctgtctgcctctctgggagacagagtcaccat | CD19 CAR |
| | cagttgcagggcaagtcaggacattagtaaatatttaaattggtatcagcagaaaccagatgg | nucleotide |
| | aactgttaaactcctgatctaccatacatcaagattacactcaggagtcccatcaaggttcagtg | sequence |
| | gcagtgggtctggaacagattattctctcaccattagcaacctggagcaagaagatattgcca | |
| | cttacttttgccaacagggtaatacgcttccgtacacgttcggaggggggaccaagctggaga | |
| | tcacaggtggcggtggctcgggcggtggtgggtcgggtggcggcggatctgaggtgaaac | |
| | tgcaggagtcaggacctggcctggtggcgccctcacagagcctgtccgtcacatgcactgtc | |
| | tcaggggtctcattacccgactatggtgtaagctggattcgccagcctccacgaaagggtctg | |
| | gagtggctgggagtaatatggggtagtgaaaccacatactataattcagctctcaaatccaga | |
| | ctgaccatcatcaaggacaactccaagagccaagttttcttaaaaatgaacagtctgcaaactg | |
| | atgacacagccatttactactgtgccaaacattattactacggtggtagctatgctatggactact | |
| | ggggccaaggaacctcagtcaccgtctcctcaaccacgacgccagcgccgcgaccaccaa | |
| | caccggcgcccaccatcgcgtcgcagcccctgtccctgcgcccagaggcgtgccggccag | |
| | cggcggggggcgcagtgcacacgagggggctggacttcgcctgtgatatctacatctgggc | |
| | gcccttggccgggacttgtggggtccttctcctgtcactggttatcaccctttactgcaaacggg | |
| | gcagaaagaaactcctgtatatattcaaacaaccatttatgagaccagtacaaactactcaaga | |
| | ggaagatggctgtagctgccgatttccagaagaagaagaaggaggatgtgaactgagagtg | |
| | aagttcagcaggagcgcagacgcccccgcgtacaagcagggccagaaccagctctataac | |
| | gagctcaatctaggacgaagagaggagtacgatgttttggacaagagacgtggccgggacc | |
| | ctgagatggggggaaagccgagaaggaagaaccctcaggaaggcctgtacaatgaactgc | |
| | agaaagataagatggcggaggcctacagtgagattgggatgaaaggcgagcgccggagg | |
| | ggcaaggggcacgatggcctttaccagggtctcagtacagccaccaaggacacctacgac | |
| | gcccttcacatgcaggccctgccccctcgc | |
| 495 | MALPVTALLLPLALLLHAARPDIQMTQTTSSLSASLGDRVT | Tisagenlecleucel |
| | ISCRASQDISKYLNWYQQKPDGTVKLLIYHTSRLHSGVPSR | CD19 CAR amino |
| | FSGSGSGTDYSLTISNLEQEDIATYFCQQGNTLPYTFGGGT | acid sequence |
| | KLEITGGGGSGGGGSGGGGSEVKLQESGPGLVAPSQSLSVT | |
| | CTVSGVSLPDYGVSWIRQPPRKGLEWLGVIWGSETTYYNS | |
| | ALKSRLTIIKDNSKSQVFLKMNSLQTDDTAIYYCAKHYYY | |
| | GGSYAMDYWGQGTSVTVSSTTTPAPRPPTPAPTIASQPLSL | |
| | RPEACRPAAGGAVHTRGLDFACDIYIWAPLAGTCGVLLLS | |
| | LVITLYCKRGRKKLLYIFKQPFMRPVQTTQEEDGCSCRFPE | |
| | EEEGGCELRVKFSRSADAPAYKQGQNQLYNELNLGRREEY | |
| | DVLDKRRGRDPEMGGKPRRKNPQEGLYNELQKDKMAEA | |
| | YSEIGMKGERRRGKGHDGLYQGLSTATKDTYDALHMQAL | |
| | PPR | |
| 496 | atgctgctgctggtgaccagcctgctgctgtgcgagctgccccaccccgcctttctgctgatcc | Lisocabtagene |
| | ccgacatccagatgacccagaccacctccagcctgagcgccagcctgggcgaccgggtga | maraleucel CD19 |
| | ccatcagctgccgggccagccaggacatcagcaagtacctgaactggtatcagcagaagcc | CAR nucleotide |
| | cgacggcaccgtcaagctgctgatctaccacaccagccggctgcacagcggcgtgcccag | sequence |
| | ccggtttagcggcagcggctccggcaccgactacagcctgaccatctccaacctggaacag | |
| | gaagatatcgccacctacttttgccagcagggcaacacactgccctacacctttggcggcgg | |
| | aacaaagctggaaatcaccggcagcacctccggcagcggcaagcctggcagcggcgagg | |
| | gcagcaccaagggcgaggtgaagctgcaggaaagcggccctggcctggtggcccccagc | |
| | cagagcctgagcgtgacctgcaccgtgagcggcgtgagcctgcccgactacggcgtgagc | |
| | tggatccggcagccccccaggaagggcctggaatggctgggcgtgatctggggcagcga | |
| | gaccacctactacaacagcgccctgaagagccggctgaccatcatcaaggacaacagcaa | |
| | gagccaggtgttcctgaagatgaacagcctgcagaccgacgacaccgccatctactactgc | |
| | gccaagcactactactacggcggcagctacgccatggactactggggccagggcaccagc | |
| | gtgaccgtgagcagcgaatctaagtacggaccgccctgccccccttgccctatgttctgggtg | |
| | ctggtggtggtcggaggcgtgctggcctgctacagcctgctggtcaccgtggccttcatcatc | |
| | ttttgggtgaaacggggcagaaagaaactcctgtatatattcaaacaaccatttatgagaccag | |
| | tacaaactactcaagaggaagatggctgtagctgccgatttccagaagaagaagaaggagg | |
| | atgtgaactgcgggtgaagttcagcagaagcgccgacgcccctgcctaccagcagggcca | |
| | gaatcagctgtacaacgagctgaacctgggcagaagggaagagtacgacgtcctggataag | |
| | cggagaggccgggaccctgagatgggcggcaagcctcggcggaagaacccccaggaag | |
| | gcctgtataacgaactgcagaaagacaagatggccgaggcctacagcgagatcggcatga | |
| | agggcgagcggaggggggcaagggccacgacggcctgtatcagggcctgtccaccgc | |
| | caccaaggatacctacgacgccctgcacatgcaggccctgcccccaagg | |
| 497 | MLLLVTSLLLCELPHPAFLLIPDIQMTQTTSSLSASLGDRVTI | Lisocabtagene |
| | SCRASQDISKYLNWYQQKPDGTVKLLIYHTSRLHSGVPSRF | maraleucel CD19 |
| | SGSGSGTDYSLTISNLEQEDIATYFCQQGNTLPYTFGGGTK | CAR amino acid |
| | LEITGSTSGSGKPGSGEGSTKGEVKLQESGPGLVAPSQSLSV | sequence |
| | TCTVSGVSLPDYGVSWIRQPPRKGLEWLGVIWGSETTYYN | |
| | SALKSRLTIIKDNSKSQVFLKMNSLQTDDTAIYYCAKHYYY | |
| | GGSYAMDYWGQGTSVTVSSESKYGPPCPPCPMFWVLVVV | |
| | GGVLACYSLLVTVAFIIFWVKRGRKKLLYIFKQPFMRPVQT | |
| | TQEEDGCSCRFPEEEEGGCELRVKFSRSADAPAYQQGQNQ | |
| | LYNELNLGRREEYDVLDKRRGRDPEMGGKPRRKNPQEGL | |
| | YNELQKDKMAEAYSEIGMKGERRRGKGHDGLYQGLSTAT | |
| | KDTYDALHMQALPPR | |
| 498 | atgcttctcctggtgacaagccttctgctctgtgagttaccacacccagcattcctcctgatccc | Axicabtagene |
| | agacatccagatgacacagactacatcctccctgtctgcctctctgggagacagagtcaccat | ciloleucel CD19 |
| | cagttgcagggcaagtcaggacattagtaaatatttaaattggtatcagcagaaaccagatgg | CAR nucleotide |
| | aactgttaaactcctgatctaccatacatcaagattacactcaggagtcccatcaaggttcagtg | sequence |
| | gcagtgggtctggaacagattattctctcaccattagcaacctggagcaagaagatattgcca | |
| | cttacttttgccaacagggtaatacgcttccgtacacgttcggaggggggactaagttggaaat | |
| | aacaggctccacctctggatccggcaagcccggatctggcgagggatccaccaagggcga | |
| | ggtgaaactgcaggagtcaggacctggcctggtggcgccctcacagagcctgtccgtcaca | |
| | tgcactgtctcaggggtctcattacccgactatggtgtaagctggattcgccagcctccacgaa | |
| | agggtctggagtggctgggagtaatatggggtagtgaaaccacatactataattcagctctca | |
| | aatccagactgaccatcatcaaggacaactccaagagccaagttttcttaaaaatgaacagtct | |
| | gcaaactgatgacacagccatttactactgtgccaaacattattactacggtggtagctatgcta | |
| | tggactactggggtcaaggaacctcagtcaccgtctcctcagcggccgcaattgaagttatgt | |
| | atcctcctccttacctagacaatgagaagagcaatggaaccattatccatgtgaaagggaaac | |
| | acctttgtccaagtcccctatttcccggaccttctaagcccttttgggtgctggtggtggttgggg | |
| | gagtcctggcttgctatagcttgctagtaacagtggcctttattattttctgggtgaggagtaaga | |
| | ggagcaggctcctgcacagtgactacatgaacatgactccccgccgccccgggcccaccc | |
| | gcaagcattaccagccctatgccccaccacgcgacttcgcagcctatcgctccagagtgaag | |
| | ttcagcaggagcgcagacgcccccgcgtaccagcagggccagaaccagctctataacgag | |
| | ctcaatctaggacgaagagaggagtacgatgttttggacaagagacgtggccgggaccctg | |
| | agatggggggaaagccgagaaggaagaaccctcaggaaggcctgtacaatgaactgcag | |
| | aaagataagatggcggaggcctacagtgagattgggatgaaaggcgagcgccggagggg | |
| | caaggggcacgatggcctttaccagggtctcagtacagccaccaaggacacctacgacgcc | |
| | cttcacatgcaggccctgccccctcgc | |
| 499 | MLLLVTSLLLCELPHPAFLLIPDIQMTQTTSSLSASLGDRVTI | Axicabtagene |
| | SCRASQDISKYLNWYQQKPDGTVKLLIYHTSRLHSGVPSRF | ciloleucel CD19 |
| | SGSGSGTDYSLTISNLEQEDIATYFCQQGNTLPYTFGGGTK | CAR amino acid |
| | LEITGSTSGSGKPGSGEGSTKGEVKLQESGPGLVAPSQSLSV | sequence |
| | TCTVSGVSLPDYGVSWIRQPPRKGLEWLGVIWGSETTYYN | |
| | SALKSRLTIIKDNSKSQVFLKMNSLQTDDTAIYYCAKHYYY | |
| | GGSYAMDYWGQGTSVTVSSAAAIEVMYPPPYLDNEKSNG | |
| | TIIHVKGKHLCPSPLFPGPSKPFWVLVVVGGVLACYSLLVT | |
| | VAFIIFWVRSKRSRLLHSDYMNMTPRRPGPTRKHYQPYAP | |
| | PRDFAAYRSRVKFSRSADAPAYQQGQNQLYNELNLGRREE | |
| | YDVLDKRRGRDPEMGGKPRRKNPQEGLYNELQKDKMAE | |
| | AYSEIGMKGERRRGKGHDGLYQGLSTATKDTYDALHMQA | |
| | LPPR | |
| 500 | DIVLTQSPAILSASPGEKVTMTCRASSSVNYMDWYQKKPG | Anti-CD20 Leu16 |
| | SSPKPWIYATSNLASGVPARFSGSGSGTSYSLTISRVEAEDA | scFv entire |
| | ATYYCQQWSFNPPTFGGGTKLEIKGSTSGSGKPGSGEGSTK | sequence, with |
| | GEVQLQQSGAELVKPGASVKMSCKASGYTFTSYNMHWV | Whitlow linker |
| | KQTPGQGLEWIGAIYPGNGDTSYNQKFKGKATLTADKSSS | |
| | TAYMQLSSLTSEDSADYYCARSNYYGSSYWFFDVWGAGT | |
| | TVTVSS | |
| 501 | DIVLTQSPAILSASPGEKVTMTCRASSSVNYMDWYQKKPG | Anti-CD20 Leu16 |
| | SSPKPWIYATSNLASGVPARFSGSGSGTSYSLTISRVEAEDA | scFv light chain |
| | ATYYCQQWSFNPPTFGGGTKLEIK | variable region |
| 502 | RASSSVNYMD | Anti-CD20 Leu16 |
| | | scFv light chain |
| | | CDR1 |
| 503 | ATSNLAS | Anti-CD20 Leu16 |
| | | scFv light chain |
| | | CDR2 |
| 504 | QQWSFNPPT | Anti-CD20 Leu16 |
| | | scFv light chain |
| | | CDR3 |
| 505 | EVQLQQSGAELVKPGASVKMSCKASGYTFTSYNMHWVK | Anti-CD20 Leu16 |
| | QTPGQGLEWIGAIYPGNGDTSYNQKFKGKATLTADKSSST | scFv heavy chain |
| | AYMQLSSLTSEDSADYYCARSNYYGSSYWFFDVWGAGTT | |
| | VTVSS | |
| 506 | SYNMH | Anti-CD20 Leu16 |
| | | scFv heavy chain |
| | | CDR1 |
| 507 | AIYPGNGDTSYNQKFKG | Anti-CD20 Leu16 |
| | | scFv heavy chain |
| | | CDR2 |
| 508 | SNYYGSSYWFFDV | Anti-CD20 Leu16 |
| | | scFv heavy chain |
| | | CDR3 |
| 509 | QVQLQQSGPGLVKPSQTLSLTCAISGDSVSSNSAAWNWIR | Anti-CD22 m971 |
| | QSPSRGLEWLGRTYYRSKWYNDYAVSVKSRITINPDTSKN | scFv entire |
| | QFSLQLNSVTPEDTAVYYCAREVTGDLEDAFDIWGQGTM | sequence, with |
| | VTVSSGGGGSGGGGSGGGGSDIQMTQSPSSLSASVGDRVTI | 3xG4S linker |
| | TCRASQTIWSYLNWYQQRPGKAPNLLIYAASSLQSGVPSRF | |
| | SGRGSGTDFTLTISSLQAEDFATYYCQQSYSIPQTFGQGTKL | |
| | EIK | |
| 510 | QVQLQQSGPGLVKPSQTLSLTCAISGDSVSSNSAAWNWIR | Anti-CD22 m971 |
| | QSPSRGLEWLGRTYYRSKWYNDYAVSVKSRITINPDTSKN | scFv heavy chain |
| | QFSLQLNSVTPEDTAVYYCAREVTGDLEDAFDIWGQGTM | variable region |
| | VTVSS | |
| 511 | GDSVSSNSAA | Anti-CD22 m971 |
| | | scFv heavy chain |
| | | CDR1 |
| 512 | TYYRSKWYN | Anti-CD22 m971 |
| | | scFv heavy chain |
| | | CDR2 |
| 513 | AREVTGDLEDAFDI | Anti-CD22 m971 |
| | | scFv heavy chain |
| | | CDR3 |
| 514 | DIQMTQSPSSLSASVGDRVTITCRASQTIWSYLNWYQQRPG | Anti-CD22 m971 |
| | KAPNLLIYAASSLQSGVPSRFSGRGSGTDFTLTISSLQAEDF | scFv light chain |
| | ATYYCQQSYSIPQTFGQGTKLEIK | |
| 515 | QTIWSY | Anti-CD22 m971 |
| | | scFv light chain |
| | | CDR1 |
| 516 | AAS | Anti-CD22 m971 |
| | | scFv light chain |
| | | CDR2 |
| 517 | QQSYSIPQT | Anti-CD22 m971 |
| | | scFv light chain |
| | | CDR3 |
| 518 | QVQLQQSGPGMVKPSQTLSLTCAISGDSVSSNSVAWNWIR | Anti-CD22 m971- |
| | QSPSRGLEWLGRTYYRSTWYNDYAVSMKSRITINPDTNKN | L7 scFv entire |
| | QFSLQLNSVTPEDTAVYYCAREVTGDLEDAFDIWGQGTM | sequence, with |
| | VTVSSGGGGSGGGGSGGGGSDIQMIQSPSSLSASVGDRVTI | 3xG₄S linker |
| | TCRASQTIWSYLNWYRQRPGEAPNLLIYAASSLQSGVPSRF | |
| | SGRGSGTDFTLTISSLQAEDFATYYCQQSYSIPQTFGQGTKL | |
| | EIK | |
| 519 | QVQLQQSGPGMVKPSQTLSLTCAISGDSVSSNSVAWNWIR | Anti-CD22 m971- |
| | QSPSRGLEWLGRTYYRSTWYNDYAVSMKSRITINPDTNKN | L7 scFv heavy |
| | QFSLQLNSVTPEDTAVYYCAREVTGDLEDAFDIWGQGTM | chain variable |
| | VTVSS | region |
| 520 | GDSVSSNSVA | Anti-CD22 m971- |
| | | L7 scFv heavy |
| | | chain CDR1 |
| 521 | TYYRSTWYN | Anti-CD22 m971- |
| | | L7 scFv heavy |
| | | chain CDR2 |
| 522 | AREVTGDLEDAFDI | Anti-CD22 m971- |
| | | L7 scFv heavy |
| | | chain CDR3 |
| 523 | DIQMIQSPSSLSASVGDRVTITCRASQTIWSYLNWYRQRPG | Anti-CD22 m971- |
| | EAPNLLIYAASSLQSGVPSRFSGRGSGTDFTLTISSLQAEDF | L7 scFv light chain |
| | ATYYCQQSYSIPQTFGQGTKLEIK | variable region |
| 524 | QTIWSY | Anti-CD22 m971- |
| | | L7 scFv light chain |
| | | CDR1 |
| 525 | AAS | Anti-CD22 m971- |
| | | L7 scFv light chain |
| | | CDR2 |
| 526 | QQSYSIPQT | Anti-CD22 m971- |
| | | L7 scFv light chain |
| | | CDR3 |
| 527 | DIVLTQSPASLAMSLGKRATISCRASESVSVIGAHLIHWYQ | Anti-BCMA |
| | QKPGQPPKLLIYLASNLETGVPARFSGSGSGTDFTLTIDPVE | C11D5.3 scFv |
| | EDDVAIYSCLQSRIFPRTFGGGTKLEIKGSTSGSGKPGSGEG | entire sequence, |
| | STKGQIQLVQSGPELKKPGETVKISCKASGYTFTDYSINWV | with Whitlow |
| | KRAPGKGLKWMGWINTETREPAYAYDFRGRFAFSLETSAS | linker |
| | TAYLQINNLKYEDTATYFCALDYSYAMDYWGQGTSVTVS | |
| | S | |
| 528 | DIVLTQSPASLAMSLGKRATISCRASESVSVIGAHLIHWYQ | Anti-BCMA |
| | QKPGQPPKLLIYLASNLETGVPARFSGSGSGTDFTLTIDPVE | C11D5.3 scFv light |
| | EDDVAIYSCLQSRIFPRTFGGGTKLEIK | chain variable |
| | | region |
| 529 | RASESVSVIGAHLIH | Anti-BCMA |
| | | C11D5.3 scFv light |
| | | chain CDR1 |
| 530 | LASNLET | Anti-BCMA |
| | | C11D5.3 scFv light |
| | | chain CDR2 |
| 531 | LQSRIFPRT | Anti-BCMA |
| | | C11D5.3 scFv light |
| | | chain CDR3 |
| 532 | QIQLVQSGPELKKPGETVKISCKASGYTFTDYSINWVKRAP | Anti-BCMA |
| | GKGLKWMGWINTETREPAYAYDFRGRFAFSLETSASTAYL | C11D5.3 scFv |
| | QINNLKYEDTATYFCALDYSYAMDYWGQGTSVTVSS | heavy chain |
| | | variable region |
| 533 | DYSIN | Anti-BCMA |
| | | C11D5.3 scFv |
| | | heavy chain CDR1 |
| 534 | WINTETREPAYAYDFRG | Anti-BCMA |
| | | C11D5.3 scFv |
| | | heavy chain CDR2 |
| 535 | DYSYAMDY | Anti-BCMA |
| | | C11D5.3 scFv |
| | | heavy chain CDR3 |
| 536 | DIVLTQSPPSLAMSLGKRATISCRASESVTILGSHLIYWYQQ | Anti-BCMA |
| | KPGQPPTLLIQLASNVQTGVPARFSGSGSRTDFTLTIDPVEE | C12A3.2 scFv |
| | DDVAVYYCLQSRTIPRTFGGGTKLEIKGSTSGSGKPGSGEG | entire sequence, |
| | STKGQIQLVQSGPELKKPGETVKISCKASGYTFRHYSMNW | with Whitlow |
| | VKQAPGKGLKWMGRINTESGVPIYADDFKGRFAFSVETSA | linker |
| | STAYLVINNLKDEDTASYFCSNDYLYSLDFWGQGTALTVS | |
| | S | |
| 537 | DIVLTQSPPSLAMSLGKRATISCRASESVTILGSHLIYWYQQ | Anti-BCMA |
| | KPGQPPTLLIQLASNVQTGVPARFSGSGSRTDFTLTIDPVEE | C12A3.2 scFv light |
| | DDVAVYYCLQSRTIPRTFGGGTKLEIK | chain variable |
| | | region |
| 538 | RASESVTILGSHLIY | Anti-BCMA |
| | | C12A3.2 scFv light |
| | | chain CDR1 |
| 539 | LASNVQT | Anti-BCMA |
| | | C12A3.2 scFv light |
| | | chain CDR2 |
| 540 | LQSRTIPRT | Anti-BCMA |
| | | C12A3.2 scFv light |
| | | chain CDR3 |
| 541 | QIQLVQSGPELKKPGETVKISCKASGYTFRHYSMNWVKQA | Anti-BCMA |
| | PGKGLKWMGRINTESGVPIYADDFKGRFAFSVETSASTAY | C12A3.2 scFv |
| | LVINNLKDEDTASYFCSNDYLYSLDFWGQGTALTVSS | heavy chain |
| | | variable region |
| 542 | HYSMN | Anti-BCMA |
| | | C12A3.2 scFv |
| | | heavy chain CDR1 |
| 543 | RINTESGVPIYADDFKG | Anti-BCMA |
| | | C12A3.2 scFv |
| | | heavy chain CDR2 |
| 544 | DYLYSLDF | Anti-BCMA |
| | | C12A3.2 scFv |
| | | heavy chain CDR3 |
| 545 | EVQLLESGGGLVQPGGSLRLSCAASGFTFSSYAMSWVRQA | Anti-BCMA |
| | PGKGLEWVSSISGSGDYIYYADSVKGRFTISRDISKNTLYLQ | FHVH33 entire |
| | MNSLRAEDTAVYYCAKEGTGANSSLADYRGQGTLVTVSS | sequence |
| 546 | GFTFSSYA | Anti-BCMA |
| | | FHVH33 CDR1 |
| 547 | ISGSGDYI | Anti-BCMA |
| | | FHVH33 CDR2 |
| 548 | AKEGTGANSSLADY | Anti-BCMA |
| | | FHVH33 CDR3 |
| 549 | DIQMTQSPSSLSASVGDRVTITCRASQSISSYLNWYQQKPG | Anti-BCMA |
| | KAPKLLIYAASSLQSGVPSRFSGSGSGTDFTLTISSLQPEDFA | CT103A scFv |
| | TYYCQQKYDLLTFGGGTKVEIKGSTSGSGKPGSGEGSTKG | entire sequence, |
| | QLQLQESGPGLVKPSETLSLTCTVSGGSISSSSYYWGWIRQ | with Whitlow |
| | PPGKGLEWIGSISYSGSTYYNPSLKSRVTISVDTSKNQFSLK | linker |
| | LSSVTAADTAVYYCARDRGDTILDVWGQGTMVTVSS | |
| 550 | DIQMTQSPSSLSASVGDRVTITCRASQSISSYLNWYQQKPG | Anti-BCMA |
| | KAPKLLIYAASSLQSGVPSRFSGSGSGTDFTLTISSLQPEDFA | CT103A scFv light |
| | TYYCQQKYDLLTFGGGTKVEIK | chain variable |
| | | region |
| 551 | QSISSY | Anti-BCMA |
| | | CT103A scFv light |
| | | chain CDR1 |
| 552 | AAS | Anti-BCMA |
| | | CT103A scFv light |
| | | chain CDR2 |
| 553 | QQKYDLLT | Anti-BCMA |
| | | CT103A scFv light |
| | | chain CDR3 |
| 554 | QLQLQESGPGLVKPSETLSLTCTVSGGSISSSSYYWGWIRQ | Anti-BCMA |
| | PPGKGLEWIGSISYSGSTYYNPSLKSRVTISVDTSKNQFSLK | CT103A scFv |
| | LSSVTAADTAVYYCARDRGDTILDVWGQGTMVTVSS | heavy chain |
| | | variable region |
| 555 | GGSISSSSYY | Anti-BCMA |
| | | CT103A scFv |
| | | heavy chain CDR1 |
| 556 | ISYSGST | Anti-BCMA |
| | | CT103A scFv |
| | | heavy chain CDR2 |
| 557 | ARDRGDTILDV | Anti-BCMA |
| | | CT103A scFv |
| | | heavy chain CDR3 |
| 558 | atggccttaccagtgaccgccttgctcctgccgctggccttgctgctccacgccgccaggcc | Exemplary BCMA |
| | ggacatccagatgacccagtctccatcctccctgtctgcatctgtaggagacagagtcaccat | CAR nucleotide |
| | cacttgccgggcaagtcagagcattagcagctatttaaattggtatcagcagaaaccagggaa | sequence |
| | agcccctaagctcctgatctatgctgcatccagtttgcaaagtggggtcccatcaaggttcagt | |
| | ggcagtggatctgggacagatttcactctcaccatcagcagtctgcaacctgaagattttgcaa | |
| | cttactactgtcagcaaaaatacgacctcctcacttttggcggagggaccaaggttgagatcaa | |
| | aggcagcaccagcggctccggcaagcctggctctggcgagggcagcacaaagggacag | |
| | ctgcagctgcaggagtcgggcccaggactggtgaagccttcggagaccctgtccctcacct | |
| | gcactgtctctggtggctccatcagcagtagtagttactactggggctggatccgccagcccc | |
| | cagggaaggggctggagtggattgggagtatctcctatagtgggagcacctactacaaccc | |
| | gtccctcaagagtcgagtcaccatatccgtagacacgtccaagaaccagttctccctgaagct | |
| | gagttctgtgaccgccgcagacacggcggtgtactactgcgccagagatcgtggagacacc | |
| | atactagacgtatggggtcagggtacaatggtcaccgtcagctcattcgtgcccgtgttcctgc | |
| | ccgccaaacctaccaccacccctgcccctagacctcccaccccagccccaacaatcgccag | |
| | ccagcctctgtctctgcggcccgaagcctgtagacctgctgccggcggagccgtgcacacc | |
| | agaggcctggacttcgcctgcgacatctacatctgggcccctctggccggcacctgtggcgt | |
| | gctgctgctgagcctggtgatcaccctgtactgcaaccaccggaacaaacggggcagaaag | |
| | aaactcctgtatatattcaaacaaccatttatgagaccagtacaaactactcaagaggaagatg | |
| | gctgtagctgccgatttccagaagaagaagaaggaggatgtgaactgagagtgaagttcagc | |
| | agatccgccgacgcccctgcctaccagcagggacagaaccagctgtacaacgagctgaac | |
| | ctgggcagacgggaagagtacgacgtgctggacaagcggagaggccgggaccccgaga | |
| | tgggcggaaagcccagacggaagaacccccaggaaggcctgtataacgaactgcagaaa | |
| | gacaagatggccgaggcctacagcgagatcggcatgaagggcgagcggaggcgcggca | |
| | agggccacgatggcctgtaccagggcctgagcaccgccaccaaggacacctacgacgcc | |
| | ctgcacatgcaggccctgccccccaga | |
| 559 | MALPVTALLLPLALLLHAARPDIQMTQSPSSLSASVGDRVT | Exemplary BCMA |
| | ITCRASQSISSYLNWYQQKPGKAPKLLIYAASSLQSGVPSRF | CAR amino acid |
| | SGSGSGTDFTLTISSLQPEDFATYYCQQKYDLLTFGGGTKV | sequence |
| | EIKGSTSGSGKPGSGEGSTKGQLQLQESGPGLVKPSETLSLT | |
| | CTVSGGSISSSSYYWGWIRQPPGKGLEWIGSISYSGSTYYNP | |
| | SLKSRVTISVDTSKNQFSLKLSSVTAADTAVYYCARDRGD | |
| | TILDVWGQGTMVTVSSFVPVFLPAKPTTTPAPRPPTPAPTIA | |
| | SQPLSLRPEACRPAAGGAVHTRGLDFACDIYIWAPLAGTC | |
| | GVLLLSLVITLYCNHRNKRGRKKLLYIFKQPFMRPVQTTQE | |
| | EDGCSCRFPEEEEGGCELRVKFSRSADAPAYQQGQNQLYN | |
| | ELNLGRREEYDVLDKRRGRDPEMGGKPRRKNPQEGLYNE | |
| | LQKDKMAEAYSEIGMKGERRRGKGHDGLYQGLSTATKDT | |
| | YDALHMQALPPR | |

What is claimed:

1. A lipid particle, comprising:
(a) a lipid bilayer;
(b) a paramyxovirus glycoprotein (G protein) or a biologically active portion thereof; and
(c) a variant Nipah virus F glycoprotein (NiV-F) comprising a modified cytoplasmic tail, wherein the modified cytoplasmic tail comprises:
(i) a heterologous cytoplasmic tail or a truncated portion thereof from a glycoprotein from another virus or a virus-associated protein, wherein the variant NiV-F is a chimeric protein;
(ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or
(iii) a modified NiV-F cytoplasmic that comprises a modified endocytosis motif,
wherein the G protein or the biologically active portion thereof and the variant NiV-F protein are exposed on the outside of the lipid bilayer.

2. The lipid particle of claim 1, wherein the lipid bilayer is derived from a membrane of a host cell used for producing a retroviral vector or retrovirus-like particle, optionally a lentiviral vector or lentiviral-like particle.

3. The lipid particle of claim 2, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

4. A pseudotyped lentiviral particle, comprising:
(a) a paramyxovirus glycoprotein (G) or a biologically active portion thereof; and
(b) a variant Nipah virus F glycoprotein (NiV-F) comprising a modified cytoplasmic tail, wherein the modified cytoplasmic tail comprises:
(i) a heterologous cytoplasmic tail or a truncated portion thereof from a glycoprotein from another virus or a virus-associated protein, wherein the variant NiV-F is a chimeric protein;
(ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or
(iii) a modified NiV-F cytoplasmic that comprises a modified endocytosis motif,
wherein the G protein or the biologically active portion thereof and the variant NiV-F protein are exposed on the outside of the lipid bilayer.

5. The lipid particle or pseudotyped lentiviral particle of any of claims 1-4, wherein the variant NiV-F protein exhibits fusogenic activity with a target cell upon binding of the G protein to a target molecule on the target cell.

6. The lipid particle or pseudotyped lentiviral particle of any of claims 1-5, wherein the variant NiV-F protein comprises an F0 precursor or is a proteolytically cleaved form thereof comprising F1 and F2 subunits.

7. The lipid particle or pseudotyped lentiviral particle of claim 6, wherein the proteolytically cleaved form is a cathepsin L cleavage product.

8. The lipid particle or pseudotyped lentiviral particle of any of claims 1-7, wherein the variant Niv-F protein comprises a modified F1 subunit containing the modified cytoplasmic tail and an F2 subunit in which (1) the F1 subunit is a modified F1 composed of the sequence of the modified cytoplasmic tail directly linked to the C-terminus of the sequence set forth in SEQ ID NO:383, and (2) the F2 subunit is set forth as SEQ ID NO:365.

9. The lipid particle or pseudotyped lentiviral particle of any of claims 1-8, wherein the variant NiV-F comprises in order from N-terminus to C-terminus an extracellular domain, a transmembrane domain and the modified cytoplasmic tail.

10. The lipid particle or pseudotyped lentiviral particle of any of claims 1-6, wherein the extracellular domain and transmembrane domain of the variant NiV-F are from a wild-type NiV-F or a biologically active variant thereof.

11. The lipid particle or pseduotyped lentiviral particle of any of claims 1-7, wherein the extracellular domain and transmembrane domain of the variant NiV-F comprise the sequence set forth in SEQ ID NO:2, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:2.

12. The lipid particle or pseudotyped lentiviral particle of any of claims 1-11, wherein the extracellular domain and transmembrane domain of the variant NiV-F comprise the sequence set forth in SEQ ID NO:2.

13. The lipid particle or pseudotyped lentiviral particle of any of claims 1-12, wherein the modified cytoplasmic tail is a truncated NiV-F cytoplasmic tail that has a deletion of from 23 to 27 contiguous amino acid residues at or near the C-terminus of the wild-type Nipah virus F protein cytoplasmic tail set forth in SEQ ID NO:4.

14. The lipid particle or pseduotyped lentiviral particle of any of claims 1-13, the modified cytoplasmic tail is a truncated NiV-G cytoplasmic tail that has a deletion of at or about 23 amino acid residues at or near the C-terminus of the wild-type Nipah virus cytoplasmic tail set forth in SEQ ID NO: 4.

15. The lipid particle or pseudotyped lentiviral particle of any of claims 1-14, wherein the modified cytoplasmic tail is a truncated NiV-F cytoplasmic tail set forth in SEQ ID NO:27.

16. The lipid particle or pseudotyped lentiviral particle of any of claims 1-14, wherein the variant NiV-F comprises the sequence set forth in SEQ ID NO:306, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:306.

17. The lipid particle or pseudotyped lentiviral particle of any of claims 1-16, wherein the variant NiV-F comprises the sequence set forth in SEQ ID NO:306.

18. The lipid particle or pseudotyped lentiviral particle of any of claims 1-12, wherein the variant NiV-F is a chimeric protein and the modified cytoplasmic tail comprises a heterologous cytoplasmic tail or a truncated portion thereof from a glycoprotein from another virus.

19. The lipid particle or pseudotyped lentiviral particle of any of claims 1-12 and 18, wherein the other virus is a member of the Kingdom Orthornavirae.

20. The lipid particle or pseudotyped lentiviral particle of any of claims 1-12, 18 and 19, wherein the other virus is a member of the family Paramyxoviridae, Rhabdoviridae, Arenaviridae, or Retroviridae.

21. The lipid particle or pseudotyped lentiviral particle of any of claims 1-12 and 18-20, wherein the other virus is a member of the family Paramyxoviridae.

22. The lipid particle or pseudotyped lentiviral particle of claim 21, wherein the other virus is a Hendra virus, Cedar virus, Canine distemper virus, Parainfluenza virus, Measles virus, Newcastle disease virus, or Sendai virus.

23. The lipid particle or pseudotyped lentiviral particle of any of claims 1-12 and 18-22, wherein the other virus is Measles virus and the glycoprotein is a Measles virus fusion (F) protein (MvF).

24. The lipid particle or pseudotyped lentiviral particle of any of claims 1-12 and 18-23, wherein the heterologous cytoplasmic tail is a truncated MvF cytoplasmic tail that has a deletion of up to 32 contiguous amino acid residues at or near the C-terminus of the wild-type MvF cytoplasmic tail set forth in SEQ ID NO: 125.

25. The lipid particle or pseudotyped lentiviral particle of any of claims 1-12 and 18-24, wherein the heterologous cytoplasmic tail is a truncated MvF cytoplasmic tail that has a deletion of at or about or up to 26 contiguous amino acid residues at or near the C-terminus of the wild-type MvF cytoplasmic tail set forth in SEQ ID NO: 125.

26. The lipid particle or pseudotyped lentiviral particle of any of claims 1-12 and 18-25, wherein the modified cytoplasmic tail comprises the heterologous cytoplasmic tail set forth in SEQ ID NO:133.

27. The lipid particle or pseudotyped lentiviral particle of any of claims 1-12 and 18-25, wherein the variant NiV-F comprises the sequence of amino acids set forth in SEQ ID NO:307, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:307.

28. The lipid particle or pseudotyped lentiviral particle of any of claims 1-12 and 18-27, wherein the variant NiV-F comprises the sequence of amino acids set forth in SEQ ID NO: 307.

29. The lipid particle or pseudotyped lentiviral particle of any of claims 1-12 and 18-22, wherein the other virus is Newcastle Disease Virus (NDV) and the glycoprotein is a NDV F protein.

30. The lipid particle or pseudotyped lentiviral particle of any of claims 1-12, 18-22 and 29, wherein the heterologous cytoplasmic tail is a truncated NDV F protein cytoplasmic tail that has a deletion of up to 25 contiguous amino acid residues at or near the C-terminus of the wild-type NDV F protein cytoplasmic tail set forth in SEQ ID NO: 141.

31. The lipid particle or pseudotyped lentiviral particle of any of claims 1-12, 18-22, 29 and 30, wherein the heterologous cytoplasmic tail is a truncated NDV F protein cytoplasmic tail that has a deletion of at or about or up to 17 contiguous amino acid residues at or near the C-terminus of the wild-type NDV F protein cytoplasmic tail set forth in SEQ ID NO: 141.

32. The lipid particle or pseudotyped lentiviral particle of any of claims 1-12 and 18-22 and 29-31, wherein the modified cytoplasmic tail comprises the heterologous cytoplasmic tail set forth in SEQ ID NO:147.

33. The lipid particle or pseudotyped lentiviral particle of any of claims 1-12, 18-22 and 29-32, wherein the variant NiV-F comprises the sequence of amino acids set forth in SEQ ID NO:308, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:308.

34. The lipid particle or pseudotyped lentiviral particle of any of claims 1-12, 18-22 and 29-33, wherein the variant NiV-F comprises the sequence of amino acids set forth in SEQ ID NO: 308.

35. The lipid particle or pseudotyped lentiviral particle of any of claims 1-12, 18-22, 29 and 30, wherein the heterologous cytoplasmic tail is a truncated NDV F protein cytoplasmic tail that has a deletion of at or about or up to 20 contiguous amino acid residues at or near the C-terminus of the wild-type NDV F protein cytoplasmic tail set forth in SEQ ID NO: 141.

36. The lipid particle or pseudotyped lentiviral particle of any of claims 1-12 and 18-22 and 29, 30 and 35, wherein the modified cytoplasmic tail comprises the heterologous cytoplasmic tail set forth in SEQ ID NO:150.

37. The lipid particle or pseudotyped lentiviral particle of any of claims 1-12, 18-22, 29, 30, 35 and 36, wherein the variant NiV-F comprises the sequence of amino acids set forth in SEQ ID NO:309, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:309.

38. The lipid particle or pseudotyped lentiviral particle of any of claims 1-12, 18-22, 29, 30 and 35-37, wherein the variant NiV-G comprises the sequence of amino acids set forth in SEQ ID NO:309.

39. The lipid particle or pseudotyped lentiviral particle of any of claims 1-12 and 18-22, wherein the other virus is Hendra virus (HeV) and the glycoprotein is a HeV F protein.

40. The lipid particle or pseudotyped lentiviral particle of any of claims 1-12, 18-22 and 39, wherein the heterologous cytoplasmic tail is a truncated HeV F cytoplasmic tail that has a deletion of up to 26 contiguous amino acid residues at or near the C-terminus of the wild-type HeV F cytoplasmic tail set forth in SEQ ID NO: 59.

41. The lipid particle or pseudotyped lentiviral particle of any of claims 1-12, 18-22, 39 and 40, wherein the heterologous cytoplasmic tail is a truncated HeV F cytoplasmic tail that has a deletion of at or about or up to 22 contiguous amino acid residues at or near the C-terminus of the wild-type HeV F cytoplasmic tail set forth in SEQ ID NO: 59.

42. The lipid particle or pseudotyped lentiviral particle of any of claims 1-12, 18-22 and 39-41, wherein the modified cytoplasmic tail comprises the heterologous cytoplasmic tail set forth in SEQ ID NO:80.

43. The lipid particle or pseudotyped lentiviral particle of any of claims 1-12, 18-22 and 39-41, wherein the variant NiV-F comprises the sequence of amino acids set forth in SEQ ID NO:310, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:310.

44. The lipid particle or pseudotyped lentiviral particle of any of claims 1-12, 18-22 and 39-43, wherein the variant NiV-F comprises the sequence of amino acids set forth in SEQ ID NO: 310.

45. The lipid particle or pseudotyped lentiviral particle of any one of claims 1-12 and 18-22, wherein the modified cytoplasmic tail comprises replacement of the attachment motif of the NiV-F cytoplasmic tail with a cytoplasmic tail or a truncated portion thereof form the attachment protein of another paramyxovirus.

46. The lipid particle or pseudotyped lentiviral particle of claim 45, wherein the paramyxovirus is a Nipah virus, Hendra virus, or Measles virus.

47. The lipid particle or pseudotyped lentiviral particle of claim 45 or claim 46, wherein the attachment protein is a G protein, H protein or HN protein.

48. The lipid particle or pseudotyped lentiviral particle of any of claims 45-47, wherein the modified cytoplasmic tail comprises the heterologous cytoplasmic tail set forth in any one of SEQ ID NOS: 210-222.

49. The lipid particle or pseudotyped lentiviral particle of any of claims 1-48, further comprising a modified ectodomain comprising a modified protease cleavage site.

50. The lipid particle or pseudotyped lentiviral particle of claim 49, wherein the modified cleavage site comprises replacement of the cleavage sequence NNTHDLVGDVRLAGV (SEQ ID NO:311) with a modified furin cleavage sequence set forth in any one of SEQ ID NOS: 313-327.

51. The lipid particle or pseudotyped lentiviral particle of claim 49, wherein the modified cleavage site comprises replacement of the cleavage sequence VGDVR (SEQ ID NO:339) with a modified cleavage sequence from another paramyxovirus, optionally wherein the modified cleavage sequence from another paramyxovirus comprises a cathepsin L cleavage site.

52. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a modified ectodomain, wherein the modified ectodomain comprises a modified cleavage site selected from:
(i) replacement of the cleavage sequence NNTHDLVGDVRLAGV (SEQ ID NO:311) with a modified furin cleavage sequence; or
(ii) replacement of the cleavage sequence VGDVR (SEQ ID NO:339) with a modified cleavage sequence from another paramyxovirus, optionally wherein the modified cleavage sequence from another paramyxovirus comprises a cathepsin L cleavage site.

53. The lipid particle of claim 52, wherein the lipid bilayer is derived from a membrane of a host cell used for producing a retroviral vector or retrovirus-like particle, optionally a lentiviral vector or lentiviral-like particle.

54. The lipid particle of claim 53, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

55. A pseudotyped lentiviral particle, comprising:
(a) a paramyxovirus glycoprotein (G) or a biologically active portion thereof; and
(b) a variant Nipah virus F glycoprotein (NiV-F) comprising a modified ectodomain, wherein the modified ectodomain comprises a modified cleavage site selected from:
(i) replacement of the cleavage sequence NNTHDLVGDVRLAGV (SEQ ID NO:311) with a modified furin cleavage sequence; or; or
(ii) replacement of the cleavage sequence VGDVR (SEQ ID NO:329) VGDVR (SEQ ID NO:339) with a modified cleavage sequence from another paramyxovirus, optionally wherein the cleavage sequence is a cathepsin L cleavage site.

56. The lipid particle or pseudotyped lentiviral particle of any of claims 49-55, wherein the variant NiV-F protein comprises an F0 precursor or is a proteolytically cleaved form thereof comprising F1 and F2 subunits.

57. The lipid particle or pseudotyped lentiviral particle of claim 52 or claim 56, wherein the replacement cleavage sequence comprises a cathepsin L cleavage site and the proteolytically cleaved form is a cathepsin L cleavage product.

58. The lipid particle or pseudotyped lentiviral particle of claim 52 or claim 56, wherein the replacement cleavage sequence comprises a modified furin cleavage site and the proteolytically cleaved from is a furin cleavage product.

59. The lipid particle or pseudotyped lentiviral vector of any of claims 49-58, wherein the replacement is in the sequence of amino acids set forth in SEQ ID NO: 1 or SEQ ID NO:320, or a mature form thereof lacking the signal peptide (amino acids 1-26).

60. The lipid particle or pseudotyped lentiviral vector of any of claims 50, 51 and 53-59, wherein the furin cleavage sequence is set forth in any one of SEQ ID NOS: 313-327.

61. The lipid particle or psuedotyped lentiviral vector of any of claims 50, 51 and 53-60, wherein the variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26).

62. The lipid particle or pseudotyped lentiviral vector of any of claims 50, 52 and 53-60, wherein the modified cleavage site from another paramyxovirus is set forth in any one of SEQ ID NOS: 329-338.

63. The lipid particle or pseudotyped lentiviral vector of any of claims 50, 52, 53-60 and 62, wherein the variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26).

64. The lipid particle or lentiviral vector of any of claims 50, 51, 53-60, 62 and 63, wherein the variant NiV-F comprises the sequence of amino acids set forth in SEQ ID NO:239, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:239, or is a mature form thereof lacking the signal peptide (e.g. amino acids 1-26).

65. The lipid particle or lentiviral vector of any of claims 50, 51, 53-60, 62, 63 and 64, wherein the variant NiV-F comprises the sequence of amino acids set forth in SEQ ID NO: 239.

66. The lipid particle or lentiviral vector of any of claims 1-65, wherein the variant NiV-F comprises a heterologous signal sequence compared to the signal sequence of wild-type NiV-F.

67. The lipid particle or lentiviral vector of any of claims 1-66, wherein the variant NiV-F is encoded by a polynucleotide sequence comprising a sequence that encodes a heterologous signal sequence compared to the encoded signal sequence of wild-type NiV-F.

68. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a heterologous signal sequence, or encoded by a polynucleotide sequence comprising a sequence that encodes a heterologous signal sequence, compared to the wild-type signal sequence of NiV-F.

69. The lipid particle of claim 68, wherein the lipid bilayer is derived from a membrane of a host cell used for producing a retroviral vector or retrovirus-like particle, optionally a lentiviral vector or lentiviral-like particle.

70. The lipid particle of claim 69, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

71. A pseudotyped lentiviral particle, comprising:
(a) a paramyxovirus glycoprotein (G) or a biologically active portion thereof; and
(b) a variant Nipah virus F glycoprotein (NiV-F) comprising a heterologous signal sequence, or encoded by a polynucleotide sequence comprising a sequence that encodes a heterologous signal sequence, compared to the wild-type signal sequence of NiV-F.

72. The lipid particle or pseudotyped lentiviral particle of any of claims 68-71, wherein the variant NiV-F protein comprises an F0 precursor or is a proteolytically cleaved form thereof comprising F1 and F2 subunits.

73. The lipid particle or pseudotyped lentiviral particle of claim 72, wherein the proteolytically cleaved form is a cathepsin L cleavage product.

74. The lipid particle or pseudotyped lentiviral vector of any of claims 68-73, wherein the heterologous signal sequence is from another virus or is a mammalian signal sequence.

75. The lipid particle or pseudotyped lentiviral vector of claim 74, wherein the other virus is a paramyxovirus, optionally a henipavirus.

76. The lipid particle or pseudotyped lentiviral vector of any of claims 66-75, wherein the heterologous signal sequence is a HevF signal sequence, a CevF signal sequence, a HPIV2 signal sequence, a MevF signal sequence, a NDV-F signal sequence, or a SevF signal sequence.

77. The lipid particle or pseudotyped lentiviral vector of any of claims 66-76, wherein the heterologous signal sequence is set forth in any one of SEQ ID NOS: 340-345.

78. The lipid particle or pseudotyped lentiviral vector of any of claims 66-77, wherein the other virus is Parainfluenza virus and the heterologous signal sequence is the Human Parainfluenza Virus 2 (HPIV2) signal sequence, optionally set forth in SEQ ID NO: 342.

79. The lipid particle or pseudotyped lentiviral vector of any of claims 66-78, wherein the variant NiV-F comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.

80. The lipid particle or pseudotyped lentiviral vector of any of claims 66-79, wherein the variant NiV-F is encoded by a nucleotide sequence that encodes a variant NiV-F that comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.

81. The lipid particle or lentiviral vector of any of claims 66-80, wherein the variant NiV-F comprises the sequence of amino acids set forth in SEQ ID NO: 251.

82. The lipid particle or pseudotyped lentiviral vector of any of claims 66-74, wherein the other virus is HIV-1, Baboon endogenous retrovirus, Cocal virus, Ebola virus, Gibon ape leukemia virus, Murine leukemia virus, or Vesicular stomatitis virus.

83. The lipid particle or pseudotyped lentiviral vector of any of claims 66-74 and 82, wherein the heterologous signal sequence is a HIV1-Env signal sequence, BaEV signal sequence, Cocal signal sequence, EboV signal sequence, GaLV signal sequence, MLV-A signal sequence, or VSVG signal sequence.

84. The lipid particle or pseudotyped lentiviral vector of any of claims 66-74, 82 and 83, wherein the heterologous signal sequence is set forth in any one of SEQ ID NOS: 346-352.

85. The lipid particle or pseudotyped lentiviral vector of any of claims 66-74, and 82-84, wherein the other virus is Vesicular stomatitis virus (VSV) and the heterologous signal sequence is the VSV G glycoprotein (VSV-G) signal sequence, optionally set forth in SEQ ID NO: 352.

86. The lipid particle or pseudotyped lentiviral vector of any of claims 66-74, and 82-85, wherein the variant NiV-F comprises the sequence of amino acids set forth in SEQ ID NO:261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261.

87. The lipid particle or pseudotyped lentiviral vector of any of claims 66-74, and 82-86, wherein the variant NiV-F is encoded by a nucleotide sequence that encodes a variant NiV-F that comprises the sequence of amino acids set forth in SEQ ID NO:261 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261.

88. The lipid particle or pseudotyped lentiviral vector of any of claims 66-74, and 82-87, wherein the variant NiV-F comprises the sequence of amino acids set forth in SEQ ID NO: 261.

89. The lipid particle or pseudotyped lentiviral vector of any of claims 66-74, wherein the mammalian signal sequence is a signal sequence from a human protein.

90. The lipid particle or pseudotyped lentiviral vector of any of claims 66-74 and 89, wherein the heterologous signal sequence is a Prolactin signal sequence, IgGk-L signal sequence, Albumin signal sequence, CD5 signal sequence, Trypsinogen signal sequence orIL2 signal sequence.

91. The lipid particle or pseudotyped lentiviral vector of any of claims 66-74, 89 and 90, wherein the heterologous signal sequence is set forth in any one of SEQ ID NOS: 353-358.

92. The lipid particle or lentiviral vector of any of claims 1-91, wherein the variant NiV-F comprises a heterologous or modified transmembrane domain compared to the transmembrane domain of wild-type NiV-F.

93. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a heterologous or modified transmembrane domain compared to wild-type NiV-F.

94. The lipid particle of claim 93, wherein the lipid bilayer is derived from a membrane of a host cell used for producing a retroviral vector or retrovirus-like particle, optionally a lentiviral vector or lentiviral-like particle.

95. The lipid particle of claim 94, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

96. A pseudotyped lentiviral particle, comprising:
(a) a paramyxovirus glycoprotein (G) or a biologically active portion thereof; and
(b) a variant Nipah virus F glycoprotein (NiV-F) comprising a heterologous or modified transmembrane domain compared to wild-type NiV-F.

97. The lipid particle or pseudotyped lentiviral particle of any of claims 93-96, wherein the variant NiV-F protein comprises an F0 precursor or is a proteolytically cleaved form thereof comprising F1 and F2 subunits.

98. The lipid particle or pseudotyped lentiviral particle of claim 97, wherein the proteolytically cleaved form is a cathepsin L cleavage product.

99. The lipid particle or pseudotyped lentiviral particle of any of claims 93-98, wherein the transmembrane domain is a heterologous transmembrane domain from Hendra virus, optionally wherein the transmembrane domain is set forth in SEQ ID NO: 361.

100. The lipid particle or pseudotyped lentiviral particle of any of claims 93-99, wherein the transmembrane domain is a modified transmembrane domain containing the amino acid replacement S490A, Y498A, S504A or I516V, corresponding to numbering of positions set forth in SEQ ID NO:1, optionally wherein the transmembrane domain is set forth in any one of SEQ ID NOS: 359, 360, 362 or 363.

101. The lipid particle or pseudotyped lentiviral vector of any of claims 93-100, wherein the variant NiV-F comprises the sequence of amino acids set forth in any one of SEQ ID NO: 268-272 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 268-272.

102. The lipid particle or pseudotyped lentiviral particle of any of claims 1-101, which further comprises a hyperfusogenic mutation.

103. The lipid particle or pseudotyped lentiviral particle of claim 102, wherein the hyperfusogenic mutation is introduced at a N-glycosylation site with respect to amino acid positions 64, 67, 99, 414 and/or 464 of SEQ ID NO. 1.

104. The lipid particle or pseudotyped lentiviral particle of claim 102 or 103, wherein the hyperfusogenic mutation is one or more of N64Q, N67Q, N99Q, N414Q and/or N464Q, with reference to numbering set forth in SEQ ID NO:1

105. The lipid particle or pseudotyped lentiviral particle of claim 104, wherein the one or more mutations are selected from the group consisting of N64Q, N67Q, N99Q, N414Q, N464Q, N67Q/N99Q, N67Q/N414Q, N67Q/N464Q, N99Q/N414Q, N99Q/N464Q, N414Q/N464Q, N67Q/N99Q/N414Q, N67Q/N414Q/N464Q, N99Q/N414Q/N464Q, or N67Q/N99Q/N414Q/N464Q, N64Q/N99Q, N64Q/N99Q/N464Q, N64Q/N67Q/N99Q, and N64Q/N67Q/N99Q/N464Q.

106. The lipid particle or pseudotyped lentiviral particle of claim 102, wherein the hyperfusogenic mutation is introduced at a hexamer stability site with respect to amino acid positions 393 and/or 109 of SEQ ID NO. 1.

107. The lipid particle or pseudotyped lentiviral particle of claims 102 or 106, wherein the hyperfusogenic mutation is one or more of R109L, Q393L or R109L and Q393L, with reference to numbering set forth in SEQ ID NO:1.

108. A lipid particle, comprising:
(a) a lipid bilayer;
(b) a paramyxovirus glycoprotein (G) or a biologically active portion thereof; and
(c) a variant Nipah virus F glycoprotein (NiV-F) comprising a hyperfusogenic mutation selected from:
a mutation that is introduced at a N-glycosylation site with respect to amino acid positions 64, 67, 99, 414 and/or 464 of SEQ ID NO. 1; and/or
a mutation that is introduced at a hexamer stability site with respect to amino acid positions 393 and/or 109 of SEQ ID NO. 1.

109. The lipid particle of any of claims 106, 107 or 108, wherein the hyperfusogenic mutation is introduced at a hexamer stability site with respect to amino acid positions 393 of SEQ ID NO. 1, optionally wherein the mutation is a substitution Q393L.

110. The lipid particle of claim 108 or claim 109, wherein the lipid bilayer is derived from a membrane of a host cell used for producing a retroviral vector or retrovirus-like particle, optionally a lentiviral vector or lentiviral-like particle.

111. The lipid particle of claim 110, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

112. A pseudotyped lentiviral particle, comprising:
(a) a paramyxovirus glycoprotein (G) or a biologically active portion thereof; and
(b) a variant Nipah virus F glycoprotein (NiV-F) comprising a hyperfusogenic mutation selected from:
a mutation that is introduced at a N-glycosylation site with respect to amino acid positions 64, 67, 99, 414, and/or 464 of SEQ ID NO. 1; and/or
a mutation that is introduced at a hexamer stability site with respect to amino acid positions 393 and/or 109 of SEQ ID NO. 1.

113. The lipid particle or pseudotyped lentiviral particle of any of claims 108-112, wherein the variant NiV-F protein comprises an F0 precursor or is a proteolytically cleaved form thereof comprising F1 and F2 subunits.

114. The lipid particle or pseudotyped lentiviral particle of claim 113, wherein the proteolytically cleaved form is a cathepsin L cleavage product.

115. The lipid particle or pseudotyped lentiviral vector of any of claims 102-114, comprising:
a mutation N67Q, N99Q, N414Q or N464Q or any combination thereof; and/or
a mutation R109L or Q393L or a combination thereof.

116. The lipid particle or pseudotyped lentiviral vector of any of claims 102-115, wherein the hyperfusogenic mutation is in the sequence of amino acids set forth in SEQ ID NO:1, SEQ ID NO:2, SEQ ID NO:302 or SEQ ID NO:303.

117. The lipid particle or pseudotyped lentiviral vector of any of claims 1-116, wherein the variant NiV-F comprises the sequence of amino acids set forth in SEQ ID NO:247, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:247.

118. The lipid particle or pseudotyped lentiviral vector of any of claims 1-117, wherein the variant NiV-F comprises the sequence of amino acids set forth in SEQ ID NO: 247.

119. The lipid particle or pseudotyped lentiviral particle of any of claims 1-116, wherein the variant NiV-F comprises the sequence of amino acids set forth in SEQ ID NO:292, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:292.

120. The lipid particle or pseudotyped lentiviral particle of any of claims 1-116 or 119, wherein the variant NiV-F comprises the sequence of amino acids set forth in SEQ ID NO: 292.

121. The lipid particle or pseudotyped lentiviral particle of any of claims 1-120, wherein the G protein or the biologically active portion thereof is a wild-type Nipah virus G (NiV-G) protein or a Hendra virus G protein or is a functionally active variant or biologically active portion thereof.

122. The lipid particle or pseudotyped lentiviral particle of any of claims 1-121, wherein the G protein or the biologically active portion thereof is a wild-type NiV-G protein or a functionally active variant or a biologically active portion thereof.

123. The lipid particle or pseudotyped lentiviral particle of any of claims 1-122, wherein the G protein is a biologically active portion of wild-type NiV-G that is truncated in which the G protein lacks up to 34 contiguous amino acids at or near the N-terminus of the wild-type NiV-G set forth in SEQ ID NO:305.

124. The lipid particle or pseudotyped lentiviral particle of any of claims 1-123, wherein the G-protein is a NiV-G functionally active variant or a biologically active portion thereof that exhibits reduced binding to Ephrin B2 or Ephrin B3.

125. The lipid particle or pseudotyped lentiviral particle of claim 124, wherein the NiV-G functionally active variant or a biologically active portion thereof protein comprises: one or more amino acid substitutions corresponding to amino acid substitutions selected from the group consisting of E501A, W504A, Q530A and E533A with reference to numbering set forth in SEQ ID NO:305.

126. The lipid particle or pseudotyped lentiviral particle of claim 124 or claim 125, wherein the NiV-G functionally active variant or a biologically active portion thereof comprises amino acid substitutions E501A, W504A, Q530A and E533A with reference to numbering set forth in SEQ ID NO:305.

127. The lipid particle or pseudotyped lentiviral particle of any of claims 122-126, wherein the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301.

128. The lipid particle or pseudotyped lentiviral particle of any of claims 122-127, wherein the NiV-G functionally active variant or a biologically active portion thereof comprises the amino acid sequence set forth in SEQ ID NO: 301.

129. The lipid particle or pseudotyped lentiviral particle of any of claims 122-128, wherein the G protein is linked to a binding domain that binds to a target cell surface molecule on a target cell.

130. The lipid particle or pseudotyped lentiviral particle of claim 129, wherein the binding domain is attached to the C-terminus of the G protein.

131. The lipid particle or pseudotyped lentiviral particle of claim 129 or claim 130, wherein the cell surface molecule is a protein, glycan, lipid or low molecular weight molecule.

132. The lipid particle or pseudotyped lentiviral particle of any of claims 129-131, wherein the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells.

133. The lipid particle or pseudotyped lentiviral particle of any of claims 129-132, wherein the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell.

134. The lipid particle or pseudotyped lentiviral particle of any of claims 129-133 wherein the target cell is a hepatocyte.

135. The lipid particle or pseudotyped lentiviral particle of any of claims 129-133 and 134, wherein the binding domain binds to a cell surface molecule selected from the group consisting of ASGR1, ASGR2 and TM4SF.

136. The lipid particle or pseudotyped lentiviral particle of any of claims 129-133, wherein the target cell is a T cell.

137. The lipid particle or pseudotyped lentiviral particle of any of claims 129-133 and 136, wherein the binding domain binds to a cell surface molecule selected from the group consisting of CD3, CD4 or CD8.

138. The lipid particle or pseudotyped lentiviral particle of any of claims 129-137, wherein the binding domain is an antibody or antigen-binding fragment, a single domain antibody or a DARPin.

139. The lipid particle or pseudotyped lentiviral particle of any of claims 129-138, wherein the binding domain is a single domain antibody that is a VHH or is a single chain variable fragment (scFv).

140. The lipid particle or pseudotyped lentiviral particle of any of claims 129-139 wherein the binding domain is attached to the G protein via a linker.

141. The lipid particle or pseudotyped lentiviral particle of claim 140, wherein the linker is a peptide linker.

142. The lipid particle or pseudotyped lentiviral particle of claim 141, wherein the peptide linker is 2 to 65 amino acids in length.

143. The lipid particle or pseudotyped lentiviral particle of claim 141 or claim 142, wherein the peptide linker is a flexible linker that comprises GS, GGS, GGGGS (SEQ ID NO:287), GGGGGS (SEQ ID NO:288) or combinations thereof.

144. The lipid particle or pseudotyped lentiviral particle of any of claims 141-143, wherein the peptide linker is selected from: (GGS)n, wherein n is 1 to 10; (GGGGS)n (SEQ ID NO:289), wherein n is 1 to 10; or (GGGGGS)n (SEQ ID NO:290), wherein n is 1 to 6.

145. The lipid particle or pseudotyped lentiviral vector of any of claims 1-144 that is replication defective.

146. The lipid particle or pseudotyped lentiviral vector of any of claims 1-145 prepared by a method comprising transducing a producer cell with packaging plasmids that encode a Gag-pol, Rev, Tat and the variant NiVG and the F protein.

147. The lipid particle or pseudotyped lentiviral vector of any of claims 1-146, wherein the particle further comprises a viral nucleic acid.

148. The lipid particle or pseudotyped lentiviral vector of claim 147, wherein the viral nucleic acid comprises one or more of (e.g., all of) the following nucleic acid sequences: 5′ LTR (e.g., comprising US and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising US and lacking a functional U3).

149. The lipid particle or pseudotyped lentiviral vector of any of claims 1-147, wherein the particle is devoid of viral genomic DNA.

150. The lipid particle or pseudotyped lentiviral particle of any of claims 1-149, wherein the particle is produced as a preparation with increased titer compared to a reference particle preparation that is similarly produced but with incorporation of the full-length NiV-F protein set forth in SEQ ID NO: 1, optionally wherein the lipid particle or pseudotyped lentiviral particle is a lentivirus vector.

151. The lipid particle or pseudotyped lentiviral particle of any of claims 1-150, wherein the particle is produced as a preparation with increased titer compared to a reference particle preparation that is similarly produced but with incorporation of the truncated NiV-F protein comprising the sequence set forth in SEQ ID NO:303 or encoded by a polynucleotide that encodes the sequence set forth in SEQ ID NO:302, optionally wherein the lipid particle or psuedotyped lentiviral particle is a lentivirus vector.

152. The lipid particle or pseudotyped lentiviral particle of claim 150 or claim 151, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

153. The lipid particle or pseudotyped lentiviral particle of any of claims 150-152, wherein the titer is increased by about or greater than about 2.0-fold.

154. The lipid particle or pseudotyped lentiviral particle of any of claims 150-152, wherein the titer is increased by about or greater than about 3.0-fold.

155. The lipid particle or pseudotyped lentiviral particle of any of claims 150-152, wherein the titer is increased by about or greater than about 4.0-fold.

156. The lipid particle or pseudotyped lentiviral particle of any of claims 150-152, wherein the titer is increased by about or greater than about 5.0-fold.

157. The lipid particle or pseudotyped lentiviral particle of any of claims 1-156, further comprising an exogenous agent for delivery to a target cell.

158. The lipid particle or pseudotyped lentiviral particle of claim 157, wherein the exogenous agent is present in the lumen.

159. The lipid particle or pseudotyped lentiviral particle of claim 157 or claim 158, wherein the exogenous agent is a protein or a nucleic acid, optionally wherein the nucleic acid is a DNA or RNA.

160. The lipid particle or pseudotyped lentiviral particle of any of claims 157-159, wherein the exogenous agent is a nucleic acid encoding a cargo for delivery to the target cell.

161. The lipid particle or lentiviral vector of any of claims 157-160, wherein the exogenous agent is or encodes a therapeutic agent or a diagnostic agent.

162. The lipid particle or pseudotyped lentiviral particle of any of claims 157-161, wherein the exogenous agent encodes a membrane protein, optionally wherein the membrane protein is an antigen receptor for targeting cells expressed by or associated with a disease or condition.

163. The lipid particle or pseudotyped lentiviral particle of claim 162, wherein the membrane protein is a chimeric antigen receptor (CAR).

164. The lipid particle or pseudotyped lentiviral particle of any of claims 157-163, wherein the target cell is a T cell.

165. The lipid particle or lentiviral vector of any of claims 157-161, wherein the exogenous agent is a nucleic acid comprising a payload gene for correcting a genetic deficiency, optionally a genetic deficiency in the target cell, optionally wherein the genetic deficiency is associated with a liver cell or a hepatocyte.

166. The lipid particle or lentiviral vector of any of claims 157-165, wherein binding of the G protein to a cell surface molecule on the target cell mediates fusion of the particle with the target cell and delivery of the exogenous agent to the target cell.

167. The lipid particle or pseudotyped lentiviral particle of any of claims 157-166, wherein at or greater than 10%, 20%, 30%, 40%, 50%, 60% of the target cells are delivered the exogenous agent.

168. The lipid particle or lentiviral vector of any of claims 157-167, wherein delivery of the exogenous cell to the target cell is increased compared to a reference particle preparation that is similarly produced but in which the NiV-F protein is the full-length NiV-F protein set forth in SEQ ID NO: 1, optionally wherein the lipid particle or pseudotyped lentiviral particle is a lentivirus vector.

169. The lipid particle or pseudotyped lentiviral particle of any of claims 157-167, wherein delivery of the exogenous cell to the target cell is increased compared to a reference particle preparation that is similarly produced but in which the F protein is the truncated NiV-F comprising the sequence set forth in SEQ ID NO:303 or encoded by a polynucleotide that encodes the sequence set forth in SEQ ID NO:302, optionally wherein the lipid particle or pseudotyped lentiviral particle is a lentivirus vector.

170. The lipid particle or pseudotyped lentiviral particle of any of claims 157-169, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

171. A polynucleotide comprising a nucleic acid molecule encoding a variant Nipah virus F glycoprotein (NiV-F) comprising a modified cytoplasmic tail, wherein the modified cytoplasmic tail comprises:
(i) a heterologous cytoplasmic tail or a truncated portion thereof from a glycoprotein from another virus or a virus-associated protein, wherein the variant NiV-F is a chimeric protein;
(ii) a truncated NiV-F cytoplasmic tail that has a deletion of between 23 and 27 contiguous amino acid residues at or near the C-terminus of the wild-type NiV-F protein cytoplasmic tail set forth in SEQ ID NO:4, with the proviso that the truncated NiV-F does not have a deletion of 25 contiguous amino acids; and/or
(iii) a modified NiV-F cytoplasmic that comprises a modified endocytosis motif.

172. The polynucleotide of claim 171, wherein the variant NiV-F protein comprises an F0 precursor or is a proteolytically cleaved form thereof comprising F1 and F2 subunits.

173. The polynucleotide of claim 172, wherein the proteolytically cleaved form is a cathepsin L cleavage product.

174. The polynucleotide of any of claims 171-173, wherein the variant Niv-F protein comprises a modified F1 subunit containing the modified cytoplasmic tail and an F2 subunit in which (1) the F1 subunit is a modified F1 composed of the sequence of the modified cytoplasmic tail directly linked to the C-terminus of the sequence set forth in SEQ ID NO:383, and (2) the F2 subunit is set forth as SEQ ID NO:365.

175. The polynucleotide of any of claims 171-174, wherein the variant NiV-F comprises in order from N-terminus to C-terminus the an extracellular domain, a transmembrane domain and the modified cytoplasmic tail.

176. The polynucleotide of claim 175, wherein the extracellular domain and transmembrane domain of the variant NiV-F are from a wild-type NiV-F or a biologically active variant thereof.

177. The lipid particle or pseduotyped lentiviral particle of claim 175 or claim 176, wherein the extracellular domain and transmembrane domain of the variant NiV-F comprise the sequence set forth in SEQ ID NO:2, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:2.

178. The polynucleotide of any of claims 175-177, wherein the extracellular domain and transmembrane domain of the variant NiV-F comprise the sequence set forth in SEQ ID NO:2.

179. The polynucleotide of any of claims 174-178, wherein the modified cytoplasmic tail comprises a heterologous cytoplasmic tail set forth in any one of SEQ ID NOS: 80, 133, 147 and 150.

180. The polynucleotide of any of claims 174-179, wherein the encoded variant NiV-F comprises the sequence of amino acids set forth in any one of SEQ ID NOS: 307, 308, 309, and 310.

181. The polynucleotide of any of claims 174-178, wherein the modified cytoplasmic tail is a truncated NiV-F cytoplasmic tail set forth in any one of SEQ ID NOS: 27.

182. The polynucleotide of any of claims 174-178 and 181, wherein the encoded variant NiV-F comprises the sequence of amino acids set forth in any one of SEQ ID NOS: 306.

183. A polynucleotide comprising a nucleic acid molecule encoding a variant Nipah virus F glycoprotein (NiV-F) comprising a modified ectodomain, wherein the modified ectodomain comprises a modified cleavage site selected from:
(i) replacement of the cleavage sequence NNTHDLVGDVRLAGV (SEQ ID NO:311) with a modified furin cleavage sequence; or
(ii) replacement of the cleavage sequence VGDVR (SEQ ID NO:339) with a modified cleavage sequence from another paramyxovirus, optionally wherein the cleavage sequence is a cathepsin L cleavage site;
wherein the replacement is in the sequence of amino acids set forth in SEQ ID NO: 1 or SEQ ID NO:320, or a mature form thereof lacking the signal peptide (amino acids 1-26).

184. The polynucleotide of claim 183, wherein the furin cleavage sequence is set forth in any one of SEQ ID NOS: 313-327.

185. The polynucleotide of claim 183 or claim 184, wherein the encoded variant NiV-F comprises the sequence of amino acids set forth in any of SEQ ID NOS: 224-238 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 224-238 and contains the cleavage site set forth in any one of SEQ ID NOS: 224-238, respectively, or is a mature form thereof lacking the signal peptide (e.g. lacking amino acids 1-26).

186. The polynucleotide of claim 183, wherein the modified cleavage site from another paramyxovirus is set forth in any one of SEQ ID NOS: 329-338.

187. The polynucleotide of claim 183 or claim 186, wherein the encoded variant NiV-F comprises the sequence of amino acids set forth in any of SEQ ID NOS: 239-248 or a sequence that exhibits at least at or about at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOS: 239-248 and contains the cleavage site set forth in any one of SEQ ID NOS: 329-338, respectively, or is a mature form thereof lacking the signal peptide (e.g. lacking amino acids 1-26).

188. The polynucleotide of claim 183, claim 186 or claim 187, wherein the encoded variant NiV-F comprises the sequence of amino acids set forth in SEQ ID NO:239, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:239, or is a mature form thereof lacking the signal peptide (e.g. amino acids 1-26).

189. The polynucleotide of any of claims 183 and 186-188, wherein the encoded variant NiV-F comprises the sequence of amino acids set forth in SEQ ID NO: 239.

190. A polynucleotide comprising a nucleic acid molecule encoding a variant Nipah virus F glycoprotein (NiV-F) comprising a heterologous signal sequence compared to the wild-type signal sequence of NiV-F.

191. The polynucleotide of claim 190, wherein the heterologous signal sequence is from another virus or is a mammalian signal sequence.

192. The polynucleotide of claim 191, wherein the other virus is a paramyxovirus, optionally a henipavirus.

193. The polynucleotide of any of claims 190-192, wherein the heterologous signal sequence is a HevF signal sequence, a CevF signal sequence, a HPIV2 signal sequence, a MevF signal sequence, a NDV-F signal sequence, or a SevF signal sequence.

194. The polynucleotide of any of claims 190-193, wherein the heterologous signal sequence is set forth in any one of SEQ ID NOS: 340-345.

195. The polynucleotide of any of claims 190-191, wherein the other virus is Parainfluenza virus and the heterologous signal sequence is the Human Parainfluenza Virus 2 (HPIV2) signal sequence, optionally set forth in SEQ ID NO: 342.

196. The polynucleotide of any of claims 190, 191 and 195, wherein the encoded variant NiV-F comprises the sequence of amino acids set forth in SEQ ID NO:251, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:251.

197. The polynucleotide of any of claims 190, 191, 195 and 196, wherein the encoded variant NiV-F comprises the sequence of amino acids set forth in SEQ ID NO: 251.

198. The polynucleotide of any of claims 190-191, wherein the other virus is HIV-1, Baboon endogenous retrovirus, Cocal virus, Ebola virus, Gibon ape leukemia virus, Murine leukemia virus, or Vesicular stomatitis virus.

199. The polynucleotide of any of claims 190-191 and 198, wherein the heterologous signal sequence is a HIV1-Env signal sequence, BaEV signal sequence, Cocal signal sequence, EboV signal sequence, GaLV signal sequence, MLV-A signal sequence, or VSVG signal sequence

200. The polynucleotide of any of claims 190-192, 198 and 199, wherein the heterologous signal sequence is set forth in any one of SEQ ID NOS: 346-352.

201. The polynucleotide of any of claims 190-191 and 198-199, wherein the other virus is Vesicular stomatitis virus (VSV) and the heterologous signal sequence is the VSV G glycoprotein (VSV-G) signal sequence, optionally set forth in SEQ ID NO: 352.

202. The polynucleotide of any of claims 190-191 and 198-201, wherein the encoded variant NiV-F comprises the sequence of amino acids set forth in SEQ ID NO:261, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:261.

203. The polynucleotide of any of claims 190-191 and 198-202, wherein the encoded variant NiV-F comprises the sequence of amino acids set forth in SEQ ID NO: 261.

204. The polynucleotide of any of claims 190-191, wherein the mammalian signal sequence is a signal sequence from a human protein.

205. The polynucleotide of any of claims 190-191 and 204, wherein the heterologous signal sequence is a Prolactin signal sequence, IgGk-L signal sequence, Albumin signal sequence, CD5 signal sequence, Trypsinogen signal sequence orIL2 signal sequence.

206. The polynucleotide of any of claims 190-192, 204 and 205, wherein the heterologous signal sequence is set forth in any one of SEQ ID NOS: 353-358.

207. A polynucleotide comprising a nucleic acid molecule encoding a variant Nipah virus F glycoprotein (NiV-F) comprising a heterologous or modified transmembrane domain compared to wild-type NiV-F.

208. The polynucleotide of claim 207, wherein the transmembrane domain is a heterologous transmembrane domain from Hendra virus, optionally wherein the transmembrane domain is set forth in SEQ ID NO: 361.

209. The polynucleotide of claim 207 or claim 208, wherein the transmembrane domain is a modified transmembrane domain containing the amino acid replacement S490A, Y498A, S504A or I516V, corresponding to numbering of positions set forth in SEQ ID NO:1, optionally wherein the transmembrane domain is set forth in any one of SEQ ID NOS: 359, 360, 362 or 363.

210. The polynucleotide of any of claims 207-209, wherein the encoded variant NiV-F comprises the sequence of amino acids set forth in any one of SEQ ID NO: 268-272 or a sequence that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 268-272.

211. A polynucleotide comprising a nucleic acid molecule encoding a variant Nipah virus F glycoprotein (NiV-F) comprising a hyperfusogenic mutation selected from:
a mutation that is introduced at a N-glycosylation site with respect to amino acid positions 64, 67, and/or 99 of SEQ ID NO. 1; and/or
a mutation that is introduced at a hexamer stability site with respect to amino acid positions 393 and/or 109 of SEQ ID NO. 1.

212. The polynucleotide of claim 211, wherein the encoded variant NiV-F comprises the sequence of amino acids set forth in SEQ ID NO:247, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:247.

213. The polynucleotide of claim 211 or claim 212, wherein the variant NiV-F comprises the sequence of amino acids set forth in SEQ ID NO: 247.

214. The polynucleotide of any of claims 211-213, wherein the encoded variant NiV-F comprises the sequence of amino acids set forth in SEQ ID NO:292, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO:292.

215. The polynucleotide of any of claims 211-214, wherein the encoded variant NiV-F comprises the sequence of amino acids set forth in SEQ ID NO: 292.

216. The polynucleotide of any of claims 171-215, wherein the nucleic acid sequence is a first nucleic acid sequence and the polynucleotide further comprises a second nucleic acid sequence encoding a paramyxovirus fusion (G) protein molecule or a biologically active portion thereof or functionally active variant thereof.

217. The polynucleotide of claim 216, wherein the G protein or the biologically active portion thereof is a wild-type Nipah virus G (NiV-G) protein or a Hendra virus G protein or is a functionally active variant or biologically active portion thereof.

218. The polynucleotide of claim 216 or claim 217, wherein the G protein or the biologically active portion thereof is a wild-type NiV-G protein or a functionally active variant or a biologically active portion thereof.

219. The polynucleotide of any of claims 216-218, wherein the G protein is a biologically active portion of wild-type NiV-G that is truncated in which the G protein lacks up to 34 contiguous amino acids at or near the N-terminus of the wild-type NiV-G set forth in SEQ ID NO:305

220. The polynucleotide of any of claims 216-219, thereof that exhibits reduced binding to Ephrin B2 or Ephrin B3.

221. The polynucleotide of claim 220, wherein the NiV-G functionally active variant or a biologically active portion thereof protein comprises: one or more amino acid substitutions corresponding to amino acid substitutions selected from the group consisting of E501A, W504A, Q530A and E533A with reference to numbering set forth in SEQ ID NO:305.

222. The polynucleotide of claim 220 or claim 221, wherein the NiV-G functionally active variant or a biologically active portion thereof comprises amino acid substitutions E501A, W504A, Q530A and E533A with reference to numbering set forth in SEQ ID NO:305.

223. The polynucleotide of any of claims 216-222, wherein the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301.

224. The polynucleotide of any of claims 216-223, wherein the NiV-G functionally active variant or a biologically active portion thereof comprises the amino acid sequence set forth in SEQ ID NO: 301.

225. The polynucleotide of any of claims 216-224, wherein the G protein is linked to a binding domain that binds to a target cell surface molecule on a target cell.

226. The polynucleotide of claim 225, wherein the binding domain is attached to the C-terminus of the G protein.

227. The polynucleotide of claim 225 or claim 226, wherein the cell surface molecule is a protein, glycan, lipid or low molecular weight molecule.

228. The polynucleotide of any of claims 225-227, wherein the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells.

229. The polynucleotide of any of claims 225-228, wherein the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell.

230. The polynucleotide of any of claims 225-229, wherein the target cell is a hepatocyte.

231. The polynucleotide of any of claims 225-230, wherein the binding domain binds to a cell surface molecule selected from the group consisting of ASGR1, ASGR2 and TM4SF.

232. The polynucleotide of any of claims 225-229, wherein the target cell is a T cell.

233. The polynucleotide of any of claims 225-229 and 232, wherein the binding domain binds to a cell surface molecule selected from the group consisting of CD3, CD4 or CD8.

234. The polynucleotide of any of claims 225-233, wherein the binding domain is an antibody or antigen-binding fragment, a single domain antibody or a DARPin.

235. The polynucleotide of any of claims 225-234, wherein the binding domain is a single domain antibody that is a VHH or is a single chain variable fragment (scFv).

236. The polynucleotide of any of claims 225-235, wherein the binding domain is attached to the G protein via a linker.

237. The polynucleotide of claim 236, wherein the linker is a peptide linker.

238. The polynucleotide of claim 237, wherein the peptide linker is 2 to 65 amino acids in length.

239. The polynucleotide of claim 237 or claim 238, wherein the peptide linker is a flexible linker that comprises GS, GGS, GGGGS (SEQ ID NO:287), GGGGGS (SEQ ID NO:288) or combinations thereof.

240. The polynucleotide of any of claims 237-239, wherein the peptide linker is selected from: (GGS)n, wherein n is 1 to 10; (GGGGS)n (SEQ ID NO:289), wherein n is 1 to 10; or (GGGGGS)n (SEQ ID NO:290), wherein n is 1 to 6.

241. The polynucleotide of any of claims 216-240, wherein the polynucleotide comprises an IRES or a sequence encoding a linking peptide between the first and second nucleic acid sequences, optionally, wherein the linking peptide is a self-cleaving peptide or a peptide that causes ribosome skipping, optionally a T2A peptide.

242. The polynucleotide of any of claims 171-241, further comprising at least one promoter that is operatively linked to control expression of the nucleic acid, optionally expression of the first nucleic acid sequence and the second nucleic acid sequence.

243. A vector, comprising the polynucleotide of any of claims 171-242.

244. The vector of claim 243, wherein the vector is a mammalian vector, viral vector or artificial chromosome, optionally wherein the artificial chromosome is a bacterial artificial chromosome (BAC).

245. A plasmid, comprising the polynucleotide of any of claims 171-242.

246. The plasmid of claim 245, further comprising one or more nucleic acids encoding proteins for lentivirus production.

247. A cell comprising the polynucleotide of any of claims 171-242 or the vector of claim 243 or claim 244, or the plasmid of claim 245 or 246.

248. A method of making a lipid particle comprising a variant Nipah virus F protein and, optionally a paramyxovirus G protein, comprising:
a) providing a cell that comprises the polynucleotide of any of claims 171-242 or the vector of claim 243 or claim 244, or the plasmid of claim 245 or claim 246;
b) culturing the cell under conditions that allow for production of a lipid particle, and
c) separating, enriching, or purifying the lipid particle from the cell, thereby making the lipid particle.

249. A method of making a pseudotyped lentiviral vector, comprising:
a) providing a producer cell that comprises a lentiviral viral nucleic acid(s), and the polynucleotide of any of claims 171-242 or the vector of claim 243 or claim 244, or the plasmid of claim 245 or claim 246;
b) culturing the cell under conditions that allow for production of the lentiviral vector, and
c) separating, enriching, or purifying the lentiviral vector from the cell, thereby making the pseudotyped lentiviral vector.

250. The method of claim 248 or claim 249, wherein prior to step (b) the method further comprises providing the cell a polynucleotide encoding a henipavirus F protein molecule or biologically active portion thereof.

251. The method of any of claims 248-250, wherein the cell is a mammalian cell.

252. The method of any of claims 248-251, wherein the cell is a producer cell comprising viral nucleic acid, optionally retroviral nucleic acid or lentiviral nucleic acid, and the lipid particle is a viral particle or a viral-like particle, optionally a retroviral particle or a retroviral-like particle, optionally a lentiviral particle or lentiviral-like particle.

253. A producer cell comprising the polynucleotide of any of claims 171-242 or the vector of claim 243 or claim 244, or the plasmid of claim 245 or claim 246.

254. The producer cell of claim 253, further comprising nucleic acid encoding a paramyxovirus G protein or a biologically active portion thereof.

255. The producer cell of claim 254, wherein the G protein or the biologically active portion thereof is a wild-type Nipah virus G (NiV-G) protein or a Hendra virus G protein or is a functionally active variant or biologically active portion thereof.

256. The producer cell of claim 254 or claim 255, wherein the G protein or the biologically active portion thereof is a wild-type NiV-G protein or a functionally active variant or a biologically active portion thereof.

257. The producer cell of any of claims 254-256, wherein the G protein is a biologically active portion of wild-type NiV-G that is truncated in which the G protein lacks up to 34 contiguous amino acids at or near the N-terminus of the wild-type NiV-G set forth in SEQ ID NO:305.

258. The producer cell of any of claims 254-257, thereof that exhibits reduced binding to Ephrin B2 or Ephrin B3.

259. The producer cell of claim 258, wherein the NiV-G is a functionally active variant or a biologically active portion thereof protein comprising: one or more amino acid substitutions corresponding to amino acid substitutions selected from the group consisting of E501A, W504A, Q530A and E533A with reference to numbering set forth in SEQ ID NO:305.

260. The producer cell of claim 258 or claim 259, wherein the NiV-G functionally active variant or a biologically active portion thereof comprises amino acid substitutions E501A, W504A, Q530A and E533A with reference to numbering set forth in SEQ ID NO:305.

261. The producer cell of any of claims 253-260, wherein the NiV-G functionally active variant or a biologically active portion thereof comprises the amino acid sequence set forth in SEQ ID NO: 301 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:301.

262. The producer cell of any of claims 254-261, wherein the NiV-G functionally active variant or a biologically active portion thereof comprises the amino acid sequence set forth in SEQ ID NO: 301.

263. The producer cell of any of claims 254-262, wherein the G protein is linked to a binding domain that binds to a target cell surface molecule on a target cell.

264. The producer cell of any of claims 253-263, wherein the cell further comprises a viral nucleic acid, optionally wherein the viral nucleic acid is a lentiviral nucleic acid.

265. A lipid particle or pseudotyped lentiviral vector produced by the method of any of claims 244-248 or from the producer cell of any of claims 253-264.

266. A composition comprising a plurality of lipid particles or a plurality of lentiviral vectors of any of claims 1-170 and 265.

267. The composition of claim 266, further comprising a pharmaceutically acceptable carrier.

268. A method of transducing a cell comprising transducing a cell with a lentiviral vector of any of claims 1-170 and 165 or a composition comprising a lentiviral vector or plurality of lentiviral vectors of claim 166 or claim 167.

269. A method of delivering an exogenous agent to a subject (e.g., a human subject), the method comprising administering to the subject the lipid particle or lentiviral vector of any of claims 1-170 and 265 or a composition comprising the lipid particle or lentiviral vector of any of claims 1-170 and 265 or a composition comprising a lentiviral vector or plurality of lentiviral vectors of claim 266 or claim 267, wherein lipid particle or lentiviral vector comprise the exogenous agent.

270. A method of delivering an exogenous agent to a target cell, the method contacting a target cell with the lipid particle or lentiviral vector of any of claims 1-170 and 265 or a composition comprising the lipid particle or lentiviral vector of any of claims 1-170 and 265 or a composition comprising a lentiviral vector or plurality of lentiviral vectors of claim 266 or claim 267, wherein the lipid particle or lentiviral vector comprise the exogenous agent.

271. The method of claim 270, wherein the contacting transduces the cell with lentiviral vector or the lipid particle.

272. The method of claim 270 or claim 271, wherein the contacting is in vivo in a subject.

273. A method of treating a disease or disorder in a subject (e.g., a human subject), the method comprising administering to the subject a lipid particle of any of claims or the lentiviral vector of any of claims 1-170 and 265 or the composition of claim 266 or claim 267.

274. A method of fusing a mammalian cell to a lipid particle, the method comprising administering to the subject a lipid particle or the lentiviral vector of any of claims 1-170 and 265 or the composition of claim 266 or claim 267.

275. The method of claim 274, wherein the fusing of the mammalian cell to the lipid particle delivers an exogenous agent to a subject (e.g., a human subject).
